(19) United States
(12) Patent Application Publication
(10) Pub. No.: US 2012/0030841 A1
(43) Pub. Date: Feb. 2, 2012

(54) Isoxazoline compounds for combating invertebrate pests

(75) Inventors: Karsten Koerber, Eppelheim (DE); Florian Kaiser, Mannhiem (DE); Wolfgang von Deyn, Neustadt (DE); Matthias Pohlman, Freinsheim (DE); Steffen Gross, Ludwigshafen (DE); Prashant Deshmukh, Mannheim (DE); Joachim Dickhaut, Heidelberg (DE); Nina Gertrud Bandur, Mannheim (DE); Deborah L. Culbertson, Fuquay Varina, NC (US); Douglas D. Anspaugh, Apex, NC (US); Franz-Josef Braun, Durham, NC (US); Cecille Ebuenga, Los Banos (PH); Nancy B. Rankl, Cary, NC (US)

(73) Assignee: BASF SE, Ludwigshafen (DE)

(21) Appl. No.: 13/260,788

(22) PCT Filed: Mar. 31, 2010
(86) PCT No.: PCT/EP2010/054287

§ 371 Date: Sep. 28, 2011

Related U.S. Application Data
(60) Provisional application No. 61/165,694, filed on Apr. 1, 2009.
(60) Provisional application No. 61/265,480, filed on Dec. 1, 2009.

(51) Int. Cl. A01N 43/80; A61K 31/4709; A01P 7/04; A01P 7/00; A61P 33/14; A01P 7/02; C07D 413/04; A01H 5/00

(52) U.S. Cl. 800/298; 546/167; 514/314

(57) ABSTRACT

The present invention relates to isoxazoline compounds which are useful for combating or controlling invertebrate pests, in particular arthropod pests and nematodes. The invention also relates to a method for controlling invertebrate pests by using these compounds and to plant propagation material and to an agricultural and a veterinary composition comprising said compounds.

The present invention relates to isoxazoline compounds which are useful for combating or controlling invertebrate pests, in particular arthropod pests and nematodes. The invention also relates to a method for controlling invertebrate pests by using these compounds and to plant propagation material and to an agricultural and a veterinary composition comprising said compounds.

Invertebrate pests and in particular arthropods and nematodes destroy growing and harvested crops and attack wooden dwelling and commercial structures, causing large economic loss to the food supply and to property. While a large number of pesticidal agents are known, due to the ability of target pests to develop resistance to said agents, there is an ongoing need for new agents for combating invertebrate pests, in particular insects, arachnids and nematodes.

Related insecticidal aryl isoxazolines are described in US 2007/0066617, WO 2007/105814, WO 2007/079162, WO 2008/154528 WO2009/022746, WO 2009/002809 and WO 2009/112275. However, these documents do not describe compounds having the characteristic substituents and substituents' arrangement as claimed in the present invention.

It is an object of the present invention to provide compounds that have a good pesticidal activity, in particular insecticidal activity, and show a broad activity spectrum against a large number of different invertebrate pests, especially against difficult to control arthropod pests and/or nematodes.

It has been found that these objectives can be achieved by isoxazoline compounds of the formula I below, by their steroisomers and by their salts, in particular their agriculturally or veterinarily acceptable salts.

Therefore, in a first aspect, the invention relates to isoxazoline compounds of formula I

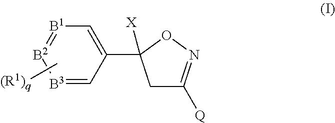

wherein
Q is an aromatic or heteroaromatic radical of the formula II

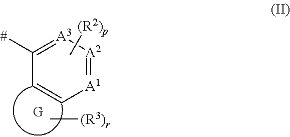

G is a condensed phenyl ring or is a condensed 5-, 6-, 7- or 8-membered saturated, partially unsaturated or completely unsaturated heterocyclic ring containing 1, 2 or 3 heteroatoms selected from O, S and N as ring members;
A¹, A² and A³ are N or CH, with the proviso that at most two of A¹, A² and A³ are N;
B¹, B² and B³ are N or CH, with the proviso that at most two of B¹, B² and B³ are N;
X is selected from the group consisting of C₁-C₄-alkyl, C₁-C₄-haloalkyl, C₁-C₄-alkoxy-C₁-C₄-alkyl, C₁-C₄-haloalkoxy-C₁-C₄-alkyl, C₂-C₄-alkenyl, C₂-C₄-haloalkenyl, C₂-C₄-alkynyl, C₂-C₄-haloalkynyl, C₃-C₆-cycloalkyl and C₃-C₆-halocycloalkyl;
each R¹ is independently selected from the group consisting of halogen, cyano, azido, nitro, —SCN, SF₅, C₁-C₆-alkyl which may be partially or fully halogenated and/or may be substituted by one or more radicals R⁴, C₃-C₈-cycloalkyl which may be partially or fully halogenated and/or may be substituted by one or more radicals R⁵, C₂-C₆-alkenyl which may be partially or fully halogenated and/or may be substituted by one or more radicals R⁴, C₂-C₆-alkynyl which may be partially or fully halogenated and/or may be substituted by one or more radicals R⁴, —Si(R¹⁴)₂R¹³, —OR⁷, —OS(O)ₙR⁷, —S(O)ₙOR⁷, —SR⁷, —S(O)ₘR⁷, —S(O)ₙN(R⁸)R⁹, —N(R⁸)R⁹, —N(R⁸)C(═O)R⁶, —C(═O)R⁶, —C(═O)OR⁷, —C(═S)R⁶, —C(═S)OR⁷, —C(═NR⁸)R⁶, —C(═O)N(R⁸)R⁹, —C(═S)N(R⁸)R⁹, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
each R² is independently selected from the group consisting of halogen; cyano; azido; nitro; —SCN; SF₅; C₁-C₆-alkyl which may be partially or fully halogenated and/or may be substituted by one or more radicals R⁴; C₃-C₈-cycloalkyl which may be partially or fully halogenated and/or may be substituted by one or more radicals R⁵; C₂-C₆-alkenyl which may be partially or fully halogenated and/or may be substituted by one or more radicals R⁴; C₂-C₆-alkynyl which may be partially or fully halogenated and/or may be substituted by one or more radicals R⁴; —Si(R¹⁴)₂R¹³; —OR⁷; —OS(O)ₙR⁷; —S(O)ₙOR⁷; —S(O)ₘR⁷; —S(O)ₙN(R⁸)R⁹; —N(R⁸)R⁹; —N(R⁸)C(═O)R⁶; —C(═O)R⁶; —C(═O)OR⁷; —C(═S)R⁶, —C(═S)OR⁷, —C(═NR⁸)R⁶; —C(═O)N(R⁸)R⁹; —C(═S)N(R⁸)R⁹; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
   with the proviso that R² is not halogen, cyano, nitro, methyl which carries one halogen atom or carries one group OH or one methylcarbonyloxy group; OH, methoxy, —OS(O)ₙR⁷, —NH₂, —CHO, C₁-C₆-alkylcarbonyl; —C(═O)OR⁷, wherein R⁷ is hydrogen, C₁-C₆-alkyl or benzyl; —C(═O)R⁶, wherein R⁶ is —N(R⁸)R⁹; —C(═S)R⁶, wherein R⁶ is —N(R⁸)R⁹, —C(═NR⁸)R⁶, —C(═O)N(R⁸)R⁹ or —C(═S)N(R⁸)R⁹; if A¹, A² and A³ are CH and if simultaneously R² is bound to A¹;
   or two radicals R² bound on adjacent carbon atoms may be together a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR⁸—, —CH₂CH═N—, —CH═CH—NR⁸—, —OCH═N— and —SCH═N—, thus forming, together with the carbon atoms to which they are bound, a 5- or 6-membered ring, where the hydrogen atoms of the above groups may be replaced by one or more substituents selected from halogen, methyl, halomethyl, hydroxyl, methoxy and halomethoxy or one or more CH₂ groups of the above groups may be replaced by a C═O group;
each R³ is independently selected from the group consisting of halogen, cyano, azido, nitro; —SCN; —SF₅, C₁-C₆-alkyl which may be partially or fully halogenated and/or may be substituted by one or more radicals R⁴; C₃-C₈-cycloalkyl which may be partially or fully halogenated and/or may be substituted by one or more radicals R⁵; C₂-C₆-alkenyl which may be partially or fully halogenated and/or may be substituted by one or more radicals R⁴; C₂-C₆-alkynyl which may be partially or fully halogenated and/or may be substituted by one or more radicals R⁴; —Si(R¹⁴)₂R¹³; —OR⁷; —OS(O)ₘR⁷; —S(O)ₙOR⁷; —SR⁷; —S(O)ₘR⁷; —S(O)ₙN(R⁸)R⁹; —N(R⁸)R⁹; —N(R⁸)C(═O)R⁶; —C(═O)R⁶; —C(═O)OR⁷; —C(═S)R⁶, —C(═S)OR⁷, —C(═NR⁸)R⁶; —C(═O)N(R⁸)R⁹; —C(═S)N(R⁸)R⁹; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
   or two radicals R³ bound on adjacent carbon atoms may be together a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR⁸—, —CH₂CH═N—, —CH═CH—NR⁸—, —OCH═N— and —SCH═N—, thus forming, together with the carbon atoms to which they are bound, a 5- or 6-membered ring, where the hydrogen atoms of the above groups may be replaced by one or more substituents selected from halogen, methyl, halomethyl, hydroxyl, methoxy and halomethoxy or one or more CH₂ groups of the above groups may be replaced by a C═O group;
each R⁴ is independently selected from the group consisting of cyano, azido, nitro, —SCN, —SF₅, C₃-C₈-cycloalkyl, C₃-C₈-halocycloalkyl, C₃-C₈-cycloalkyl substituted by a phenyl group or substituted by a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the phenyl ring or the heterocyclyl ring may be substituted by one or more radicals R¹⁰; —Si(R¹⁴)₂R¹³, —OR⁷, —OSO₂R⁷, —SO₂OR⁷, —SR⁷, —S(O)ₘR⁷, —S(O)ₙN(R⁸)R⁹, —N(R⁸)R⁹, —C(═O)N(R⁸)R⁹, —C(═S)N(R⁸)R⁹, —C(═O)OR⁷, —C(═O)R⁶, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
   or two geminally bound radicals R⁴ together form a group selected from ═CR¹¹R¹², ═S(O)ₘR⁷, ═S(O)ₘN(R⁸)R⁹, ═NR⁸, ═NOR⁷ and ═NNR⁸;
   or two radicals R⁴, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members;

each R⁵ is independently selected from the group consisting of cyano, azido, nitro, —SCN, —SF₅, C₁-C₆-haloalkyl, C₁-C₆-alkoxy-C₁-C₆-alkyl, C₁-C₆-haloalkoxy-C₁-C₈-alkyl, C₃-C₈-cycloalkyl, C₃-C₈-halocycloalkyl, C₃-C₈-cycloalkyl-C₁-C₄-alkyl, C₃-C₈-halocycloalkyl-C₁-C₄-alkyl, C₂-C₆-alkenyl, C₂-C₆-haloalkenyl, C₂-C₆-alkynyl, C₂-C₆-haloalkynyl, —Si(R¹⁴)₂R¹³, —OSO₂R⁷, —SO₂OR⁷, —SR⁷, —S(O)ₘR⁷, —S(O)ₙN(R⁸)R⁹, —N(R⁸)R⁹, —C(═O)N(R⁸)R⁹, —C(═S)N(R⁸)R⁹, —C(═O)OR⁷, —C(═O)R⁶, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
or two geminally bound radicals R⁵ together form a group selected from ═CR¹¹R¹², ═S(O)ₘR⁷, ═S(O)ₘN(R⁸)R⁹, ═NR⁸, ═NOR⁷ and ═NNR⁸;
or two radicals R⁵, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members;

each R⁶ is independently selected from the group consisting of hydrogen, cyano, azido, C₁-C₆-alkyl, C₁-C₆-haloalkyl, C₁-C₆-alkoxy-C₁-C₆-alkyl, C₁-C₆-haloalkoxy-C₁-C₈-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, —CH₂—(C₃-C₆-cycloalkyl), —CH₂—(C₃-C₆-halocycloalkyl), —CH₂—S(O)ₙ—(C₁-C₄-alkyl), —CH₂—S(O)ₙ—(C₁-C₄-haloalkyl), —CH₂-phenyl, —CH₂—S(O)ₙ-phenyl, —OSO₂R⁷, —SR⁷, —S(O)ₘR⁷, —S(O)ₙN(R⁸)R⁹, —N(R⁸)R⁹, —C(═O)N(R⁸)R⁹, —C(═S)N(R⁸)R⁹, —C(═O)OR⁷, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
each R⁷ is independently selected from the group consisting of hydrogen, cyano, C₁-C₆-alkyl which may be partially or fully halogenated and/or may be substituted by one or more radicals R¹⁸, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₃-C₈-cycloalkyl-C₁-C₄-alkyl, C₃-C₈-halocycloalkyl-C₁-C₄-alkyl, C₂-C₆-alkenyl, C₂-C₆-haloalkenyl, C₂-C₆-alkynyl, C₂-C₆-haloalkynyl, —Si(R¹⁴)₂R¹³, —SR⁸, —S(O)ₙ—, R¹⁷, —S(O)ₙN(R⁸)R⁹, —N(R⁸)R⁹, —N═CR¹⁵R¹⁶, —C(═O)R¹⁷, —C(═O)N(R⁸)R⁹, —C(═S)N(R⁸)R⁹, —C(═O)OR¹⁷, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
   with the proviso that R⁷ is not C₁-C₆-alkoxy or C₁-C₆-haloalkoxy if it is bound to an oxygen atom;
R⁸ and R⁹, independently of each other and independently of each occurrence, are selected from the group consisting of hydrogen, C₁-C₆-alkyl which may be partially or fully halogenated and/or may be substituted by one or more radicals R¹⁸, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₃-C₈-cycloalkyl-C₁-C₄-alkyl, C₃-C₈-halocycloalkyl-C₁-C₄-alkyl, C₂-C₆-alkenyl, C₂-C₆-haloalkenyl, C₂-C₆-alkynyl, C₂-C₆-haloalkynyl, —C(═O)R¹⁷, —C(═O)OR¹⁷, —C(═O)N(R²⁰)R²¹, —C(O)—CH₂—S(O)ₙ—(C₁-C₄-alkyl), —C(O)—CH₂—S(O)ₙ—(C₁-C₄-haloalkyl), phenylcarbonyl, —C(O)—CH₂-phenyl, —C(O)—CH₂—S(O)ₙ-phenyl, phenyl, where the phenyl moiety in the 4 last-mentioned radicals may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰;
each R¹⁰ is independently selected from the group consisting of halogen, cyano, azido, nitro, —SCN, —SF₅, C₁-C₁₀-alkyl which may be partially or fully halogenated and/or may be substituted by one or more radicals R¹⁸, C₃-C₈-cycloalkyl which may be partially or fully halogenated and/or may be substituted by one or more radicals R¹⁹, C₂-C₁₀-alkenyl which may be partially or fully halogenated and/or may be substituted by one or more radicals R¹⁸, C₂-C₁₀-alkynyl which may be partially or fully halogenated and/or may be substituted by one or more radicals R¹⁸, —Si(R¹⁴)₂R¹³, —OR¹⁷, —OS(O)ₙR¹⁷, —SR¹⁷, —S(O)ₘR¹⁷, —S(O)ₙN(R²⁰)R²¹, —N(R²⁰)R²¹, —C(═O)R¹⁷, —C(═O)OR¹⁷, —C(═NR²⁰)R²¹, —C(═O)N(R²⁰)R²¹, —C(═S)N(R²⁰)R²¹, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from N, O, S, NO, SO and SO₂, as ring members, which may be substituted by one or more radicals independently selected from halogen, cyano, nitro, C₁-C₆-alkyl, C₁-C₆-haloalkyl, C₁-C₆-alkoxy and C₁-C₆-haloalkoxy;

or two radicals R¹⁰ bound on adjacent atoms together form a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR²⁰—, —CH₂CH═N—, —CH═CH—NR²⁰—, —OCH═N— and —SCH═N—, thus forming, together with the atoms to which they are bound, a 5- or 6-membered ring, where the hydrogen atoms of the above groups may be replaced by one or more substituents selected from halogen, methyl, halomethyl, hydroxyl, methoxy and halomethoxy or one or more CH₂ groups of the above groups may be replaced by a C═O group;
R¹¹ and R¹², independently of each other and independently of each occurrence, are selected from the group consisting of hydrogen, halogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, phenyl which may be substituted by 1, 2, 3, 4, or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, which may be substituted by one or more radicals R¹⁰;
R¹³ and R¹⁴, independently of each other and independently of each occurrence, are selected from the group consisting of C₁-C₄-alkyl, C₃-C₆-cycloalkyl, C₁-C₄-alkoxy-C₁-C₄-alkyl, phenyl and benzyl;
R¹⁵ and R¹⁶, independently of each other and independently of each occurrence, are selected from the group consisting of C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₁-C₆-alkoxy-C₁-C₆-alkyl, C₁-C₆-haloalkoxy-C₁-C₆-alkyl, phenyl which may be substituted by 1, 2, 3, 4, or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, which may be substituted by one or more radicals R¹⁰;
each R¹⁷ is independently selected from the group consisting of hydrogen, C₁-C₆-alkyl, C₁-C₆-haloalkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, C₃-C₈-cycloalkyl-C₁-C₄-alkyl, C₃-C₈-halocycloalkyl-C₁-C₄-alkyl, C₁-C₆-alkoxy-C₁-C₆-alkyl, C₁-C₆-haloalkoxy-C₁-C₆-alkyl, phenyl and benzyl, where the phenyl moieties in the two last-mentioned radicals may be substituted by 1, 2 or 3 radicals selected from halogen, cyano, nitro, C₁-C₄-alkyl, C₁-C₄-haloalkyl, C₁-C₄-alkoxy and C₁-C₄-haloalkoxy;
each R¹⁸ is independently is selected from the group consisting of cyano; azido, nitro, —SCN, SF₅, C₃-C₈-cycloalkyl, C₃-C₈-halocycloalkyl, —Si(R¹⁴)₂R¹³, —OR¹⁷, —OSO₂R¹⁷, —SR¹⁷, —S(O)ₘR¹⁷, —S(O)ₙN(R²⁰)R²¹, —N(R²⁰)R²¹, —C(═O)N(R²⁰)R²¹, —C(═S)N(R²⁰)R²¹, —C(═O)OR¹⁷, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
each R¹⁹ is independently is selected from the group consisting of cyano; azido, nitro, —SCN, SF₅, C₁-C₆-alkyl, C₁-C₆-haloalkyl, C₁-C₆-alkoxy-C₁-C₆-alkyl, C₁-C₆-haloalkoxy-C₁-C₆-alkyl, C₂-C₆-alkenyl, C₂-C₆-haloalkenyl, C₂-C₆-alkynyl, C₂-C₆-haloalkynyl, C₃-C₈-cycloalkyl, C₃-C₈-halocycloalkyl, —Si(R¹⁴)₂R¹³, —OR¹⁷, —OSO₂R¹⁷, —SR¹⁷, —S(O)ₘR¹⁷, —S(O)ₙN(R²⁰)R²¹, —N(R²⁰)R²¹, —C(═O)N(R²⁰)R²¹, —C(═S)N(R²⁰)R²¹, —C(═O)OR¹⁷, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
R²⁰ and R²¹, independently of each other and independently of each occurrence, are selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₃-C₈-cycloalkyl-C₁-C₄-alkyl, C₃-C₈-halocycloalkyl-C₁-C₄-alkyl, C₂-C₆-alkenyl, C₂-C₆-haloalkenyl, C₂-C₆-alkynyl, C₂-C₆-haloalkynyl, —C(═O)N(R²²)R²³, —C(═O)R¹⁷, —C(═O)OR¹⁷, phenyl, benzyl, where the phenyl moieties in the two last-mentioned radicals may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;
   or R²⁰ and R²¹, together with the nitrogen atom to which they are bound, may form a 3-, 4-, 5-, 6- or 7-membered saturated, partially unsaturated or aromatic heterocyclic ring which may additionally containing 1 or 2 further heteroatoms or heteroatom groups selected from N, O, S, NO, SO and SO₂, as ring members, where the heterocyclic ring may be substituted by one or more radicals selected from halogen, C₁-C₆-alkyl, C₁-C₆-haloalkyl, C₁-C₆-alkoxy and C₁-C₆-haloalkoxy;
R²² and R²³, independently of each other and independently of each occurrence, are selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₃-C₈-cycloalkyl-C₁-C₄-alkyl, C₃-C₈-halocycloalkyl-C₁-C₄-alkyl, C₂-C₆-alkenyl, C₂-C₆-haloalkenyl, C₂-C₆-alkynyl, C₂-C₆-haloalkynyl, aminocarbonyl, C₁-C₄-alkylaminocarbonyl, di-(C₁-C₄-alkyl)-aminocarbonyl, —C(═O)R¹⁷, —C(═O)OR¹⁷, phenyl, benzyl, where the phenyl moieties in the two last-mentioned radicals may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy;

each m is independently 1 or 2;

each n is independently 0, 1 or 2;

p is 0, 1, 2 or 3;

q is 0, 1, 2, 3, 4 or 5;

r is 0, 1, 2, 3, 4 or 5; and

# is the attachment point to the remainder of the molecule;

and the stereoisomers and agriculturally or veterinarily acceptable salts thereof.

The present invention also provides an agricultural composition comprising at least one compound of the formula I as defined herein and/or an agriculturally acceptable salt thereof and at least one liquid or solid carrier.

The present invention also provides a veterinary composition comprising at least one compound of the formula I as defined herein and/or a veterinarily acceptable salt thereof and at least one liquid or solid carrier.

The present invention also provides a method for controlling invertebrate pests which method comprises treating the pests, their food supply, their habitat or their breeding ground or a cultivated plant, plant propagation materials (such as seed), soil, area, material or environment in which the pests are growing or may grow, or the materials, cultivated plants, plant propagation materials (such as seed), soils, surfaces or spaces to be protected from pest attack or infestation with a pesticidally effective amount of a compound of formula I or a salt thereof as defined herein.

The present invention also relates to plant propagation material, in particular seed, comprising at least one compound of formula I and/or an agriculturally acceptable salt thereof as defined herein.

The present invention further relates to a method for treating or protecting an animal from infestation or infection by parasites which comprises bringing the animal in contact with a parasiticidally effective amount of a compound of the formula I or a veterinarily acceptable salt thereof as defined herein. Bringing the animal in contact with the compound I, its salt or the veterinary composition of the invention means applying or administering it to the animal.

The term “steroisomers” encompasses both optical isomers, such as enantiomers or diastereomers, the latter existing due to more than one center of chirality in the molecule, as well as geometrical isomers (cis/trans isomers).

Depending on the substitution pattern, the compounds of the formula I may have one or more centers of chirality, in which case they are present as mixtures of enantiomers or diastereomers. One center of chirality is the carbon ring atom of the isoxazoline ring carrying radical X. The invention provides both the pure enantiomers or diastereomers and their mixtures and the use according to the invention of the pure enantiomers or diastereomers of the compound I or its mixtures. Suitable compounds of the formula I also include all possible geometrical stereoisomers (cis/trans isomers) and mixtures thereof.

The compounds of the present invention may be amorphous or may exist in one or more different crystalline states (polymorphs) which may have a different macroscopic properties such as stability or show different biological properties such as activities.

The present invention includes both amorphous and crystalline compounds of the formula I, mixtures of different crystalline states of the respective compound I, as well as amorphous or crystalline salts thereof.

Salts of the compounds of the formula I are preferably agriculturally and veterinarily acceptable salts. They can be formed in a customary method, e.g. by reacting the compound with an acid of the anion in question if the compound of formula I has a basic functionality or by reacting an acidic compound of formula I with a suitable base.

Suitable agriculturally acceptable salts are especially the salts of those cations or the acid addition salts of those acids whose cations and anions, respectively, do not have any adverse effect on the action of the compounds according to the present invention. Suitable cations are in particular the ions of the alkali metals, preferably lithium, sodium and potassium, of the alkaline earth metals, preferably calcium, magnesium and barium, and of the transition metals, preferably manganese, copper, zinc and iron, and also ammonium (NH⁴⁺) and substituted ammonium in which one to four of the hydrogen atoms are replaced by C₁-C₄-alkyl, C₁-C₄-hydroxyalkyl, C₁-C₄-alkoxy, C₁-C₄-alkoxy-C₁-C₄-alkyl, hydroxy-C₁-C₄-alkoxy-C₁-C₄-alkyl, phenyl or benzyl. Examples of substituted ammonium ions comprise methylammonium, isopropylammonium, dimethylammonium, diisopropylammonium, trimethylammonium, tetramethylammonium, tetraethylammonium, tetrabutylammonium, 2-hydroxyethylammonium, 2-(2-hydroxy-ethoxy)ethylammonium, bis(2-hydroxyethyl)ammonium, benzyltrimethylammonium and benzl-triethylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogen sulfate, sulfate, dihydrogen phosphate, hydrogen phosphate, phosphate, nitrate, hydrogen carbonate, carbonate, hexafluorosilicate, hexafluorophosphate, benzoate, and the anions of C₁-C₄-alkanoic acids, preferably formate, acetate, propionate and butyrate. They can be formed by reacting a compound of formulae I with an acid of the corresponding anion, preferably of hydrochloric acid, hydrobromic acid, sulfuric acid, phosphoric acid or nitric acid.

By the term “veterinarily acceptable salts” is meant salts of those cations or anions which are known and accepted in the art for the formation of salts for veterinary use. Suitable acid addition salts, e.g. formed by compounds of formula I containing a basic nitrogen atom, e.g. an amino group, include salts with inorganic acids, for example hydrochlorids, sulphates, phosphates, and nitrates and salts of organic acids for example acetic acid, maleic acid, dimaleic acid, fumaric acid, difumaric acid, methane sulfenic acid, methane sulfonic acid, and succinic acid.

The term “invertebrate pest” as used herein encompasses animal populations, such as insects, arachnids and nematodes, which may attack plants, thereby causing substantial damage to the plants attacked, as well as ectoparasites which may infest animals, in particular warm blooded animals such as e.g. mammals or birds, or other higher animals such as reptiles, amphibians or fish, thereby causing substantial damage to the animals infested.

The term “plant propagation material” as used herein includes all the generative parts of the plant such as seeds and vegetative plant material such as cuttings and tubers (e.g. potatoes), which can be used for the multiplication of the plant. This includes seeds, roots, fruits, tubers, bulbs, rhizomes, shoots, sprouts and other parts of plants. Seedlings and young plants, which are to be transplanted after germination or after emergence from soil, may also be included. These plant propagation materials may be treated prophylactically with a plant protection compound either at or before planting or transplanting.

The term “plants” comprises any types of plants including “non-cultivated plants” and in particular “cultivated plants”.

The term “non-cultivated plants” refers to any wild type species or related species or related genera of a cultivated plant.

The term “cultivated plants” as used herein includes plants which have been modified by breeding, mutagenesis or genetic engineering. Genetically modified plants are plants, which genetic material has been so modified by the use of recombinant DNA techniques that under natural circumstances cannot readily be obtained by cross breeding, mutations or natural recombination. Typically, one or more genes have been integrated into the genetic material of a genetically modified plant in order to improve certain properties of the plant. Such genetic modifications also include but are not limited to targeted post-transitional modification of protein(s) (oligo- or polypeptides) poly for example by glycosylation or polymer additions such as prenylated, acetylated or farnesylated moieties or PEG moieties (e.g. as disclosed in Biotechnol Prog. 2001 July-August; 17(4):720-8, Protein Eng Des Sel. 2004 January; 17(1):57-66, Nat. Protoc. 2007; 2(5):1225-35, Curr. Opin. Chem. Biol. 2006 October; 10(5):487-91. Epub 2006 Aug. 28, Biomaterials. 2001 March; 22(5):405-17, Bioconjug Chem. 2005 January-February; 16(1):113-21).

The term “cultivated plants” as used herein further includes plants that have been rendered tolerant to applications of specific classes of herbicides, such as hydroxy-phenylpyruvate dioxygenase (HPPD) inhibitors; acetolactate synthase (ALS) inhibitors, such as sulfonyl ureas (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073) or imidazolinones (see e.g. U.S. Pat. No. 6,222,100, WO 01/82685, WO 00/26390, WO 97/41218, WO 98/02526, WO 98/02527, WO 04/106529, WO 05/20673, WO 03/14357, WO 03/13225, WO 03/14356, WO 04/16073); enolpyruvylshikimate-3-phosphate synthase (EPSPS) inhibitors, such as glyphosate (see e.g. WO 92/00377); glutamine synthetase (GS) inhibitors, such as glufosinate (see e.g. EP-A-C242236, EP-A-242246) or oxynil herbicides (see e.g. U.S. Pat. No. 5,559,024) as a result of conventional methods of breeding or genetic engineering. Several cultivated plants have been rendered tolerant to herbicides by conventional methods of breeding (mutagenesis), for example Clearfield® summer rape (Canola) being tolerant to imidazolinones, e.g. imazamox. Genetic engineering methods have been used to render cultivated plants, such as soybean, cotton, corn, beets and rape, tolerant to herbicides, such as glyphosate and glufosinate, some of which are commercially available under the trade names RoundupReady® (glyphosate) and LibertyLink® (glufosinate).

The term “cultivated plants” as used herein further includes plants that are by the use of recombinant DNA techniques capable to synthesize one or more insecticidal proteins, especially those known from the bacterial genus bacillus, particularly from bacillus thuringiensis, such as ä-endotoxins, e.g. CryIA(b), CryIA(c), CryIF, CryIF(a2), CryIIA(b), CryIIIA, CryIIIB(b1) or Cry9c; vegetative insecticidal proteins (VIP), e.g. VIP1, VIP2, VIP3 or VIP3A; insecticidal proteins of bacteria colonizing nematodes, for example Photorhabdus spp. or Xenorhabdus spp.; toxins produced by animals, such as scorpion toxins, arachnid toxins, wasp toxins, or other insect-specific neurotoxins; toxins produced by fungi, such Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxysteroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilben synthase, bibenzyl synthase, chitinases or glucanases. In the context of the present invention these insecticidal proteins or toxins are to be understood expressly also as pre-toxins, hybrid proteins, truncated or otherwise modified proteins. Hybrid proteins are characterized by a new combination of protein domains, (see, for example WO 02/015701). Further examples of such toxins or genetically-modified plants capable of synthesizing such toxins are dis-closed, for example, in EP-A 374 753, WO 93/007278, WO 95/34656, EP-A 427 529, EP-A 451 878, WO 03/018810 and WO 03/052073. The methods for producing such genetically modified plants are generally known to the person skilled in the art and are described, for example, in the publications mentioned above. These insecticidal proteins contained in the genetically modified plants impart to the plants producing these proteins protection from harmful pests from certain taxonomic groups of arthropods insects, particularly to beetles (Coleoptera), flies (Diptera), and butterflies and moths (Lepidoptera) and to plant parasitic nematodes (Nematoda).

The term “cultivated plants” as used herein further includes plants that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, for example EP-A 0 392 225), plant disease resistance genes (for example potato cultivars, which express resistance genes acting against Phytophthora infestans derived from the mexican wild potato Solanum bulbocastanum) or T4-lyso-zym (e.g. potato cultivars capable of synthesizing these proteins with increased resistance against bacteria such as Erwinia amylvora). The methods for producing such genetically modified plants are generally known to the person skilled in the art and are described, for example, in the publications mentioned above.

The term “cultivated plants” as used herein further includes plants that are by the use of recombinant DNA techniques capable to synthesize one or more proteins to increase the productivity (e.g. bio mass production, grain yield, starch content, oil content or protein content), tolerance to drought, salinity or other growth-limiting environmental factors or tolerance to pests and fungal, bacterial or viral pathogens of those plants.

The term “cultivated plants” as used herein further includes plants that contain by the use of recombinant DNA techniques a modified amount of substances of content or new substances of content, specifically to improve human or animal nutrition, for example oil crops that produce health-promoting long-chain omega-3 fatty acids or unsaturated omega-9 fatty acids (e.g. Nexera® rape).

The term “cultivated plants” as used herein further includes plants that contain by the use of recombinant DNA techniques a modified amount of substances of content or new substances of content, specifically to improve raw material production, for example potatoes that produce increased amounts of amylopectin (e.g. Amflora® potato).

The organic moieties mentioned in the above definitions of the variables are—like the term halogen—collective terms for individual listings of the individual group members. The prefix Cₙ-Cₘ indicates in each case the possible number of carbon atoms in the group.

The term halogen denotes in each case fluorine, bromine, chlorine or iodine, in particular fluorine, chlorine or bromine.

The term “C₁-C₁₀-alkyl” as used herein and in the alkyl moieties of alkoxy, alkylthio, alkylsulfinyl, alkylsulfonyl, alkylcarbonyl, alkoxycarbonyl and the like refers to saturated straight-chain or branched hydrocarbon radicals having 1 to 2 (“C₁-C₂-alkyl”), 1 to 4 (“C₁-C₄-alkyl”), 1 to 6 (“C₁-C₆-alkyl”), 1 to 8 (“C₁-C₈-alkyl”) or 1 to 10 (“C₁-C₁₀-alkyl”) carbon atoms. C₁-C₂-Alkyl is methyl or ethyl. C₁-C₄-Alkyl is additionally propyl, isopropyl, butyl, 1-methylpropyl(sec-butyl), 2-methylpropyl(isobutyl) or 1,1-dimethylethyl (tert-butyl). C₁-C₆-Alkyl is additionally also, for example, pentyl, 1-methylbutyl, 2-methylbutyl, 3-methylbutyl, 2,2-dimethylpropyl, 1-ethylpropyl, 1,1-dimethylpropyl, 1,2-dimethylpropyl, hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, or 1-ethyl-2-methylpropyl. C₁-C₆-Alkyl is additionally also, for example, heptyl, octyl, 2-ethylhexyl and positional isomers thereof. C₁-C₁₀-Alkyl is additionally also, for example, nonyl, decyl and positional isomers thereof.

The term “C₁-C₁₀-haloalkyl” as used herein, which is also expressed as “C₁-C₁₀-alkyl which is partially or fully halogenated”, refers to straight-chain or branched alkyl groups having 1 to 2 (“C₁-C₂-haloalkyl”), 1 to 4 (“C₁-C₄-haloalkyl”), 1 to 6 (“C₁-C₆-haloalkyl”), 1 to 8 (“C₁-C₈-haloalkyl”) or 1 to 10 (“C₁-C₁₀-haloalkyl”) carbon atoms (as mentioned above), where some or all of the hydrogen atoms in these groups are replaced by halogen atoms as mentioned above: in particular C₁-C₂-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl or 1,1,1-trifluoroprop-2-yl.

“Halomethyl” is methyl in which 1, 2 or 3 of the hydrogen atoms are replaced by halogen atoms. Examples are bromomethyl, chloromethyl, fluoromethyl, dichloromethyl, trichloromethyl, difluoromethyl, trifluoromethyl, chlorofluoromethyl, dichlorofluoromethyl, chlorodifluoromethyl and the like.

The term “C₂-C₁₀-alkenyl” as used herein and in the alkenyl moiety of alkenyloxy and the like refers to monounsaturated straight-chain or branched hydrocarbon radicals having 2 to 4 (“C₂-C₄-alkenyl”), 2 to 6 (“C₂-C₆-alkenyl”), 2 to 8 (“C₂-C₈-alkenyl”), 3 to 8 (“C₃-C₈-alkenyl”), 2 to 10 (“C₂-C₁₀-alkenyl”) or 3 to 10 (“C₃-C₁₀-alkenyl”) carbon atoms and a double bond in any position, for example C₂-C₄-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl or 2-methyl-2-propenyl; C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, 1-ethyl-2-methyl-2-propenyl and the like, or C₂-C₁₀-alkenyl, such as the radicals mentioned for C₂-C₆-alkenyl and additionally 1-heptenyl, 2-heptenyl, 3-heptenyl, 1-octenyl, 2-octenyl, 3-octenyl, 4-octenyl, 1-nonenyl, 2-nonenyl, 3-nonenyl, 4-nonenyl, 1-decenyl, 2-decenyl, 3-decenyl, 4-decenyl, 5-decenyl and the positional isomers thereof.

The term “C₂-C₁₀-haloalkenyl” as used herein, which is also expressed as “C₁-C₁₀-alkenyl which is partially or fully halogenated”, and the haloalkenyl moieties in haloalkenyloxy, haloalkenylcarbonyl and the like refers to unsaturated straight-chain or branched hydrocarbon radicals having 2 to 4 (“C₂-C₄-haloalkenyl”), 2 to 6 (“C₂-C₆-haloalkenyl”), 2 to 8 (“C₂-C₆-haloalkenyl”) or 2 to 10 (“C₂-C₁₀-haloalkenyl”) carbon atoms and a double bond in any position (as mentioned above), where some or all of the hydrogen atoms in these groups are replaced by halogen atoms as mentioned above, in particular fluorine, chlorine and bromine, for example chlorovinyl, chloroallyl and the like.

The term “C₂-C₁₀-alkynyl” as used herein and the alkynyl moieties in alkynyloxy, alkynylcarbonyl and the like refers to straight-chain or branched hydrocarbon groups having 2 to 4 (“C₂-C₄-alkynyl”), 2 to 6 (“C₂-C₆-alkynyl”), 2 to 8 (“C₂-C₈-alkynyl”), 3 to 8 (“C₃-C₈-alkynyl”), 2 to 10 (“C₂-C₁₀-alkynyl”) or 3 to 10 (“C₃-C₈-alkynyl”) carbon atoms and one or two triple bonds in any position, for example C₂-C₄-alkynyl, such as ethynyl, 1-propynyl, 2-propynyl, 1-butynyl, 2-butynyl, 3-butynyl, 1-methyl-2-propynyl and the like, C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, 1-ethyl-1-methyl-2-propynyl and the like;

The term “C₂-C₁₀-haloalkynyl” as used herein, which is also expressed as “C₁-C₁₀-alkynyl which is partially or fully halogenated”, and the haloalkynyl moieties in haloalkynyloxy, haloalkynylcarbonyl and the like refers to unsaturated straight-chain or branched hydrocarbon radicals having 2 to 4 (“C₂-C₄-haloalkynyl”), 3 to 4 (“C₃-C₄-haloalkynyl”), 2 to 6 (“C₂-C₆-haloalkynyl”), 3 to 6 (“C₃-C₆-haloalkynyl”), 2 to 8 (“C₂-C₈-haloalkynyl”), 3 to 8 (“C₃-C₈-haloalkynyl”), 2 to 10 (“C₂-C₁₀-haloalkynyl”) or 3 to 10 (“C₃-C₁₀-haloalkynyl”) carbon atoms and one or two triple bonds in any position (as mentioned above), where some or all of the hydrogen atoms in these groups are replaced by halogen atoms as mentioned above, in particular fluorine, chlorine and bromine;

The term “C₃-C₈-cycloalkyl” as used herein refers to mono- or bi- or polycyclic saturated hydrocarbon radicals having 3 to 8, in particular 3 to 6 carbon atoms (“C₃-C₆-cycloalkyl”). Examples of monocyclic radicals having 3 to 6 carbon atoms comprise cyclopropyl, cyclobutyl, cyclopentyl and cyclohexyl. Examples of monocyclic radicals having 3 to 8 carbon atoms comprise cyclopropyl, cyclobutyl, cyclopentyl, cyclohexyl, cycloheptyl and cyclooctyl. Examples of bicyclic radicals having 7 or 8 carbon atoms comprise bicyclo[2.2.1]heptyl, bicyclo[3.1.1]heptyl, bicyclo[2.2.2]octyl and bicyclo[3.2.1]octyl.

The term “C₃-C₈-halocycloalkyl” as used herein, which is also expressed as “C₃-C₈-cycloalkyl which is partially or fully halogenated”, and the halocycloalkyl moieties in halocycloalkoxy, halocycloalkylcarbonyl and the like refers to mono- or bi- or polycyclic saturated hydrocarbon groups having 3 to 8 (“C₃-C₈-halocycloalkyl”) or preferably 3 to 6 (“C₃-C₆-halocycloalkyl”) carbon ring members (as mentioned above) in which some or all of the hydrogen atoms are replaced by halogen atoms as mentioned above, in particular fluorine, chlorine and bromine.

The term “C₃-C₈-cycloalkyl-C₁-C₄-alkyl” refers to a C₃-C₈-cycloalkyl group as defined above which is bound to the remainder of the molecule via a C₁-C₄-alkyl group, as defined above. Examples are cyclopropylmethyl, cyclopropylethyl, cyclopropylpropyl, cyclobutylmethyl, cyclobutylethyl, cyclobutylpropyl, cyclopentylmethyl, cyclopentylethyl, cyclopentylpropyl, cyclohexylmethyl, cyclohexylethyl, cyclohexylpropyl, and the like.

The term “C₃-C₈-halocycloalkyl-C₁-C₄-alkyl” refers to a C₃-C₈-halocycloalkyl group as defined above which is bound to the remainder of the molecule via a C₁-C₄-alkyl group, as defined above.

The term “C₁-C₂-alkoxy” is a C₁-C₂-alkyl group, as defined above, attached via an oxygen atom. The term “C₁-C₄-alkoxy” is a C₁-C₄-alkyl group, as defined above, attached via an oxygen atom. The term “C₁-C₆-alkoxy” is a C₁-C₆-alkyl group, as defined above, attached via an oxygen atom. The term “C₁-C₁₀-alkoxy” is a C₁-C₁₀-alkyl group, as defined above, attached via an oxygen atom. C₁-C₂-Alkoxy is methoxy or ethoxy. C₁-C₄-Alkoxy is additionally, for example, n-propoxy, 1-methylethoxy(isopropoxy), butoxy, 1-methylpropoxy(sec-butoxy), 2-methylpropoxy(isobutoxy) or 1,1-dimethylethoxy(tertbutoxy). C₁-C₆-Alkoxy is additionally, for example, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy. C₁-C₈-Alkoxy is additionally, for example, heptyloxy, octyloxy, 2-ethylhexyloxy and positional isomers thereof. C₁-C₁₀-Alkoxy is additionally, for example, nonyloxy, decyloxy and positional isomers thereof.

The term “C₁-C₂-haloalkoxy” is a C₁-C₂-haloalkyl group, as defined above, attached via an oxygen atom. The term “C₁-C₄-haloalkoxy” is a C₁-C₄-haloalkyl group, as defined above, attached via an oxygen atom. The term “C₁-C₆-haloalkoxy” is a C₁-C₆-haloalkyl group, as defined above, attached via an oxygen atom. The term “C₁-C₁₀-haloalkoxy” is a C₁-C₁₀-haloalkyl group, as defined above, attached via an oxygen atom. C₁-C₂-Haloalkoxy is, for example, OCH₂F, OCHF₂, OCF₃, OCH₂Cl, OCHCl₂, OCCl₃, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy or OC₂F₅. C₁-C₄-Haloalkoxy is additionally, for example, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, OCH₂—C₂F₅, OCF₂—C₂F_{5,1}-(CH₂F)-2-fluoroethoxy, 1-(CH₂Cl)-2-chloroethoxy, 1-(CH₂Br)-2-bromoethoxy, 4-fluorobutoxy, 4-chlorobutoxy, 4-bromobutoxy or nonafluorobutoxy. C₁-C₆-Haloalkoxy is additionally, for example, 5-fluoropentoxy, 5-chloropentoxy, 5-brompentoxy, 5-iodopentoxy, undecafluoropentoxy, 6-fluorohexoxy, 6-chlorohexoxy, 6-bromohexoxy, 6-iodohexoxy or dodecafluorohexoxy.

The term “C₁-C₂-alkylthio” is a C₁-C₂-alkyl group, as defined above, attached via a sulfur atom. The term “C₁-C₄-alkylthio” is a C₁-C₄-alkyl group, as defined above, attached via a sulfur atom. The term “C₁-C₆-alkylthio” is a C₁-C₆-alkyl group, as defined above, attached via a sulfur atom. The term “C₁-C₁₀-alkylthio” is a C₁-C₁₀-alkyl group, as defined above, attached via a sulfur atom. C₁-C₂-Alkylthio is methylthio or ethylthio. C₁-C₄-Alkylthio is additionally, for example, n-propylthio, 1-methylethylthio(isopropylthio), butylthio, 1-methylpropylthio(sec-butylthio), 2-methylpropylthio(isobutylthio) or 1,1-dimethylethylthio(tert-butylthio). C₁-C₆-Alkylthio is additionally, for example, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio. C₁-C₈-Alkylthio is additionally, for example, heptylthio, octylthio, 2-ethylhexylthio and positional isomers thereof. C₁-C₁₀-Alkylthio is additionally, for example, nonylthio, decylthio and positional isomers thereof.

The term “C₁-C₂-haloalkylthio” is a C₁-C₂-haloalkyl group, as defined above, attached via a sulfur atom. The term “C₁-C₄-haloalkylthio” is a C₁-C₄-haloalkyl group, as defined above, attached via a sulfur atom. The term “C₁-C₆-haloalkylthio” is a C₁-C₆-haloalkyl group, as defined above, attached via a sulfur atom. The term “C₁-C₁₀-haloalkylthio” is a C₁-C₁₀-haloalkyl group, as defined above, attached via a sulfur atom. C₁-C₂-Haloalkylthio is, for example, SCH₂F, SCHF₂, SCF₃, SCH₂Cl, SCHCl₂, SCCl₃, chlorofluoromethylthio, dichlorofluoromethylthio, chlorodifluoromethylthio, 2-fluoroethylthio, 2-chloroethylthio, 2-bromoethylthio, 2-iodoethylthio, 2,2-difluoroethylthio, 2,2,2-trifluoroethylthio, 2-chloro-2-fluoroethylthio, 2-chloro-2,2-difluoroethylthio, 2,2-dichloro-2-fluoroethylthio, 2,2,2-trichloroethylthio or SC₂F₅. C₁-C₄-Haloalkylthio is additionally, for example, 2-fluoropropylthio, 3-fluoropropylthio, 2,2-difluoropropylthio, 2,3-difluoropropylthio, 2-chloropropylthio, 3-chloropropylthio, 2,3-dichloropropylthio, 2-bromopropylthio, 3-bromopropylthio, 3,3,3-trifluoropropylthio, 3,3,3-trichloropropylthio, SCH₂—C₂F₅, SCF₂—C₂F₅, 1-(CH₂F)-2-fluoroethylthio, 1-(CH₂Cl)-2-chloroethylthio, 1-(CH₂Br)-2-bromoethylthio, 4-fluorobutylthio, 4-chlorobutylthio, 4-bromobutylthio or nonafluorobutylthio. C₁-C₆-Haloalkylthio is additionally, for example, 5-fluoropentylthio, 5-chloropentylthio, 5-brompentylthio, 5-iodopentylthio, undecafluoropentylthio, 6-fluorohexylthio, 6-chlorohexylthio, 6-bromohexylthio, 6-iodohexylthio or dodecafluorohexylthio.

The term “C₁-C₂-alkylsulfinyl” is a C₁-C₂-alkyl group, as defined above, attached via a sulfinyl [S(O)] group. The term “C₁-C₄-alkylsulfinyl” is a C₁-C₄-alkyl group, as defined above, attached via a sulfinyl [S(O)] group. The term “C₁-C₆-alkylsulfinyl” is a C₁-C₆-alkyl group, as defined above, attached via a sulfinyl [S(O)] group. The term “C₁-C₁₀-alkylsulfinyl” is a C₁-C₁₀-alkyl group, as defined above, attached via a sulfinyl [S(O)] group. C₁-C₂-Alkylsulfinyl is methylsulfinyl or ethylsulfinyl. C₁-C₄-Alkylsulfinyl is additionally, for example, n-propylsulfinyl, 1-methylethylsulfinyl(isopropylsulfinyl), butylsulfinyl, 1-methylpropylsulfinyl(sec-butylsulfinyl), 2-methylpropylsulfinyl(isobutylsulfinyl) or 1,1-dimethylethylsulfinyl(tert-butylsulfinyl). C₁-C₆-Alkylsulfinyl is additionally, for example, pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl. C₁-C₈-Alkylsulfinyl is additionally, for example, heptylsulfinyl, octylsulfinyl, 2-ethylhexylsulfinyl and positional isomers thereof. C₁-C₁₀-Alkylsulfinyl is additionally, for example, nonylsulfinyl, decylsulfinyl and positional isomers thereof.

The term “C₁-C₂-haloalkylsulfinyl” is a C₁-C₂-haloalkyl group, as defined above, attached via a sulfinyl [S(O)] group. The term “C₁-C₄-haloalkylsulfinyl” is a C₁-C₄-haloalkyl group, as defined above, attached via a sulfinyl [S(O)] group. The term “C₁-C₆-haloalkylsulfinyl” is a C₁-C₆-haloalkyl group, as defined above, attached via a sulfinyl [S(O)] group. The term “C₁-C₁₀-haloalkylsulfinyl” is a C₁-C₁₀-haloalkyl group, as defined above, attached via a sulfinyl [S(O)] group. C₁-C₂-Haloalkylsulfinyl is, for example, S(O)CH₂F, S(O)CHF₂, S(O)CF₃, S(O)CH₂Cl, S(O)CHCl₂, S(O)CCl₃, chlorofluoromethylsulfinyl, dichlorofluoromethylsulfinyl, chlorodifluoromethylsulfinyl, 2-fluoroethylsulfinyl, 2-chloroethylsulfinyl, 2-bromoethylsulfinyl, 2-iodoethylsulfinyl, 2,2-difluoroethylsulfinyl, 2,2,2-trifluoroethylsulfinyl, 2-chloro-2-fluoroethylsulfinyl, 2-chloro-2,2-difluoroethylsulfinyl, 2,2-dichloro-2-fluoroethylsulfinyl, 2,2,2-trichloroethylsulfinyl or S(O)C₂F₅. C₁-C₄-Haloalkylsulfinyl is additionally, for example, 2-fluoropropylsulfinyl, 3-fluoropropylsulfinyl, 2,2-difluoropropylsulfinyl, 2,3-difluoropropylsulfinyl, 2-chloropropylsulfinyl, 3-chloropropylsulfinyl, 2,3-dichloropropylsulfinyl, 2-bromopropylsulfinyl, 3-bromopropylsulfinyl, 3,3,3-trifluoropropylsulfinyl, 3,3,3-trichloropropylsulfinyl, S(O)CH₂—C₂F₅, S(O)CF₂—C₂F₅, 1-(CH₂F)-2-fluoroethylsulfinyl, 1-(CH₂Cl)-2-chloroethylsulfinyl, 1-(CH₂Br)-2-bromoethylsulfinyl, 4-fluorobutylsulfinyl, 4-chlorobutylsulfinyl, 4-bromobutylsulfinyl or nonafluorobutylsulfinyl. C₁-C₆-Haloalkylsulfinyl is additionally, for example, 5-fluoropentylsulfinyl, 5-chloropentylsulfinyl, 5-brompentylsulfinyl, 5-iodopentylsulfinyl, undecafluoropentylsulfinyl, 6-fluorohexylsulfinyl, 6-chlorohexylsulfinyl, 6-bromohexylsulfinyl, 6-iodohexylsulfinyl or dodecafluorohexylsulfinyl.

The term “C₁-C₂-alkylsulfonyl” is a C₁-C₂-alkyl group, as defined above, attached via a sulfonyl [S(O)₂] group. The term “C₁-C₄-alkylsulfonyl” is a C₁-C₄-alkyl group, as defined above, attached via a sulfonyl [S(O)₂] group. The term “C₁-C₆-alkylsulfonyl” is a C₁-C₆-alkyl group, as defined above, attached via a sulfonyl [S(O)₂] group. The term “C₁-C₁₀-alkylsulfonyl” is a C₁-C₁₀-alkyl group, as defined above, attached via a sulfonyl [S(O)₂] group. C₁-C₂-Alkylsulfonyl is methylsulfonyl or ethylsulfonyl. C₁-C₄-Alkylsulfonyl is additionally, for example, n-propylsulfonyl, 1-methylethylsulfonyl(isopropylsulfonyl), butylsulfonyl, 1-methylpropylsulfonyl(sec-butylsulfonyl), 2-methylpropylsulfonyl(isobutylsulfonyl) or 1,1-dimethylethylsulfonyl(tert-butylsulfonyl). C₁-C₆-Alkylsulfonyl is additionally, for example, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl. C₁-C₈-Alkylsulfonyl is additionally, for example, heptylsulfonyl, octylsulfonyl, 2-ethylhexylsulfonyl and positional isomers thereof. C₁-C₁₀-Alkylsulfonyl is additionally, for example, nonylsulfonyl, decylsulfonyl and positional isomers thereof.

The term “C₁-C₂-haloalkylsulfonyl” is a C₁-C₂-haloalkyl group, as defined above, attached via a sulfonyl [S(O)₂] group. The term “C₁-C₄-haloalkylsulfonyl” is a C₁-C₄-haloalkyl group, as defined above, attached via a sulfonyl [S(O)₂] group. The term “C₁-C₆-haloalkylsulfonyl” is a C₁-C₆-haloalkyl group, as defined above, attached via a sulfonyl [S(O)₂] group. The term “C₁-C₁₀-haloalkylsulfonyl” is a C₁-C₁₀-haloalkyl group, as defined above, attached via a sulfonyl [S(O)₂] group. C₁-C₂-Haloalkylsulfonyl is, for example, S(O)₂CH₂F, S(O)₂CHF₂, S(O)₂CF₃, S(O)₂CH₂Cl, S(O)₂CHCl₂, S(O)₂CCl₃, chlorofluoromethylsulfonyl, dichlorofluoromethylsulfonyl, chlorodifluoromethylsulfonyl, 2-fluoroethylsulfonyl, 2-chloroethylsulfonyl, 2-bromoethylsulfonyl, 2-iodoethylsulfonyl, 2,2-difluoroethylsulfonyl, 2,2,2-trifluoroethylsulfonyl, 2-chloro-2-fluoroethylsulfonyl, 2-chloro-2,2-difluoroethylsulfonyl, 2,2-dichloro-2-fluoroethylsulfonyl, 2,2,2-trichloroethylsulfonyl or S(O)₂C₂F₅. C₁-C₄-Haloalkylsulfonyl is additionally, for example, 2-fluoropropylsulfonyl, 3-fluoropropylsulfonyl, 2,2-difluoropropylsulfonyl, 2,3-difluoropropylsulfonyl, 2-chloropropylsulfonyl, 3-chloropropylsulfonyl, 2,3-dichloropropylsulfonyl, 2-bromopropylsulfonyl, 3-bromopropylsulfonyl, 3,3,3-trifluoropropylsulfonyl, 3,3,3-trichloropropylsulfonyl, S(O)₂CH₂—C₂F₅, S(O)₂CF₂—C₂F₅, 1-(CH₂F)-2-fluoroethylsulfonyl, 1-(CH₂Cl)-2-chloroethylsulfonyl, 1-(CH₂Br)-2-bromoethylsulfonyl, 4-fluorobutylsulfonyl, 4-chlorobutylsulfonyl, 4-bromobutylsulfonyl or nonafluorobutylsulfonyl. C₁-C₆-Haloalkylsulfonyl is additionally, for example, 5-fluoropentylsulfonyl, 5-chloropentylsulfonyl, 5-brompentylsulfonyl, 5-iodopentylsulfonyl, undecafluoropentylsulfonyl, 6-fluorohexylsulfonyl, 6-chlorohexylsulfonyl, 6-bromohexylsulfonyl, 6-iodohexylsulfonyl or dodecafluorohexylsulfonyl.

The term “3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members” as used herein refers to monocyclic radicals, the monocyclic radicals being saturated, partially unsaturated or aromatic. The heterocyclic radical may be attached to the remainder of the molecule via a carbon ring member or via a nitrogen ring member.

Examples of 3-, 4-, 5-, 6- or 7-membered saturated heterocyclyl include: Oxiranyl, aziridinyl, azetidinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-pyrazolidinyl, 4-pyrazolidinyl, 5-pyrazolidinyl, 2-imidazolidinyl, 4-imidazolidinyl, 2-oxazolidinyl, 4-oxazolidinyl, 5-oxazolidinyl, 3-isoxazolidinyl, 4-isoxazolidinyl, 5-isoxazolidinyl, 2-thiazolidinyl, 4-thiazolidinyl, 5-thiazolidinyl, 3-isothiazolidinyl, 4-isothiazolidinyl, 5-isothiazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2-tetrahydropyranyl, 4-tetrahydropyranyl, 1,3-dioxan-5-yl, 1,4-dioxan-2-yl, 2-piperidinyl, 3-piperidinyl, 4-piperidinyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl, 2-morpholinyl, 3-morpholinyl, 2-thiomorpholinyl, 3-thiomorpholinyl, 1-oxothiomorpholin-2-yl, 1-oxothiomorpholin-3-yl, 1,1-dioxothiomorpholin-2-yl, 1,1-dioxothiomorpholin-3-yl, hexahydroazepin-1-, -2-, -3- or -4-yl, hexahydrooxepinyl, hexahydro-1,3-diazepinyl, hexahydro-1,4-diazepinyl, hexahydro-1,3-oxazepinyl, hexahydro-1,4-oxazepinyl, hexahydro-1,3-dioxepinyl, hexahydro-1,4-dioxepinyl and the like.

Examples of 3-, 4-, 5-, 6- or 7-membered partially unsaturated heterocyclyl include: 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 2-isothiazolin-4-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-, 3-, 4-, 5- or 6-di- or tetrahydropyridinyl, 3-di- or tetrahydropyridazinyl, 4-di- or tetrahydropyridazinyl, 2-di- or tetrahydropyrimidinyl, 4-di- or tetrahydropyrimidinyl, 5-di- or tetrahydropyrimidinyl, di- or tetrahydropyrazinyl, 1,3,5-di- or tetrahydrotriazin-2-yl, 1,2,4-di- or tetrahydrotriazin-3-yl, 2,3,4,5-tetrahydro[1H]azepin-1-, -2-, -3-, -4-, -5-, -6- or -7-yl, 3,4,5,6-tetrahydro[2H]azepin-2-, -3-, -4-, -5-, -6- or -7-yl, 2,3,4,7-tetrahydro-[1H]azepin-1-, -2-, -3-, -4-, -5-, -6- or -7-yl, 2,3,6,7-tetrahydro[1H]azepin-1-, -2-, -3-, -4-, -5-, -6- or -7-yl, tetrahydrooxepinyl, such as 2,3,4,5-tetrahydro[1H]oxepin-2-, -3-, -4-, -5-, -6- or -7-yl, 2,3,4,7-tetrahydro[1H]oxepin-2-, -3-, -4-, -5-, -6- or -7-yl, 2,3,6,7-tetrahydro[1H]oxepin-2-, -3-, -4-, -5-, -6- or -7-yl, tetrahydro-1,3-diazepinyl, tetrahydro-1,4-diazepinyl, tetrahydro-1,3-oxazepinyl, tetrahydro-1,4-oxazepinyl, tetrahydro-1,3-dioxepinyl and tetrahydro-1,4-dioxepinyl.

3-, 4-, 5-, 6- or 7-membered aromatic heterocyclyl is 5- or 6-membered aromatic heterocyclyl (hetaryl). Examples are: 2-furyl, 3-furyl, 2-thienyl, 3-thienyl, 2-pyrrolyl, 3-pyrrolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,3,4-triazol-2-yl, 2-pyridinyl, 3-pyridinyl, 4-pyridinyl, 3-pyridazinyl, 4-pyridazinyl, 2-pyrimidinyl, 4-pyrimidinyl, 5-pyrimidinyl and 2-pyrazinyl.

A 5-, 6-, 7- or 8-membered completely unsaturated heterocyclic ring containing 1, 2 or 3 heteroatoms selected from O, S and N as ring members contains the maximum number of possible double bonds in the ring. In the case of 5- or 6-membered rings, these are heteroaromatic. In the case of 7- and 8-membered rings, these cannot be aromatic, but can be homoaromatic (7-membered ring, 3 double bonds) or can have 4 double bonds (8-membered ring).

C₂-C₇-alkylene is divalent branched or preferably unbranched saturated aliphatic chain having 2 to 7 carbon atoms, for example —CH₂CH₂, —CH(CH₃)—, —CH₂CH₂CH₂—, —CH(CH₃)CH₂—, —CH₂CH(CH₃)—, —CH₂CH₂CH₂CH₂—, —CH₂CH₂CH₂CH₂CH₂—, —CH₂CH₂CH₂CH₂CH₂CH₂—, and —CH₂CH₂CH₂CH₂CH₂CH₂CH₂—.

The remarks made below concerning preferred embodiments of the variables of the compounds of formula I, especially with respect to their substituents X, Y, A¹, A², A³, B¹, B², B³, R¹, R², R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, m, n, p, q and r, the features of the use and method according to the invention and of the composition of the invention are valid both on their own and, in particular, in every possible combination with each other.

As a matter of course, the q radicals R¹ replace a hydrogen atom on a carbon ring atom. For instance, if B¹, B² or B³ is defined to be CH and if this position is to be substituted by a radical R¹, then B¹, B² or B³ is of course C—R¹. If there is more than one radical R¹, these can be the same or different.

R² is a substituent on that ring of the condensed system Q which contains A¹, A² and A³ as ring members.

As a matter of course, the p radicals R² replace a hydrogen atom on a carbon ring atom. For instance, if A¹, A² or A³ is defined to be CH and if this position is to be substituted by a radical R², then A¹, A² or A³ is of course C—R². If there is more than one radical R², these can be the same or different.

Preferably, at most two of A¹, A² and A³ are N. More preferably, at most one of A¹, A² and A³ is N. In one embodiment, A¹, A² and A³ are CH. In an alternative embodiment, A¹ and A³ are CH and A² is N. In an alternative embodiment, A¹ and A² are CH and A³ is N. In an alternative embodiment, A² and A³ are CH and A¹ is N. In particular, A¹, A² and A³ are CH or A¹ is N and A² and A³ are CH. Specifically, A¹ is N and A² and A³ are CH.

In a preferred embodiment, the ring comprising the groups A¹, A² and A³ as ring members carries 0, 1 or 2 and more preferably 0 or 1 substituent R². In other words, p is preferably 0, 1 or 2 and more preferably 0 or 1. In case p is 1, and in case A¹ is CH, then R² is preferably bound to the position of A¹. However, particularly preferably, p is 0.

In case p is 2 and two substituents R² bound on adjacent carbon atoms, together with the carbon atoms to which they are bound, form a fused ring, they preferably form together a group selected from —CH₂CH₂CH₂CH₂— and —CH═CH—CH═CH— and more preferably —CH═CH—CH═CH—, thus yielding a fused phenyl ring.

Preferably, at most one of B¹, B² and B³ is N. More preferably, B¹, B² and B³ are CH or

B¹ and B² are CH and B³ is N. Specifically, B¹, B² and B³ are CH.

q is preferably 0, 1, 2 or 3, more preferably 1, 2 or 3, even more preferably 2 or 3 and in particular 2. If q is 3 and B¹, B² and B³ are CH, then the three substituents R¹ are preferably bound in the positions of B¹, B² and B³; B¹, B² and B³ thus being C—R¹. If q is 2 and B¹, B² and B³ are CH, then the two substituents R¹ are preferably bound in the positions of B¹ and B³; B¹ and B³ thus being C—R¹. B² in this case is preferably CH. In case B¹ and B² are CH and B³ is N, q is preferably 1. In this case, R¹ is preferably bound in the position of B¹, B¹ thus being C—R¹. Specifically, q is 2 and B¹, B² and B³ are CH, and the two substituents R¹ are bound in the positions of B¹ and B³; B¹ and B³ thus being C—R¹ and B² being CH.

X is preferably selected from the group consisting of C₁-C₄-alkyl, C₁-C₄-haloalkyl, C₁-C₄-alkoxy-C₁-C₄-alkyl, C₁-C₄-haloalkoxy-C₁-C₄-alkyl, C₃-C₆-cycloalkyl and C₃-C₆-halocycloalkyl. More preferably, X is selected from the group consisting of C₁-C₄-alkyl, C₁-C₄-haloalkyl, C₃-C₆-cycloalkyl and C₃-C₆-halocycloalkyl. Even more preferably, X is selected from the group consisting of C₁-C₄-alkyl and C₁-C₄-haloalkyl. In particular, X is C₁-C₄-haloalkyl, specifically C₁-C₂-haloalkyl and more specifically halomethyl, in particular fluoromethyl, such as fluoromethyl, difluoromethyl and trifluoromethyl, and is very specifically trifluoromethyl.

R³ is a substituent on the “G” ring of the condensed system Q.

Preferably, G is a condensed phenyl ring, a condensed 5-membered heteroaromatic ring containing 1 heteroatom selected from O, S and N and optionally 1 or 2 further nitrogen atoms as ring members or a condensed 6-membered heteroaromatic ring containing 1, 2 or 3 nitrogen atoms as ring members.

Thus, preferred groups Q are selected from radicals of following formulae:

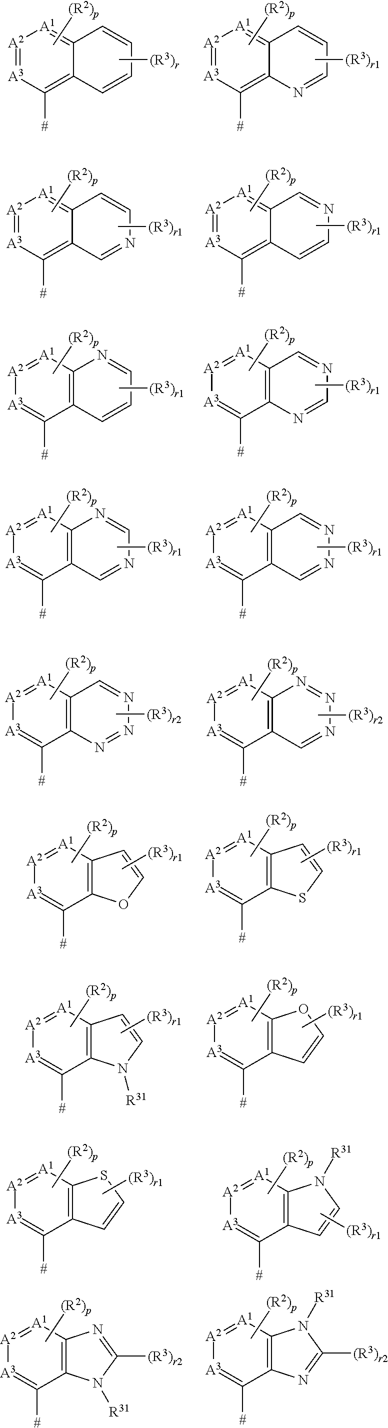

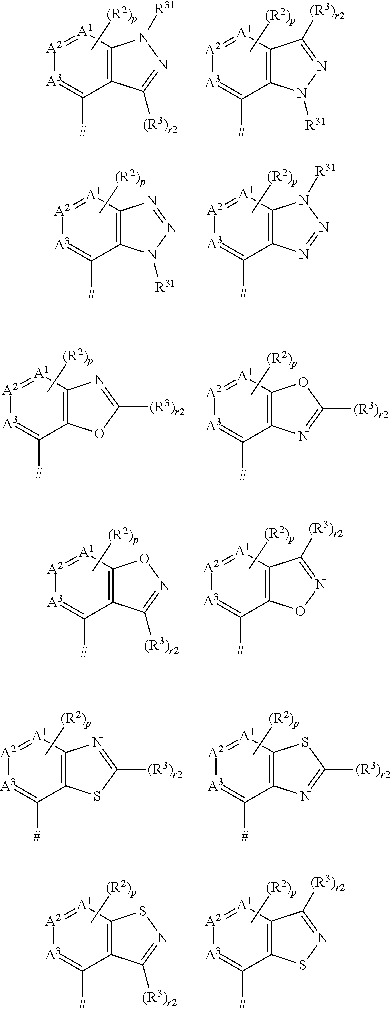

wherein
A¹, A² and A³ have one of the above-given general or, preferably, preferred meanings;
R² and R³ have one of the above-given general meanings or, preferably, have one of the below-given preferred meanings;
R³¹ is hydrogen or has one of the above general meanings or, preferably, one of the above meanings given for R⁸, or, more preferably, one of the below preferred meanings given for R³;
p and r have one of the above-given general meanings or, preferably, have one of the above- or below-given preferred meanings;
r¹ is 0, 1 or 2;
r² is 0 or 1; and
# is the attachment point to the remainder of the molecule.

More preferred groups Q are selected from radicals of following formulae:

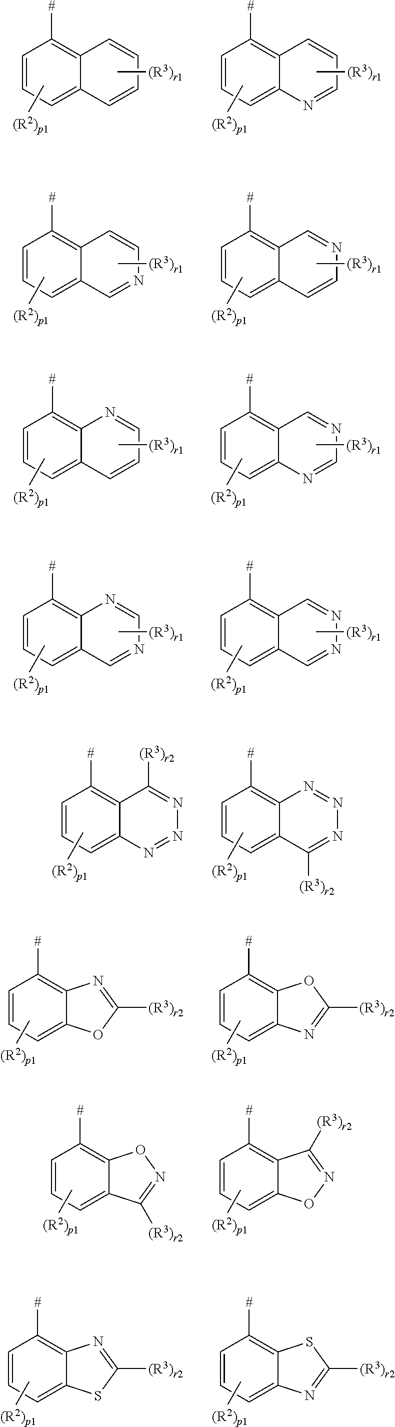

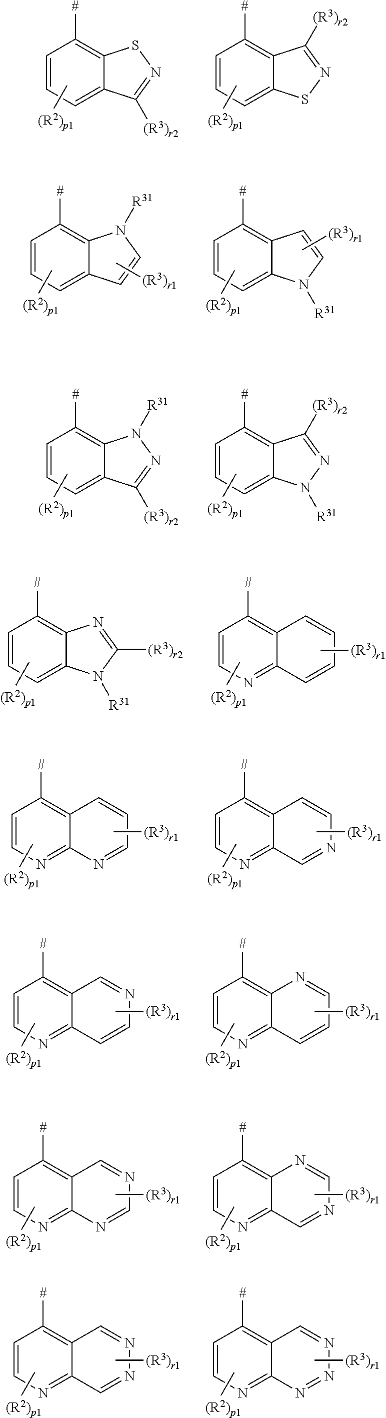

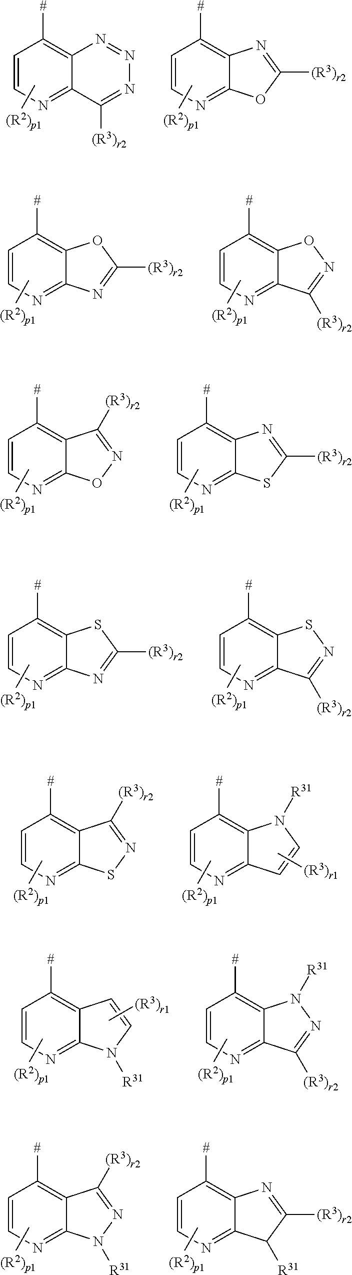

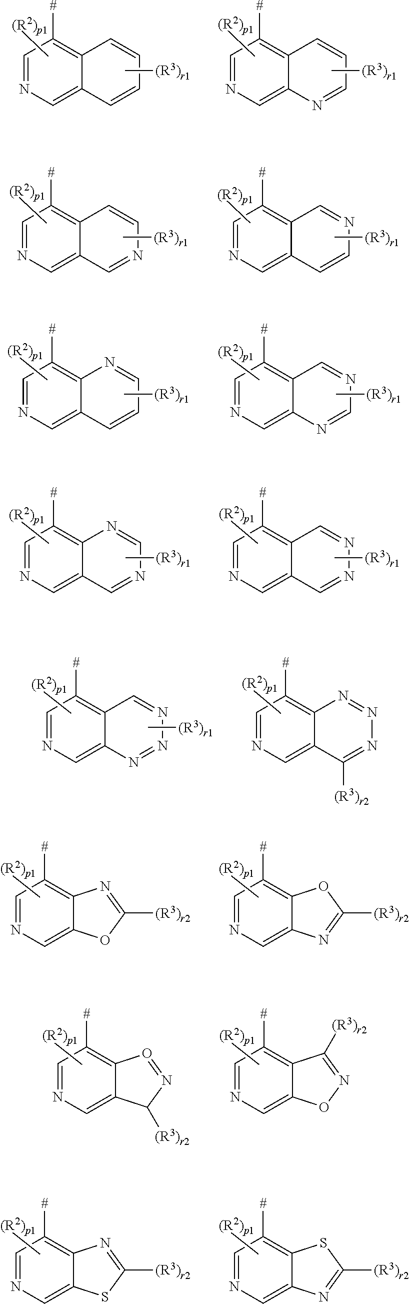

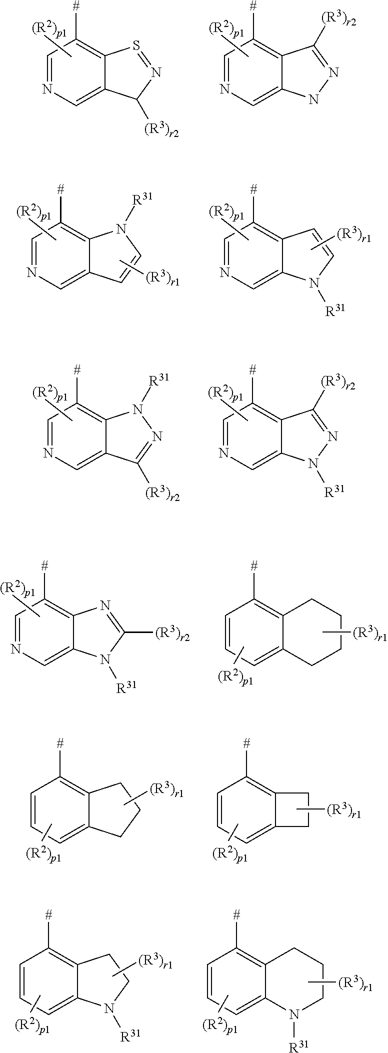

where
R² and R³ have one of the above-given general meanings or, preferably, have one of the below-given preferred meanings;
R³¹ has one of the meanings given above for R⁸ and is preferably independently selected from the group consisting of hydrogen, C₁-C₈-alkyl, C₁-C₆-haloalkyl, C₁-C₆-alkyl substituted by a phenyl group or substituted by a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, formyl, C₁-C₆-alkylcarbonyl, C₁-C₆-haloalkylcarbonyl, C₃-C₆-cycloalkylcarbonyl, C₃-C₆-halocycloalkylcarbonyl, —C(O)—CH₂—(C₃-C₈-cycloalkyl), —C(O)—CH₂—(C₃-C₆-halocycloalkyl), C₁-C₆-alkoxycarbonyl, C₁-C₆-haloalkoxycarbonyl, —C(O)—CH₂—S(O)ₙ—(C₁-C₄-alkyl), —C(O)—CH₂—S(O)ₙ—(C₁-C₄-haloalkyl), phenylcarbonyl, —C(O)—CH₂-phenyl, —C(O)—CH₂—S(O)ₙ-phenyl, phenyl, where the phenyl moiety in the 4 last-mentioned radicals may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰), or, more preferably, has one of the below preferred meanings given for R³;
p¹ is 0, 1 or 2;
r¹ is 0, 1 or 2;
r² is 0 or 1; and
# is the attachment point to the remainder of the molecule.

Even more preferred groups Q are selected from radicals of following formulae:

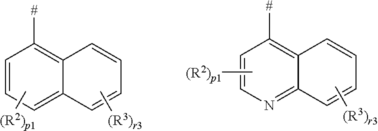

where
R² and R³ have one of the above-given general meanings or, preferably, have one of the below-given preferred meanings;
p¹ is 0, 1 or 2, preferably 0 or 1 and specifically 0;
r³ is 0, 1 or 2, preferably 0 or 1 and specifically 1; and
# is the attachment point to the remainder of the molecule.

Specifically, Q has the formula

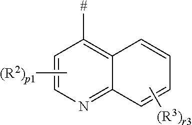

where
R² and R³ have one of the above-given general meanings or, preferably, have one of the below-given preferred meanings;
p¹ is 0, 1 or 2, preferably 0 or 1 and specifically 0;
r³ is 0, 1 or 2, preferably 0 or 1 and specifically 1; and
# is the attachment point to the remainder of the molecule.

Preferably, each R¹ is independently selected from the group consisting of halogen, cyano, nitro, —SCN, SF₅, C₁-C₆-alkyl, C₁-C₆-alkyl which may be partially or fully halogenated and/or may be substituted by one or more, e.g. 1, 2, 3 or 4, preferably 1 or 2, more preferably 1, radicals R⁴, C₃-C₈-cycloalkyl which may be partially or fully halogenated and/or may be substituted by one or more, e.g. 1, 2, 3 or 4, preferably 1 or 2, more preferably 1, radicals R⁵, C₂-C₆-alkenyl which may be partially or fully halogenated and/or may be substituted by one or more, e.g. 1, 2, 3 or 4, preferably 1 or 2, more preferably 1, radicals R⁴, C₂-C₆-alkynyl which may be partially or fully halogenated and/or may be substituted by one or more, e.g. 1, 2, 3 or 4, preferably 1 or 2, more preferably 1, radicals R⁴, Si(R¹⁴)₂R¹³, OR⁷, OS(O)ₙR⁷, S(O)ₘR⁷, NR⁸R⁹, N(R⁸)C(═O)R⁶, C(═O)R⁶, C(═O)OR⁷, C(═NR⁸)R⁶, C(═S)NR⁶, phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰,

where R⁴, R⁵, R⁶, R⁷, R⁸, R⁹, R¹⁰, R¹³ and R¹⁴ have one of the meanings given above or in particular one of the preferred meanings given below.

More preferably, each R¹ is independently selected from the group consisting of halogen, cyano, nitro, C₁-C₆-alkyl, C₁-C₆-alkyl which may be partially or fully halogenated and/or may be substituted by one or more, e.g. 1, 2, 3 or 4, preferably 1 or 2, more preferably 1, radicals R⁴, OR⁷, phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰,

where R⁴, R⁵, R⁶, R⁷ and R¹⁰ have one of the meanings given above or in particular one of the preferred meanings given below.

Even more preferably, each R¹ is independently selected from the group consisting of halogen, cyano, nitro, hydroxyl, C₁-C₄-alkyl, C₁-C₄-haloalkyl, C₁-C₄-alkoxy and C₁-C₄-haloalkoxy, particularly preferably from halogen, C₁-C₄-alkyl, C₁-C₄-haloalkyl, C₁-C₄-alkoxy and C₁-C₄-haloalkoxy, in particular from halogen, C₁-C₄-alkyl and C₁-C₂-haloalkyl, more particularly halogen and CF₃, and is specifically halogen, more specifically chlorine.

Preferably, each R² is independently selected from the group consisting of halogen; cyano; azido; nitro; —SCN; SF₅; C₁-C₆-alkyl which may be partially or fully halogenated and/or may be substituted by one or more, e.g. 1, 2, 3 or 4, preferably 1 or 2, more preferably 1, radicals R⁴; C₃-C₈-cycloalkyl which may be partially or fully halogenated and/or may be substituted by one or more, e.g. 1, 2, 3 or 4, preferably 1 or 2, more preferably 1, radicals R⁵; C₂-C₆-alkenyl which may be partially or fully halogenated and/or may be substituted by one or more, e.g. 1, 2, 3 or 4, preferably 1 or 2, more preferably 1, radicals R⁴; C₂-C₆-alkynyl which may be partially or fully halogenated and/or may be substituted by one or more, e.g. 1, 2, 3 or 4, preferably 1 or 2, more preferably 1, radicals R⁴; —Si(R¹⁴)₂R¹³; —OR⁷; —OS(O)ₙR⁷; —SR⁷; —S(O)ₘR⁷; —S(O)ₙN(R⁸)R⁹; —N(R⁸)R⁹; —N(R⁸)C(═O)R⁶; —C(═O)R⁶, wherein R⁶ is different from —N(R⁸)R⁹; —C(═O)OR⁷; —C(═S)R⁶, wherein R⁶ is different from —N(R⁸)R⁹; —C(═S)OR⁷, phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰;

with the proviso that R² is not halogen, cyano, nitro, methyl which carries one halogen atom or carries one group OH or one methylcarbonyloxy group; OH, methoxy, —OS(O)ₙR⁷, —NH₂, —CHO, C₁-C₆-alkylcarbonyl; or —C(═O)OR⁷, wherein R⁷ is hydrogen, C₁-C₆-alkyl or benzyl; if A¹, A² and A³ are CH and if simultaneously R² is bound to A¹;

or two radicals R² bound on adjacent carbon atoms may be together a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR⁸—, —CH₂CH═N—, —CH═CH—NR⁸—, —OCH═N—, and —SCH═N—, thus forming, together with the carbon atoms to which they are bound, a 5- or 6-membered ring, where the hydrogen atoms of the above groups may be replaced by one or more, e.g. 1, 2, 3 or 4, preferably 1 or 2, more preferably 1, substituents selected from halogen, methyl, halomethyl, hydroxyl, methoxy and halomethoxy or one or more, e.g. 1 or 2, CH₂ groups of the above groups may be replaced by a C═O group,

where R⁴, R⁵, R⁶, R⁷, R⁸, R⁹, R¹⁰, R¹³ and R¹⁴ have one of the meanings given above or in particular one of the preferred meanings given below.

If two radicals R² bound on adjacent carbon atoms together form a bridging group, this is preferably selected from —CH₂CH₂CH₂CH₂— and —CH═CH—CH═CH— and is more preferably —CH═CH—CH═CH—.

More preferably, each R² is independently selected from the group consisting of halogen; cyano; azido; nitro; —SCN; SF₅; C₁-C₆-alkyl which may be partially or fully halogenated and/or may be substituted by one or more, e.g. 1, 2, 3 or 4, preferably 1 or 2, more preferably 1, radicals R⁴; C₃-C₆-cycloalkyl which may be partially or fully halogenated and/or may be substituted by one or more, e.g. 1, 2, 3 or 4, preferably 1 or 2, more preferably 1, radicals R⁵; C₂-C₆-alkenyl which may be partially or fully halogenated and/or may be substituted by one or more, e.g. 1, 2, 3 or 4, preferably 1 or 2, more preferably 1, radicals R⁴; C₂-C₆-alkynyl which may be partially or fully halogenated and/or may be substituted by one or more, e.g. 1, 2, 3 or 4, preferably 1 or 2, more preferably 1, radicals R⁴; —Si(R¹⁴)₂R¹³; —OR⁷; —OS(O)ₙR⁷; —SR⁷; —S(O)ₙN(R⁸)R⁹; —N(R⁸)R⁹; —N(R⁸)C(═O)R⁶; —C(═O)R⁶, wherein R⁶ is different from —N(R⁸)R⁹; —C(═O)OR⁷; —C(═S)R⁶, wherein R⁶ is different from —N(R⁸)R⁹; —C(═S)OR⁷, phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰;

where R⁴, R⁵, R⁶, R⁷, R⁸, R⁹, R¹⁰, R¹³ and R¹⁴ have one of the meanings given above or in particular one of the preferred meanings given below.

Even more preferably, each R² is independently selected from the group consisting of azido; —SCN; SF₅; C₁-C₆-alkyl; C₁-C₆-alkyl which is substituted by one or more, e.g. 1, 2, 3 or 4, preferably 1 or 2, more preferably 1, radicals R⁴ different from OR⁷; C₃-C₈-cycloalkyl which may be partially or fully halogenated and/or may be substituted by one or more, e.g. 1, 2, 3 or 4, preferably 1 or 2, more preferably 1, radicals R⁵; C₂-C₆-alkenyl which may be partially or fully halogenated and/or may be substituted by one or more, e.g. 1, 2, 3 or 4, preferably 1 or 2, more preferably 1, radicals R⁴; C₂-C₆-alkynyl which may be partially or fully halogenated and/or may be substituted by one or more, e.g. 1, 2, 3 or 4, preferably 1 or 2, more preferably 1, radicals R⁴; —Si(R¹⁴)₂R¹³; wherein R⁷ is not hydrogen or C₁-C₆-alkyl; —SR⁷; —S(O)ₙN(R⁸)R⁹; —N(R⁸)C(═O)R⁶; —C(═S)OR⁷, phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰;

where R⁴, R⁵, R⁶, R⁷, R⁸, R⁹, R¹⁰, R¹³ and R¹⁴ have one of the meanings given above or in particular one of the preferred meanings given below.

Particularly preferably, each R² is independently selected from the group consisting of —SCN; —SF₅; C₁-C₆-alkyl; C₁-C₆-alkyl which is substituted by one or more, e.g. 1, 2, 3 or 4, preferably 1 or 2, more preferably 1, radicals R⁴ different from OR⁷; C₃-C₈-cycloalkyl which may be partially or fully halogenated and/or may be substituted by one or more, e.g. 1, 2, 3 or 4, preferably 1 or 2, more preferably 1, radicals R⁵; C₂-C₆-alkenyl which may be partially or fully halogenated and/or may be substituted by one or more, e.g. 1, 2, 3 or 4, preferably 1 or 2, more preferably 1, radicals R⁴; C₂-C₆-alkynyl which may be partially or fully halogenated and/or may be substituted by one or more, e.g. 1, 2, 3 or 4, preferably 1 or 2, more preferably 1, radicals R⁴; —Si(R¹⁴)₂R¹³; —OR⁷, wherein R⁷ is not hydrogen or C₁-C₆-alkyl; —SR⁷; —S(O)ₘR⁷; —S(O)ₙN(R⁸)R⁹; —N(R⁸)C(═O)R⁶; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰;

where R⁴, R⁵, R⁶, R⁷, R⁸, R⁹, R¹⁰, R¹³ and R¹⁴ have one of the meanings given above or in particular one of the preferred meanings given below.

Alternatively, R² is preferably bound on A² and/or A³ and each R² is independently selected from halogen; cyano; nitro; —SCN; C₁-C₆-alkyl which may be partially or fully halogenated and/or may be substituted by one or more, e.g. 1, 2, 3 or 4, preferably 1 or 2, more preferably 1, radicals R⁴; C₃-C₈-cycloalkyl which may be partially or fully halogenated and/or may be substituted by one or more, e.g. 1, 2, 3 or 4, preferably 1 or 2, more preferably 1, radicals R⁵; —OR⁷; —OS(O)ₙR⁷; —SR⁷; —S(O)ₙR⁷; —S(O)ₙN(R⁸)R⁹; —N(R⁸)R⁹; —N(R⁸)C(═O)R⁶; —C(═O)R⁶; —C(═O)OR⁷; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰; and more preferably, R² is bound on A² and/or A³ and each R² is independently selected from halogen, cyano, hydroxyl, C₁-C₄-alkyl, C₁-C₄-haloalkyl, C₁-C₄-alkoxy, C₁-C₄-haloalkoxy, phenyl which may be substituted by 1, 2 or 3 radicals R¹⁰; and a 5- or 6-membered heteroaromatic ring containing 1, 2 or 3 heteroatoms selected from N, O and S as ring members, where the heteroaromatic ring may be substituted by 1 or 2 radicals R¹⁰; where R¹⁰ has one of the meanings given above or in particular one of the preferred meanings given below.

p is preferably 0, 1 or 2, more preferably 0 or 1 and specifically 0.

Preferably, each R³ is independently selected from halogen; cyano; nitro; —SCN; SF₅; C₁-C₆-alkyl which may be partially or fully halogenated and/or may be substituted by one or more, e.g. 1, 2, 3 or 4, preferably 1 or 2, more preferably 1, radicals R⁴; C₃-C₈-cycloalkyl which may be partially or fully halogenated and/or may be substituted by one or more, e.g. 1, 2, 3 or 4, preferably 1 or 2, more preferably 1, radicals R⁵; C₂-C₆-alkenyl which may be partially or fully halogenated and/or may be substituted by one or more, e.g. 1, 2, 3 or 4, preferably 1 or 2, more preferably 1, radicals R⁴; C₂-C₆-alkynyl which may be partially or fully halogenated and/or may be substituted by one or more, e.g. 1, 2, 3 or 4, preferably 1 or 2, more preferably 1, radicals R⁴;—Si(R¹⁴)₂R¹³; —OR⁷; —OS(O)ₙR⁷; —SR⁷; —S(O)ₘR⁷; —S(O)ₙN(R⁸)R⁹; —C(═NR⁸)R⁶; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰;

or two radicals R³ bound on adjacent carbon atoms may be together a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR⁸—, —CH₂CH═N—, —CH═CH—NR⁸—, —OCH═N—, and —SCH═N—, thus forming, together with the carbon atoms to which they are bound, a 5- or 6-membered ring, where the hydrogen atoms of the above groups may be replaced by one or more, e.g. 1, 2, 3 or 4, preferably 1 or 2, more preferably 1, substituents selected from halogen, methyl, halomethyl, hydroxyl, methoxy and halomethoxy or one or more, e.g. 1 or 2, CH₂ groups of the above groups may be replaced by a C═O group,

where R⁴, R⁵, R⁶, R⁷, R⁸, R⁹, R¹⁰, R¹³ and R¹⁴ have one of the meanings given above or in particular one of the preferred meanings given below.

If two radicals R³ bound on adjacent carbon atoms together form a bridging group, this is preferably selected from —CH₂CH₂CH₂CH₂— and —CH═CH—CH═CH— and is more preferably —CH═CH—CH═CH—.

More preferably, each R³ is independently selected from halogen; cyano; nitro; —SCN; C₁-C₆-alkyl which may be partially or fully halogenated and/or may be substituted by one or more, e.g. 1, 2, 3 or 4, preferably 1 or 2, more preferably 1, radicals R⁴; C₃-C₈-cycloalkyl which may be partially or fully halogenated and/or may be substituted by one or more, e.g. 1, 2, 3 or 4, preferably 1 or 2, more preferably 1, radicals R⁵; —OR⁷; —C(═O)OR⁷; —C(═O)N(R⁸)R⁹; —OS(O)ₘR⁷; —S(O)ₙOR⁷; —SR⁷; —S(O)ₘR⁷; —S(O)ₙN(R⁸)R⁹; —C(═NR⁸)R⁶; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰;

where R⁴, R⁵, R⁶, R⁷, R⁸, R⁹ and R¹⁰ have one of the meanings given above or in particular one of the preferred meanings given below.

Even more preferably, each R³ is independently selected from halogen; cyano; nitro; —SCN; C₁-C₆-alkyl which may be partially or fully halogenated and/or may be substituted by one or more, e.g. 1, 2, 3 or 4, preferably 1 or 2, more preferably 1, radicals R⁴; C₃-C₈-cycloalkyl which may be partially or fully halogenated and/or may be substituted by one or more, e.g. 1, 2, 3 or 4, preferably 1 or 2, more preferably 1, radicals R⁵; —OR⁷; —C(═O)OR⁷; —C(═O)N(R⁸)R⁹; —OS(O)ₘR⁷; —S(O)ₘOR⁷; —SR⁷; —S(O)ₙR⁷; —S(O)ₙN(R⁸)R⁹; —C(═NR⁸)R⁶; phenyl which may be substituted by 1, 2, 3, 4 or 5 radicals R¹⁰ and a 5- or 6-membered heteroaromatic ring containing 1, 2 or 3 heteroatoms selected from N, O and S as ring members, where the heteroaromatic ring may be substituted by 1 or 2 radicals R¹⁰;

where R⁴, R⁵, R⁶, R⁷, R⁸, R⁹ and R¹⁰ have one of the meanings given above or in particular one of the preferred meanings given below.

Particularly preferably, each R³ is independently selected from halogen, cyano, C₁-C₄-alkyl which may be substituted by one or more, e.g. 1, 2, 3 or 4, preferably 1 or 2, more preferably 1, radicals R⁴, C₁-C₄-haloalkyl, —OR⁷, —C(═O)OR⁷; —C(═O)N(R⁸)R⁹; —OS(O)₂R⁷; —S(O)₂OR⁷; —S(O)₂R⁷; —S(O)ₙN(R⁸)R⁹; and —C(═NR⁸)R⁶, where R⁴, R⁶, R⁷, R⁸ and R⁹ have one of the meanings given above or in particular one of the preferred meanings given below.

In particular, each R³ is independently selected from halogen, cyano, C₁-C₄-alkyl which may be substituted by one or more radicals R⁴, C₁-C₄-haloalkyl, —OR⁷, —C(═O)OR⁷, —C(═O)N(R⁸)R⁹; —OS(O)₂R⁷; —S(O)₂OR⁷; —S(O)₂R⁷; —S(O)₂N(R⁸)R⁹; and —C(═NR⁸)R⁶, where R⁴, R⁶, R⁷, R⁸ and R⁹ have one of the meanings given above or in particular one of the preferred meanings given below.

r is preferably 0, 1 or 2, more preferably 0 or 1 and specifically 1.

R⁴ is preferably selected from the group consisting of cyano, azido, nitro, —SCN, SF₅, C₃-C₈-cycloalkyl which may be substituted by phenyl, C₃-C₈-halocycloalkyl, —Si(R¹⁴)₂R¹³, —OR⁷, —OSO₂R⁷, —SR⁷, —S(O)ₘR⁷, —S(O)ₙN(R⁸)R⁹, —N(R⁸)R⁹, —C(═O)N(R⁸)R⁹, —C(═S)N(R⁸)R⁹, —C(═O)OR⁷, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. by 1, 2, 3, 4 or 5, preferably 1, 2 or 3, more preferably by 1 or 2 and in particular 1, radicals R¹⁰; or two geminally bound radicals R⁴ together form a group selected from ═CR¹¹R¹², ═S(O)ₘR⁷, ═S(O)ₘN(R⁸)R⁹, ═NR⁸, ═NOR⁷ and ═NNR⁸; or two radicals R⁴, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members,

where R⁷, R⁸, R⁹, R¹⁰, R¹¹, R¹², R¹³ and R¹⁴ have one of the meanings given above or in particular one of the preferred meanings given below.

R⁴ is more preferably selected from the group consisting of cyano, C₃-C₈-cycloalkyl which may be substituted by phenyl, C₃-C₈-halocycloalkyl, —OR⁷, —SR⁷, —C(═O)N(R⁸)R⁹, —N(R⁸)R⁹, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. by 1, 2, 3, 4 or 5, preferably 1, 2 or 3, more preferably by 1 or 2 and in particular 1, radicals R¹⁰; where R⁷, R⁸, R⁹ and R¹⁰ have one of the meanings given above or in particular one of the preferred meanings given below.

R⁴ is even more preferably selected from the group consisting of cyano, C₃-C₆-cycloalkyl which may be substituted by phenyl, C₃-C₆-halocycloalkyl, C₁-C₄-alkoxy, C₁-C₄-haloalkoxy, C₁-C₄-alkylthio, C₁-C₄-haloalkylthio, —C(═O)N(R⁸)R⁹, —N(R⁸)R⁹, phenyl which may be substituted by 1, 2, 3, 4 or 5 radicals R¹⁰, and a 5- or 6-membered heteroaromatic ring containing 1, 2 or 3 heteroatoms selected from N, O and S, as ring members, where the heteroaromatic ring may be substituted by one or more, e.g. by 1, 2, 3, 4 or 5, preferably 1, 2 or 3, more preferably by 1 or 2 and in particular 1, radicals R¹⁰;

where R⁸, R⁹ and R¹⁰ have one of the meanings given above or in particular one of the preferred meanings given below.

R⁴ is in particular selected from the group consisting of C₃-C₆-cycloalkyl which may be substituted by phenyl, phenyl which may be substituted by 1, 2, 3, 4 or 5, preferably 1, 2 or 3 radicals R¹⁰, —C(═O)N(R⁸)R⁹, —N(R⁸)R⁹, and a 5- or 6-membered heteroaromatic ring containing 1, 2 or 3 heteroatoms selected from N, O and S, as ring members, where the heteroaromatic ring may be substituted by one or more, e.g. by 1, 2, 3, 4 or 5, preferably 1, 2 or 3, more preferably by 1 or 2 and in particular 1, radicals R¹⁰; where R⁸, R⁹ and R¹⁰ have one of the meanings given above or in particular one of the preferred meanings given below.

Especially in the definition of R³, R⁴ is —N(R⁸)R⁹, where R⁸ and R⁹ have one of the meanings given above or in particular one of the preferred meanings given below. Preferably, R⁸ is selected from hydrogen, C₁-C₆-alkyl, formyl, C₁-C₆-alkylcarbonyl, C₁-C₆-haloalkylcarbonyl, C₁-C₆-alkoxycarbonyl, C₁-C₆-haloalkoxycarbonyl, —C(O)—CH₂—S(O)ₙ—(C₁-C₄-alkyl) and —C(O)—CH₂—S(O)ₙ—(C₁-C₄-haloalkyl), and R⁹ has one of the meanings given above or in particular one of the preferred meanings given below and is in particular hydrogen or C₁-C₄-alkyl and specifically hydrogen.

R⁵ is preferably selected from the group consisting of halogen, cyano, azido, nitro, —SCN, SF₅, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, —Si(R¹⁴)₂R¹³, —OR⁷, —OSO₂R⁷, —SR⁷, —S(O)ₘR⁷, —S(O)ₙN(R⁸)R⁹, —N(R⁸)R⁹, —C(═O)N(R⁸)R⁹, —C(═S)N(R⁸)R⁹, —C(═O)OR⁷, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰;

or two geminally bound radicals R⁵ together form a group selected from ═CR¹¹R¹², ═S(O)ₘR⁷, ═S(O)ₘN(R⁸)R⁹, ═NR⁸, ═NOR⁷ and ═NNR⁸;

or two radicals R⁵, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from N, O, S, NO, SO and SO₂, as ring members,

where R⁷, R⁸, R⁹, R¹⁰, R¹¹, R¹², R¹³ and R¹⁴ have one of the meanings given above or in particular one of the preferred meanings given below.

R⁵ is more preferably selected from the group consisting of halogen, cyano, C₁-C₆-alkyl, C₁-C₆-haloalkyl, C₁-C₆-alkoxy-C₁-C₆-alkyl, —OR⁷, —OSO₂R⁷, —SR⁷, —S(O)ₘR⁷, —S(O)ₙN(R⁸)R⁹, —N(R⁸)R⁹, —C(═O)N(R⁸)R⁹, —C(═S)N(R⁸)R⁹, —C(═O)OR⁷, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. by 1, 2, 3, 4 or 5, preferably 1, 2 or 3, more preferably by 1 or 2 and in particular 1, radicals R¹⁰;

where R⁷, R⁸, R⁹ and R¹⁰ have one of the meanings given above or in particular one of the preferred meanings given below.

R⁵ is even more preferably selected from the group consisting of halogen, C₁-C₄-alkyl, C₁-C₃-haloalkyl, C₁-C₄-alkoxy and C₁-C₃-haloalkoxy. In particular, R⁵ is selected from halogen, C₁-C₄-alkyl and C₁-C₃-haloalkyl.

R⁶ is preferably selected from the group consisting of hydrogen, C₁-C₆-alkyl, C₁-C₆-haloalkyl, C₁-C₆-alkoxy-C₁-C₆-alkyl, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, —SR⁷, —N(R⁸)R⁹, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. by 1, 2, 3, 4 or 5, preferably 1, 2 or 3, more preferably by 1 or 2 and in particular 1, radicals R¹⁰;

where R¹, R⁸, R⁹ and R¹⁰ have one of the meanings given above or in particular one of the preferred meanings given below.

R⁶ is more preferably selected from the group consisting of C₁-C₆-alkyl, C₁-C₆-haloalkyl, C₃-C₈-cycloalkyl, C₃-C₈-halocycloalkyl, C₁-C₆-alkoxy, C₁-C₆-haloalkoxy, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. by 1, 2, 3, 4 or 5, preferably 1, 2 or 3, more preferably by 1 or 2 and in particular 1, radicals R¹⁰;

where R¹⁰ has one of the meanings given above or in particular one of the preferred meanings given below.

R⁶ is even more preferably selected from the group consisting of C₁-C₆-alkyl, C₁-C₆-haloalkyl, C₃-C₈-cycloalkyl, C₃-C₈-halocycloalkyl, C₁-C₆-alkoxy, C₁-C₆-haloalkoxy, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. by 1, 2, 3, 4 or 5, preferably 1, 2 or 3, more preferably by 1 or 2 and in particular 1, radicals R¹⁰;

where R¹⁰ has one of the meanings given above or in particular one of the preferred meanings given below.

R⁶ is particularly preferably selected from the group consisting of hydrogen, C₁-C₄-alkyl, C₁-C₃-haloalkyl, C₃-C₆-cycloalkyl, C₃-C₆-halocycloalkyl, C₁-C₄-alkoxy, C₁-C₃-haloalkoxy, phenyl which may be substituted by 1, 2, 3, 4 or 5 radicals R¹⁰, and a 5- or 6-membered heteroaromatic ring containing 1, 2 or 3 heteroatoms selected from N, O and S, as ring members, where the heteroaromatic ring may be substituted by one or more, e.g. by 1, 2, 3, 4 or 5, preferably 1, 2 or 3, more preferably by 1 or 2 and in particular 1, radicals R¹⁰;

where R¹⁰ has one of the meanings given above or in particular one of the preferred meanings given below.

Preferably, each R⁷ is independently selected from the group consisting of hydrogen, C₁-C₆-alkyl which may be substituted by a group —C(O)NR⁸R⁹, C₁-C₆-haloalkyl, C₃-C₈-cycloalkyl, C₃-C₈-halocycloalkyl, C₃-C₈-cycloalkyl-C₁-C₄-alkyl, phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰, where R¹⁰ has one of the meanings given above or in particular one of the preferred meanings given below.

More preferably, each R⁷ is independently selected from the group consisting of hydrogen, C₁-C₆-alkyl which may be substituted by a group —C(O)NR⁸R⁹, C₁-C₆-haloalkyl, phenyl which may be substituted by 1, 2, 3, 4 or 5 radicals R¹⁰; and a 5- or 6-membered heteroaromatic ring containing 1, 2 or 3 heteroatoms selected from N, O and S, as ring members, where the heteroaromatic ring may be substituted by one or more, e.g. 1, 2, 3 or 4, preferably 1 or 2, more preferably 1, radicals R¹⁰; where R¹⁰ has one of the meanings given above or in particular one of the preferred meanings given below.

Even more preferably, each R⁷ is independently selected from the group consisting of hydrogen, C₁-C₄-alkyl, —CH₂—C(O)NR⁸R⁹, C₁-C₃-haloalkyl, phenyl which may be substituted by 1, 2, 3, 4 or 5 radicals R¹⁰; and a 5- or 6-membered heteroaromatic ring containing 1, 2 or 3 heteroatoms selected from N, O and S, as ring members, where the heteroaromatic ring may be substituted by one or more, e.g. 1, 2, 3 or 4, preferably 1 or 2, more preferably 1, radicals R¹⁰; where R¹⁰ has one of the meanings given above or in particular one of the preferred meanings given below.

Especially in the definition of R³, R⁷ is selected from hydrogen and C₁-C₆-alkyl and preferably from hydrogen and C₁-C₄-alkyl.

R⁸ is independently of each occurrence preferably selected from the group consisting of hydrogen, C₁-C₆-alkyl, C₁-C₆-haloalkyl, C₁-C₄-alkyl substituted by a phenyl group or substituted by a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, formyl, C₁-C₆-alkylcarbonyl, C₁-C₆-haloalkylcarbonyl, C₃-C₆-cycloalkylcarbonyl, C₃-C₆-halocycloalkylcarbonyl, —C(O)—CH₂—(C₃-C₆-cycloalkyl), —C(O)—CH₂—(C₃-C₆-halocycloalkyl), C₁-C₆-alkoxycarbonyl, C₁-C₆-haloalkoxycarbonyl, —C(O)—CH₂—S(O)ₙ—(C₁-C₄-alkyl), —C(O)—CH₂—S(O)ₙ—(C₁-C₄-haloalkyl), phenylcarbonyl, —C(O)—CH₂-phenyl, —C(O)—CH₂—S(O)ₙ-phenyl, phenyl, where the phenyl moiety in the 4 last-mentioned radicals may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰.

R⁹ is independently of each occurrence preferably selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₈-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals R¹⁰.

R⁸ is independently of each occurrence more preferably selected from the group consisting of hydrogen, C₁-C₆-alkyl, C₃-C₆-cycloalkyl, C₃-C₆-halocycloalkyl, formyl, C₁-C₆-alkylcarbonyl, C₁-C₆-haloalkylcarbonyl, C₃-C₆-cycloalkylcarbonyl, C₃-C₆-halocycloalkylcarbonyl, —C(O)—CH₂—(C₃-C₆-cycloalkyl), —C(O)—CH₂—(C₃-C₆-halocycloalkyl), C₁-C₆-alkoxycarbonyl, C₁-C₆-haloalkoxycarbonyl, —C(O)—CH₂—S(O)ₙ—(C₁-C₄-alkyl), —C(O)—CH₂—S(O)ₙ—(C₁-C₄-haloalkyl), phenylcarbonyl, —C(O)—CH₂-phenyl, —C(O)—CH₂—S(O)ₙ-phenyl, phenyl, where the phenyl moieties in the 4 last-mentioned radicals may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰; where R¹⁰ has one of the meanings given above or in particular one of the preferred meanings given below.

R⁹ is independently of each occurrence more preferably selected from the group consisting of hydrogen, C₁-C₆-alkyl, C₃-C₈-cycloalkyl, C₃-C₈-halocycloalkyl, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰; where R¹⁰ has one of the meanings given above or in particular one of the preferred meanings given below.

R⁸ is independently of each occurrence even more preferably selected from the group consisting of hydrogen, C₁-C₆-alkyl, formyl, C₁-C₆-alkylcarbonyl, C₁-C₆-haloalkylcarbonyl, C₃-C₆-cycloalkylcarbonyl, C₃-C₆-halocycloalkylcarbonyl, —C(O)—CH₂—(C₃-C₆-cycloalkyl), —C(O)—CH₂—(C₃-C₆-halocycloalkyl), C₁-C₆-alkoxycarbonyl, C₁-C₆-haloalkoxycarbonyl, —C(O)—CH₂—SO₂—(C₁-C₄-alkyl), —C(O)—CH₂—SO₂—(C₁-C₄-haloalkyl), phenylcarbonyl, —C(O)—CH₂-phenyl, —C(O)—CH₂—SO₂-phenyl, phenyl which may be substituted by 1, 2, 3, 4 or 5 radicals R¹⁰, and a 5- or 6-membered heteroaromatic ring containing 1, 2 or 3 heteroatoms selected from N, O and S, as ring members, where the heteroaromatic ring may be substituted by one or more, e.g. 1, 2, 3 or 4, preferably 1 or 2, more preferably 1, radicals R¹⁰; where R¹⁰ has one of the meanings given above or in particular one of the preferred meanings given below.

R⁹ is independently of each occurrence even more preferably selected from the group consisting of hydrogen, C₁-C₆-alkyl, C₁-C₆-haloalkyl, phenyl which may be substituted by 1, 2, 3, 4 or 5 radicals R¹⁰, and a 5- or 6-membered heteroaromatic ring containing 1, 2 or 3 heteroatoms selected from N, O and S, as ring members, where the heteroaromatic ring may be substituted by one or more, e.g. 1, 2, 3 or 4, preferably 1 or 2, more preferably 1, radicals R¹⁰; where R¹⁰ has one of the meanings given above or in particular one of the preferred meanings given below.

In particular, R⁸ and R⁹ are independently of each other and independently of each occurrence selected from the group consisting of hydrogen, C₁-C₄-alkyl and C₁-C₄-haloalkyl. More particularly, R⁹, independently of each occurrence, is selected from hydrogen and C₁-C₄-alkyl, in particular from hydrogen, methyl and ethyl and specifically from hydrogen and methyl.

Especially in the definition of R³ being —C(═O)N(R⁸)R⁹, R⁸ is selected from hydrogen, C₁-C₆-alkyl, C₁-C₆-haloalkyl and C₁-C₄-alkyl substituted by a phenyl group or substituted by a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members and R⁹ has one of the general meanings given above or in particular one of the preferred meanings given above and is preferably hydrogen or C₁-C₄-alkyl and in particular hydrogen.

Preferably, each R¹⁰ is independently selected from the group consisting of halogen, cyano, C₁-C₁₀-alkyl which may be partially or fully halogenated and/or may be substituted by one or more, e.g. 1, 2, 3 or 4, preferably 1 or 2, more preferably 1, radicals R¹⁸, C₃-C₈-cycloalkyl which may be partially or fully halogenated and/or may be substituted by one or more, e.g. 1, 2, 3 or 4, preferably 1 or 2, more preferably 1, radicals R¹⁹, —OR¹⁷, —OS(O)ₙR¹⁷, —SR¹⁷, —S(O)ₘR¹⁷, —S(O)ₙN(R²⁰)R²¹, —N(R²⁰)R²¹, —C(═O)R¹⁷, —C(═O)OR¹⁷, —C(═O)N(R²⁰)R²¹, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from N, O, S, NO, SO and SO₂, as ring members, which may be substituted by one or more, e.g. 1, 2, 3 or 4, preferably 1 or 2, more preferably 1, radicals independently selected from halogen, cyano, nitro, C₁-C₆-alkyl, C₁-C₆-haloalkyl, C₁-C₆-alkoxy and C₁-C₆-haloalkoxy;

or two radicals R¹⁰ bound on adjacent atoms together form a group selected from —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, and —O(CH₂)O—, thus forming, together with the atoms to which they are bound, a 5- or 6-membered ring, where the hydrogen atoms of the above groups may be replaced by one or more, e.g. 1, 2, 3 or 4, preferably 1 or 2, more preferably 1, substituents selected from halogen, methyl, halomethyl, hydroxyl, methoxy and halomethoxy or one or more, e.g. 1 or 2, CH₂ groups of the above groups may be replaced by a C═O group,

where R¹⁷, R¹⁸, R¹⁹, R²⁰ and R²¹ have one of the general meanings given above or in particular one of the preferred meanings given below.

More preferably, each R¹⁰ is independently selected from the group consisting of halogen, cyano, C₁-C₁₀-alkyl which may be partially or fully halogenated and/or may be substituted by one or more, e.g. 1, 2, 3 or 4, preferably 1 or 2, more preferably 1, radicals R¹⁸, —OR¹⁷, —N(R²⁰)R²¹, —C(═O)R¹⁷, —C(═OR¹⁷, —C(═O)N(R²⁰)R²¹, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from N, O, S, NO, SO and SO₂, as ring members, which may be substituted by one or more, e.g. 1, 2, 3 or 4, preferably 1 or 2, more preferably 1, radicals independently selected from halogen, cyano, nitro, C₁-C₆-alkyl, C₁-C₆-haloalkyl, C₁-C₆-alkoxy and C₁-C₆-haloalkoxy;

where R¹⁷, R¹⁸, R¹⁹, R²⁰ and R²¹ have one of the general meanings given above or in particular one of the preferred meanings given below.

Even more preferably, each R¹⁰ is independently selected from the group consisting of halogen, C₁-C₄-alkyl, C₁-C₄-haloalkyl, C₁-C₄-alkoxy and C₁-C₄-haloalkoxy. In particular, each R¹⁰ is independently selected from the group consisting of halogen, C₁-C₄-alkyl and C₁-C₄-haloalkyl and is specifically halogen, more specifically chlorine.

Preferably, R¹¹ and R¹² are, independently of each other and independently of each occurrence, selected from the group consisting of hydrogen, halogen, C₁-C₆-alkyl and C₁-C₆-haloalkyl. More preferably, R¹¹ and R¹² are, independently of each other and independently of each occurrence, selected from the group consisting of hydrogen, halogen and C₁-C₆-alkyl and in particular from the group consisting of hydrogen and halogen. Specifically, they are hydrogen.

Preferably, R¹³ and R¹⁴ are, independently of each other and independently of each occurrence, selected from C₁-C₄-alkyl and are in particular methyl.

Preferably, R¹⁵ and R¹⁶ are, independently of each other and independently of each occurrence, selected from the group consisting of C₁-C₆-alkyl, C₁-C₆-haloalkyl and phenyl which may be substituted by 1, 2, 3, 4, or 5 radicals R¹⁰; where R¹⁰ has one of the general or in particular one of the preferred meanings given above.

Preferably, each R¹⁷ is independently selected from the group consisting of hydrogen, C₁-C₆-alkyl, C₁-C₆-haloalkyl, C₃-C₈-cycloalkyl, C₃-C₈-halocycloalkyl, phenyl and benzyl. More preferably, each R¹⁷ is independently selected from the group consisting of hydrogen, C₁-C₆-alkyl, C₁-C₆-haloalkyl and phenyl and is in particular C₁-C₄-alkyl or C₁-C₃-haloalkyl.

Preferably, each R¹⁸ is independently selected from the group consisting of cyano; C₃-C₈-cycloalkyl, C₃-C₈-halocycloalkyl, —OR¹⁷, —OSO₂R¹⁷, —SR¹⁷, —S(O)ₘR¹⁷, —S(O)ₙN(R²⁰)R²¹, —N(R²⁰)R²¹, —C(═O)N(R²⁰)R²¹, —C(═S)N(R²⁰)R²¹, —C(═O)OR¹⁷, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy; where R¹⁷, R²⁰ and R²¹ have one of the general or in particular one of the preferred meanings given above or below.

Preferably, each R¹⁹ is independently selected from the group consisting of cyano; C₁-C₄-alkyl, C₁-C₄-haloalkyl, —OR¹⁷, —OSO₂R¹⁷, —SR¹⁷, —S(O)ₘR¹⁷, —S(O)ₙN(R²⁰)R²¹, —N(R²⁰)R²¹, —C(═O)N(R²⁰)R²¹, —C(═S)N(R²⁰) R²¹, —C(═O)OR¹⁷, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy; where R¹⁷, R²⁰ and R²¹ have one of the general or in particular one of the preferred meanings given above or below.

Preferably, R²⁰ and R²¹ are independently of each other and independently of each occurrence selected from the group consisting of hydrogen, C₁-C₆-alkyl, C₁-C₆-haloalkyl, C₃-C₈-cycloalkyl-C₁-C₄-alkyl, C₃-C₈-halocycloalkyl-C₁-C₄-alkyl, C₂-C₆-alkenyl, C₂-C₆-haloalkenyl, C₂-C₆-alkynyl, C₂-C₆-haloalkynyl, —C(═O)N(R²²)R²³, —C(═O)R¹⁷, —C(═O)OR¹⁷, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, benzyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy; where R¹⁷, R²² and R²³ have one of the general or in particular one of the preferred meanings given above or below.

More preferably, R²⁰ and R²¹ are independently of each other and independently of each occurrence selected from the group consisting of hydrogen, C₁-C₆-alkyl, C₁-C₆-haloalkyl, C₃-C₈-cycloalkyl-methyl, —C(═O)R¹⁷, phenyl and benzyl, where R¹⁷ is as defined above or is in particular C₁-C₆-alkyl, C₁-C₆-haloalkyl, phenyl or benzyl.

Preferably, R²² and R²³ are independently of each other and independently of each occurrence selected from the group consisting of hydrogen, C₁-C₆-alkyl, C₁-C₆-haloalkyl, C₃-C₈-cycloalkyl-C₁-C₄-alkyl, C₃-C₈-halocycloalkyl-C₁-C₄-alkyl, C₂-C₆-alkenyl, C₂-C₆-haloalkenyl, C₂-C₆-alkynyl, C₂-C₆-haloalkynyl, C₁-C₄-alkylcarbonyl, C₁-C₄-haloalkylcarbonyl, phenyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, benzyl which may be substituted by 1, 2, 3, 4 or 5 radicals independently selected from halogen, cyano, nitro, C₁-C₆-alkyl, C₁-C₆-haloalkyl, C₁-C₆-alkoxy and C₁-C₆-haloalkoxy, and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more radicals independently selected from halogen, cyano, nitro, C₁-C₆-alkyl, C₁-C₆-haloalkyl, C₁-C₆-alkoxy and C₁-C₆-haloalkoxy.

More preferably, R²² and R²³ are independently of each other and independently of each occurrence selected from the group consisting of hydrogen, C₁-C₆-alkyl, C₁-C₆-haloalkyl, C₃-C₈-cycloalkyl-methyl, C₁-C₄-alkylcarbonyl, C₁-C₄-haloalkylcarbonyl, phenyl and benzyl.

One particularly preferred embodiment of the invention refers to compounds of the formula IA

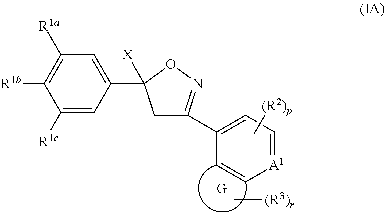

wherein
A¹, X, G, R², R³, r and p have one of the general or in particular one of the preferred meanings given above; and R^{1a}, R^{1b}, R^{1c} are hydrogen or have one of the general or in particular one of the preferred meanings given above for R¹.

Preferred compounds IA are compounds IA.1

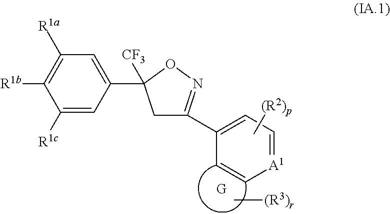

wherein
A¹ is N or CH and preferably N;
R², R³, r and p have one of the general or in particular one of the preferred meanings given above; and R^{1a}, R^{1b}, R^{1c} are hydrogen or have one of the general or in particular one of the preferred meanings given above for R¹; and
G is a condensed phenyl ring, a condensed 5-membered heteroaromatic ring containing 1 heteroatom selected from O, S and N and optionally 1 or 2 further nitrogen atoms as ring members or a condensed 6-membered heteroaromatic ring containing 1, 2 or 3 nitrogen atoms as ring members.

Preferably, in compounds IA and IA.1
R^{1a}, R^{1c} are, independently of each other, selected from the group consisting of hydrogen, halogen, cyano, nitro, SCN, SF₅, C₁-C₆-alkyl which may be partially or fully halogenated and/or may be substituted by one or more, e.g. 1, 2, 3 or 4, preferably 1 or 2, more preferably 1, radicals R⁴, C₃-C₈-cycloalkyl which may be partially or fully halogenated and/or may be substituted by one or more, e.g. 1, 2, 3 or 4, preferably 1 or 2, more preferably 1, radicals R⁵, OR⁷, SR⁷, S(O)ₘR⁷, NR⁸R⁹, —C(═O)R⁶, —C(═O)OR⁷, C(═NR⁸)R⁶, C(═S)NR⁶; and
R^{1b} is selected from the group consisting of hydrogen, halogen, cyano, nitro, —SCN, SF₅, C₁-C₆-alkyl which may be partially or fully halogenated and/or may be substituted by one or more, e.g. 1, 2, 3 or 4, preferably 1 or 2, more preferably 1, radicals R⁴, C₃-C₈-cycloalkyl which may be partially or fully halogenated and/or may be substituted by one or more, e.g. 1, 2, 3 or 4, preferably 1 or 2, more preferably 1, radicals R⁵, C₂-C₆-alkenyl which may be partially or fully halogenated and/or may be substituted by one or more, e.g. 1, 2, 3 or 4, preferably 1 or 2, more preferably 1, radicals R⁴, C₂-C₆-alkynyl which may be partially or fully halogenated and/or may be substituted by one or more, e.g. 1, 2, 3 or 4, preferably 1 or 2, more preferably 1, radicals R⁴, Si(R¹⁴)₂R¹³, OR⁷, SW, OS(O)ₙR⁷, S(O)ₘR⁷, NR⁸R⁹, N(R⁸)C(═O)R⁶, C(═O)R⁶, C(═O)OR⁷, C(═NR⁸)R⁶, C(═S)NR⁶, phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from N, O, S, NO, SO and SO₂, as ring members, where the heterocyclic ring may be substituted by one or more, e.g. 1, 2, 3 or 4, preferably 1 or 2, more preferably 1, radicals R¹⁰,
   where R⁴, R⁵, R⁶, R⁷, R⁸, R⁹, R¹⁰, R¹³ and R¹⁴ have one of the general or in particular one of the preferred meanings given above.

More preferably, in compounds IA and IA.1,
R^{1a}, R^{1b} and R^{1c} are, independently of each other, selected from the group consisting of hydrogen, halogen, cyano, C₁-C₄-alkyl, C₁-C₄-haloalkyl, C₃-C₈-cycloalkyl, C₃-C₈-halocycloalkyl, C₁-C₄-alkoxy and C₁-C₄-haloalkoxy, even more preferably from hydrogen, halogen, C₁-C₄-alkyl and C₁-C₄-haloalkyl, in particular from hydrogen, halogen, and C₁-C₃-haloalkyl.

Specifically, in compounds IA and IA.1, R^{1a}, R^{1b} and R^{1c} are, independently of each other, selected from the group consisting of hydrogen and halogen, specifically hydrogen and chlorine. Even more specifically, R^{1a} and R^{1c} are halogen, specifically chlorine, and R^{1b} is hydrogen.

Specifically, in compounds IA and IA.1,
G is a condensed phenyl ring, a condensed 5-membered heteroaromatic ring containing 1 heteroatom selected from O, S and N and optionally 1 or 2 further nitrogen atoms as ring members or a condensed 6-membered heteroaromatic ring containing 1, 2 or 3 nitrogen atoms as ring members;
X in compounds IA and IB is halomethyl and specifically CF₃;
R^{1a}, R^{1b} and R^{1c} are, independently of each other, selected from the group consisting of hydrogen, halogen, cyano, C₁-C₄-alkyl, C₁-C₄-haloalkyl, C₃-C₈-cycloalkyl, C₃-C₈-halocycloalkyl, C₁-C₄-alkoxy and C₁-C₄-haloalkoxy, preferably from hydrogen, halogen, C₁-C₄-alkyl and C₁-C₄-haloalkyl, more preferably from hydrogen, halogen, and C₁-C₃-haloalkyl, even more preferably from hydrogen and halogen, and in particular from hydrogen and chlorine;
R² is independently selected from halogen; hydroxyl, C₁-C₄-alkyl, C₁-C₄-haloalkyl, C₁-C₄-alkoxy, C₁-C₄-haloalkoxy, phenyl which may be substituted by 1, 2 or 3 radicals R¹⁰; and a 5- or 6-membered heteroaromatic ring containing 1, 2 or 3 heteroatoms selected from N, O and S as ring members, where the heteroaromatic ring may be substituted by 1 or 2 radicals R¹⁰; where R¹⁰ has one of the general or in particular one of the preferred meanings given above;
each R³ is independently selected from halogen, cyano, C₁-C₄-alkyl which may be substituted by one radical R⁴, C₁-C₄-haloalkyl, OR⁷, —C(═O)OR⁷, —C(═O)N(R⁸)R⁹, —S(O)₂R⁷, —OS(O)₂R⁷, —S(O)₂OR⁷, —S(O)₂N(R⁸)R⁹, and —C(═NR⁸)R⁶, wherein
   R⁴ is selected from cyano, —C(═O)N(R⁸)R⁹, —N(R⁸)R⁹, C₃-C₆-cyclopropyl, phenyl and a 5- or 6-membered hetaryl ring containing 1, 2 or 3 heteroatoms selected from O, S and N;
      R⁷ is hydrogen, C₁-C₄-alkyl or C₁-C₄-haloalkyl;
   R⁶ is hydrogen or C₁-C₄-alkyl;
   R⁸ in the group —C(═NR⁸)R⁶ is C₁-C₄-alkoxy or C₁-C₄-haloalkoxy; in the group —C(═O)N(R⁸)R⁹ and —S(O)₂N(R⁸)R⁹ is hydrogen, C₁-C₄-alkyl which may carry one group R⁴, C₁-C₄-haloalkyl, C₁-C₄-alkoxy or C₁-C₄-haloalkoxy, where R⁴ is phenyl or a 5- or 6-membered heteroaryl ring having 1 or 2 heteroatoms selected from O, S and N as ring members; and in the group —N(R⁸)R⁹ is hydrogen, C₁-C₄-alkyl, C₁-C₄-haloalkyl, C₁-C₄-alkoxy, C₁-C₄-haloalkoxy, formyl, C₁-C₄-alkylcarbonyl, C₁-C₄-haloalkylcarbonyl C₃-C₆-cycloalkylcarbonyl, C₃-C₄-halocycloalkylcarbonyl, —C(O)—CH₂—(C₃-C₆-cycloalkyl), —C(O)-phenyl, —C(O)—CH₂-phenyl, —C(O)—CH₂—SO₂—(C₁-C₄-alkyl), —C(O)—CH₂—SO₂—(C₁-C₄-haloalkyl), —C(O)—CH₂—S—(C₁-C₄-alkyl), —C(O)—CH₂—S—(C₁-C₄-haloalkyl), —C(O)—CH₂—SO₂-phenyl, —C(O)—CH₂—S-phenyl, C₁-C₄-alkoxycarbonyl or C₁-C₄-haloalkoxycarbonyl;
   R⁹ in the group —C(═O)N(R⁸)R⁹ is hydrogen, C₁-C₄-alkyl, C₁-C₄-haloalkyl, C₁-C₄-alkoxy or C₁-C₄-haloalkoxy; and in the group —N(R⁸)R⁹ is hydrogen, C₁-C₄-alkyl, C₁-C₄-haloalkyl, C₁-C₄-alkoxy or C₁-C₄-haloalkoxy;
      p is 0 or 1; and
      r is 0, 1 or 2.

Examples of preferred compounds are compounds of the following formulae Ia.1 and Ia.2, where the variables have one of the general or preferred meanings given above. Examples of preferred compounds are the individual compounds compiled in the tables 1 to 312000 below, where the variable Q has the meanings given below in formulae II.1 to II.132. Moreover, the meanings mentioned below for Q and for the individual variables in the tables are per se, independently of the combination in which they are mentioned, a particularly preferred embodiment of the substituents in question.

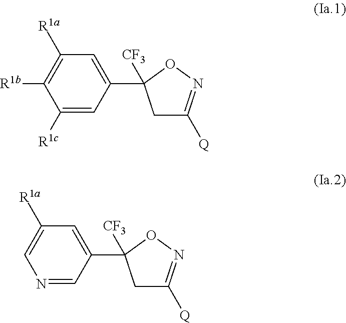

### Q:

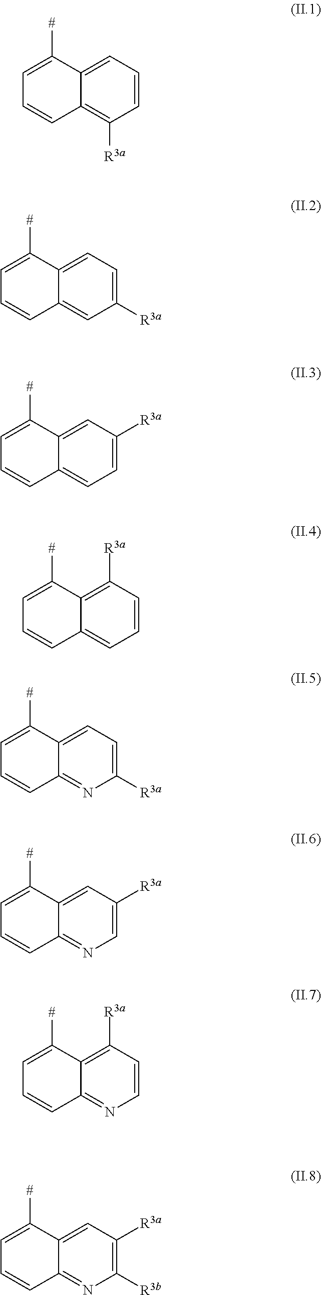

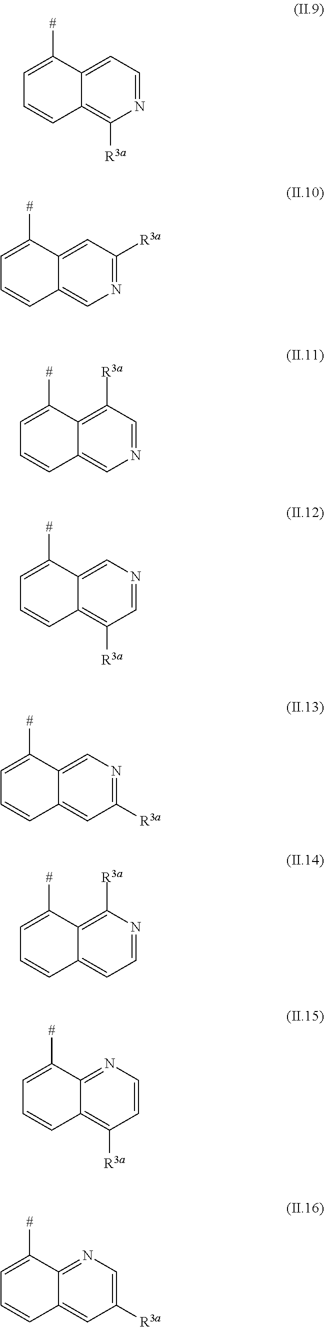

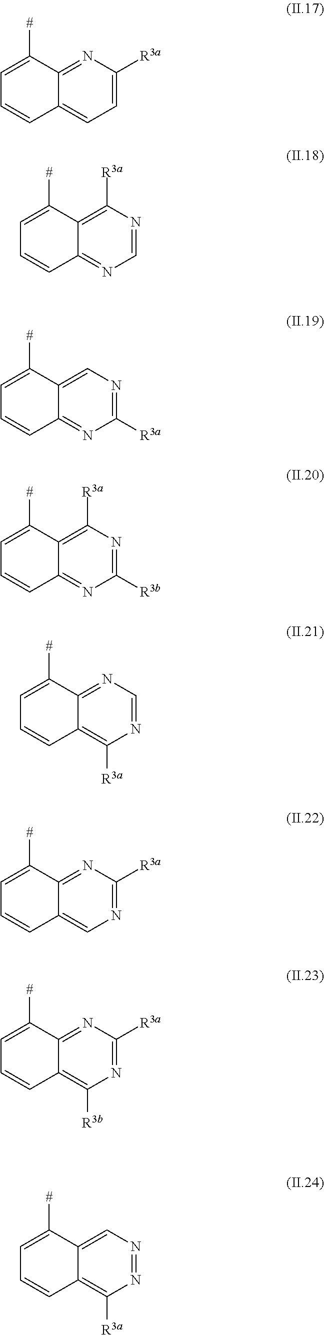

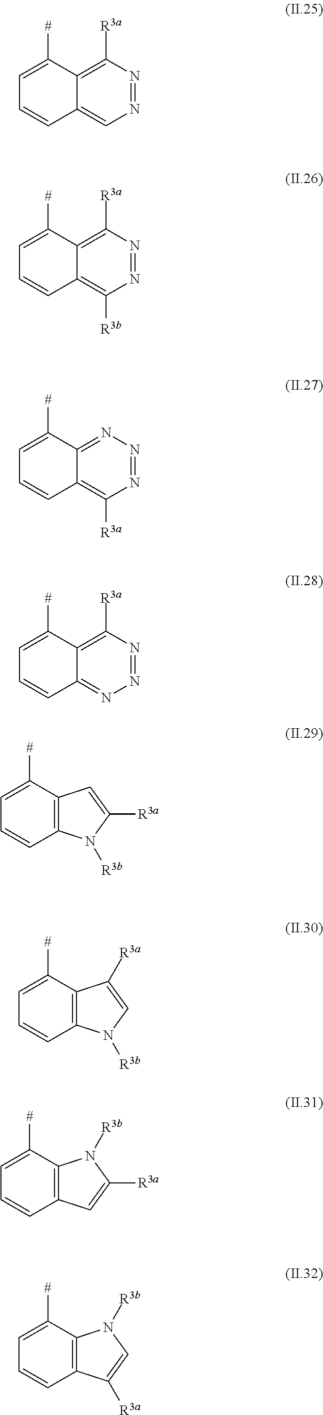

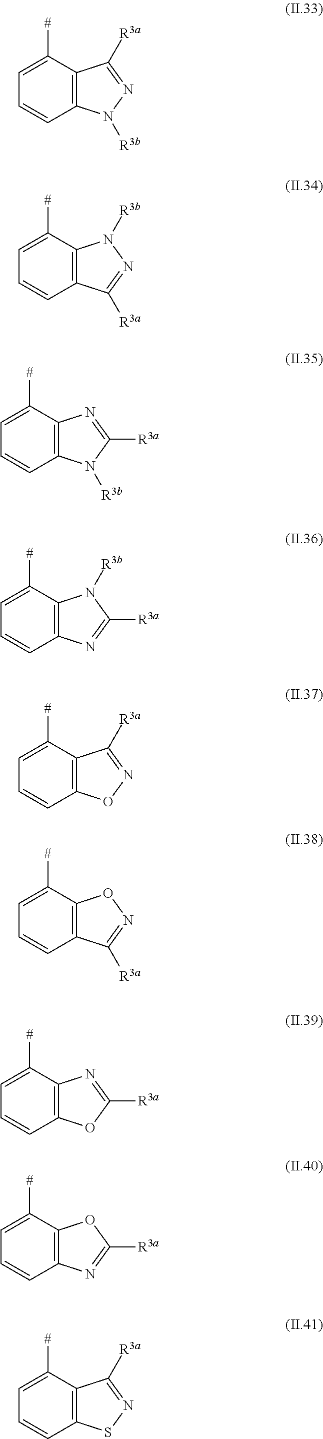

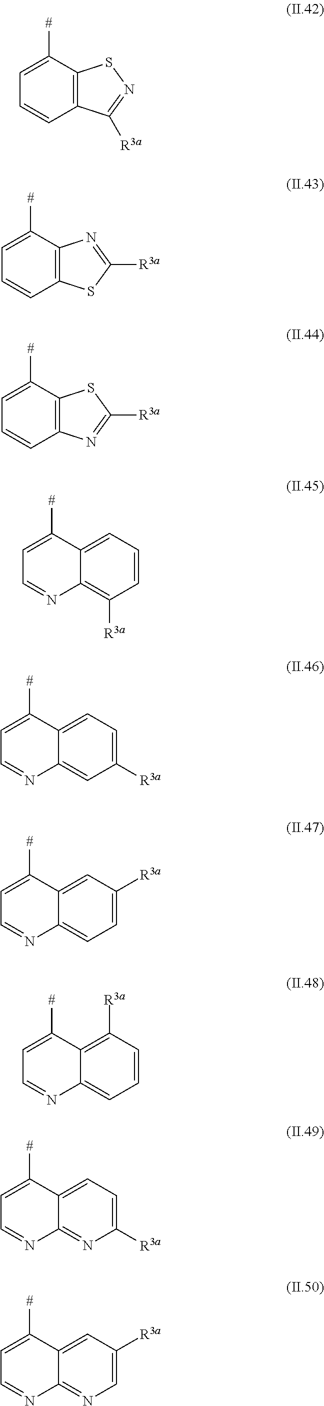

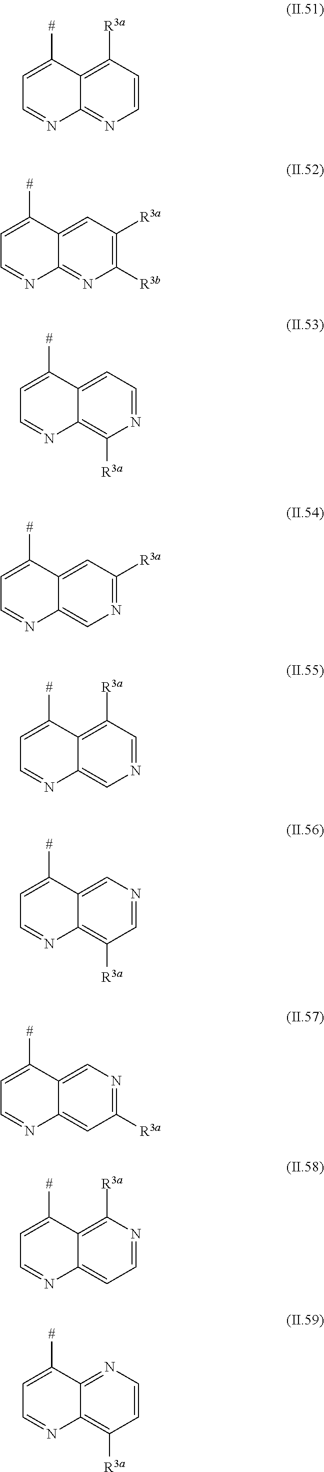

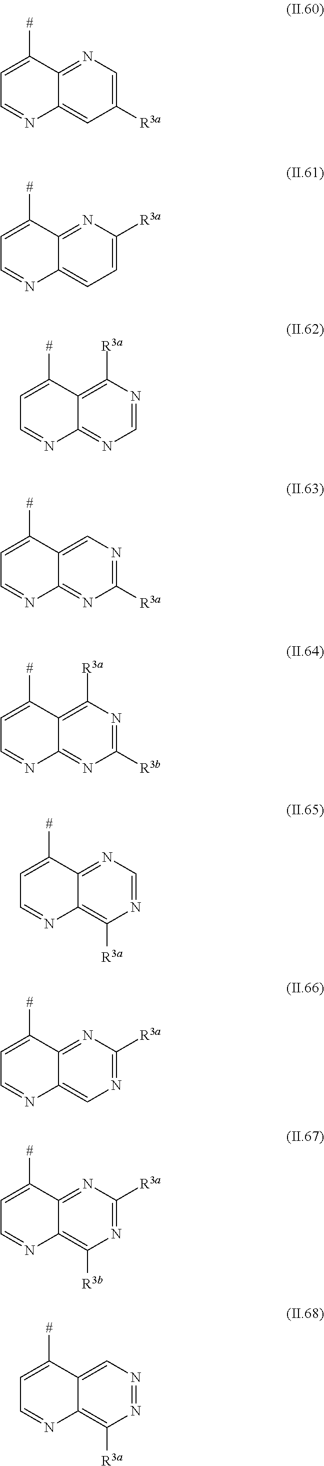

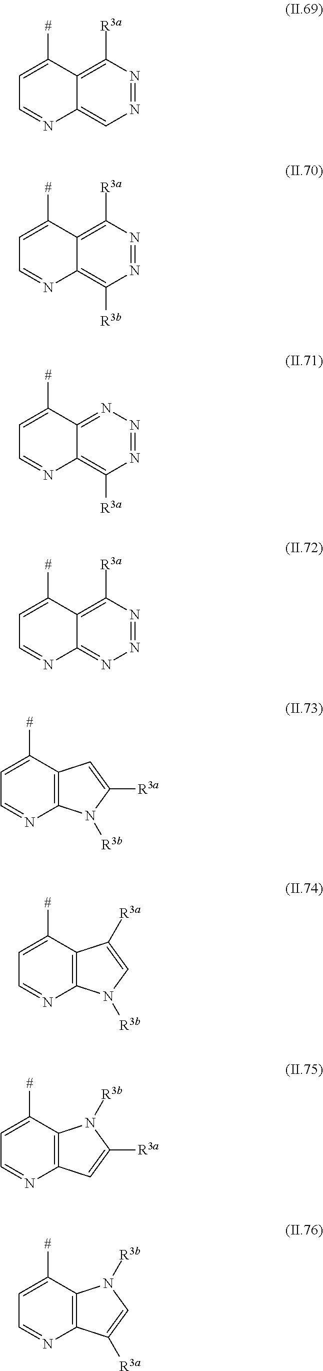

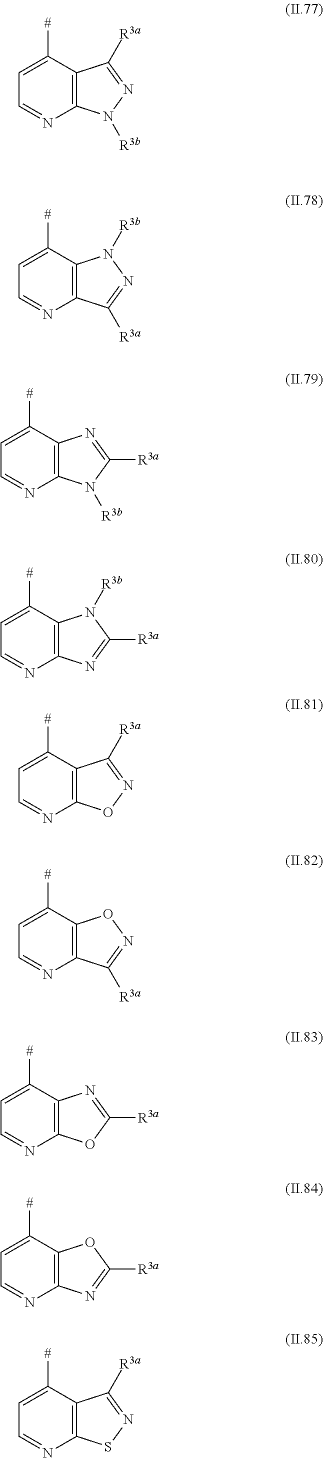

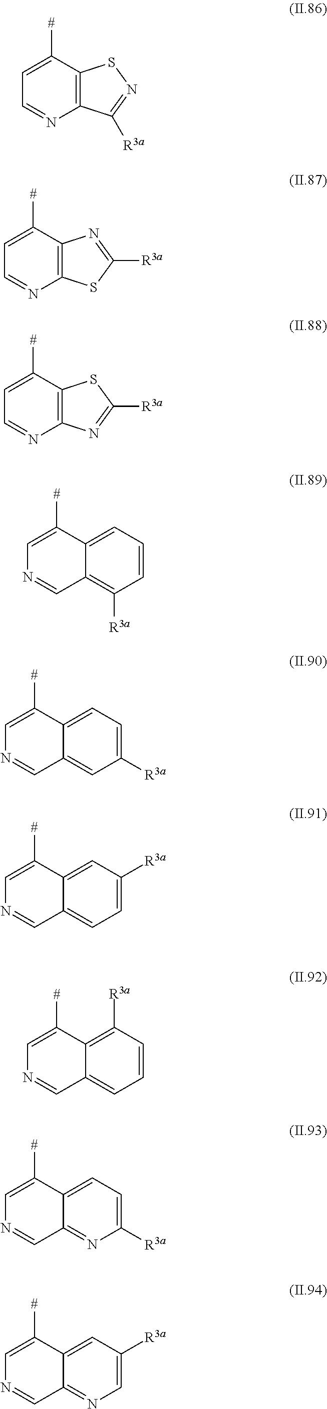

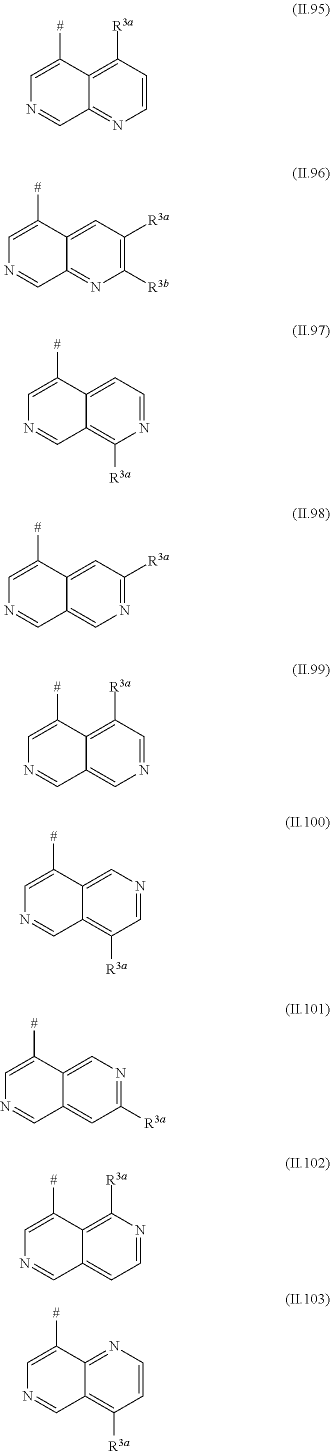

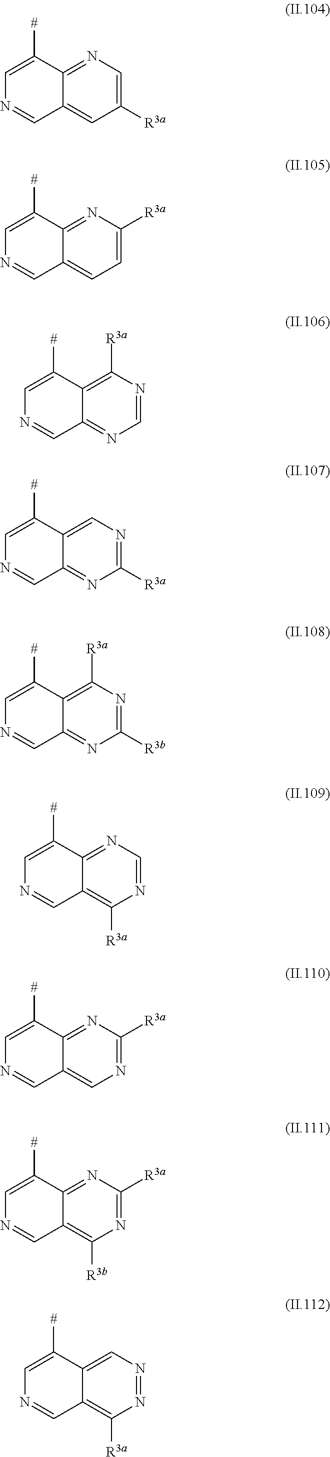

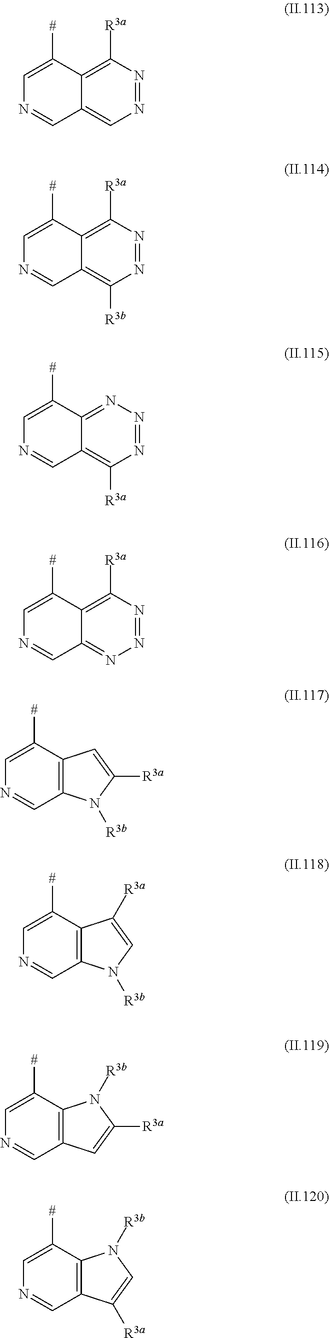

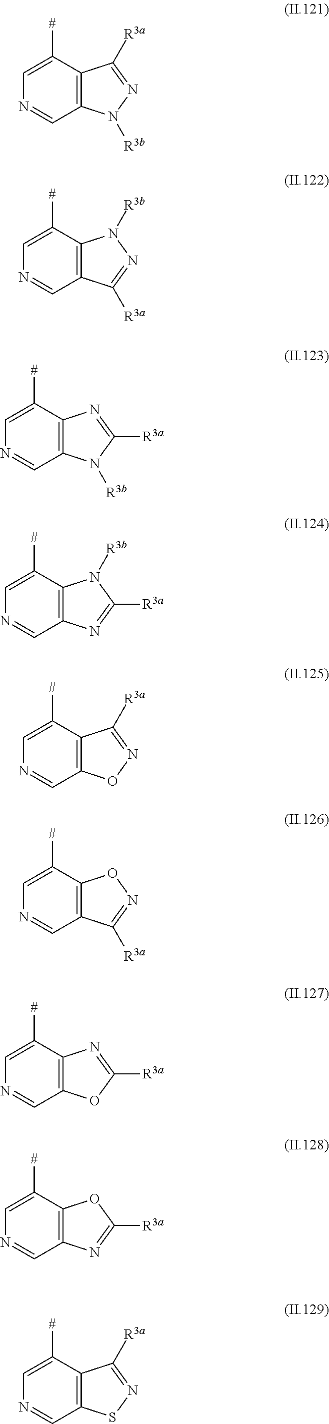

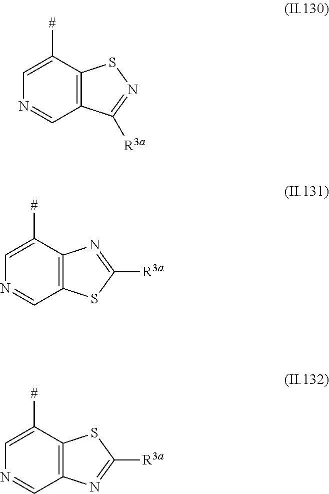

### Table 1

Compounds of the formula Ia.1 in which R^{1a} and R^{1c} are chlorine, R^{1b} is H and Q is radical of formula II.1, wherein R^{3a} is H

### Table 2

Compounds of the formula Ia.1 in which R^{1a} and R^{1c} are bromine, R^{1b} is H and Q is radical of formula II.1, wherein R^{3a} is H

### Table 3

Compounds of the formula Ia.1 in which R^{1a} and R^{1c} are fluorine, R^{1b} is H and Q is radical of formula II.1, wherein R^{3a} is H

### Table 4

Compounds of the formula Ia.1 in which R^{1a} and R^{1c} are methyl, R^{1b} is H and Q is radical of formula II.1, wherein R^{3a} is H

### Table 5

Compounds of the formula Ia.1 in which R^{1a} and R^{1c} are CF₃, R^{1b} is H and Q is radical of formula II.1, wherein R^{3a} is H

### Table 6

Compounds of the formula Ia.1 in which R^{1a} and R^{1b} are chlorine, R^{1c} is H and Q is radical of formula II.1, wherein R^{3a} is H

### Table 7

Compounds of the formula Ia.1 in which R^{1a} is CF₃ and R^{1b} and R^{1c} are H and Q is radical of formula II.1, wherein R^{3a} is H

### Table 8

Compounds of the formula Ia.1 in which R^{1a}, R^{1b} and R^{1c} are chlorine and Q is radical of formula II.1, wherein R^{3a} is H

### Table 9

Compounds of the formula Ia.1 in which R^{1a}, R^{1b} and R^{1c} are fluorine and Q is radical of formula II.1, wherein R^{3a} is H

### Table 10

Compounds of the formula Ia.1 in which R^{1a}, R^{1b} and R^{1c} are methyl and Q is radical of formula II.1, wherein R^{3a} is H

### Tables 11 to 20

Compounds of the formula Ia.1 in which the combination of R^{1a}R^{1b} and R^{1c} is as defined in any of Tables 1 to 10 and Q is radical of formula II.1, wherein R^{3a} is F

### Tables 21 to 30

Compounds of the formula Ia.1 in which the combination of R^{1a}, Rib and R^{1c} is as defined in any of Tables 1 to 10 and Q is radical of formula II.1, wherein R^{3a} is Cl

### Tables 31 to 40

Compounds of the formula Ia.1 in which the combination of R^{1a}, R^{1b} and R^{1c} is as defined in any of Tables 1 to 10 and Q is radical of formula II.1, wherein R^{3a} is Br

### Tables 41 to 50

Compounds of the formula Ia.1 in which the combination of R^{1a}, R^{1b} and R^{1c} is as defined in any of Tables 1 to 10 and Q is radical of formula II.1, wherein R^{3a} is I

### Tables 51 to 60

Compounds of the formula Ia.1 in which the combination of R^{1a}, R^{1b} and R^{1c} is as defined in any of Tables 1 to 10 and Q is radical of formula II.1, wherein R^{3a} is CN

### Tables 61 to 70

Compounds of the formula Ia.1 in which the combination of R^{1a}, Rib and R^{1c} is as defined in any of Tables 1 to 10 and Q is radical of formula II.1, wherein R^{1a} is methyl

### Tables 71 to 80

Compounds of the formula Ia.1 in which the combination of R^{1a}, R^{1b} and R^{1c} is as defined in any of Tables 1 to 10 and Q is radical of formula II.1, wherein R^{3a} is ethyl

### Tables 81 to 90

Compounds of the formula Ia.1 in which the combination of R^{1a}, R^{1b} and R^{1c} is as defined in any of Tables 1 to 10 and Q is radical of formula II.1, wherein R^{3a} is n-propyl

### Tables 91 to 100

Compounds of the formula Ia.1 in which the combination of R^{1a}, R^{1b} and R^{1c} is as defined in any of Tables 1 to 10 and Q is radical of formula II.1, wherein R^{3a} is isopropyl

### Tables 101 to 110

Compounds of the formula Ia.1 in which the combination of R^{1a}, R^{1b} and R^{1c} is as defined in any of Tables 1 to 10 and Q is radical of formula II.1, wherein R^{3a} is —CH₂-cylopropyl

### Tables 111 to 120

Compounds of the formula Ia.1 in which the combination of R^{1a}, R^{1b} and R^{1c} is as defined in any of Tables 1 to 10 and Q is radical of formula II.1, wherein R^{3a} is —CH₂-cyloprop-2-yl-phenyl

### Tables 121 to 130

Compounds of the formula Ia.1 in which the combination of R^{1a}, R^{1b} and R^{1c} is as defined in any of Tables 1 to 10 and Q is radical of formula II.1, wherein R^{3a} is —CH₂-phenyl

### Tables 131 to 140

Compounds of the formula Ia.1 in which the combination of R^{1a}, R^{1b} and R^{1c} is as defined in any of Tables 1 to 10 and Q is radical of formula II.1, wherein R^{3a} is —CH₂-pyridin-2-yl

### Tables 141 to 150

Compounds of the formula Ia.1 in which the combination of R^{1a}, R^{1b} and R^{1c} is as defined in any of Tables 1 to 10 and Q is radical of formula II.1, wherein R^{3a} is —CF₃

### Tables 151 to 160

Compounds of the formula Ia.1 in which the combination of R^{1a}, R^{1b} and R^{1c} is as defined in any of Tables 1 to 10 and Q is radical of formula II.1, wherein R^{3a} is —CH₂CF₃

### Tables 161 to 170

Compounds of the formula Ia.1 in which the combination of R^{1a}, R^{1b} and R^{1c} is as defined in any of Tables 1 to 10 and Q is radical of formula II.1, wherein R^{3a} is —CH₂CH₂CF₃

### Tables 171 to 180

Compounds of the formula Ia.1 in which the combination of R^{1a}, R^{1b} and R^{1c} is as defined in any of Tables 1 to 10 and Q is radical of formula II.1, wherein R^{3a} is methoxy

### Tables 181 to 190

Compounds of the formula Ia.1 in which the combination of R^{1a}, R^{1b} and R^{1c} is as defined in any of Tables 1 to 10 and Q is radical of formula II.1, wherein R^{1a} is ethoxy

### Tables 191 to 200

Compounds of the formula Ia.1 in which the combination of R^{1a}, R^{1b} and R^{1c} is as defined in any of Tables 1 to 10 and Q is radical of formula II.1, wherein R³⁸ is propoxy

### Tables 201 to 210

Compounds of the formula Ia.1 in which the combination of R^{1a}, R^{1b} and R^{1c} is as defined in any of Tables 1 to 10 and Q is radical of formula II.1, wherein R^{3a} is isopropoxy

### Tables 211 to 220

Compounds of the formula Ia.1 in which the combination of R^{1a}, R^{1b} and R^{1c} is as defined in any of Tables 1 to 10 and Q is radical of formula II.1, wherein R^{3a} is difluoromethoxy

### Tables 221 to 230

Compounds of the formula Ia.1 in which the combination of R^{1a}, R^{1b} and R^{1c} is as defined in any of Tables 1 to 10 and Q is radical of formula II.1, wherein R^{3a} is trifluoromethoxy

### Tables 231 to 240

Compounds of the formula Ia.1 in which the combination of R^{1a}, R^{1b} and R^{1c} is as defined in any of Tables 1 to 10 and Q is radical of formula II.1, wherein R³⁸ is —O—CH₂—C(O)—NH₂

### Tables 241 to 250

Compounds of the formula Ia.1 in which the combination of R^{1a}, R^{1b} and R^{1c} is as defined in any of Tables 1 to 10 and Q is radical of formula II.1, wherein R³⁸ is —O—CH₂—C(O)—NHCH₃

### Tables 251 to 260

Compounds of the formula Ia.1 in which the combination of R^{1a}, R^{1b} and R^{1c} is as defined in any of Tables 1 to 10 and Q is radical of formula II.1, wherein R^{3a} is —O—CH₂—C(O)—N(CH₃)₂

### Tables 261 to 270

Compounds of the formula Ia.1 in which the combination of R^{1a}, R^{1b} and R^{1c} is as defined in any of Tables 1 to 10 and Q is radical of formula II.1, wherein R^{1a} is —O—CH₂—C(O)—NHCH₂CH₃

### Tables 271 to 280

Compounds of the formula Ia.1 in which the combination of R^{1a}, R^{1b} and R^{1c} is as defined in any of Tables 1 to 10 and Q is radical of formula II.1, wherein R^{3a} is —O—CH₂—C(O)—N(CH₂CH₃)₂

### Tables 281 to 290

Compounds of the formula Ia.1 in which the combination of R^{1a}, R^{1b} and R^{1c} is as defined in any of Tables 1 to 10 and Q is radical of formula II.1, wherein R^{3a} is —O—CH₂—C(O)—NHCF₃

### Tables 291 to 300

Compounds of the formula Ia.1 in which the combination of R^{1a}, R^{1b} and R^{1c} is as defined in any of Tables 1 to 10 and Q is radical of formula II.1, wherein R^{1a} is —O—CH₂—C(O)—N(CF₃)₂

### Tables 301 to 310

Compounds of the formula Ia.1 in which the combination of R^{1a}, R^{1b} and R^{1c} is as defined in any of Tables 1 to 10 and Q is radical of formula II.1, wherein R^{3a} is —O—CH₂—C(O)—NHCH₂CF₃

### Tables 311 to 320

Compounds of the formula Ia.1 in which the combination of R^{1a}, R^{1b} and R^{1c} is as defined in any of Tables 1 to 10 and Q is radical of formula II.1, wherein R^{3a} is —O—CH₂—C(O)—N(CH₂CF₃)₂

### Tables 321 to 330

Compounds of the formula Ia.1 in which the combination of R^{1a}, R^{1b} and R^{1c} is as defined in any of Tables 1 to 10 and Q is radical of formula II.1, wherein R^{1a} is —O—CH₂—C(O)—NH(CH₂-cyclopropyl)

### Tables 331 to 340

Compounds of the formula Ia.1 in which the combination of R^{1a}, R^{1b} and R^{1c} is as defined in any of Tables 1 to 10 and Q is radical of formula II.1, wherein R^{3a} is —C(═NOCH₃)H

### Tables 341 to 350

Compounds of the formula Ia.1 in which the combination of R^{1a}, R^{1b} and R^{1c} is as defined in any of Tables 1 to 10 and Q is radical of formula II.1, wherein R^{1a} is —C(═NOCH₃)CH₃

### Tables 351 to 360

Compounds of the formula Ia.1 in which the combination of R^{1a}, R^{1b} and R^{1c} is as defined in any of Tables 1 to 10 and Q is radical of formula II.1, wherein R^{1a} is —C(═NOCH₂CH₃)H

### Tables 361 to 370

Compounds of the formula Ia.1 in which the combination of R^{1a}, R^{1b} and R^{1c} is as defined in any of Tables 1 to 10 and Q is radical of formula II.1, wherein R^{1a} is —C(═NOCH₂CH₃)CH₃

### Tables 371 to 380

Compounds of the formula Ia.1 in which the combination of R^{1a}, R^{1b} and R^{1c} is as defined in any of Tables 1 to 10 and Q is radical of formula II.1, wherein R^{3a} is —C(═NOCF₃)H

### Tables 381 to 390

Compounds of the formula Ia.1 in which the combination of R^{1a}, R^{1b} and R^{1c} is as defined in any of Tables 1 to 10 and Q is radical of formula II.1, wherein R^{1a} is —C(═NOCF₃)CH₃

### Tables 391 to 400

Compounds of the formula Ia.1 in which the combination of R^{1a}, R^{1b} and R^{1c} is as defined in any of Tables 1 to 10 and Q is radical of formula II.1, wherein R^{3a} is —C(═NOCH₂CF₃)H

### Tables 401 to 410

Compounds of the formula Ia.1 in which the combination of R^{1a}, R^{1b} and R^{1c} is as defined in any of Tables 1 to 10 and Q is radical of formula II.1, wherein R^{1a} is —C(═NOCH₂CF₃)CH₃

### Tables 411 to 420

Compounds of the formula Ia.1 in which the combination of R^{1a}, R^{1b} and R^{1c} is as defined in any of Tables 1 to 10 and Q is radical of formula II.1, wherein R^{1a} is —CH₂NH₂

### Tables 421 to 430

Compounds of the formula Ia.1 in which the combination of R^{1a}, R^{1b} and R^{1c} is as defined in any of Tables 1 to 10 and Q is radical of formula II.1, wherein R^{3a} is —CH₂NHCH₃

### Tables 431 to 440

Compounds of the formula Ia.1 in which the combination of R^{1a}, R^{1b} and R^{1c} is as defined in any of Tables 1 to 10 and Q is radical of formula II.1, wherein R^{3a} is —CH₂N(CH₃)₂

### Tables 441 to 450

Compounds of the formula Ia.1 in which the combination of R^{1a}, R^{1b} and R^{1c} is as defined in any of Tables 1 to 10 and Q is radical of formula II.1, wherein R^{1a} is —CH₂NH—C(O)—CH₃

### Tables 451 to 460

Compounds of the formula Ia.1 in which the combination of R^{1a}, R^{1b} and R^{1c} is as defined in any of Tables 1 to 10 and Q is radical of formula II.1, wherein R^{3a} is —CH₂NH—C(O)—CH₂CH₃

### Tables 461 to 470

Compounds of the formula Ia.1 in which the combination of R^{1a}, R^{1b} and R^{1c} is as defined in any of Tables 1 to 10 and Q is radical of formula II.1, wherein R^{1a} is —CH₂NH—C(O)—CH₂CH₂CH₃

### Tables 471 to 480

Compounds of the formula Ia.1 in which the combination of R^{1a}, R^{1b} and R^{1c} is as defined in any of Tables 1 to 10 and Q is radical of formula II.1, wherein R^{1a} is —CH₂NH—C(O)—CH(CH₃)₂

### Tables 481 to 490

Compounds of the formula Ia.1 in which the combination of R^{1a}, R^{1b} and R^{1c} is as defined in any of Tables 1 to 10 and Q is radical of formula II.1, wherein R^{3a} is —CH₂NH—C(O)—CH₂CH₂CH₂CH₃

### Tables 491 to 500

Compounds of the formula Ia.1 in which the combination of R^{1a}, R^{1b} and R^{1c} is as defined in any of Tables 1 to 10 and Q is radical of formula II.1, wherein R^{1a} is —CH₂NH—C(O)—C(CH₃)₃

### Tables 501 to 510

Compounds of the formula Ia.1 in which the combination of R^{1a}, R^{1b} and R^{1c} is as defined in any of Tables 1 to 10 and Q is radical of formula II.1, wherein R^{3a} is —CH₂NH—C(O)—CH₂CF₃

### Tables 511 to 520

Compounds of the formula Ia.1 in which the combination of R^{1a}, R^{1b} and R^{1c} is as defined in any of Tables 1 to 10 and Q is radical of formula II.1, wherein R^{3a} is —CH₂NH—C(O)—CH₂SO₂CH₃

### Tables 521 to 530

Compounds of the formula Ia.1 in which the combination of R^{1a}, R^{1b} and R^{1c} is as defined in any of Tables 1 to 10 and Q is radical of formula II.1, wherein R^{1a} is —CH₂NH—C(O)—CH₂SCF₃

### Tables 531 to 540

Compounds of the formula in which the combination of R^{1a}, R^{1b} and R^{1c} is as defined in any of Tables 1 to 10 and Q is radical of formula II.1, wherein R^{3a} is —CH₂NH—C(O)-cyclopropyl

### Tables 541 to 550

Compounds of the formula Ia.1 in which the combination of R^{1a}, R^{1b} and R^{1c} is as defined in any of Tables 1 to 10 and Q is radical of formula II.1, wherein R^{3a} is —CH₂NH—C(O)—CH₂-cyclopropyl

### Tables 551 to 560

Compounds of the formula Ia.1 in which the combination of R^{1a}, R^{1b} and R^{1c} is as defined in any of Tables 1 to 10 and Q is radical of formula II.1, wherein R^{3a} is —CH₂NH—C(O)-phenyl

### Tables 561 to 570

Compounds of the formula Ia.1 in which the combination of R^{1a}, R^{1b} and R^{1c} is as defined in any of Tables 1 to 10 and Q is radical of formula II.1, wherein R^{3a} is —CH₂NH—C(O)—CH₂-phenyl

### Tables 571 to 580

Compounds of the formula Ia.1 in which the combination of R^{1a}, R^{1b} and R^{1c} is as defined in any of Tables 1 to 10 and Q is radical of formula II.1, wherein R^{3a} is —CH₂NH—C(O)—OCH₃

### Tables 581 to 590

Compounds of the formula Ia.1 in which the combination of R^{1a}, R^{1b} and R^{1c} is as defined in any of Tables 1 to 10 and Q is radical of formula II.1, wherein R^{3a} is OH

### Tables 591 to 600

Compounds of the formula Ia.1 in which the combination of R^{1a}, R^{1b} and R^{1c} is as defined in any of Tables 1 to 10 and Q is radical of formula II.1, wherein R^{3a} is COOH

### Tables 601 to 610

Compounds of the formula Ia.1 in which the combination of R^{1a}, R^{1b} and R^{1c} is as defined in any of Tables 1 to 10 and Q is radical of formula II.1, wherein R^{3a} is COOCH₃

### Tables 611 to 620

Compounds of the formula Ia.1 in which the combination of R^{1a}, R^{1b} and R^{1c} is as defined in any of Tables 1 to 10 and Q is radical of formula II.1, wherein R^{3a} is COOCH₂CH₃

### Tables 621 to 630

Compounds of the formula Ia.1 in which the combination of R^{1a}, R^{1b} and R^{1c} is as defined in any of Tables 1 to 10 and Q is radical of formula II.1, wherein R^{3a} is COOCH₂CH₂CH₃

### Tables 631 to 640

Compounds of the formula Ia.1 in which the combination of R^{1a}, R^{1b} and R^{1c} is as defined in any of Tables 1 to 10 and Q is radical of formula II.1, wherein R^{3a} is CONH₂

### Tables 641 to 650

Compounds of the formula Ia.1 in which the combination of R^{1a}, R^{1b} and R^{1c} is as defined in any of Tables 1 to 10 and Q is radical of formula II.1, wherein R^{3a} is CONHCH₃

### Tables 651 to 660

Compounds of the formula Ia.1 in which the combination of R^{1a}, R^{1b} and R^{1c} is as defined in any of Tables 1 to 10 and Q is radical of formula II.1, wherein R^{3a} is CON(CH₃)₂

### Tables 661 to 670

Compounds of the formula Ia.1 in which the combination of R^{1a}, R^{1b} and R^{1c} is as defined in any of Tables 1 to 10 and Q is radical of formula II.1, wherein R^{3a} is CONHCH₂CH₃

### Tables 671 to 680

Compounds of the formula Ia.1 in which the combination of R^{1a}, R^{1b} and R^{1c} is as defined in any of Tables 1 to 10 and Q is radical of formula II.1, wherein R^{3a} is CON(CH₂CH₃)₂

### Tables 681 to 690

Compounds of the formula Ia.1 in which the combination of R^{1a}, R^{1b} and R^{1c} is as defined in any of Tables 1 to 10 and Q is radical of formula II.1, wherein R^{3a} is CONHCF₃

### Tables 691 to 700

Compounds of the formula Ia.1 in which the combination of R^{1a}, R^{1b} and R^{1c} is as defined in any of Tables 1 to 10 and Q is radical of formula II.1, wherein R^{3a} is CON(CF₃)₂

### Tables 701 to 710

Compounds of the formula Ia.1 in which the combination of R^{1a}, R^{1b} and R^{1c} is as defined in any of Tables 1 to 10 and Q is radical of formula II.1, wherein R^{3a} is CONHCH₂CF₃

### Tables 711 to 720

Compounds of the formula Ia.1 in which the combination of R^{1a}, R^{1b} and R^{1c} is as defined in any of Tables 1 to 10 and Q is radical of formula II.1, wherein R^{3a} is CON(CH₂CF₃)₂

### Tables 721 to 730

Compounds of the formula Ia.1 in which the combination of R^{1a}, R^{1b} and R^{1c} is as defined in any of Tables 1 to 10 and Q is radical of formula II.1, wherein R^{3a} is CONH-Phenyl

### Tables 731 to 740

Compounds of the formula Ia.1 in which the combination of R^{1a}, R^{1b} and R^{1c} is as defined in any of Tables 1 to 10 and Q is radical of formula II.1, wherein R^{3a} is CONH-Benzyl

### Tables 741 to 750

Compounds of the formula Ia.1 in which the combination of R^{1a}, R^{1b} and R^{1c} is as defined in any of Tables 1 to 10 and Q is radical of formula II.1, wherein R^{1a} is CONH-2-Pyridyl

### Tables 751 to 760

Compounds of the formula Ia.1 in which the combination of R^{1a}, R^{1b} and R^{1c} is as defined in any of Tables 1 to 10 and Q is radical of formula II.1, wherein R^{3a} is SO₂—CH₆

### Tables 761 to 770

Compounds of the formula Ia.1 in which the combination of R^{1a}, R^{1b} and R^{1c} is as defined in any of Tables 1 to 10 and Q is radical of formula II.1, wherein R^{3a} is SO₂—CH₂CH₃

### Tables 771 to 780

Compounds of the formula Ia.1 in which the combination of R^{1a}, R^{1b} and R^{1c} is as defined in any of Tables 1 to 10 and Q is radical of formula II.1, wherein R^{3a} is SO₂—CF₃

### Tables 781 to 790

Compounds of the formula Ia.1 in which the combination of R^{1a}, R^{1b} and R^{1c} is as defined in any of Tables 1 to 10 and Q is radical of formula II.1, wherein R^{3a} is SO₂-Phenyl

### Tables 791 to 800

Compounds of the formula Ia.1 in which the combination of R^{1a}, R^{1b} and R^{1c} is as defined in any of Tables 1 to 10 and Q is radical of formula II.1, wherein R^{3a} is SO₂-4-Tolyl

### Tables 801 to 810

Compounds of the formula Ia.1 in which the combination of R^{1a}, R^{1b} and R^{1c} is as defined in any of Tables 1 to 10 and Q is radical of formula II.1, wherein R^{3a} is —OSO₂—CH₃

### Tables 811 to 820

Compounds of the formula Ia.1 in which the combination of R^{1a}, R^{1b} and R^{1c} is as defined in any of Tables 1 to 10 and Q is radical of formula II.1, wherein R^{3a} is —OSO₂—CH₂CH₃

### Tables 821 to 830

Compounds of the formula Ia.1 in which the combination of R^{1a}, R^{1b} and R^{1c} is as defined in any of Tables 1 to 10 and Q is radical of formula II.1, wherein R^{3a} is —OSO₂—CF₃

### Tables 831 to 840

Compounds of the formula Ia.1 in which the combination of R^{1a}, R^{1b} and R^{1c} is as defined in any of Tables 1 to 10 and Q is radical of formula II.1, wherein R^{3a} is —OSO₂-Phenyl

### Tables 841 to 850

Compounds of the formula Ia.1 in which the combination of R^{1a}, R^{1b} and R^{1c} is as defined in any of Tables 1 to 10 and Q is radical of formula II.1, wherein R^{3a} is —OSO₂-4-Tolyl

### Tables 851 to 860

Compounds of the formula Ia.1 in which the combination of R^{1a}, R^{1b} and R^{1c} is as defined in any of Tables 1 to 10 and Q is radical of formula II.1, wherein R^{3a} is —SO₂O—CH₃

### Tables 861 to 870

Compounds of the formula Ia.1 in which the combination of R^{1a}, R^{1b} and R^{1c} is as defined in any of Tables 1 to 10 and Q is radical of formula II.1, wherein R^{3a} is —SO₂O—CH₂CH₃

### Tables 871 to 880

Compounds of the formula Ia.1 in which the combination of R^{1a}, R^{1b} and R^{1c} is as defined in any of Tables 1 to 10 and Q is radical of formula II.1, wherein R^{3a} is —OSO₂O—CF₃

### Tables 881 to 890

Compounds of the formula Ia.1 in which the combination of R^{1a}, R^{1b} and R^{1c} is as defined in any of Tables 1 to 10 and Q is radical of formula II.1, wherein R^{3a} is —OSO₂O-Phenyl

### Tables 891 to 900

Compounds of the formula Ia.1 in which the combination of R^{1a}, R^{1b} and R^{1c} is as defined in any of Tables 1 to 10 and Q is radical of formula II.1, wherein R^{3a} is —OSO₂O-4-Tolyl

### Tables 901 to 910

Compounds of the formula Ia.1 in which the combination of R^{1a}, R^{1b} and R^{1c} is as defined in any of Tables 1 to 10 and Q is radical of formula II.1, wherein R^{1a} is SO₂—NH₂

### Tables 911 to 920

Compounds of the formula Ia.1 in which the combination of R^{1a}, R^{1b} and R^{1c} is as defined in any of Tables 1 to 10 and Q is radical of formula II.1, wherein R^{1a} is SO₂—NHCH₃

### Tables 921 to 930

Compounds of the formula Ia.1 in which the combination of R^{1a}, R^{1b} and R^{1c} is as defined in any of Tables 1 to 10 and Q is radical of formula II.1, wherein R^{3a} is SO₂—N(CH₃)₂

### Tables 931 to 940

Compounds of the formula Ia.1 in which the combination of R^{1a}, R^{1b} and R^{1c} is as defined in any of Tables 1 to 10 and Q is radical of formula II.1, wherein R^{3a} is SO₂—NHCH₂CH₃

### Tables 941 to 950

Compounds of the formula Ia.1 in which the combination of R^{1a}, R^{1b} and R^{1c} is as defined in any of Tables 1 to 10 and Q is radical of formula II.1, wherein R^{3a} is SO₂—NH(CH₂CH₃)₂

### Tables 951 to 960

Compounds of the formula Ia.1 in which the combination of R^{1a}, R^{1b} and R^{1c} is as defined in any of Tables 1 to 10 and Q is radical of formula II.1, wherein R^{3a} is SO₂—NHCF₃

### Tables 961 to 970

Compounds of the formula Ia.1 in which the combination of R^{1a}, R^{1b} and R^{1c} is as defined in any of Tables 1 to 10 and Q is radical of formula II.1, wherein R^{1a} is SO₂—N(CF₃)₂

### Tables 971 to 980

Compounds of the formula Ia.1 in which the combination of R^{1a}, R^{1b} and R^{1c} is as defined in any of Tables 1 to 10 and Q is radical of formula II.1, wherein R^{3a} is SO₂—NHCH₂CF₃

### Tables 981 to 990

Compounds of the formula Ia.1 in which the combination of R^{1a}, R^{1b} and R^{1c} is as defined in any of Tables 1 to 10 and Q is radical of formula II.1, wherein R^{3a} is SO₂—NH(CH₂CF₃)₂

### Tables 991 to 1000

Compounds of the formula Ia.1 in which the combination of R^{1a}, R^{1b} and R^{1c} is as defined in any of Tables 1 to 10 and Q is radical of formula II.1, wherein R^{3a} is SO₂—NH-Phenyl

### Tables 1001 to 2000

Compounds of the formula Ia.1 in which the combination of R^{1a}, R^{1b}, R^{1c} and R^{3a} is as defined in any of Tables 1 to 1000 and Q is radical of formula II.2

### Tables 2001 to 3000

Compounds of the formula Ia.1 in which the combination of R^{1a}, R^{1b}, R^{1c} and R^{3a} is as defined in any of Tables 1 to 1000 and Q is radical of formula II.3

### Tables 3001 to 4000

Compounds of the formula Ia.1 in which the combination of R^{1a}, R^{1b}, R^{1c} and R^{3a} is as defined in any of Tables 1 to 1000 and Q is radical of formula II.4

### Tables 4001 to 5000

Compounds of the formula Ia.1 in which the combination of R^{1a}, R^{1b}, R^{1c} and R^{3a} is as defined in any of Tables 1 to 1000 and Q is radical of formula II.5

### Tables 5001 to 6000

Compounds of the formula Ia.1 in which the combination of R^{1a}, R^{1b}, R^{1c} and R^{3a} is as defined in any of Tables 1 to 1000 and Q is radical of formula II.6

### Tables 6001 to 7000

Compounds of the formula Ia.1 in which the combination of R^{1a}, R^{1b}, R^{1c} and R^{3a} is as defined in any of Tables 1 to 1000 and Q is radical of formula II.7

### Tables 7001 to 8000

Compounds of the formula Ia.1 in which the combination of R^{1a}, R^{1b}, R^{1c} and R^{3a} is as defined in any of Tables 1 to 1000 and Q is radical of formula II.8, wherein R^{3b} is Cl

### Tables 8001 to 9000

Compounds of the formula Ia.1 in which the combination of R^{1a}, R^{1b}, R^{1c} and R^{3a} is as defined in any of Tables 1 to 1000 and Q is radical of formula II.8, wherein R^{3b} is CN

### Tables 9001 to 10000

Compounds of the formula Ia.1 in which the combination of R^{1a}, R^{1b}, R^{1c} and R^{3a} is as defined in any of Tables 1 to 1000 and Q is radical of formula II.8, wherein R^{3b} is OH

### Tables 10001 to 11000

Compounds of the formula Ia.1 in which the combination of R^{1a}, R^{1b}, R^{1c} and R^{3a} is as defined in any of Tables 1 to 1000 and Q is radical of formula II.8, wherein R^{3b} is methoxy

### Tables 11001 to 12000

Compounds of the formula Ia.1 in which the combination of R^{1a}, R^{1b}, R^{1c} and R^{3a} is as defined in any of Tables 1 to 1000 and Q is radical of formula II.9

### Tables 12001 to 13000

Compounds of the formula Ia.1 in which the combination of R^{1a}, Rib, R^{1c} and R^{3a} is as defined in any of Tables 1 to 1000 and Q is radical of formula II.10

### Tables 13001 to 14000

Compounds of the formula Ia.1 in which the combination of R^{1a}, R^{1b}, R^{1c} and R^{3a} is as defined in any of Tables 1 to 1000 and Q is radical of formula II.11

### Tables 14001 to 15000

Compounds of the formula Ia.1 in which the combination of R^{1a}, R^{1b}, R^{1c} and R^{3a} is as defined in any of Tables 1 to 1000 and Q is radical of formula II.12

### Tables 15001 to 16000

Compounds of the formula Ia.1 in which the combination of R^{1a}, Rib, R^{1c} and R^{3a} is as defined in any of Tables 1 to 1000 and Q is radical of formula II.13

### Tables 16001 to 17000

Compounds of the formula Ia.1 in which the combination of R^{1a}, R^{1b}, R^{1c} and R^{3a} is as defined in any of Tables 1 to 1000 and Q is radical of formula II.14

### Tables 17001 to 18000

Compounds of the formula Ia.1 in which the combination of R^{1a}, R^{1b}, R^{1c} and R^{3a} is as defined in any of Tables 1 to 1000 and Q is radical of formula II.15

### Tables 18001 to 19000

Compounds of the formula Ia.1 in which the combination of R^{1a}, R^{1b}, R^{1c} and R^{3a} is as defined in any of Tables 1 to 1000 and Q is radical of formula II.16

### Tables 19001 to 20000

Compounds of the formula Ia.1 in which the combination of R^{1a}, R^{1b}, R^{1c} and R^{3a} is as defined in any of Tables 1 to 1000 and Q is radical of formula II.17

### Tables 20001 to 21000

Compounds of the formula Ia.1 in which the combination of R^{1a}, R^{1b}, R^{1c} and R^{3a} is as defined in any of Tables 1 to 1000 and Q is radical of formula II.18

### Tables 21001 to 22000

Compounds of the formula Ia.1 in which the combination of R^{1a}, Rib, R^{1c} and R^{3a} is as defined in any of Tables 1 to 1000 and Q is radical of formula II.19

### Tables 22001 to 23000

Compounds of the formula Ia.1 in which the combination of R^{1a}, R^{1b}, R^{1c} and R^{3a} is as defined in any of Tables 1 to 1000 and Q is radical of formula II.20, wherein R^{3b} is Cl

### Tables 23001 to 24000

Compounds of the formula Ia.1 in which the combination of R^{1a}, R^{1b}, R^{1c} and R^{3a} is as defined in any of Tables 1 to 1000 and Q is radical of formula II.20, wherein R^{3b} is CN

### Tables 24001 to 25000

Compounds of the formula Ia.1 in which the combination of R^{1a}, R^{1b}, R^{1c} and R^{3a} is as defined in any of Tables 1 to 1000 and Q is radical of formula II.20, wherein R^{3b} is OH

### Tables 25001 to 26000

Compounds of the formula Ia.1 in which the combination of R^{1a}, R^{1b}, R^{1c} and R^{3a} is as defined in any of Tables 1 to 1000 and Q is radical of formula II.20, wherein R^{3b} is methoxy

### Tables 26001 to 27000

Compounds of the formula Ia.1 in which the combination of R^{1a}, R^{1b}, R^{1c} and R^{3a} is as defined in any of Tables 1 to 1000 and Q is radical of formula II.21

### Tables 27001 to 28000

Compounds of the formula Ia.1 in which the combination of R^{1a}, R^{1b}, R^{1c} and R^{1a} is as defined in any of Tables 1 to 1000 and Q is radical of formula II.22

### Tables 28001 to 29000

Compounds of the formula Ia.1 in which the combination of R^{1a}, R^{1b}, R^{1c} and R^{3a} is as defined in any of Tables 1 to 1000 and Q is radical of formula II.23, wherein R^{3b} is Cl

### Tables 29001 to 30000

Compounds of the formula Ia.1 in which the combination of R^{1a}, R^{1b}, R^{1c} and R^{3a} is as defined in any of Tables 1 to 1000 and Q is radical of formula II.23, wherein R^{3b} is CN

### Tables 30001 to 31000

Compounds of the formula Ia.1 in which the combination of R^{1a}, R^{1b}, R^{1c} and R^{3a} is as defined in any of Tables 1 to 1000 and Q is radical of formula II.23, wherein R^{3b} is OH

### Tables 31001 to 32000

Compounds of the formula Ia.1 in which the combination of R^{1a}, R^{1b}, R^{1c} and R^{3a} is as defined in any of Tables 1 to 1000 and Q is radical of formula II.23, wherein R^{3b} is methoxy

### Tables 32001 to 33000

Compounds of the formula Ia.1 in which the combination of R^{1a}, R^{1b}, R^{1c} and R^{3a} is as defined in any of Tables 1 to 1000 and Q is radical of formula II.24

### Tables 33001 to 34000

Compounds of the formula Ia.1 in which the combination of R^{1a}, R^{1b}, R^{1c} and R^{1a} is as defined in any of Tables 1 to 1000 and Q is radical of formula II.25

### Tables 34001 to 35000

Compounds of the formula Ia.1 in which the combination of R^{1a}, R^{1b}, R^{1c} and R^{3a} is as defined in any of Tables 1 to 1000 and Q is radical of formula II.26, wherein R^{3b} is Cl

### Tables 35001 to 36000

Compounds of the formula Ia.1 in which the combination of R^{1a}, R^{1b}, R^{1c} and R^{3a} is as defined in any of Tables 1 to 1000 and Q is radical of formula II.26, wherein R^{3b} is CN

### Tables 36001 to 37000

Compounds of the formula Ia.1 in which the combination of R^{1a}, R^{1b}, R^{1c} and R^{3a} is as defined in any of Tables 1 to 1000 and Q is radical of formula II.26, wherein R^{3b} is OH

### Tables 37001 to 38000

Compounds of the formula Ia.1 in which the combination of R^{1a}, R^{1b}, R^{1c} and R^{3a} is as defined in any of Tables 1 to 1000 and Q is radical of formula II.26, wherein R^{3b} is methoxy

### Tables 38001 to 39000

Compounds of the formula Ia.1 in which the combination of R^{1a}, R^{1b}, R^{1c} and R^{3a} is as defined in any of Tables 1 to 1000 and Q is radical of formula II.27

### Tables 39001 to 40000

Compounds of the formula Ia.1 in which the combination of R^{1a}, R^{1b}, R^{1c} and R^{3a} is as defined in any of Tables 1 to 1000 and Q is radical of formula II.28

### Tables 40001 to 41000

Compounds of the formula Ia.1 in which the combination of R^{1a}, R^{1b}, R^{1c} and R^{3a} is as defined in any of Tables 1 to 1000 and Q is radical of formula II.29, wherein R^{3b} is H

### Tables 41001 to 42000

Compounds of the formula Ia.1 in which the combination of R^{1a}, R^{1b}, R^{1c} and R^{3a} is as defined in any of Tables 1 to 1000 and Q is radical of formula II.29, wherein R^{3b} is methyl

### Tables 42001 to 43000

Compounds of the formula Ia.1 in which the combination of R^{1a}, R^{1b}, R^{1c} and R^{3a} is as defined in any of Tables 1 to 1000 and Q is radical of formula II.29, wherein R^{3b} is ethyl

### Tables 43001 to 44000

Compounds of the formula Ia.1 in which the combination of R^{1a}, R^{1b}, R^{1c} and R^{3a} is as defined in any of Tables 1 to 1000 and Q is radical of formula II.29, wherein R^{3b} is n-propyl

### Tables 44001 to 45000

Compounds of the formula Ia.1 in which the combination of R^{1a}, R^{1b}, R^{1c} and R^{3a} is as defined in any of Tables 1 to 1000 and Q is radical of formula II.29, wherein R^{3b} is isopropyl

### Tables 45001 to 46000

Compounds of the formula Ia.1 in which the combination of R^{1a}, R^{1b}, R^{1c} and R^{3a} is as defined in any of Tables 1 to 1000 and Q is radical of formula II.29, wherein R^{3b} is benzyl

### Tables 46001 to 47000

Compounds of the formula Ia.1 in which the combination of R^{1a}, R^{1b}, R^{1c} and R^{3a} is as defined in any of Tables 1 to 1000 and Q is radical of formula II.29, wherein R^{3b} is acetyl

### Tables 47001 to 48000

Compounds of the formula Ia.1 in which the combination of R^{1a}, R^{1b}, R^{1c} and R^{3a} is as defined in any of Tables 1 to 1000 and Q is radical of formula II.30, wherein R^{3b} is H

### Tables 48001 to 49000

Compounds of the formula Ia.1 in which the combination of R^{1a}, R^{1b}, R^{1c} and R^{3a} is as defined in any of Tables 1 to 1000 and Q is radical of formula II.30, wherein R^{3b} is methyl

### Tables 49001 to 50000

Compounds of the formula Ia.1 in which the combination of R^{1a}, R^{1b}, R^{1c} and R^{3a} is as defined in any of Tables 1 to 1000 and Q is radical of formula II.30, wherein R^{3b} is ethyl

### Tables 50001 to 51000

Compounds of the formula Ia.1 in which the combination of R^{1a}, R^{1b}, R^{1c} and R^{3a} is as defined in any of Tables 1 to 1000 and Q is radical of formula II.30, wherein R^{3b} is n-propyl

### Tables 51001 to 52000

Compounds of the formula Ia.1 in which the combination of R^{1a}, R^{1b}, R^{1c} and R^{3a} is as defined in any of Tables 1 to 1000 and Q is radical of formula II.30, wherein R^{3b} is isopropyl

### Tables 52001 to 53000

Compounds of the formula Ia.1 in which the combination of R^{1a}, R^{1b}, R^{1c} and R^{3a} is as defined in any of Tables 1 to 1000 and Q is radical of formula II.30, wherein R^{3b} is benzyl

### Tables 53001 to 54000

Compounds of the formula Ia.1 in which the combination of R^{1a}, R^{1b}, R^{1c} and R^{3a} is as defined in any of Tables 1 to 1000 and Q is radical of formula II.30, wherein R^{3b} is acetyl

### Tables 54001 to 55000

Compounds of the formula Ia.1 in which the combination of R^{1a}, R^{1b}, R^{1c} and R^{3a} is as defined in any of Tables 1 to 1000 and Q is radical of formula II.31, wherein R^{3b} is H

### Tables 55001 to 56000

Compounds of the formula Ia.1 in which the combination of R^{1a}, R^{1b}, R^{1c} and R^{3a} is as defined in any of Tables 1 to 1000 and Q is radical of formula II.31, wherein R^{3b} is methyl

### Tables 56001 to 57000

Compounds of the formula Ia.1 in which the combination of R^{1a}, R^{1b}, R^{1c} and R^{3a} is as defined in any of Tables 1 to 1000 and Q is radical of formula II.31, wherein R^{3b} is ethyl

### Tables 57001 to 58000

Compounds of the formula Ia.1 in which the combination of R^{1a}, R^{1b}, R^{1c} and R^{3a} is as defined in any of Tables 1 to 1000 and Q is radical of formula II.31, wherein R^{3b} is n-propyl

### Tables 58001 to 59000

Compounds of the formula Ia.1 in which the combination of R^{1a}, R^{1b}, R^{1c} and R^{3a} is as defined in any of Tables 1 to 1000 and Q is radical of formula II.31, wherein R^{3b} is isopropyl

### Tables 59001 to 60000

Compounds of the formula Ia.1 in which the combination of R^{1a}, R^{1b}, R^{1c} and R^{3a} is as defined in any of Tables 1 to 1000 and Q is radical of formula II.31, wherein R^{3b} is benzyl

### Tables 60001 to 61000

Compounds of the formula Ia.1 in which the combination of R^{1a}, R^{1b}, R^{1c} and R^{3a} is as defined in any of Tables 1 to 1000 and Q is radical of formula II.31, wherein R^{3b} is acetyl

### Tables 61001 to 62000

Compounds of the formula Ia.1 in which the combination of R^{1a}, R^{1b}, R^{1c} and R^{3a} is as defined in any of Tables 1 to 1000 and Q is radical of formula II.32, wherein R^{3b} is H

### Tables 62001 to 63000

Compounds of the formula Ia.1 in which the combination of R^{1a}, R^{1b}, R^{1c} and R^{3a} is as defined in any of Tables 1 to 1000 and Q is radical of formula II.32, wherein R^{3b} is methyl

### Tables 63001 to 64000

Compounds of the formula Ia.1 in which the combination of R^{1a}, R^{1b}, R^{1c} and R^{3a} is as defined in any of Tables 1 to 1000 and Q is radical of formula II.32, wherein R^{3b} is ethyl

### Tables 64001 to 65000

Compounds of the formula Ia.1 in which the combination of R^{1a}, R^{1b}, R^{1c} and R^{3a} is as defined in any of Tables 1 to 1000 and Q is radical of formula II.32, wherein R^{3b} is n-propyl

### Tables 65001 to 66000

Compounds of the formula Ia.1 in which the combination of R^{1a}, R^{1b}, R^{1c} and R^{3a} is as defined in any of Tables 1 to 1000 and Q is radical of formula II.32, wherein R^{3b} is isopropyl

### Tables 66001 to 67000

Compounds of the formula Ia.1 in which the combination of R^{1a}, R^{1b}, R^{1c} and R^{3a} is as defined in any of Tables 1 to 1000 and Q is radical of formula II.32, wherein R^{3b} is benzyl

### Tables 67001 to 68000

Compounds of the formula Ia.1 in which the combination of R^{1a}, R^{1b}, R^{1c} and R^{3a} is as defined in any of Tables 1 to 1000 and Q is radical of formula II.32, wherein R^{3b} is acetyl

### Tables 68001 to 69000

Compounds of the formula Ia.1 in which the combination of R^{1a}, R^{1b}, R^{1c} and R^{3a} is as defined in any of Tables 1 to 1000 and Q is radical of formula II.33, wherein R^{3b} is H

### Tables 69001 to 70000

Compounds of the formula Ia.1 in which the combination of R^{1a}, R^{1b}, R^{1c} and R^{3a} is as defined in any of Tables 1 to 1000 and Q is radical of formula II.33, wherein R^{3b} is methyl

### Tables 70001 to 71000

Compounds of the formula Ia.1 in which the combination of R^{1a}, R^{1b}, R^{1c} and R^{3a} is as defined in any of Tables 1 to 1000 and Q is radical of formula II.33, wherein R^{3b} is ethyl

### Tables 71001 to 72000

Compounds of the formula Ia.1 in which the combination of R^{1a}, R^{1b}, R^{1c} and R^{3a} is as defined in any of Tables 1 to 1000 and Q is radical of formula II.33, wherein R^{3b} is n-propyl

### Tables 72001 to 73000

Compounds of the formula Ia.1 in which the combination of R^{1a}, R^{1b}, R^{1c} and R^{3a} is as defined in any of Tables 1 to 1000 and Q is radical of formula II.33, wherein R^{3b} is isopropyl

### Tables 73001 to 74000

Compounds of the formula Ia.1 in which the combination of R^{1a}, R^{1b}, R^{1c} and R^{3a} is as defined in any of Tables 1 to 1000 and Q is radical of formula II.33, wherein R^{3b} is benzyl

### Tables 74001 to 75000

Compounds of the formula Ia.1 in which the combination of R^{1a}, R^{1b}, R^{1c} and R^{3a} is as defined in any of Tables 1 to 1000 and Q is radical of formula II.33, wherein R^{3b} is acetyl

### Tables 75001 to 76000

Compounds of the formula Ia.1 in which the combination of R^{1a}, R^{1b}, R^{1c} and R^{3a} is as defined in any of Tables 1 to 1000 and Q is radical of formula II.34, wherein R^{3b} is H

### Tables 76001 to 77000

Compounds of the formula Ia.1 in which the combination of R^{1a}, R^{1b}, R^{1c} and R^{3a} is as defined in any of Tables 1 to 1000 and Q is radical of formula II.34, wherein R^{3b} is methyl

### Tables 77001 to 78000

Compounds of the formula Ia.1 in which the combination of R^{1a}, R^{1b}, R^{1c} and R^{3a} is as defined in any of Tables 1 to 1000 and Q is radical of formula II.34, wherein R^{3b} is ethyl

### Tables 78001 to 79000

Compounds of the formula Ia.1 in which the combination of R^{1a}, R^{1b}, R^{1c} and R^{3a} is as defined in any of Tables 1 to 1000 and Q is radical of formula II.34, wherein R^{3b} is n-propyl

### Tables 79001 to 80000

Compounds of the formula Ia.1 in which the combination of R^{1a}, R^{1b}, R^{1c} and R^{3a} is as defined in any of Tables 1 to 1000 and Q is radical of formula II.34, wherein R^{3b} is isopropyl

### Tables 80001 to 81000

Compounds of the formula Ia.1 in which the combination of R^{1a}, R^{1b}, R^{1c} and R^{3a} is as defined in any of Tables 1 to 1000 and Q is radical of formula II.34, wherein R^{3b} is benzyl

### Tables 81001 to 82000

Compounds of the formula Ia.1 in which the combination of R^{1a}, R^{1b}, R^{1c} and R^{3a} is as defined in any of Tables 1 to 1000 and Q is radical of formula II.34, wherein R^{3b} is acetyl

### Tables 82001 to 83000

Compounds of the formula Ia.1 in which the combination of R^{1a}, R^{1b}, R^{1c} and R^{3a} is as defined in any of Tables 1 to 1000 and Q is radical of formula II.35, wherein R^{3b} is H

### Tables 83001 to 84000

Compounds of the formula Ia.1 in which the combination of R^{1a}, R^{1b}, R^{1c} and R^{3a} is as defined in any of Tables 1 to 1000 and Q is radical of formula II.35, wherein R^{3b} is methyl

### Tables 84001 to 85000

Compounds of the formula Ia.1 in which the combination of R^{1a}, R^{1b}, R^{1c} and R^{3a} is as defined in any of Tables 1 to 1000 and Q is radical of formula II.35, wherein R^{3b} is ethyl

### Tables 85001 to 86000

Compounds of the formula Ia.1 in which the combination of R^{1a}, R^{1b}, R^{1c} and R^{3a} is as defined in any of Tables 1 to 1000 and Q is radical of formula II.35, wherein R^{3b} is n-propyl

### Tables 86001 to 87000

Compounds of the formula Ia.1 in which the combination of R^{1a}, R^{1b}, R^{1c} and R^{3a} is as defined in any of Tables 1 to 1000 and Q is radical of formula II.35, wherein R^{3b} is isopropyl

### Tables 87001 to 88000

Compounds of the formula Ia.1 in which the combination of R^{1a}, R^{1b}, R^{1c} and R^{3a} is as defined in any of Tables 1 to 1000 and Q is radical of formula II.35, wherein R^{3b} is benzyl

### Tables 88001 to 89000

Compounds of the formula Ia.1 in which the combination of R^{1a}, R^{1b}, R^{1c} and R^{1a} is as defined in any of Tables 1 to 1000 and Q is radical of formula II.35, wherein R^{3b} is acetyl

### Tables 89001 to 90000

Compounds of the formula Ia.1 in which the combination of R^{1a}, R^{1b}, R^{1c} and R^{3a} is as defined in any of Tables 1 to 1000 and Q is radical of formula II.36, wherein R^{3b} is H

### Tables 90001 to 91000

Compounds of the formula Ia.1 in which the combination of R^{1a}, R^{1b}, R^{1c} and R^{3a} is as defined in any of Tables 1 to 1000 and Q is radical of formula II.36, wherein R^{3b} is methyl

### Tables 91001 to 92000

Compounds of the formula Ia.1 in which the combination of R^{1a}, R^{1b}, R^{1c} and R^{3a} is as defined in any of Tables 1 to 1000 and Q is radical of formula II.36, wherein R^{3b} is ethyl

### Tables 92001 to 93000

Compounds of the formula Ia.1 in which the combination of R^{1a}, R^{1b}, R^{1c} and R^{3a} is as defined in any of Tables 1 to 1000 and Q is radical of formula II.36, wherein R^{3b} is n-propyl

### Tables 93001 to 94000

Compounds of the formula Ia.1 in which the combination of R^{1a}, R^{1b}, R^{1c} and R^{3a} is as defined in any of Tables 1 to 1000 and Q is radical of formula II.36, wherein R^{3b} is isopropyl

### Tables 94001 to 95000

Compounds of the formula Ia.1 in which the combination of R^{1a}, R^{1b}, R^{1c} and R^{3a} is as defined in any of Tables 1 to 1000 and Q is radical of formula II.36, wherein R^{3b} is benzyl

### Tables 95001 to 96000

Compounds of the formula Ia.1 in which the combination of R^{1a}, R^{1b}, R^{1c} and R^{1a} is as defined in any of Tables 1 to 1000 and Q is radical of formula II.36, wherein R^{3b} is acetyl

### Tables 96001 to 97000

Compounds of the formula Ia.1 in which the combination of R^{1a}, R^{1b}, R^{1c} and R^{3a} is as defined in any of Tables 1 to 1000 and Q is radical of formula II.37

### Tables 97001 to 98000

Compounds of the formula Ia.1 in which the combination of R^{1a}, R^{1b}, R^{1c} and R^{3a} is as defined in any of Tables 1 to 1000 and Q is radical of formula II.38

### Tables 98001 to 99000

Compounds of the formula Ia.1 in which the combination of R^{1a}, R^{1b}, R^{1c} and R^{3a} is as defined in any of Tables 1 to 1000 and Q is radical of formula II.39

### Tables 99001 to 10000

Compounds of the formula Ia.1 in which the combination of R^{1a}, R^{1b}, R^{1c} and R^{3a} is as defined in any of Tables 1 to 1000 and Q is radical of formula II.40

### Tables 100001 to 101000

Compounds of the formula Ia.1 in which the combination of R^{1a}, R^{1b}, R^{1c} and R^{3a} is as defined in any of Tables 1 to 1000 and Q is radical of formula II.41

### Tables 101001 to 102000

Compounds of the formula Ia.1 in which the combination of R^{1a}, R^{1b}, R^{1c} and R^{3a} is as defined in any of Tables 1 to 1000 and Q is radical of formula II.42

### Tables 102001 to 103000

Compounds of the formula Ia.1 in which the combination of R^{1a}, R^{1b}, R^{1c} and R^{3a} is as defined in any of Tables 1 to 1000 and Q is radical of formula II.43

### Tables 103001 to 104000

Compounds of the formula Ia.1 in which the combination of R^{1a}, R^{1b}, R^{1c} and R^{3a} is as defined in any of Tables 1 to 1000 and Q is radical of formula II.44

### Tables 104001 to 105000

Compounds of the formula Ia.1 in which the combination of R^{1a}, R^{1b}, R^{1c} and R^{3a} is as defined in any of Tables 1 to 1000 and Q is radical of formula II.45

### Tables 105001 to 106000

Compounds of the formula Ia.1 in which the combination of R^{1a}, R^{1b}, R^{1c} and R^{3a} is as defined in any of Tables 1 to 1000 and Q is radical of formula II.46

### Tables 106001 to 107000

Compounds of the formula Ia.1 in which the combination of R^{1a}, R^{1b}, R^{1c} and R^{1a} is as defined in any of Tables 1 to 1000 and Q is radical of formula II.47

### Tables 107001 to 108000

Compounds of the formula Ia.1 in which the combination of R^{1a}, R^{1b}, R^{1c} and R^{3a} is as defined in any of Tables 1 to 1000 and Q is radical of formula II.48

### Tables 108001 to 109000

Compounds of the formula Ia.1 in which the combination of R^{1a}, R^{1b}, R^{1c} and R^{3a} is as defined in any of Tables 1 to 1000 and Q is radical of formula II.49

### Tables 109001 to 110000

Compounds of the formula Ia.1 in which the combination of R^{1a}, R^{1b}, R^{1c} and R^{3a} is as defined in any of Tables 1 to 1000 and Q is radical of formula II.50

### Tables 110001 to 111000

Compounds of the formula Ia.1 in which the combination of R^{1a}, R^{1b}, R^{1c} and R^{3a} is as defined in any of Tables 1 to 1000 and Q is radical of formula II.51

### Tables 111001 to 112000

Compounds of the formula Ia.1 in which the combination of R^{1a}, R^{1b}, R^{1c} and R^{3a} is as defined in any of Tables 1 to 1000 and Q is radical of formula II.52, wherein R^{3b} is Cl

### Tables 112001 to 113000

Compounds of the formula Ia.1 in which the combination of R^{1a}, R^{1b}, R^{1c} and R^{3a} is as defined in any of Tables 1 to 1000 and Q is radical of formula II.52, wherein R^{3b} is CN

### Tables 113001 to 114000

Compounds of the formula Ia.1 in which the combination of R^{1a}, R^{1b}, R^{1c} and R^{3a} is as defined in any of Tables 1 to 1000 and Q is radical of formula II.52, wherein R^{3b} is OH

### Tables 114001 to 115000

Compounds of the formula Ia.1 in which the combination of R^{1a}, R^{1b}, R^{1c} and R^{3a} is as defined in any of Tables 1 to 1000 and Q is radical of formula II.52, wherein R^{3b} is methoxy

### Tables 115001 to 116000

Compounds of the formula Ia.1 in which the combination of R^{1a}, R^{1b}, R^{1c} and R^{3a} is as defined in any of Tables 1 to 1000 and Q is radical of formula II.53

### Tables 116001 to 117000

Compounds of the formula Ia.1 in which the combination of R^{1a}, R^{1b}, R^{1c} and R^{3a} is as defined in any of Tables 1 to 1000 and Q is radical of formula II.54

### Tables 117001 to 118000

Compounds of the formula Ia.1 in which the combination of R^{1a}, R^{1b}, R^{1c} and R^{3a} is as defined in any of Tables 1 to 1000 and Q is radical of formula II.55

### Tables 118001 to 119000

Compounds of the formula Ia.1 in which the combination of R^{1a}, R^{1b}, R^{1c} and R^{3a} is as defined in any of Tables 1 to 1000 and Q is radical of formula II.56

### Tables 119001 to 120000

Compounds of the formula Ia.1 in which the combination of R^{1a}, R^{1b}, R^{1c} and R^{3a} is as defined in any of Tables 1 to 1000 and Q is radical of formula II.57

### Tables 120001 to 121000

Compounds of the formula Ia.1 in which the combination of R^{1a}, R^{1b}, R^{1c} and R^{3a} is as defined in any of Tables 1 to 1000 and Q is radical of formula II.58

### Tables 121001 to 122000

Compounds of the formula Ia.1 in which the combination of R^{1a}, R^{1b}, R^{1c} and R^{3a} is as defined in any of Tables 1 to 1000 and Q is radical of formula II.59

### Tables 122001 to 123000

Compounds of the formula Ia.1 in which the combination of R¹⁰, R^{1b}, R^{1c} and R^{3a} is as defined in any of Tables 1 to 1000 and Q is radical of formula II.60

### Tables 123001 to 124000

Compounds of the formula Ia.1 in which the combination of R^{1a}, R^{1b}, R^{1c} and R^{3a} is as defined in any of Tables 1 to 1000 and Q is radical of formula II.61

### Tables 124001 to 125000

Compounds of the formula Ia.1 in which the combination of R¹⁰, R^{1b}, R^{1c} and R^{3a} is as defined in any of Tables 1 to 1000 and Q is radical of formula II.62

### Tables 125001 to 126000

Compounds of the formula Ia.1 in which the combination of R^{1a}, R^{1b}, R^{1c} and R^{3a} is as defined in any of Tables 1 to 1000 and Q is radical of formula II.63

### Tables 126001 to 127000

Compounds of the formula Ia.1 in which the combination of R^{1a}, R^{1b}, R^{1c} and R^{3a} is as defined in any of Tables 1 to 1000 and Q is radical of formula II.64, wherein R^{3b} is Cl

### Tables 127001 to 128000

Compounds of the formula Ia.1 in which the combination of R^{1a}, R^{1b}, R^{1c} and R^{3a} is as defined in any of Tables 1 to 1000 and Q is radical of formula II.64, wherein R^{3b} is CN

### Tables 128001 to 129000

Compounds of the formula Ia.1 in which the combination of R^{1a}, R^{1b}, R^{1c} and R^{3a} is as defined in any of Tables 1 to 1000 and Q is radical of formula II.64, wherein R^{3b} is OH

### Tables 129001 to 130000

Compounds of the formula Ia.1 in which the combination of R^{1a}, R^{1b}, R^{1c} and R^{3a} is as defined in any of Tables 1 to 1000 and Q is radical of formula II.64, wherein R^{3b} is methoxy

### Tables 130001 to 131000

Compounds of the formula Ia.1 in which the combination of R^{1a}, R^{1b}, R^{1c} and R^{3a} is as defined in any of Tables 1 to 1000 and Q is radical of formula II.65

### Tables 131001 to 132000

Compounds of the formula Ia.1 in which the combination of R^{1a}, R^{1b}, R^{1c} and R^{1a} is as defined in any of Tables 1 to 1000 and Q is radical of formula II.66

### Tables 132001 to 133000

Compounds of the formula Ia.1 in which the combination of R^{1a}, R^{1b}, R^{1c} and R^{3a} is as defined in any of Tables 1 to 1000 and Q is radical of formula II.67, wherein R^{3b} is Cl

### Tables 133001 to 134000

Compounds of the formula Ia.1 in which the combination of R^{1a}, R^{1b}, R^{1c} and R^{3a} is as defined in any of Tables 1 to 1000 and Q is radical of formula II.67, wherein R^{3b} is CN

### Tables 134001 to 135000

Compounds of the formula Ia.1 in which the combination of R^{1a}, R^{1b}, R^{1c} and R^{3a} is as defined in any of Tables 1 to 1000 and Q is radical of formula II.67, wherein R^{3b} is OH

### Tables 135001 to 136000

Compounds of the formula Ia.1 in which the combination of R^{1a}, R^{1b}, R^{1c} and R^{3a} is as defined in any of Tables 1 to 1000 and Q is radical of formula II.67, wherein R^{3b} is methoxy

### Tables 136001 to 137000

Compounds of the formula Ia.1 in which the combination of R^{1a}, R^{1b}, R^{1c} and R^{3a} is as defined in any of Tables 1 to 1000 and Q is radical of formula II.68

### Tables 137001 to 138000

Compounds of the formula Ia.1 in which the combination of R^{1a}, R^{1b}, R^{1c} and R^{3a} is as defined in any of Tables 1 to 1000 and Q is radical of formula II.69

### Tables 138001 to 139000

Compounds of the formula Ia.1 in which the combination of R^{1a}, R^{1b}, R^{1c} and R^{3a} is as defined in any of Tables 1 to 1000 and Q is radical of formula II.70, wherein R^{3b} is Cl

### Tables 139001 to 140000

Compounds of the formula Ia.1 in which the combination of R^{1a}, R^{1b}, R^{1c} and R^{3a} is as defined in any of Tables 1 to 1000 and Q is radical of formula II.70, wherein R^{3b} is CN

### Tables 140001 to 141000

Compounds of the formula Ia.1 in which the combination of R^{1a}, R^{1b}, R^{1c} and R^{3a} is as defined in any of Tables 1 to 1000 and Q is radical of formula II.70, wherein R^{3b} is OH

### Tables 141001 to 142000

Compounds of the formula Ia.1 in which the combination of R^{1a}, R^{1b}, R^{1c} and R^{3a} is as defined in any of Tables 1 to 1000 and Q is radical of formula II.70, wherein R^{3b} is methoxy

### Tables 142001 to 143000

Compounds of the formula Ia.1 in which the combination of R^{1a}, R^{1b}, R^{1c} and R^{3a} is as defined in any of Tables 1 to 1000 and Q is radical of formula II.71

### Tables 143001 to 144000

Compounds of the formula Ia.1 in which the combination of R^{1a}, R^{1b}, R^{1c} and R^{3a} is as defined in any of Tables 1 to 1000 and Q is radical of formula II.72

### Tables 144001 to 145000

Compounds of the formula Ia.1 in which the combination of R^{1a}, R^{1b}, R^{1c} and R^{3a} is as defined in any of Tables 1 to 1000 and Q is radical of formula II.73, wherein R^{3b} is H

### Tables 145001 to 146000

Compounds of the formula Ia.1 in which the combination of R^{1a}, R^{1b}, R^{1c} and R^{3a} is as defined in any of Tables 1 to 1000 and Q is radical of formula II.73, wherein R^{3b} is methyl

### Tables 146001 to 147000

Compounds of the formula Ia.1 in which the combination of R^{1a}, R^{1b}, R^{1c} and R^{3a} is as defined in any of Tables 1 to 1000 and Q is radical of formula II.73, wherein R^{3b} is ethyl

### Tables 147001 to 148000

Compounds of the formula Ia.1 in which the combination of R^{1a}, R^{1b}, R^{1c} and R^{3a} is as defined in any of Tables 1 to 1000 and Q is radical of formula II.73, wherein R^{3b} is n-propyl

### Tables 148001 to 149000

Compounds of the formula Ia.1 in which the combination of R^{1a}, R^{1b}, R^{1c} and R^{3a} is as defined in any of Tables 1 to 1000 and Q is radical of formula II.73, wherein R^{3b} is isopropyl

### Tables 149001 to 150000

Compounds of the formula Ia.1 in which the combination of R^{1a}, R^{1b}, R^{1c} and R^{3a} is as defined in any of Tables 1 to 1000 and Q is radical of formula II.73, wherein R^{3b} is benzyl

### Tables 150001 to 151000

Compounds of the formula Ia.1 in which the combination of R^{1a}, R^{1b}, R^{1c} and R^{3a} is as defined in any of Tables 1 to 1000 and Q is radical of formula II.73, wherein R^{3b} is acetyl

### Tables 151001 to 152000

Compounds of the formula Ia.1 in which the combination of R^{1a}, R^{1b}, R^{1c} and R^{3a} is as defined in any of Tables 1 to 1000 and Q is radical of formula II.74, wherein R^{3b} is H

### Tables 152001 to 153000

Compounds of the formula Ia.1 in which the combination of R^{1a}, R^{1b}, R^{1c} and R^{3a} is as defined in any of Tables 1 to 1000 and Q is radical of formula II.74, wherein R^{3b} is methyl

### Tables 153001 to 154000

Compounds of the formula Ia.1 in which the combination of R^{1a}, R^{1b}, R^{1c} and R^{3a} is as defined in any of Tables 1 to 1000 and Q is radical of formula II.74, wherein R^{3b} is ethyl

### Tables 154001 to 155000

Compounds of the formula Ia.1 in which the combination of R^{1a}, R^{1b}, R^{1c} and R^{3a} is as defined in any of Tables 1 to 1000 and Q is radical of formula II.74, wherein R^{3b} is n-propyl

### Tables 155001 to 156000

Compounds of the formula Ia.1 in which the combination of R^{1a}, R^{1b}, R^{1c} and R^{3a} is as defined in any of Tables 1 to 1000 and Q is radical of formula II.74, wherein R^{3b} is isopropyl

### Tables 156001 to 157000

Compounds of the formula Ia.1 in which the combination of R^{1a}, R^{1b}, R^{1c} and R^{3a} is as defined in any of Tables 1 to 1000 and Q is radical of formula II.74, wherein R^{3b} is benzyl

### Tables 157001 to 158000

Compounds of the formula Ia.1 in which the combination of R^{1a}, R^{1b}, R^{1c} and R^{3a} is as defined in any of Tables 1 to 1000 and Q is radical of formula II.74, wherein R^{3b} is acetyl

### Tables 158001 to 159000

Compounds of the formula Ia.1 in which the combination of R^{1a}, R^{1b}, R^{1c} and R^{3a} is as defined in any of Tables 1 to 1000 and Q is radical of formula II.75, wherein R^{3b} is H

### Tables 159001 to 160000

Compounds of the formula Ia.1 in which the combination of R^{1a}, R^{1b}, R^{1c} and R^{3a} is as defined in any of Tables 1 to 1000 and Q is radical of formula II.75, wherein R^{3b} is methyl

### Tables 160001 to 161000

Compounds of the formula Ia.1 in which the combination of R^{1a}, R^{1b}, R^{1c} and R^{3a} is as defined in any of Tables 1 to 1000 and Q is radical of formula II.75, wherein R^{3b} is ethyl

### Tables 161001 to 162000

Compounds of the formula Ia.1 in which the combination of R^{1a}, R^{1b}, R^{1c} and R^{3a} is as defined in any of Tables 1 to 1000 and Q is radical of formula II.75, wherein R^{3b} is n-propyl

### Tables 162001 to 163000

Compounds of the formula Ia.1 in which the combination of R^{1a}, R^{1b}, R^{1c} and R^{3a} is as defined in any of Tables 1 to 1000 and Q is radical of formula II.75, wherein R^{3b} is isopropyl

### Tables 163001 to 164000

Compounds of the formula Ia.1 in which the combination of R^{1a}, R^{1b}, R^{1c} and R^{3a} is as defined in any of Tables 1 to 1000 and Q is radical of formula II.75, wherein R^{3b} is benzyl

### Tables 164001 to 165000

Compounds of the formula Ia.1 in which the combination of R^{1a}, R^{1b}, R^{1c} and R^{3a} is as defined in any of Tables 1 to 1000 and Q is radical of formula II.75, wherein R^{3b} is acetyl

### Tables 165001 to 166000

Compounds of the formula Ia.1 in which the combination of R^{1a}, R^{1b}, R^{1c} and R^{3a} is as defined in any of Tables 1 to 1000 and Q is radical of formula II.76, wherein R^{3b} is H

### Tables 166001 to 167000

Compounds of the formula Ia.1 in which the combination of R^{1a}, R^{1b}, R^{1c} and R^{3a} is as defined in any of Tables 1 to 1000 and Q is radical of formula II.76, wherein R^{3b} is methyl

### Tables 167001 to 168000

Compounds of the formula Ia.1 in which the combination of R^{1a}, R^{1b}, R^{1c} and R^{3a} is as defined in any of Tables 1 to 1000 and Q is radical of formula II.76, wherein R^{3b} is ethyl

### Tables 168001 to 169000

Compounds of the formula Ia.1 in which the combination of R^{1a}, R^{1b}, R^{1c} and R^{3a} is as defined in any of Tables 1 to 1000 and Q is radical of formula II.76, wherein R^{3b} is n-propyl

### Tables 169001 to 170000

Compounds of the formula Ia.1 in which the combination of R^{1a}, R^{1b}, R^{1c} and R^{3a} is as defined in any of Tables 1 to 1000 and Q is radical of formula II.76, wherein R^{3b} is isopropyl

### Tables 170001 to 171000

Compounds of the formula Ia.1 in which the combination of R^{1a}, R^{1b}, R^{1c} and R^{3a} is as defined in any of Tables 1 to 1000 and Q is radical of formula II.76, wherein R^{3b} is benzyl

### Tables 171001 to 172000

Compounds of the formula Ia.1 in which the combination of R^{1a}, R^{1b}, R^{1c} and R^{3a} is as defined in any of Tables 1 to 1000 and Q is radical of formula II.76, wherein R^{3b} is acetyl

### Tables 172001 to 173000

Compounds of the formula Ia.1 in which the combination of R^{1a}, R^{1b}, R^{1c} and R^{3a} is as defined in any of Tables 1 to 1000 and Q is radical of formula II.77, wherein R^{3b} is H

### Tables 173001 to 174000

Compounds of the formula Ia.1 in which the combination of R^{1a}, R^{1b}, R^{1c} and R^{3a} is as defined in any of Tables 1 to 1000 and Q is radical of formula II.77, wherein R^{3b} is methyl

### Tables 174001 to 175000

Compounds of the formula Ia.1 in which the combination of R^{1a}, R^{1b}, R^{1c} and R^{3a} is as defined in any of Tables 1 to 1000 and Q is radical of formula II.77, wherein R^{3b} is ethyl

### Tables 175001 to 176000

Compounds of the formula Ia.1 in which the combination of R^{1a}, R^{1b}, R^{1c} and R^{3a} is as defined in any of Tables 1 to 1000 and Q is radical of formula II.77, wherein R^{3b} is n-propyl

### Tables 176001 to 177000

Compounds of the formula Ia.1 in which the combination of R^{1a}, R^{1b}, R^{1c} and R^{3a} is as defined in any of Tables 1 to 1000 and Q is radical of formula II.77, wherein R^{3b} is isopropyl

### Tables 177001 to 178000

Compounds of the formula Ia.1 in which the combination of R^{1a}, R^{1b}, R^{1c} and R^{3a} is as defined in any of Tables 1 to 1000 and Q is radical of formula II.77, wherein R^{3b} is benzyl

### Tables 178001 to 179000

Compounds of the formula Ia.1 in which the combination of R^{1a}, R^{1b}, R^{1c} and R^{3a} is as defined in any of Tables 1 to 1000 and Q is radical of formula II.77, wherein R^{3b} is acetyl

### Tables 179001 to 180000

Compounds of the formula Ia.1 in which the combination of R^{1a}, R^{1b}, R^{1c} and R^{3a} is as defined in any of Tables 1 to 1000 and Q is radical of formula II.78, wherein R^{3b} is H

### Tables 180001 to 181000

Compounds of the formula Ia.1 in which the combination of R^{1a}, R^{1b}, R^{1c} and R^{3a} is as defined in any of Tables 1 to 1000 and Q is radical of formula II.78, wherein R^{3b} is methyl

### Tables 181001 to 182000

Compounds of the formula Ia.1 in which the combination of R^{1a}, R^{1b}, R^{1c} and R^{3a} is as defined in any of Tables 1 to 1000 and Q is radical of formula II.78, wherein R^{3b} is ethyl

### Tables 182001 to 183000

Compounds of the formula Ia.1 in which the combination of R^{1a}, R^{1b}, R^{1c} and R^{3a} is as defined in any of Tables 1 to 1000 and Q is radical of formula II.78, wherein R^{3b} is n-propyl

### Tables 183001 to 184000

Compounds of the formula Ia.1 in which the combination of R^{1a}, R^{1b}, R^{1c} and R^{3a} is as defined in any of Tables 1 to 1000 and Q is radical of formula II.78, wherein R^{3b} is isopropyl

### Tables 184001 to 185000

Compounds of the formula Ia.1 in which the combination of R^{1a}, R^{1b}, R^{1c} and R^{3a} is as defined in any of Tables 1 to 1000 and Q is radical of formula II.78, wherein R^{3b} is benzyl

### Tables 185001 to 186000

Compounds of the formula Ia.1 in which the combination of R^{1a}, R^{1b}, R^{1c} and R^{3a} is as defined in any of Tables 1 to 1000 and Q is radical of formula II.78, wherein R^{3b} is acetyl

### Tables 186001 to 187000

Compounds of the formula Ia.1 in which the combination of R^{1a}, R^{1b}, R^{1c} and R^{3a} is as defined in any of Tables 1 to 1000 and Q is radical of formula II.79, wherein R^{3b} is H

### Tables 187001 to 188000

Compounds of the formula Ia.1 in which the combination of R^{1a}, R^{1b}, R^{1c} and R^{3a} is as defined in any of Tables 1 to 1000 and Q is radical of formula II.79, wherein R^{3b} is methyl

### Tables 188001 to 189000

Compounds of the formula Ia.1 in which the combination of R^{1a}, R^{1b}, R^{1c} and R^{3a} is as defined in any of Tables 1 to 1000 and Q is radical of formula II.79, wherein R^{3b} is ethyl

### Tables 189001 to 190000

Compounds of the formula Ia.1 in which the combination of R^{1a}, R^{1b}, R^{1c} and R^{3a} is as defined in any of Tables 1 to 1000 and Q is radical of formula II.79, wherein R^{3b} is n-propyl

### Tables 190001 to 191000

Compounds of the formula Ia.1 in which the combination of R^{1a}, R^{1b}, R^{1c} and R^{1a} is as defined in any of Tables 1 to 1000 and Q is radical of formula II.79, wherein R^{3b} is isopropyl

### Tables 191001 to 192000

Compounds of the formula Ia.1 in which the combination of R^{1a}, R^{1b}, R^{1c} and R^{3a} is as defined in any of Tables 1 to 1000 and Q is radical of formula II.79, wherein R^{3b} is benzyl

### Tables 192001 to 193000

Compounds of the formula Ia.1 in which the combination of R^{1a}, R^{1b}, R^{1c} and R^{3a} is as defined in any of Tables 1 to 1000 and Q is radical of formula II.79, wherein R^{3b} is acetyl

### Tables 193001 to 194000

Compounds of the formula Ia.1 in which the combination of R^{1a}, R^{1b}, R^{1c} and R^{3a} is as defined in any of Tables 1 to 1000 and Q is radical of formula II.80, wherein R^{3b} is H

### Tables 194001 to 195000

Compounds of the formula Ia.1 in which the combination of R^{1a}, R^{1b}, R^{1c} and R^{3a} is as defined in any of Tables 1 to 1000 and Q is radical of formula II.80, wherein R^{3b} is methyl

### Tables 195001 to 196000

Compounds of the formula Ia.1 in which the combination of R^{1a}, R^{1b}, R^{1c} and R^{3a} is as defined in any of Tables 1 to 1000 and Q is radical of formula II.80, wherein R^{3b} is ethyl

### Tables 196001 to 197000

Compounds of the formula Ia.1 in which the combination of R^{1a}, R^{1b}, R^{1c} and R^{3a} is as defined in any of Tables 1 to 1000 and Q is radical of formula II.80, wherein R^{3b} is n-propyl

### Tables 197001 to 198000

Compounds of the formula Ia.1 in which the combination of R^{1a}, R^{1b}, R^{1c} and R^{3a} is as defined in any of Tables 1 to 1000 and Q is radical of formula II.80, wherein R^{3b} is isopropyl

### Tables 198001 to 199000

Compounds of the formula Ia.1 in which the combination of R^{1a}, R^{1b}, R^{1c} and R^{3a} is as defined in any of Tables 1 to 1000 and Q is radical of formula II.80, wherein R^{3b} is benzyl

### Tables 199001 to 200000

Compounds of the formula Ia.1 in which the combination of R^{1a}, R^{1b}, R^{1c} and R^{3a} is as defined in any of Tables 1 to 1000 and Q is radical of formula II.80, wherein R^{3b} is acetyl

### Tables 200001 to 201000

Compounds of the formula Ia.1 in which the combination of R^{1a}, R^{1b}, R^{1c} and R^{3a} is as defined in any of Tables 1 to 1000 and Q is radical of formula II.81

### Tables 201001 to 202000

Compounds of the formula Ia.1 in which the combination of R^{1a}, R^{1b}, R^{1c} and R^{3a} is as defined in any of Tables 1 to 1000 and Q is radical of formula II.82

### Tables 202001 to 203000

Compounds of the formula Ia.1 in which the combination of R^{1a}, R^{1b}, R^{1c} and R^{3a} is as defined in any of Tables 1 to 1000 and Q is radical of formula II.83

### Tables 203001 to 204000

Compounds of the formula Ia.1 in which the combination of R^{1a}, R^{1b}, R^{1c} and R^{3a} is as defined in any of Tables 1 to 1000 and Q is radical of formula II.84

### Tables 204001 to 205000

Compounds of the formula Ia.1 in which the combination of R^{1a}, R^{1b}, R^{1c} and R^{3a} is as defined in any of Tables 1 to 1000 and Q is radical of formula II.85

### Tables 205001 to 206000

Compounds of the formula Ia.1 in which the combination of R^{1a}, R^{1b}, R^{1c} and R^{3a} is as defined in any of Tables 1 to 1000 and Q is radical of formula II.86

### Tables 206001 to 207000

Compounds of the formula Ia.1 in which the combination of R^{1a}, R^{1b}, R^{1c} and R^{3a} is as defined in any of Tables 1 to 1000 and Q is radical of formula II.87

### Tables 207001 to 208000

Compounds of the formula Ia.1 in which the combination of R^{1a}, R^{1b}, R^{1c} and R^{3a} is as defined in any of Tables 1 to 1000 and Q is radical of formula II.88

### Tables 208001 to 209000

Compounds of the formula Ia.1 in which the combination of R^{1a}, R^{1b}, R^{1c} and R^{3a} is as defined in any of Tables 1 to 1000 and Q is radical of formula II.89

### Tables 209001 to 210000

Compounds of the formula Ia.1 in which the combination of R^{1a}, R^{1b}, R^{1c} and R^{3a} is as defined in any of Tables 1 to 1000 and Q is radical of formula II.90

### Tables 210001 to 211000

Compounds of the formula Ia.1 in which the combination of R^{1a}, R^{1b}, R^{1c} and R^{3a} is as defined in any of Tables 1 to 1000 and Q is radical of formula II.91

### Tables 211001 to 212000

Compounds of the formula Ia.1 in which the combination of R^{1a}, R^{1b}, R^{1c} and R^{3a} is as defined in any of Tables 1 to 1000 and Q is radical of formula II.92

### Tables 212001 to 213000

Compounds of the formula Ia.1 in which the combination of R^{1a}, R^{1b}, R^{1c} and R^{1a} is as defined in any of Tables 1 to 1000 and Q is radical of formula II.93

### Tables 213001 to 214000

Compounds of the formula Ia.1 in which the combination of R^{1a}, R^{1b}, R^{1c} and R^{3a} is as defined in any of Tables 1 to 1000 and Q is radical of formula II.94

### Tables 214001 to 215000

Compounds of the formula Ia.1 in which the combination of R^{1a}, R^{1b}, R^{1c} and R^{3a} is as defined in any of Tables 1 to 1000 and Q is radical of formula II.95

### Tables 215001 to 216000

Compounds of the formula Ia.1 in which the combination of R^{1a}, R^{1b}, R^{1c} and R^{3a} is as defined in any of Tables 1 to 1000 and Q is radical of formula II.96, wherein R^{3b} is Cl

### Tables 216001 to 217000

Compounds of the formula Ia.1 in which the combination of R^{1a}, R^{1b}, R^{1c} and R^{3a} is as defined in any of Tables 1 to 1000 and Q is radical of formula II.96, wherein R^{3b} is CN

### Tables 217001 to 218000

Compounds of the formula Ia.1 in which the combination of R^{1a}, R^{1b}, R^{1c} and R^{3a} is as defined in any of Tables 1 to 1000 and Q is radical of formula II.96, wherein R^{3b} is OH

### Tables 218001 to 219000

Compounds of the formula Ia.1 in which the combination of R^{1a}, R^{1b}, R^{1c} and R^{3a} is as defined in any of Tables 1 to 1000 and Q is radical of formula II.96, wherein R^{3b} is methoxy

### Tables 219001 to 220000

Compounds of the formula Ia.1 in which the combination of R^{1a}, R^{1b}, R^{1c} and R^{3a} is as defined in any of Tables 1 to 1000 and Q is radical of formula II.97

### Tables 220001 to 221000

Compounds of the formula Ia.1 in which the combination of R^{1a}, R^{1b}, R^{1c} and R^{3a} is as defined in any of Tables 1 to 1000 and Q is radical of formula II.98

### Tables 221001 to 222000

Compounds of the formula Ia.1 in which the combination of R^{1a}, R^{1b}, R^{1c} and R^{3a} is as defined in any of Tables 1 to 1000 and Q is radical of formula II.99

### Tables 222001 to 223000

Compounds of the formula Ia.1 in which the combination of R^{1a}, R^{1b}, R^{1c} and R^{3a} is as defined in any of Tables 1 to 1000 and Q is radical of formula II.100

### Tables 223001 to 224000

Compounds of the formula Ia.1 in which the combination of R^{1a}, R^{1b}, R^{1c} and R^{3a} is as defined in any of Tables 1 to 1000 and Q is radical of formula II.101

### Tables 224001 to 225000

Compounds of the formula Ia.1 in which the combination of R^{1a}, R^{1b}, R^{1c} and R^{3a} is as defined in any of Tables 1 to 1000 and Q is radical of formula II.102

### Tables 225001 to 226000

Compounds of the formula Ia.1 in which the combination of R^{1a}, R^{1b}, R^{1c} and R^{3a} is as defined in any of Tables 1 to 1000 and Q is radical of formula II.103

### Tables 226001 to 227000

Compounds of the formula Ia.1 in which the combination of R^{1a}, R^{1b}, R^{1c} and R^{3a} is as defined in any of Tables 1 to 1000 and Q is radical of formula II.104

### Tables 227001 to 228000

Compounds of the formula Ia.1 in which the combination of R^{1a}, R^{1b}, R^{1c} and R^{3a} is as defined in any of Tables 1 to 1000 and Q is radical of formula II.105

### Tables 228001 to 229000

Compounds of the formula Ia.1 in which the combination of R^{1a}, R^{1b}, R^{1c} and R^{3a} is as defined in any of Tables 1 to 1000 and Q is radical of formula II.106

### Tables 229001 to 230000

Compounds of the formula Ia.1 in which the combination of R^{1a}, R^{1b}, R^{1c} and R^{3a} is as defined in any of Tables 1 to 1000 and Q is radical of formula II.107

### Tables 230001 to 231000

Compounds of the formula Ia.1 in which the combination of R^{1a}, R^{1b}, R^{1c} and R^{3a} is as defined in any of Tables 1 to 1000 and Q is radical of formula II.108, wherein R^{3b} is Cl

### Tables 231001 to 232000

Compounds of the formula Ia.1 in which the combination of R^{1a}, R^{1b}, R^{1c} and R^{3a} is as defined in any of Tables 1 to 1000 and Q is radical of formula II.108, wherein R^{3b} is CN

### Tables 232001 to 233000

Compounds of the formula Ia.1 in which the combination of R^{1a}, R^{1b}, R^{1c} and R^{3a} is as defined in any of Tables 1 to 1000 and Q is radical of formula II.108, wherein R^{3b} is OH

### Tables 233001 to 234000

Compounds of the formula Ia.1 in which the combination of R^{1a}, R^{1b}, R^{1c} and R^{3a} is as defined in any of Tables 1 to 1000 and Q is radical of formula II.108, wherein R^{3b} is methoxy

### Tables 234001 to 235000

Compounds of the formula Ia.1 in which the combination of R^{1a}, R^{1b}, R^{1c} and R^{3a} is as defined in any of Tables 1 to 1000 and Q is radical of formula II.109

### Tables 235001 to 236000

Compounds of the formula Ia.1 in which the combination of R^{1a}, R^{1b}, R^{1c} and R^{3a} is as defined in any of Tables 1 to 1000 and Q is radical of formula II.110

### Tables 236001 to 237000

Compounds of the formula Ia.1 in which the combination of R^{1a}, R^{1b}, R^{1c} and R^{3a} is as defined in any of Tables 1 to 1000 and Q is radical of formula II.111, wherein R^{3b} is Cl

### Tables 237001 to 238000

Compounds of the formula Ia.1 in which the combination of R^{1a}, R^{1b}, R^{1c} and R^{3a} is as defined in any of Tables 1 to 1000 and Q is radical of formula II.111, wherein R^{3b} is CN

### Tables 238001 to 239000

Compounds of the formula Ia.1 in which the combination of R^{1a}, R^{1b}, R^{1c} and R^{3a} is as defined in any of Tables 1 to 1000 and Q is radical of formula II.111, wherein R^{3b} is OH

### Tables 239001 to 240000

Compounds of the formula Ia.1 in which the combination of R^{1a}, R^{1b}, R^{1c} and R^{3a} is as defined in any of Tables 1 to 1000 and Q is radical of formula II.111, wherein R^{3b} is methoxy

### Tables 240001 to 241000

Compounds of the formula Ia.1 in which the combination of R^{1a}, R^{1b}, R^{1c} and R^{3a} is as defined in any of Tables 1 to 1000 and Q is radical of formula II.112

### Tables 241001 to 242000

Compounds of the formula Ia.1 in which the combination of R^{1a}, R^{1b}, R^{1c} and R^{3a} is as defined in any of Tables 1 to 1000 and Q is radical of formula II.113

### Tables 242001 to 243000

Compounds of the formula Ia.1 in which the combination of R^{1a}, R^{1b}, R^{1c} and R^{3a} is as defined in any of Tables 1 to 1000 and Q is radical of formula II.114, wherein R^{3b} is Cl

### Tables 243001 to 244000

Compounds of the formula Ia.1 in which the combination of R^{1a}, R^{1b}, R^{1c} and R^{3a} is as defined in any of Tables 1 to 1000 and Q is radical of formula II.114, wherein R^{3b} is CN

### Tables 244001 to 245000

Compounds of the formula Ia.1 in which the combination of R^{1a}, R^{1b}, R^{1c} and R^{3a} is as defined in any of Tables 1 to 1000 and Q is radical of formula II.114, wherein R^{3b} is OH

### Tables 245001 to 246000

Compounds of the formula Ia.1 in which the combination of R^{1a}, R^{1b}, R^{1c} and R^{3a} is as defined in any of Tables 1 to 1000 and Q is radical of formula II.114, wherein R^{3b} is methoxy

### Tables 246001 to 247000

Compounds of the formula Ia.1 in which the combination of R^{1a}, R^{1b}, R^{1c} and R^{3a} is as defined in any of Tables 1 to 1000 and Q is radical of formula II.115

### Tables 247001 to 248000

Compounds of the formula Ia.1 in which the combination of R^{1a}, R^{1b}, R^{1c} and R^{3a} is as defined in any of Tables 1 to 1000 and Q is radical of formula II.116

### Tables 248001 to 249000

Compounds of the formula Ia.1 in which the combination of R^{1a}, R^{1b}, R^{1c} and R^{3a} is as defined in any of Tables 1 to 1000 and Q is radical of formula II.117, wherein R^{3b} is H

### Tables 249001 to 250000

Compounds of the formula Ia.1 in which the combination of R^{1a}, R^{1b}, R^{1c} and R^{3a} is as defined in any of Tables 1 to 1000 and Q is radical of formula II.117, wherein R^{3b} is methyl

### Tables 250001 to 251000

Compounds of the formula Ia.1 in which the combination of R^{1a}, R^{1b}, R^{1c} and R^{3a} is as defined in any of Tables 1 to 1000 and Q is radical of formula II.117, wherein R^{3b} is ethyl

### Tables 251001 to 252000

defined in any of Tables 1 to 1000 and Q is radical of formula II.117, wherein R^{3b} is n-propyl

### Tables 252001 to 253000

Compounds of the formula Ia.1 in which the combination of R^{1a}, R^{1b}, R^{1c} and R^{3a} is as defined in any of Tables 1 to 1000 and Q is radical of formula II.117, wherein R^{3b} is isopropyl

### Tables 253001 to 254000

Compounds of the formula Ia.1 in which the combination of R^{1a}, R^{1b}, R^{1c} and R^{3a} is as defined in any of Tables 1 to 1000 and Q is radical of formula II.117, wherein R^{3b} is benzyl

### Tables 254001 to 255000

Compounds of the formula Ia.1 in which the combination of R^{1a}, R^{1b}, R^{1c} and R^{3a} is as defined in any of Tables 1 to 1000 and Q is radical of formula II.117, wherein R^{3b} is acetyl

### Tables 255001 to 256000

Compounds of the formula Ia.1 in which the combination of R^{1a}, R^{1b}, R^{1c} and R^{3a} is as defined in any of Tables 1 to 1000 and Q is radical of formula II.118, wherein R^{3b} is H

### Tables 256001 to 257000

Compounds of the formula Ia.1 in which the combination of R^{1a}, R^{1b}, R^{1c} and R^{3a} is as defined in any of Tables 1 to 1000 and Q is radical of formula II.118, wherein R^{3b} is methyl

### Tables 257001 to 258000

Compounds of the formula Ia.1 in which the combination of R^{1a}, R^{1b}, R^{1c} and R^{3a} is as defined in any of Tables 1 to 1000 and Q is radical of formula II.118, wherein R^{3b} is ethyl

### Tables 258001 to 259000

Compounds of the formula Ia.1 in which the combination of R^{1a}, R^{1b}, R^{1c} and R^{3a} is as defined in any of Tables 1 to 1000 and Q is radical of formula II.118, wherein R^{3b} is n-propyl

### Tables 259001 to 260000

Compounds of the formula Ia.1 in which the combination of R^{1a}, R^{1b}, R^{1c} and R^{3a} is as defined in any of Tables 1 to 1000 and Q is radical of formula II.118, wherein R^{3b} is isopropyl

### Tables 260001 to 261000

Compounds of the formula Ia.1 in which the combination of R^{1a}, R^{1b}, R^{1c} and R^{3a} is as defined in any of Tables 1 to 1000 and Q is radical of formula II.118, wherein R^{3b} is benzyl

### Tables 261001 to 262000

Compounds of the formula Ia.1 in which the combination of R^{1a}, R^{1b}, R^{1c} and R^{3a} is as defined in any of Tables 1 to 1000 and Q is radical of formula II.118, wherein R^{3b} is acetyl

### Tables 262001 to 263000

Compounds of the formula Ia.1 in which the combination of R^{1a}, R^{1b}, R^{1c} and R^{3a} is as defined in any of Tables 1 to 1000 and Q is radical of formula II.119, wherein R^{3b} is H

### Tables 263001 to 264000

Compounds of the formula Ia.1 in which the combination of R^{1a}, R^{1b}, R^{1c} and R^{3a} is as defined in any of Tables 1 to 1000 and Q is radical of formula II.119, wherein R^{3b} is methyl

### Tables 264001 to 265000

Compounds of the formula Ia.1 in which the combination of R^{1a}, R^{1b}, R^{1c} and R^{1a} is as defined in any of Tables 1 to 1000 and Q is radical of formula II.31, wherein R^{3b} is ethyl

### Tables 265001 to 266000

Compounds of the formula Ia.1 in which the combination of R^{1a}, R^{1b}, R^{1c} and R^{3a} is as defined in any of Tables 1 to 1000 and Q is radical of formula II.119, wherein R^{3b} is n-propyl

### Tables 266001 to 267000

Compounds of the formula Ia.1 in which the combination of R^{1a}, R^{1b}, R^{1c} and R^{3a} is as defined in any of Tables 1 to 1000 and Q is radical of formula II.119, wherein R^{3b} is isopropyl

### Tables 267001 to 268000

Compounds of the formula Ia.1 in which the combination of R^{1a}, R^{1b}, R^{1c} and R^{1a} is as defined in any of Tables 1 to 1000 and Q is radical of formula II.119, wherein R^{3b} is benzyl

### Tables 268001 to 269000

Compounds of the formula Ia.1 in which the combination of R^{1a}, R^{1b}, R^{1c} and R^{3a} is as defined in any of Tables 1 to 1000 and Q is radical of formula II.119, wherein R^{3b} is acetyl

### Tables 269001 to 270000

Compounds of the formula Ia.1 in which the combination of R^{1a}, R^{1b}, R^{1c} and R^{3a} is as defined in any of Tables 1 to 1000 and Q is radical of formula II.120, wherein R^{3b} is H

### Tables 270001 to 271000

Compounds of the formula Ia.1 in which the combination of R^{1a}, R^{1b}, R^{1c} and R^{3a} is as defined in any of Tables 1 to 1000 and Q is radical of formula II.120, wherein R^{3b} is methyl

### Tables 271001 to 272000

Compounds of the formula Ia.1 in which the combination of R^{1a}, R^{1b}, R^{1c} and R^{3a} is as defined in any of Tables 1 to 1000 and Q is radical of formula II.120, wherein R^{3b} is ethyl

### Tables 272001 to 273000

Compounds of the formula Ia.1 in which the combination of R^{1a}, R^{1b}, R^{1c} and R^{3a} is as defined in any of Tables 1 to 1000 and Q is radical of formula II.120, wherein R^{3b} is n-propyl

### Tables 273001 to 274000

Compounds of the formula Ia.1 in which the combination of R^{1a}, R^{1b}, R^{1c} and R^{3a} is as defined in any of Tables 1 to 1000 and Q is radical of formula II.120, wherein R^{3b} is isopropyl

### Tables 274001 to 275000

Compounds of the formula Ia.1 in which the combination of R^{1a}, R^{1b}, R^{1c} and R^{1a} is as defined in any of Tables 1 to 1000 and Q is radical of formula II.120, wherein R^{3b} is benzyl

### Tables 275001 to 276000

Compounds of the formula Ia.1 in which the combination of R^{1a}, R^{1b}, R^{1c} and R^{3a} is as defined in any of Tables 1 to 1000 and Q is radical of formula II.120, wherein R^{3b} is acetyl

### Tables 276001 to 277000

Compounds of the formula Ia.1 in which the combination of R^{1a}, R^{1b}, R^{1c} and R^{3a} is as defined in any of Tables 1 to 1000 and Q is radical of formula II.121, wherein R^{3b} is H

### Tables 277001 to 278000

Compounds of the formula Ia.1 in which the combination of R^{1a}, R^{1b}, R^{1c} and R^{3a} is as defined in any of Tables 1 to 1000 and Q is radical of formula II.121, wherein R^{3b} is methyl

### Tables 278001 to 279000

Compounds of the formula Ia.1 in which the combination of R^{1a}, R^{1b}, R^{1c} and R^{1a} is as defined in any of Tables 1 to 1000 and Q is radical of formula II.121, wherein R^{3b} is ethyl

### Tables 279001 to 280000

Compounds of the formula Ia.1 in which the combination of R^{1a}, R^{1b}, R^{1c} and R^{3a} is as defined in any of Tables 1 to 1000 and Q is radical of formula II.121, wherein R^{3b} is n-propyl

### Tables 280001 to 281000

Compounds of the formula Ia.1 in which the combination of R^{1a}, R^{1b}, R^{1c} and R^{3a} is as defined in any of Tables 1 to 1000 and Q is radical of formula II.121, wherein R^{3b} is isopropyl

### Tables 281001 to 282000

Compounds of the formula Ia.1 in which the combination of R^{1a}, R^{1b}, R^{1c} and R^{3a} is as defined in any of Tables 1 to 1000 and Q is radical of formula II.121, wherein R^{3b} is benzyl

### Tables 282001 to 283000

Compounds of the formula Ia.1 in which the combination of R^{1a}, R^{1b}, R^{1c} and R^{3a} is as defined in any of Tables 1 to 1000 and Q is radical of formula II.121, wherein R^{3b} is acetyl

### Tables 283001 to 284000

Compounds of the formula Ia.1 in which the combination of R^{1a}, R^{1b}, R^{1c} and R^{3a} is as defined in any of Tables 1 to 1000 and Q is radical of formula II.122, wherein R^{3b} is H

### Tables 284001 to 285000

Compounds of the formula Ia.1 in which the combination of R^{1a}, R^{1b}, R^{1c} and R^{3a} is as defined in any of Tables 1 to 1000 and Q is radical of formula II.122, wherein R^{3b} is methyl

### Tables 285001 to 286000

Compounds of the formula Ia.1 in which the combination of R^{1a}, R^{1b}, R^{1c} and R^{3a} is as defined in any of Tables 1 to 1000 and Q is radical of formula II.122, wherein R^{3b} is ethyl

### Tables 286001 to 287000

Compounds of the formula Ia.1 in which the combination of R^{1a}, R^{1b}, R^{1c} and R^{3a} is as defined in any of Tables 1 to 1000 and Q is radical of formula II.122, wherein R^{3b} is n-propyl

### Tables 287001 to 288000

Compounds of the formula Ia.1 in which the combination of R^{1a}, R^{1b}, R^{1c} and R^{3a} is as defined in any of Tables 1 to 1000 and Q is radical of formula II.122, wherein R^{3b} is isopropyl

### Tables 288001 to 289000

Compounds of the formula Ia.1 in which the combination of R^{1a}, R^{1b}, R^{1c} and R^{3a} is as defined in any of Tables 1 to 1000 and Q is radical of formula II.122, wherein R^{3b} is benzyl

### Tables 289001 to 290000

Compounds of the formula Ia.1 in which the combination of R^{1a}, R^{1b}, R^{1c} and R^{3a} is as defined in any of Tables 1 to 1000 and Q is radical of formula II.122, wherein R^{3b} is acetyl

### Tables 290001 to 291000

Compounds of the formula Ia.1 in which the combination of R^{1a}, R^{1b}, R^{1c} and R^{1a} is as defined in any of Tables 1 to 1000 and Q is radical of formula II.123, wherein R^{3b} is H

### Tables 291001 to 292000

Compounds of the formula Ia.1 in which the combination of R^{1a}, R^{1b}, R^{1c} and R^{3a} is as defined in any of Tables 1 to 1000 and Q is radical of formula II.123, wherein R^{3b} is methyl

### Tables 292001 to 293000

Compounds of the formula Ia.1 in which the combination of R^{1a}, R^{1b}, R^{1c} and R^{1a} is as defined in any of Tables 1 to 1000 and Q is radical of formula II.123, wherein R^{3b} is ethyl

### Tables 293001 to 294000

Compounds of the formula Ia.1 in which the combination of R^{1a}, R^{1b}, R^{1c} and R^{3a} is as defined in any of Tables 1 to 1000 and Q is radical of formula II.123, wherein R^{3b} is n-propyl

### Tables 294001 to 295000

Compounds of the formula Ia.1 in which the combination of R^{1a}, R^{1b}, R^{1c} and R^{3a} is as defined in any of Tables 1 to 1000 and Q is radical of formula II.123, wherein R^{3b} is isopropyl

### Tables 295001 to 296000

Compounds of the formula Ia.1 in which the combination of R^{1a}, R^{1b}, R^{1c} and R^{3a} is as defined in any of Tables 1 to 1000 and Q is radical of formula II.123, wherein R^{3b} is benzyl

### Tables 296001 to 297000

Compounds of the formula Ia.1 in which the combination of R^{1a}, R^{1b}, R^{1c} and R^{3a} is as defined in any of Tables 1 to 1000 and Q is radical of formula II.123, wherein R^{3b} is acetyl

### Tables 297001 to 298000

Compounds of the formula Ia.1 in which the combination of R^{1a}, R^{1b}, R^{1c} and R^{3a} is as defined in any of Tables 1 to 1000 and Q is radical of formula II.124, wherein R^{3b} is H

### Tables 298001 to 299000

Compounds of the formula Ia.1 in which the combination of R^{1a}, R^{1b}, R^{1c} and R^{3a} is as defined in any of Tables 1 to 1000 and Q is radical of formula II.124, wherein R^{3b} is methyl

### Tables 299001 to 300000

Compounds of the formula Ia.1 in which the combination of R^{1a}, R^{1b}, R^{1c} and R^{3a} is as defined in any of Tables 1 to 1000 and Q is radical of formula II.124, wherein R^{3b} is ethyl

### Tables 300001 to 301000

Compounds of the formula Ia.1 in which the combination of R^{1a}, R^{1b}, R^{1c} and R^{3a} is as defined in any of Tables 1 to 1000 and Q is radical of formula II.124, wherein R^{3b} is n-propyl

### Tables 301001 to 302000

Compounds of the formula Ia.1 in which the combination of R^{1a}, R^{1b}, R^{1c} and R^{3a} is as defined in any of Tables 1 to 1000 and Q is radical of formula II.124, wherein R^{3b} is isopropyl

### Tables 302001 to 303000

Compounds of the formula Ia.1 in which the combination of R^{1a}, R^{1b}, R^{1c} and R^{3a} is as defined in any of Tables 1 to 1000 and Q is radical of formula II.124, wherein R^{3b} is benzyl

### Tables 303001 to 304000

Compounds of the formula Ia.1 in which the combination of R^{1a}, R^{1b}, R^{1c} and R^{3a} is as defined in any of Tables 1 to 1000 and Q is radical of formula II.124, wherein R^{3b} is acetyl

### Tables 304001 to 305000

Compounds of the formula Ia.1 in which the combination of R^{1a}, R^{1b}, R^{1c} and R^{3a} is as defined in any of Tables 1 to 1000 and Q is radical of formula II.125

### Tables 305001 to 306000

Compounds of the formula Ia.1 in which the combination of R^{1a}, R^{1b}, R^{1c} and R^{1a} is as defined in any of Tables 1 to 1000 and Q is radical of formula II.126

### Tables 306001 to 307000

defined in any of Tables 1 to 1000 and Q is radical of formula II.127

### Tables 307001 to 308000

Compounds of the formula Ia.1 in which the combination of R^{1a}, R^{1b}, R^{1c} and R^{3a} is as defined in any of Tables 1 to 1000 and Q is radical of formula II.128

### Tables 308001 to 309000

Compounds of the formula Ia.1 in which the combination of R^{1a}, R^{1b}, R^{1c} and R^{1a} is as defined in any of Tables 1 to 1000 and Q is radical of formula II.129

### Tables 309001 to 310000

Compounds of the formula Ia.1 in which the combination of R^{1a}, R^{1b}, R^{1c} and R^{3a} is as defined in any of Tables 1 to 1000 and Q is radical of formula II.130

### Tables 310001 to 311000

Compounds of the formula Ia.1 in which the combination of R^{1a}, R^{1b}, R^{1c} and R^{3a} is as defined in any of Tables 1 to 1000 and Q is radical of formula II.131

### Tables 311001 to 312000

Compounds of the formula Ia.1 in which the combination of R^{1a}, R^{1b}, R^{1c} and R^{1a} is as defined in any of Tables 1 to 1000 and Q is radical of formula II.132

Among the above compounds, preference is given to compounds of formula Ia.1, wherein Q is a radical of formula II.45 to 11.88. Specific preference is given to compounds of formula Ia.1, wherein Q is a radical of formula II.45.

Compounds of formula I can be prepared by one or more of the following methods and variations as described in schemes 1 to 6. The variables are as defined above formula I.

Compounds of formula (I) can be prepared by cycloaddition of styrene compounds of formula (III) with nitrile oxides derived from oximes of formula (IV) as outlined in scheme 1. The reaction typically proceeds through the intermediacy of an in situ generated hydroxamic acid chloride by reaction with chlorine, hypochloride, N-succinimide, or chloramine-T. The hydroxamic acid chloride is combined with the oxime in the presence of the styrene (III). Depending on the conditions, amine bases such as pyridine or triethylamine may be necessary. The reaction can be run in a wide variety of solvents including DMF, toluene, dichloromethane, chlorobenzene, acetonitrile, or the like.

Compounds of formula (III) can be prepared as described, for example, in WO 2007/094313.

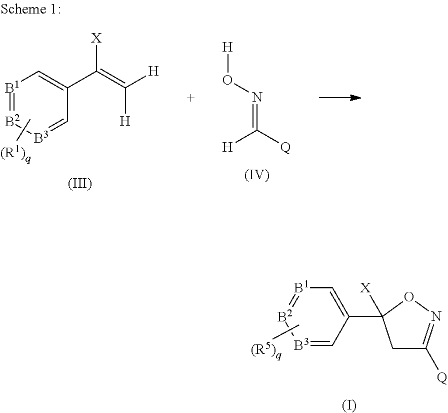

Compounds of formula (IV) can be prepared as outlined in scheme 2 by reaction of an aldehyde of formula (V) with hydroxylamine as described for example by Ostrowski, et al, Heterocycles 1996, 43, 389-96. Compounds of formula (V) can be prepared by hydrolysis of the corresponding dihalides of formula (VII) with an amine as for example described by Makosza, et al, J. Org. Chem. 1989, 54, 5094-5100. (Y is a leaving group like halogen). Compounds of formula (VII) can be prepared by halogenation of a methyl group as present in compounds of formula (VI) as for example described in WO 03/10137. Alternatively, compounds of formula (V) can be prepared from the corresponding halide of formula (VIII) by a carbonylation reaction as for example described in WO 2006/029863 (Y is a leaving group like halogen). Alternatively, compounds of formula (V) can be prepared from compounds of formula (VIII) by metalation and subsequent quenching with a formylation reagent such as dimethylformamide as for example described by Davis et al, Journal of Medicinal Chemistry 1989, 32, 1936-1942. Suitable metals for this transformations are for example magnesium, lithium, sodium, potassium, zinc, copper, mercury, tin, palladium, rhodium or ruthenium, or where applicable the salts and organic derivatives of the aforementioned.

Alternatively, compounds of formula (V) can also be prepared by oxidation of alcohols of formula (X) with an oxidizing agent such hypervalent iodine reagents, chromate salts, ruthenium salts, manganese dioxide or activated DMSO as for example described by Brak et al, Journal of the American Chemical Society 2008, 130, 6404-6410 or in WO 2006/132739 or in WO 2005/021532. Alcohols of formula (X) can be prepared by reduction of carboxylic acid derivatives of formula (IX) with a reducing agent such as complex metal hydride reagents as for example described by Brak et al, Journal of the American Chemical Society 2008, 130, 6404-6410 or in WO 2006/132739. Compounds of formula (IX) can be prepared from the corresponding halide of formula (VIII) by metalation and reaction with carbon dioxide or an alkyl chloroformiate as for example described by Mabire et al, Journal of Medicinal Chemistry 2005, 48, 2134-2153. Alternatively, Compounds of formula (IX) can be prepared from the corresponding halide of formula (VIII) by a transition catalyzed carbonylation reaction with carbon monoxide and an alcohol or water as described in WO 2008/119771.

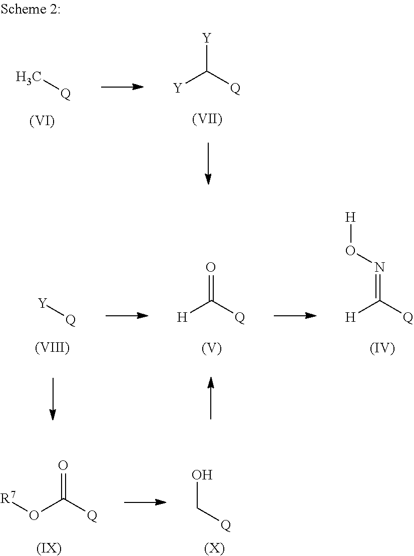

Compounds of formula (V) can also be prepared as outlined in scheme 3 from olefins of formula (XII) by either ozonolysis or a sequence of dihydroxylation and glycol cleavage as for example described WO 2006/132739. Olefins of formula (XII) can be prepared by transition metal catalyzed reaction of a compound of formula (XI) with olefins as for example described in WO 2006/132739. Alternatively, compounds of formula (XII) can be prepared from a compound of formula (XI) by metalation and reaction with a C₂-C₁₀-aldehyde followed by dehydration as for example described by Mabire et al, Journal of Medicinal Chemistry 2005, 48, 2134-2153 (J may be a leaving group as for example a halogen atom, OR⁷ or S(O)ₙR⁷).

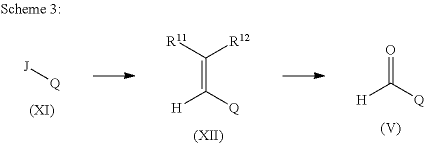

Alternatively, compounds of formula (IV) can also be prepared as outlined in scheme 4 by diazotation of amines of formula (XIV) and reaction with formoxime as for example described by Woodward et al, Advances in Chemistry Series 1958, No. 20, 22-38 or by Rey et al, Helvetica Chimica Acta 1985, 68, 1828-1834. Alternatively, compounds of formula (IV) can also be prepared from amines of formula (XIV) by treatment of the mine with formic acid and hydrogen peroxide as for example described by Kapuriya et al, Tetrahedron Letters 2008, 49, 2886-2890. Compounds of formula (XIV) can be prepared from the corresponding nitro group present in formula (XIII) by reduction with hydrogen or dissolving metals as for example described by Chezal et al, Journal of Medicinal Chemistry 2008, 51, 3133-3144 or in DE 10354860.

Compounds of formula (VIII) can also be derived from amines of formula (XIV) by Sandmayer reaction as for example described by Chezal et al, Journal of Medicinal Chemistry 2008, 51, 3133-3144.

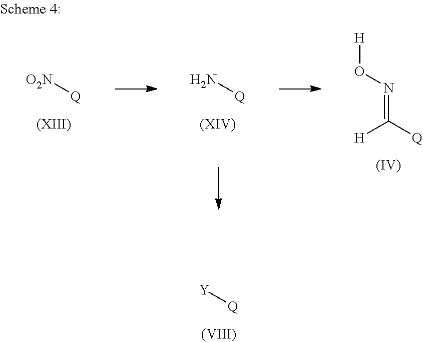

The following schemes refer to the preparation of compounds carrying substituents R³ present in radical Q. The substituent Z is —C(N═OH)—H, —C(O)—H, —NO₂, —NH₂, halogen, —CH═CR¹¹R¹², —CH₂OH, —C(O)—OR⁷, —S(O)ᵣ, R⁷, —CH₃, —CHY₂, —CN or a radical of the formula (XXI) wherein # denotes the bond in formula II.

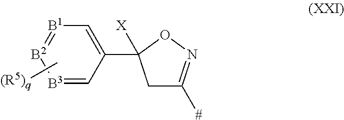

Compounds of formula (XVIII) can be prepared as outlined in scheme 5 by reaction of compounds of formula (XV) with an oxo nucleophile as for example described by Cuenca et al, Journal of Heterocyclic Chemistry 2008, 45, 1199-1201 or in WO 2008/081096 or in WO 2005/070430 (J is a leaving group like halogen, OR⁷ or S(O)ₙR⁷). Compounds of formula (XIX) can be prepared by reaction of a nitrile or a thioamide of formula (XVI) with hydroxylamine as for example described by Stephenson et al, J. Chem. Soc. 1969, 6, 861 or Goldbeck et al, Chem. Ber. 1891, 3658. Compounds of formula (XVI) can be prepared from compounds of formula (XV) as described for example in US 2007066617. Compounds of formula (XVII) can be prepared from nitriles of formula (XVI) by reduction as for example described by Langry et al, Organic Preparations and Procedures International 1994, 26, 429-438. Compounds of formula (XX) can be prepared by alkylation and/or acylation of compounds of formula (XVII) as for example described by Maayan, Tetrahedron Letters 2008, 49, 335-338.

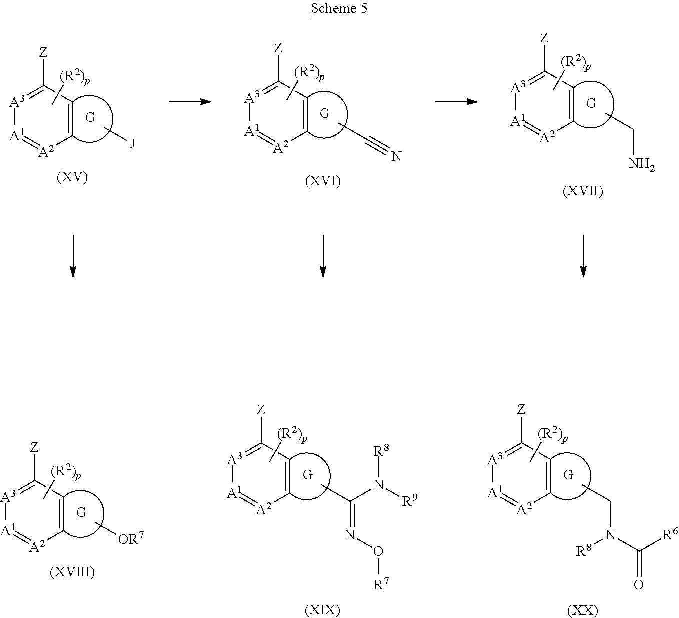

Compounds of formula (XXIV) can be prepared as outlined in scheme 6 by reaction of an ester or acid of formula (XXIII) with an amine as for example described by Kim et al, Synlett 1999, 12, 1957-1959. This process can involve the activation of the acid or ester prior to the amine coupling. Suitable activation agent for such reactions are dialkyl diimides, mixed anhydrides or conversion to the acid chloride. Alternatively, amies of the formula (XXIV) can be prepared from alkyls of the formula (XXII) by deprotonation and reaction with chloro formic acid amides as for example described by Pailloux et al, J. Org. Chem. 2007, 72, 9195-9209. Compounds of the formula (XXIII) can be prepared by deprotonation of alkyls of the formula (XXII) and reaction with chloro formic acid esters as for example described by Stevens et al, Org. Lett. 2005, 7, 4753-4756.

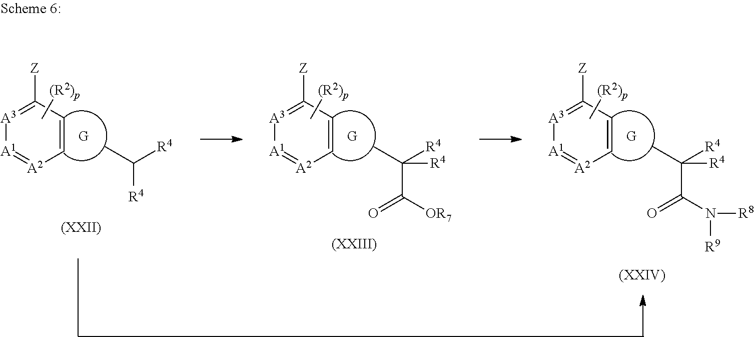

If individual compounds cannot be prepared via the above-described routes, they can be prepared by derivatization of other compounds I or by customary modifications of the synthesis routes described.

The reaction mixtures are worked up in the customary manner, for example by mixing with water, separating the phases, and, if appropriate, purifying the crude products by chromatography, for example on alumina or silica gel. Some of the intermediates and end products may be obtained in the form of colorless or pale brown viscous oils, which are freed or purified from volatile components under reduced pressure and at moderately elevated temperature. If the intermediates and end products are obtained as solids, they may be purified by recrystallization or digestion.

Due to their excellent activity, the compounds of formula I may be used for controlling invertebrate pests.

Accordingly, the present invention also provides an agricultural composition comprising at least one compound of the formula I, as defined above, a stereoisomer thereof and/or at least one agriculturally acceptable salt thereof, and at least one inert liquid and/or solid agriculturally acceptable carrier.

The present invention also provides a veterinary composition comprising at least one compound of the formula I, as defined above, a stereoisomer thereof and/or at least one veterinarily acceptable salt thereof, and at least one inert liquid and/or solid veterinarily acceptable carrier.

Such compositions may contain a single active compound of formula I or a salt thereof or a mixture of several active compounds of formula I or their salts according to the present invention. The composition according to the present invention may comprise an individual isomer or mixtures of isomers as well as individual tautomers or mixtures of tautomers.

The present invention further relates to the use of a compound as defined above, of a stereoisomer and/or of an agriculturally or veterinarily acceptable salt thereof for combating invertebrate pests.

The present invention further relates to the use of a compound as defined above, of a stereoisomer and/or of a veterinarily acceptable salt thereof, for treating or protecting an animal from infestation or infection by invertebrate pests.

Moreover the present invention also provides a method for controlling invertebrate pests which method comprises treating the pests, their food supply, their habitat or their breeding ground or a plant, plant propagation material, soil, area, material or environment in which the pests are growing or may grow, or the materials, plants, plant propagation material, soils, surfaces or spaces to be protected from invertebrate pest attack or infestation with a pesticidally effective amount of at least one compound of the formula I as defined above, a stereoisomer thereof and/or at least one agriculturally acceptable salt thereof.

Preferably, the method of the invention serves for protecting plants or plant propagation material (such as seed) and the plant which grows therefrom from animal pest attack or infestation and comprises treating the plants or the plant propagation material (such as seed) with a pesticidally effective amount of a compound of the formula I or an agriculturally acceptable salt thereof as defined above or with a pesticidally effective amount of an agricultural composition as defined above and below. The method of the invention is not limited to the protection of the “substrate” (plant, plant propagation materials, soil material etc.) which has been treated according to the invention, but also has a preventive effect, thus, for example, according protection to a plant which grows from a treated plant propagation materials (such as seed), the plant itself not having been treated.

The invention furthermore relates to plant propagation material (such as seeds), comprising at least one compound of the formula I as defined above, a stereoisomer thereof and/or at least one agriculturally acceptable salt thereof.

The invention also provides a method for treating or protecting an animal from infestation or infection by invertebrate pests which comprises bringing the animal in contact with a pesticidally effective amount of at least one compound of the formula I as defined above, a stereoisomer thereof and/or at least one veterinarily acceptable salt thereof.

The compounds of the formula I and the pestidicidal compositions comprising them are effective agents for controlling arthropod pests and nematodes. Invertebrate pests controlled by the compounds of formula I include for example:

insects from the order of the lepidopterans (Lepidoptera), for example Agrotis ypsilon, Agrotis segetum, Alabama argillacea, Anticarsia gemmatalis, Argyresthia conjugella, Autographa gamma, Bupalus piniarius, Cacoecia murinana, Capua reticulana, Chematobia brumata, Choristoneura fumiferana, Choristoneura occidentalis, Cirphis unipuncta, Cydia pomonella, Dendrolimus pini, Diaphania nitidalis, Diatraea grandiosella, Earias insulana, Elasmopalpus lignosellus, Eupoecilia ambiguella, Evetria bouliana, Feltia subterranea, Galleria mellonella, Grapholitha funebrana, Grapholitha molesta, Heliothis armigera, Heliothis virescens, Heliothis zea, Hellula undalis, Hibernia defoliaria, Hyphantria cunea, Hyponomeuta malinellus, Keiferia lycopersicella, Lamb-diva fiscellaria, Laphygma exigua, Leucoptera coffeella, Leucoptera scitella, Lithocol-letis blancardella, Lobesia botrana, Loxostege sticticalis, Lymantria dispar, Lymantria monacha, Lyonetia clerkella, Malacosoma neustria, Mamestra brassicae, Orgyia pseudotsugata, Ostrinia nubilalis, Panolis flammea, Pectinophora gossypiella, Peridroma saucia, Phalera bucephala, Phthorimaea operculella, Phyllocnistis citrella, Pieris brassicae, Plathypena scabra, Plutella xylostella, Pseudoplusia includens, Rhyacionia frustrana, Scrobipalpula absoluta, Sitotroga cerealella, Sparganothis pilleriana, Spodoptera frugiperda, Spodoptera littoralis, Spodoptera litura, Thaumatopoea pityocampa, Tortrix viridana, Trichoplusia ni and Zeiraphera canadensis;

beetles (Coleoptera), for example Agrilus sinuatus, Agriotes lineatus, Agriotes obscurus, Amphimallus solstitialis, Anisandrus dispar, Anthonomus grandis, Anthonomus pomorum, Atomaria linearis, Blastophagus piniperda, Blitophaga undata, Bruchus rufimanus, Bruchus pisorum, Bruchus lentis, Byctiscus betulae, Cassida nebulosa, Cerotoma trifurcata, Ceuthorrhynchus assimilis, Ceuthorrhynchus napi, Chaetocnema tibialis, Conoderus vespertinus, Crioceris asparagi, Diabrotica longicornis, Diabrotica 12 punctata, Diabrotica virgifera, Epilachna varivestis, Epitrix hirtipennis, Eutinobothrus brasiliensis, Hylobius abietis, Hypera brunneipennis, Hypera postica, Ips typographus, Lema bilineata, Lema melanopus, Leptinotarsa decemlineata, Limonius californicus, Lissorhoptrus oryzophilus, Melanotus communis, Meligethes aeneus, Melolontha hippocastani, Melolontha melolontha, Oulema oryzae, Ortiorrhynchus sulcatus, Otiorrhynchus ovatus, Phaedon cochleariae, Phyllotreta chrysocephala, Phyllophaga sp., Phyllopertha horticola, Phyllotreta nemorum, Phyllotreta striolata, Popillia japonica, Sitona lineatus and Sitophilus granaria; dipterans (Diptera), for example Aedes aegypti, Aedes vexans, Anastrepha ludens, Anopheles maculipennis, Ceratitis capitata, Chrysomya bezziana, Chrysomya hominivorax, Chrysomya macellaria, Contarinia sorghicola, Cordylobia anthropophaga, Culex pipiens, Dacus cucurbitae, Dacus oleae, Dasineura brassicae, Fannia canicularis, Gasterophilus intestinalis, Glossina morsitans, Haematobia irritans, Haplodiplosis equestris, Hylemyia platura, Hypoderma lineata, Liriomyza sativae, Liriomyza trifolii, Lucilia caprina, Lucilia cuprina, Lucilia sericata, Lycoria pectoralis, Mayetiola destructor, Musca domestica, Muscina stabulans, Oestrus ovis, Oscinella frit, Pegomya hysocyami, Phorbia antiqua, Phorbia brassicae, Phorbia coarctata, Rhagoletis cerasi, Rhagoletis pomonella, Tabanus bovinus, Tipula oleracea and Tipula paludosa;

thrips (Thysanoptera), e.g. Dichromothrips corbetti, Frankliniella fusca, Frankliniella occidentalis, Frankliniella tritici, Scirtothrips citri, Thrips oryzae, Thrips palmi and Thrips tabaci;

hymenopterans (Hymenoptera), e.g. Athalia rosae, Atta cephalotes, Atta sexdens, Atta texana, Hoplocampa minuta, Hoplocampa testudinea, Monomorium pharaonis, Solenopsis geminata and Solenopsis invicta;

heteropterans (Heteroptera), e.g. Acrosternum hilare, Blissus leucopterus, Cyrtopeltis notatus, Dysdercus cingulatus, Dysdercus intermedius, Eurygaster integriceps, Euschistus impictiventris, Leptoglossus phyllopus, Lygus lineolaris, Lygus pratensis, Nezara viridula, Piesma quadrata, Solubea insularis and Thyanta perditor;

homopterans (Homoptera), e.g. Acyrthosiphon onobrychis, Adelges laricis, Aphidula nasturtii, Aphis fabae, Aphis forbesi, Aphis pomi, Aphis gossypii, Aphis grossulariae, Aphis schneideri, Aphis spiraecola, Aphis sambuci, Acyrthosiphon pisum, Aulacorthum solani, Bemisia argentifolii, Brachycaudus cardui, Brachycaudus helichrysi, Brachycaudus persicae, Brachycaudus prunicola, Brevicoryne brassicae, Capitophorus horni, Cerosipha gossypii, Chaetosiphon fragaefolii, Cryptomyzus ribis, Dreyfusia nordmannianae, Dreyfusia piceae, Dysaphis radicola, Dysaulacorthum pseudosolani, Dysaphis plantaginea, Dysaphis pyri, Empoasca fabae, Hyalopterus pruni, Hyperomyzus lactucae, Macrosiphum avenae, Macrosiphum euphorbiae, Macrosiphon rosae, Megoura viciae, Melanaphis pyrarius, Metopolophium dirhodum, Myzodes persicae, Myzus ascalonicus, Myzus cerasi, Myzus persicae, Myzus varians, Nasonovia ribis-nigri, Nilaparvata lugens, Pemphigus bursarius, Perkinsiella saccharicida, Phorodon humuli, Psylla mali, Psylla piri, Rhopalomyzus ascalonicus, Rhopalosiphum maidis, Rhopalosiphum padi, Rhopalosiphum insertum, Sappaphis mala, Sappaphis mali, Schizaphis graminum, Schizoneura lanuginosa, Sitobion avenae, Sogatella furcifera Trialeurodes vaporariorum, Toxoptera aurantiiand, and Viteus vitifolii;

termites (Isoptera), e.g. Calotermes flavicollis, Leucotermes flavipes, Reticulitermes flavipes, Reticulitermes lucifugus and Termes natalensis;

orthopterans (Orthoptera), e.g. Acheta domestica, Blatta orientalis, Blattella germanica, Forficula auricularia, Gryllotalpa gryllotalpa, Locusta migratoria, Melanoplus bivittatus, Melanoplus femur-rubrum, Melanoplus mexicanus, Melanoplus sanguinipes, Melanoplus spretus, Nomadacris septemfasciata, Periplaneta americana, Schistocerca americana, Schistocerca peregrina, Stauronotus maroccanus and Tachycines asynamorus;

Arachnoidea, such as arachnids (Acarina), e.g. of the families Argasidae, Ixodidae and Sarcoptidae, such as Amblyomma americanum, Amblyomma variegatum, Argas persicus, Boophilus annulatus, Boophilus decoloratus, Boophilus microplus, Dermacentor silvarum, Hyalomma truncatum, Ixodes ricinus, Ixodes rubicundus, Ornithodorus moubata, Otobius megnini, Dermanyssus gallinae, Psoroptes ovis, Rhipicephalus appendiculatus, Rhipicephalus evertsi, Sarcoptes scabiei, and Eriophyidae spp. such as Aculus schlechtendali, Phyllocoptrata oleivora and Eriophyes sheldoni; Tarsonemidae spp. such as Phytonemus pallidus and Polyphagotarsonemus latus; Tenuipalpidaei spp. such as Brevipalpus phoenicis; Tetranychidae spp. such as Tetranychus cinnabarinus, Tetranychus kanzawai, Tetranychus pacificus, Tetranychus telarius and Tetranychus urticae, Panonychus ulmi, Panonychus citri, and oligonychus pratensis;

Siphonatera, e.g. Xenopsylla cheopsis, Ceratophyllus spp ;

The compositions and compounds of formula I are useful for the control of nematodes, especially plant parasitic nematodes such as root knot nematodes, Meloidogyne hapla, Meloidogyne incognita, Meloidogyne javanica, and other Meloidogyne species; cyst-forming nematodes, Globodera rostochiensis and other Globodera species; Heterodera avenae, Heterodera glycines, Heterodera schachtii, Heterodera trifolii, and other Heterodera species; Seed gall nematodes, Anguina species; Stem and foliar nematodes, Aphelenchoides species; Sting nematodes, Belonolaimus longicaudatus and other Belonolaimus species; Pine nematodes, Bursaphelenchus xylophilus and other Bursaphelenchus species; Ring nematodes, Criconema species, Criconemella species, Criconemoides species, Mesocriconema species; Stem and bulb nematodes, Ditylenchus destructor, Ditylenchus dipsaci and other Ditylenchus species; Awl nematodes, Dolichodorus species; Spiral nematodes, Heliocotylenchus multicinctus and other Helicotylenchus species; Sheath and sheathoid nematodes, Hemicycliophora species and Hemicriconemoides species; Hirshmanniella species; Lance nematodes, Hoploaimus species; false rootknot nematodes, Nacobbus species; Needle nematodes, Longidorus elongatus and other Longidorus species; Pin nematodes, Paratylenchus species; Lesion nematodes, Pratylenchus neglectus, Pratylenchus penetrans, Pratylenchus curvitatus, Pratylenchus goodeyi and other Pratylenchus species; Burrowing nematodes, Radopholus similis and other Radopholus species; Reniform nematodes, Rotylenchus robustus and other Rotylenchus species; Scutellonema species; Stubby root nematodes, Trichodorus primitivus and other Trichodorus species, Paratrichodorus species; Stunt nematodes, Tylenchorhynchus claytoni, Tylenchorhynchus dubius and other Tylenchorhynchus species; Citrus nematodes, Tylenchulus species; Dagger nematodes, Xiphinema species; and other plant parasitic nematode species.

In a preferred embodiment of the invention the compounds of formula I are used for controlling insects or arachnids, in particular insects of the orders Lepidoptera, Coleoptera, Thysanoptera and Homoptera and arachnids of the order Acarina. The compounds of the formula I according to the present invention are particularly useful for controlling insects of the order Thysanoptera and Homoptera.

The compounds of formula I or the pesticidal compositions comprising them may be used to protect growing plants and crops from attack or infestation by invertebrate pests, especially insects, acaridae or arachnids by contacting the plant/crop with a pesticidally effective amount of compounds of formula I. The term “crop” refers both to growing and harvested crops.

The compounds of formula I can be converted into the customary formulations, for example solutions, emulsions, suspensions, dusts, powders, pastes and granules. The use form depends on the particular intended purpose; in each case, it should ensure a fine and even distribution of the compound according to the invention.

The formulations are prepared in a known manner (see e.g. for review U.S. Pat. No. 3,060,084, EP-A 707 445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and et seq. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Germany), 2001, 2. D. A. Knowles, Chemistry and Technology of Agrochemical Formulations, Kluwer Academic Publishers, Dordrecht, 1998 (ISBN 0-7514-0443-8), for example by extending the active compound with auxiliaries suitable for the formulation of agrochemicals, such as solvents and/or carriers, if desired emulsifiers, surfactants and dispersants, preservatives, anti-foaming agents, anti-freezing agents, for seed treatment formulation also optionally colorants and/or binders and/or gelling agents.

Examples of suitable solvents are water, aromatic solvents (for example Solvesso products, xylene), paraffins (for example mineral oil fractions), alcohols (for example methanol, butanol, pentanol, benzyl alcohol), ketones (for example cyclohexanone, gamma-butyrolactone), pyrrolidones (N-methylpyrrolidone [NMP], N-octylpyrrolidone [NOP]), acetates (glycol diacetate), glycols, fatty acid dimethylamides, fatty acids and fatty acid esters. In principle, solvent mixtures may also be used.

Suitable emulsifiers are non-ionic and anionic emulsifiers (for example polyoxyethylene fatty alcohol ethers, alkylsulfonates and arylsulfonates).

Examples of dispersants are lignin-sulfite waste liquors and methylcellulose.

Suitable surfactants used are alkali metal, alkaline earth metal and ammonium salts of lignosulfonic acid, naphthalenesulfonic acid, phenolsulfonic acid, dibutylnaphthalenesulfonic acid, alkylarylsulfonates, alkyl sulfates, alkylsulfonates, fatty alcohol sulfates, fatty acids and sulfated fatty alcohol glycol ethers, furthermore condensates of sulfonated naphthalene and naphthalene derivatives with formaldehyde, condensates of naphthalene or of naphthalenesulfonic acid with phenol and formaldehyde, polyoxyethylene octylphenol ether, ethoxylated isooctylphenol, octylphenol, nonylphenol, alkylphenol polyglycol ethers, tributylphenyl polyglycol ether, tristearylphenyl polyglycol ether, alkylaryl polyether alcohols, alcohol and fatty alcohol ethylene oxide condensates, ethoxylated castor oil, polyoxyethylene alkyl ethers, ethoxylated polyoxypropylene, lauryl alcohol polyglycol ether acetal, sorbitol esters, lignosulfite waste liquors and methylcellulose.

Substances which are suitable for the preparation of directly sprayable solutions, emulsions, pastes or oil dispersions are mineral oil fractions of medium to high boiling point, such as kerosene or diesel oil, furthermore coal tar oils and oils of vegetable or animal origin, aliphatic, cyclic and aromatic hydrocarbons, for example toluene, xylene, paraffin, tetrahydronaphthalene, alkylated naphthalenes or their derivatives, methanol, ethanol, propanol, butanol, cyclohexanol, cyclohexanone, isophorone, highly polar solvents, for example dimethyl sulfoxide, N-methylpyrrolidone or water.

Also anti-freezing agents such as glycerin, ethylene glycol, propylene glycol and bactericides such as can be added to the formulation.

Suitable antifoaming agents are for example antifoaming agents based on silicon or magnesium stearate.

A suitable preservative is e.g. dichlorophen.

Seed treatment formulations may additionally comprise binders and optionally colorants.

Binders can be added to improve the adhesion of the active materials on the seeds after treatment. Suitable binders are block copolymers EO/PO surfactants but also polyvinylalcoholsl, polyvinylpyrrolidones, polyacrylates, polymethacrylates, polybutenes, polyisobutylenes, polystyrene, polyethyleneamines, polyethyleneamides, polyethyleneimines (Lupasol®, Polymin®), polyethers, polyurethans, polyvinylacetate, tylose and copolymers derived from these polymers.

Optionally, also colorants can be included in the formulation. Suitable colorants or dyes for seed treatment formulations are Rhodamin B, C.I. Pigment Red 112, C.I. Solvent Red 1, pigment blue 15:4, pigment blue 15:3, pigment blue 15:2, pigment blue 15:1, pigment blue 80, pigment yellow 1, pigment yellow 13, pigment red 112, pigment red 48:2, pigment red 48:1, pigment red 57:1, pigment red 53:1, pigment orange 43, pigment orange 34, pigment orange 5, pigment green 36, pigment green 7, pigment white 6, pigment brown 25, basic violet 10, basic violet 49, acid red 51, acid red 52, acid red 14, acid blue 9, acid yellow 23, basic red 10, basic red 108.

Examples of a gelling agent is carrageen (Satiagel®).

Powders, materials for spreading and dustable products can be prepared by mixing or concomitantly grinding the active substances with a solid carrier.

Granules, for example coated granules, impregnated granules and homogeneous granules, can be prepared by binding the active compounds to solid carriers.

Examples of solid carriers are mineral earths such as silica gels, silicates, talc, kaolin, attaclay, limestone, lime, chalk, bole, loess, clay, dolomite, diatomaceous earth, calcium sulfate, magnesium sulfate, magnesium oxide, ground synthetic materials, fertilizers, such as, for example, ammonium sulfate, ammonium phosphate, ammonium nitrate, ureas, and products of vegetable origin, such as cereal meal, tree bark meal, wood meal and nutshell meal, cellulose powders and other solid carriers.

In general, the formulations comprise from 0.01 to 95% by weight, preferably from 0.1 to 90% by weight, of the active compound(s). In this case, the active compound(s) are employed in a purity of from 90% to 100% by weight, preferably 95% to 100% by weight (according to NMR spectrum).

For seed treatment purposes, respective formulations can be diluted 2-10 fold leading to concentrations in the ready to use preparations of 0.01 to 60% by weight active compound by weight, preferably 0.1 to 40% by weight.

The compounds of formula I can be used as such, in the form of their formulations or the use forms prepared therefrom, for example in the form of directly sprayable solutions, powders, suspensions or dispersions, emulsions, oil dispersions, pastes, dustable products, materials for spreading, or granules, by means of spraying, atomizing, dusting, spreading or pouring. The use forms depend entirely on the intended purposes; they are intended to ensure in each case the finest possible distribution of the active compound(s) according to the invention.

Aqueous use forms can be prepared from emulsion concentrates, pastes or wettable powders (sprayable powders, oil dispersions) by adding water. To prepare emulsions, pastes or oil dispersions, the substances, as such or dissolved in an oil or solvent, can be homogenized in water by means of a wetter, tackifier, dispersant or emulsifier. However, it is also possible to prepare concentrates composed of active substance, wetter, tackifier, dispersant or emulsifier and, if appropriate, solvent or oil, and such concentrates are suitable for dilution with water.

The active compound concentrations in the ready-to-use preparations can be varied within relatively wide ranges. In general, they are from 0.0001 to 10%, preferably from 0.01 to 1% per weight.

The active compound(s) may also be used successfully in the ultra-low-volume process (ULV), it being possible to apply formulations comprising over 95% by weight of active compound, or even to apply the active compound without additives.

The following are examples of formulations:

1. Products for dilution with water for foliar applications. For seed treatment purposes, such products may be applied to the seed diluted or undiluted.

### A) Water-Soluble Concentrates (SL, LS)

10 parts by weight of the active compound(s) are dissolved in 90 parts by weight of water or a water-soluble solvent. As an alternative, wetters or other auxiliaries are added. The active compound(s) dissolves upon dilution with water, whereby a formulation with 10% (w/w) of active compound(s) is obtained.

### B) Dispersible Concentrates (DC)

20 parts by weight of the active compound(s) are dissolved in 70 parts by weight of cyclohexanone with addition of 10 parts by weight of a dispersant, for example polyvinylpyrrolidone. Dilution with water gives a dispersion, whereby a formulation with 20% (w/w) of active compound(s) is obtained.

### C) Emulsifiable Concentrates (EC)

15 parts by weight of the active compound(s) are dissolved in 7 parts by weight of xylene with addition of calcium dodecylbenzenesulfonate and castor oil ethoxylate (in each case 5 parts by weight). Dilution with water gives an emulsion, whereby a formulation with 15% (w/w) of active compound(s) is obtained.

### D) Emulsions (EW, EO, ES)

25 parts by weight of the active compound(s) are dissolved in 35 parts by weight of xylene with addition of calcium dodecylbenzenesulfonate and castor oil ethoxylate (in each case 5 parts by weight). This mixture is introduced into 30 parts by weight of water by means of an emulsifier machine (e.g. Ultraturrax) and made into a homogeneous emulsion. Dilution with water gives an emulsion, whereby a formulation with 25% (w/w) of active compound(s) is obtained.

### E) Suspensions (SC, OD, FS)

In an agitated ball mill, 20 parts by weight of the active compound(s) are comminuted with addition of 10 parts by weight of dispersants, wetters and 70 parts by weight of water or of an organic solvent to give a fine active compound(s) suspension. Dilution with water gives a stable suspension of the active compound(s), whereby a formulation with 20% (w/w) of active compound(s) is obtained.

### F) Water-Dispersible Granules and Water-Soluble Granules (WG, SG)

50 parts by weight of the active compound(s) are ground finely with addition of 50 parts by weight of dispersants and wetters and made as water-dispersible or water-soluble granules by means of technical appliances (for example extrusion, spray tower, fluidized bed). Dilution with water gives a stable dispersion or solution of the active compound(s), whereby a formulation with 50% (w/w) of active compound(s) is obtained.

### G) Water-Dispersible Powders and Water-Soluble Powders (WP, SP, SS, WS)

75 parts by weight of the active compound(s) are ground in a rotor-stator mill with addition of 25 parts by weight of dispersants, wetters and silica gel. Dilution with water gives a stable dispersion or solution of the active compound(s), whereby a formulation with 75% (w/w) of active compound(s) is obtained.

### H) Gel-Formulation (GF)

In an agitated ball mill, 20 parts by weight of the active compound(s) are comminuted with addition of 10 parts by weight of dispersants, 1 part by weight of a gelling agent wetters and 70 parts by weight of water or of an organic solvent to give a fine active compound(s) suspension. Dilution with water gives a stable suspension of the active compound(s), whereby a formulation with 20% (w/w) of active compound(s) is obtained.

2. Products to be applied undiluted for foliar applications. For seed treatment purposes, such products may be applied to the seed diluted or undiluted.

I) Dustable Powders (DP, DS)

5 parts by weight of the active compound(s) are ground finely and mixed intimately with 95 parts by weight of finely divided kaolin. This gives a dustable product having 5% (w/w) of active compound(s)

### J) Granules (GR, FG, GG, MG)

0.5 parts by weight of the active compound(s) is ground finely and associated with 95.5 parts by weight of carriers, whereby a formulation with 0.5% (w/w) of active compound(s) is obtained. Current methods are extrusion, spray-drying or the fluidized bed. This gives granules to be applied undiluted for foliar use.

K) ULV solutions (UL)

10 parts by weight of the active compound(s) are dissolved in 90 parts by weight of an organic solvent, for example xylene. This gives a product having 10% (w/w) of active compound(s), which is applied undiluted for foliar use.

The compounds of formula I are also suitable for the treatment of plant propagation materials (such as seed). Conventional seed treatment formulations include for example flowable concentrates FS, solutions LS, powders for dry treatment DS, water dispersible powders for slurry treatment WS, water-soluble powders SS and emulsion ES and EC and gel formulation GF. These formulations can be applied to the seed diluted or undiluted. Application to the seeds is carried out before sowing, either directly on the seeds or after having pregerminated the latter

In a preferred embodiment a FS formulation is used for seed treatment. Typically, a FS formulation may comprise 1-800 g/l of active ingredient, 1-200 g/l Surfactant, 0 to 200 g/l antifreezing agent, 0 to 400 g/l of binder, 0 to 200 g/l of a pigment and up to 1 liter of a solvent, preferably water.

Other preferred FS formulations of compounds of formula I for seed treatment comprise from 0.5 to 80 wt % of the active ingredient, from 0.05 to 5 wt % of a wetter, from 0.5 to 15 wt % of a dispersing agent, from 0.1 to 5 wt % of a thickener, from 5 to 20 wt % of an anti-freeze agent, from 0.1 to 2 wt % of an anti-foam agent, from 1 to 20 wt % of a pigment and/or a dye, from 0 to 15 wt % of a sticker/adhesion agent, from 0 to 75 wt % of a filler/vehicle, and from 0.01 to 1 wt % of a preservative.

Various types of oils, wetters, adjuvants, herbicides, fungicides, other pesticides, or bactericides may be added to the active ingredients, if appropriate just immediately prior to use (tank mix). These agents usually are admixed with the agents according to the invention in a weight ratio of 1:10 to 10:1.

The compounds of formula I are effective through both contact (via soil, glass, wall, bed net, carpet, plant parts or animal parts), and ingestion (bait, or plant part).

For use against ants, termites, wasps, flies, mosquitos, crickets, or cockroaches, compounds of formula I are preferably used in a bait composition.

The bait can be a liquid, a solid or a semisolid preparation (e.g. a gel). Solid baits can be formed into various shapes and forms suitable to the respective application e.g. granules, blocks, sticks, disks. Liquid baits can be filled into various devices to ensure proper application, e.g. open containers, spray devices, droplet sources, or evaporation sources. Gels can be based on aqueous or oily matrices and can be formulated to particular necessities in terms of stickiness, moisture retention or aging characteristics.

The bait employed in the composition is a product which is sufficiently attractive to incite insects such as ants, termites, wasps, flies, mosquitos, crickets etc. or cockroaches to eat it. The attractiveness can be manipulated by using feeding stimulants or sex pheromones. Food stimulants are chosen, for example, but not exclusively, from animal and/or plant proteins (meat-, fish- or blood meal, insect parts, egg yolk), from fats and oils of animal and/or plant origin, or mono-, oligo- or polyorganosaccharides, especially from sucrose, lactose, fructose, dextrose, glucose, starch, pectin or even molasses or honey. Fresh or decaying parts of fruits, crops, plants, animals, insects or specific parts thereof can also serve as a feeding stimulant. Sex pheromones are known to be more insect specific. Specific pheromones are described in the literature and are known to those skilled in the art.

Formulations of compounds of formula I as aerosols (e.g in spray cans), oil sprays or pump sprays are highly suitable for the non-professional user for controlling pests such as flies, fleas, ticks, mosquitos or cockroaches. Aerosol recipes are preferably composed of the active compound, solvents such as lower alcohols (e.g. methanol, ethanol, propanol, butanol), ketones (e.g. acetone, methyl ethyl ketone), paraffin hydrocarbons (e.g. kerosenes) having boiling ranges of approximately 50 to 250° C., dimethylformamide, N methylpyrrolidone, dimethyl sulphoxide, aromatic hydrocarbons such as toluene, xylene, water, furthermore auxiliaries such as emulsifiers such as sorbitol monooleate, oleyl ethoxylate having 3-7 mol of ethylene oxide, fatty alcohol ethoxylate, perfume oils such as ethereal oils, esters of medium fatty acids with lower alcohols, aromatic carbonyl compounds, if appropriate stabilizers such as sodium benzoate, amphoteric surfactants, lower epoxides, triethyl orthoformate and, if required, propellants such as propane, butane, nitrogen, compressed air, dimethyl ether, carbon dioxide, nitrous oxide, or mixtures of these gases.

The oil spray formulations differ from the aerosol recipes in that no propellants are used.

The compounds of formula I and their respective compositions can also be used in mosquito and fumigating coils, smoke cartridges, vaporizer plates or long-term vaporizers and also in moth papers, moth pads or other heat-independent vaporizer systerns.

Methods to control infectious diseases transmitted by insects (e.g. malaria, dengue and yellow fever, lymphatic filariasis, and leishmaniasis) with compounds of formula I and their respective compositions also comprise treating surfaces of huts and houses, air spraying and impregnation of curtains, tents, clothing items, bed nets, tsetse-fly trap or the like. Insecticidal compositions for application to fibers, fabric, knitgoods, nonwovens, netting material or foils and tarpaulins preferably comprise a mixture including the insecticide, optionally a repellent and at least one binder. Suitable repellents for example are N,N-diethyl-meta-toluamide (DEET), N,N-diethylphenylacetamide (DEPA), 1-(3-cyclohexan-1-yl-carbonyl)-2-methylpiperine, (2-hydroxymethylcyclohexyl)acetic acid lactone, 2-ethyl-1,3-hexandiol, indalone, Methylneodecanamide (MNDA), a pyrethroid not used for insect control such as {(+/−)-3-allyl-2-methyl-4-oxocyclopent-2-(+)-enyl-H-traris-chrysantemate (Esbiothrin), a repellent derived from or identical with plant extracts like limonene, eugenol, (+)-Eucamalol (1), (−)-1-epi-eucamalol or crude plant extracts from plants like Eucalyptus maculata, Vitex rotundifolia, Cymbopogan martinii, Cymbopogan citratus (lemon grass), Cymopogan nartdus (citronella). Suitable binders are selected for example from polymers and copolymers of vinyl esters of aliphatic acids (such as such as vinyl acetate and vinyl versatate), acrylic and methacrylic esters of alcohols, such as butyl acrylate, 2-ethylhexylacrylate, and methyl acrylate, mono- and diethylenically unsaturated hydrocarbons, such as styrene, and aliphatic diens, such as butadiene.

The impregnation of curtains and bednets is done in general by dipping the textile material into emulsions or dispersions of the active compounds of formula I or spraying them onto the nets.

Methods which can be employed for treating the seed are, in principle, all suitable seed treatment and especially seed dressing techniques known in the art, such as seed coating (e.g. seed pelleting), seed dusting and seed imbibition (e.g. seed soaking). Here, “seed treatment” refers to all methods that bring seeds and the compounds of formula I into contact with each other, and “seed dressing” to methods of seed treatment which provide the seeds with an amount of the compounds of formula I, i.e. which generate a seed comprising the compound of formula I. In principle, the treatment can be applied to the seed at any time from the harvest of the seed to the sowing of the seed. The seed can be treated immediately before, or during, the planting of the seed, for example using the “planter's box” method. However, the treatment may also be carried out several weeks or months, for example up to 12 months, before planting the seed, for example in the form of a seed dressing treatment, without a substantially reduced efficacy being observed.

Expediently, the treatment is applied to unsown seed. As used herein, the term “unsown seed” is meant to include seed at any period from the harvest of the seed to the sowing of the seed in the ground for the purpose of germination and growth of the plant.

Specifically, a procedure is followed in the treatment in which the seed is mixed, in a suitable device, for example a mixing device for solid or solid/liquid mixing partners, with the desired amount of seed treatment formulations, either as such or after previous dilution with water, until the composition is distributed uniformly on the seed. If appropriate, this is followed by a drying step.

The compounds of formula I or veterinarily acceptable salts thereof are in particular also suitable for being used for combating parasites in and on animals.

A further object of the present invention is therefore to provide new methods for controlling parasites in and on animals. Another object of the invention is to provide safer pesticides for animals. Another object of the invention is further to provide pesticides for animals that may be used in lower doses than existing pesticides. And another object of the invention is to provide pesticides for animals, which provide a long residual control of the parasites.

The invention also relates to compositions containing a parasiticidally effective amount of compounds of formula I or veterinarily acceptable salts thereof and an acceptable carrier, for combating parasites in and on animals.

The present invention also provides a method for treating, controlling, preventing and protecting animals against infestation and infection by parasites, which comprises orally, topically or parenterally administering or applying to the animals a parasiticidally effective amount of a compound of formula I or veterinarily acceptable salts thereof or a composition comprising it.

The present invention also provides a non-therapeutic method for treating, controlling, preventing and protecting animals against infestation and infection by parasites, which comprises applying to a locus a parasiticidally effective amount of a compound of formula I or the enantiomers or veterinarily acceptable salts thereof or a composition comprising it.

The invention also provides a process for the preparation of a composition for treating, controlling, preventing or protecting animals against infestation or infection by parasites which comprises including a parasiticidally effective amount of a compound of formula I or the veterinarily acceptable salts thereof or a composition comprising it.

The invention relates further to the use of compounds of formula I for treating, controlling, preventing or protecting animals against infestation or infection by parasites.

The invention relates also to the use of a compound of formula I, or a composition comprising it, for the manufacture of a medicament for the therapeutic treatment of animals against infections or infestations by parasites.

Activity of compounds against agricultural pests does not suggest their suitability for control of endo- and ectoparasites in and on animals which requires, for example, low, nonemetic dosages in the case of oral application, metabolic compatibility with the animal, low toxicity, and a safe handling.

Surprisingly, it has been found that compounds of formula I are suitable for combating endo- and ectoparasites in and on animals.

Compounds of formula I or veterinarily acceptable salts thereof and compositions comprising them are preferably used for controlling and preventing infestations and infections animals including warm-blooded animals (including humans) and fish. They are for example suitable for controlling and preventing infestations and infections in mammals such as cattle, sheep, swine, camels, deer, horses, pigs, poultry, rabbits, goats, dogs and cats, water buffalo, donkeys, fallow deer and reindeer, and also in fur-bearing animals such as mink, chinchilla and raccoon, birds such as hens, geese, turkeys and ducks and fish such as fresh- and salt-water fish such as trout, carp and eels.

Compounds of formula I or veterinarily acceptable salts thereof and compositions comprising them are preferably used for controlling and preventing infestations and infections in domestic animals, such as dogs or cats.

Infestations in warm-blooded animals and fish include, but are not limited to, lice, biting lice, ticks, nasal bots, keds, biting flies, muscoid flies, flies, myiasitic fly larvae, chiggers, gnats, mosquitoes and fleas.

The compounds of formula I or veterinarily acceptable salts thereof and compositions comprising them are suitable for systemic and/or non-systemic control of ecto- and/or endoparasites. They are active against all or some stages of development.

The compounds of formula I are especially useful for combating ectoparasites.

The compounds of formula I are especially useful for combating endoparasites.

The compounds of formula I are especially useful for combating parasites of the following orders and species, respectively:

fleas (Siphonaptera), e.g. Ctenocephalides felis, Ctenocephalides canis, Xenopsylla cheopis, Pulex irritans, Tunga penetrans, and Nosopsyllus fasciatus,

cockroaches (Blattaria-Blattodea), e.g. Blattella germanica, Blattella asahinae, Periplaneta americana, Periplaneta japonica, Periplaneta brunnea, Periplaneta fuligginosa, Periplaneta australasiae, and Blatta orientalis,

flies, mosquitoes (Diptera), e.g. Aedes aegypti, Aedes albopictus, Aedes vexans, Anastrepha ludens, Anopheles maculipennis, Anopheles crucians, Anopheles albimanus, Anopheles gambiae, Anopheles freeborni, Anopheles leucosphyrus, Anopheles minimus, Anopheles quadrimaculatus, Calliphora vicina, Chrysomya bezziana, Chrysomya hominivorax, Chrysomya macellaria, Chrysops discalis, Chrysops silacea, Chrysops atlanticus, Cochliomyia hominivorax, Cordylobia anthropophaga, Culicoides furens, Culex pipiens, Culex nigripalpus, Culex quinquefasciatus, Culex tarsalis, Culiseta inornata, Culiseta melanura, Dermatobia hominis, Fannia canicularis, Gasterophilus intestinalis, Glossina morsitans, Glossina palpalis, Glossina fuscipes, Glossina tachinoides, Haematobia irritans, Haplodiplosis equestris, Hippelates spp., Hypoderma lineata, Leptoconops torrens, Lucilia caprina, Lucilia cuprina, Lucilia sericata, Lycoria pectoralis, Mansonia spp., Musca domestica, Muscina stabulans, Oestrus ovis, Phlebotomus argentipes, Psorophora columbiae, Psorophora discolor, Prosimulium mixtum, Sarcophaga haemorrhoidalis, Sarcophaga sp., Simulium vittatum, Stomoxys calcitrans, Tabanus bovinus, Tabanus atratus, Tabanus lineola, and Tabanus similis,

lice (Phthiraptera), e.g. Pediculus humanus capitis, Pediculus humanus corporis, Pthirus pubis, Haematopinus eurysternus, Haematopinus suis, Linognathus vituli, Bovicola bovis, Menopon gallinae, Menacanthus stramineus and Solenopotes capillatus.

ticks and parasitic mites (Parasitiformes): ticks (Ixodida), e.g. Ixodes scapularis, Ixodes holocyclus, Ixodes pacificus, Rhiphicephalus sanguineus, Dermacentor andersoni, Dermacentor variabilis, Amblyomma americanum, Ambryomma maculatum, Ornithodorus hermsi, Ornithodorus turicata and parasitic mites (Mesostigmata), e.g. Ornithonyssus bacoti and Dermanyssus gallinae,

actinedida (Prostigmata) and Acaridida (Astigmata) e.g. Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., and Laminosioptes spp,

bugs (Heteropterida): Cimex lectularius, Cimex hemipterus, Reduvius senilis, Triatoma spp., Rhodnius ssp., Panstrongylus ssp. and Arilus critatus,

Anoplurida, e.g. Haematopinus spp., Linognathus spp., Pediculus spp., Phtirus spp., and Solenopotes spp,

Mallophagida (suborders Arnblycerina and Ischnocerina), e.g. Trimenopon spp., Menopon spp., Trinoton spp., Bovicola spp., Werneckiella spp., Lepikentron spp., Trichodectes spp., and Felicola spp,

### Roundworms Nematoda:

Wipeworms and Trichinosis (Trichosyringida), e.g. Trichinellidae (Trichinella spp.), (Trichuridae) Trichuris spp., Capillaria spp,

Rhabditida, e.g. Rhabditis spp, Strongyloides spp., Helicephalobus spp,

Strongylida, e.g. Strongylus spp., Ancylostoma spp., Necator americanus, Bunostomum spp. (Hookworm), Trichostrongylus spp., Haemonchus contortus., Ostertagia spp., Cooperia spp., Nematodirus spp., Dictyocaulus spp., Cyathostoma spp., Oesophagostomum spp., Stephanurus dentatus, Ollulanus spp., Chabertia spp., Stepha-nurus dentatus, Syngamus trachea, Ancylostoma spp., Uncinaria spp., Globocephalus spp., Necator spp., Metastrongylus spp., Muellerius capillaris, Protostrongylus spp., Angiostrongylus spp., Parelaphostrongylus spp. Aleurostrongylus abstrusus, and Dioctophyma renale,

Intestinal roundworms (Ascaridida), e.g. Ascaris lumbricoides, Ascaris suum, Ascaridia galli, Parascaris equorum, Enterobius vermicularis (Threadworm), Toxocara canis, Toxascaris leonine, Skrjabinema spp., and Oxyuris equi,

Camallanida, e.g. Dracunculus medinensis (guinea worm)

Spirurida, e.g. Thelazia spp. Wuchereria spp., Brugia spp., Onchocerca spp., Dirofilari spp. a, Dipetalonema spp., Setaria spp., Elaeophora spp., Spirocerca lupi, and Habronema spp.,

Thorny headed worms (Acanthocephala), e.g. Acanthocephalus spp., Macracanthorhynchus hirudinaceus and Oncicola spp,

### Planarians (Plathelminthes):

Flukes (Trematoda), e.g. Faciola spp., Fascioloides magna, Paragonimus spp., Dicrocoelium spp., Fasciolopsis buski, Clonorchis sinensis, Schistosoma spp., Trichobilharzia spp., Alaria alata, Paragonimus spp., and Nanocyetes spp,

Cercomeromorpha, in particular Cestoda (Tapeworms), e.g. Diphyllobothrium spp., Tenia spp., Echinococcus spp., Dipylidium caninum, Multiceps spp., Hymenolepis spp., Mesocestoides spp., Vampirolepis spp., Moniezia spp., Anoplocephala spp., Sirometra spp., Anoplocephala spp., and Hymenolepis spp.

The compounds of formula I and compositions containing them are particularly useful for the control of pests from the orders Diptera, Siphonaptera and Ixodida.

Moreover, the use of compounds of formula I and compositions containing them for combating mosquitoes is especially preferred.

The use of the compounds of formula I and compositions containing them for combating flies is a further preferred embodiment of the present invention.

Furthermore, the use of the compounds of formula I and compositions containing them for combating fleas is especially preferred.

The use of the compounds of formula I and of the compositions containing them for combating ticks is a further preferred embodiment of the present invention.

The compounds of formula I also are especially useful for combating endoparasites (roundworms nematoda, thorny headed worms and planarians).

The compounds of formula I can be effective through both contact (via soil, glass, wall, bed net, carpet, blankets or animal parts) and ingestion (e.g. baits).

The present invention relates to the therapeutic and the non-therapeutic use of compounds of formula I for controlling and/or combating parasites in and/or on animals.

The compounds of formula I may be used to protect the animals from attack or infestation by parasites by contacting them with a parasitically effective amount of compounds of formula I. As such, “contacting” includes both direct contact (applying the compounds/compositions directly on the parasite, e.g. also at it's locus, and optionally also administrating the compounds/composition directly on the animal) and indirect contact (applying the compounds/compositions to the locus of the parasite).

The contact of the parasite through application to its locus is an example of a non-therapeutic use of compounds of formula I.

“Locus” as defined above means the habitat, food supply, breeding ground, area, material or environment in which a parasite is growing or may grow outside of the animal. The compounds of the invention can also be applied preventively to places at which occurrence of the pests or parasites is expected.

Administration to the animal can be carried out both prophylactically and therapeutically.

Administration of the active compounds is carried out directly or in the form of suitable preparations, orally, topically/dermally or parenterally.

For oral administration to warm-blooded animals, the compounds of formula I may be formulated as animal feeds, animal feed premixes, animal feed concentrates, pills, solutions, pastes, suspensions, drenches, gels, tablets, boluses and capsules. In addition, the compounds of formulae I may be administered to the animals in their drinking water. For oral administration, the dosage form chosen should provide the animal with 0.01 mg/kg to 100 mg/kg of animal body weight per day of the compounds of formula I, preferably with 0.5 mg/kg to 100 mg/kg of animal body weight per day.

Alternatively, the compounds of formula I may be administered to animals parenterally, for example, by intraruminal, intramuscular, intravenous or subcutaneous injection. The compounds of formula I may be dispersed or dissolved in a physiologically acceptable carrier for subcutaneous injection. Alternatively, the compounds of formula I may be formulated into an implant for subcutaneous administration. In addition the compounds of formula I may be transdermally administered to animals. For parenteral administration, the dosage form chosen should provide the animal with 0.01 mg/kg to 100 mg/kg of animal body weight per day of the compounds of formula I.

The compounds of formula I may also be applied topically to the animals in the form of dips, dusts, powders, collars, medallions, sprays, shampoos, spot-on and pour-on formulations and in ointments or oil-in-water or water-in-oil emulsions. For topical application, dips and sprays usually contain 0.5 ppm to 5,000 ppm and preferably 1 ppm to 3,000 ppm of the compounds of formula I. In addition, the compounds of formula I may be formulated as ear tags for animals, particularly quadrupeds such as cattle and sheep.

Suitable preparations are:

Solutions such as oral solutions, concentrates for oral administration after dilution, solutions for use on the skin or in body cavities, pouring-on formulations, gels;

Emulsions and suspensions for oral or dermal administration; semi-solid preparations;

Formulations in which the active compound is processed in an ointment base or in an oil-in-water or water-in-oil emulsion base;

Solid preparations such as powders, premixes or concentrates, granules, pellets, tablets, boluses, capsules; aerosols and inhalants, and active compound-containing shaped articles.

Compositions suitable for injection are prepared by dissolving the active ingredient in a suitable solvent and optionally adding further ingredients such as acids, bases, buffer salts, preservatives, and solubilizers. The solutions are filtered and filled sterile.

Suitable solvents are physiologically tolerable solvents such as water, alkanols such as ethanol, butanol, benzyl alcohol, glycerol, propylene glycol, polyethylene glycols, N-methylpyrrolidone, 2-pyrrolidone, and mixtures thereof.

The active compounds can optionally be dissolved in physiologically tolerable vegetable or synthetic oils which are suitable for injection.

Suitable solubilizers are solvents which promote the dissolution of the active compound in the main solvent or prevent its precipitation. Examples are polyvinylpyrrolidone, polyvinyl alcohol, polyoxyethylated castor oil, and polyoxyethylated sorbitan ester.

Suitable preservatives are benzyl alcohol, trichlorobutanol, p-hydroxybenzoic acid esters, and n-butanol.

Oral solutions are administered directly. Concentrates are administered orally after prior dilution to the use concentration. Oral solutions and concentrates are prepared according to the state of the art and as described above for injection solutions, sterile procedures not being necessary.

Solutions for use on the skin are trickled on, spread on, rubbed in, sprinkled on or sprayed on.

Solutions for use on the skin are prepared according to the state of the art and according to what is described above for injection solutions, sterile procedures not being necessary.

Further suitable solvents are polypropylene glycol, phenyl ethanol, phenoxy ethanol, ester such as ethyl or butyl acetate, benzyl benzoate, ethers such as alkyleneglycol alkylether, e.g. dipropylenglycol monomethylether, ketons such as acetone, methylethylketone, aromatic hydrocarbons, vegetable and synthetic oils, dimethylformamide, dimethylacetamide, transcutol, solketal, propylencarbonate, and mixtures thereof.

It may be advantageous to add thickeners during preparation. Suitable thickeners are inorganic thickeners such as bentonites, colloidal silicic acid, aluminium monostearate, organic thickeners such as cellulose derivatives, polyvinyl alcohols and their copolymers, acrylates and methacrylates.

Gels are applied to or spread on the skin or introduced into body cavities. Gels are prepared by treating solutions which have been prepared as described in the case of the injection solutions with sufficient thickener that a clear material having an ointmentlike consistency results. The thickeners employed are the thickeners given above.

Pour-on formulations are poured or sprayed onto limited areas of the skin, the active compound penetrating the skin and acting systemically.

Pour-on formulations are prepared by dissolving, suspending or emulsifying the active compound in suitable skin-compatible solvents or solvent mixtures. If appropriate, other auxiliaries such as colorants, bioabsorption-promoting substances, antioxidants, light stabilizers, adhesives are added.

Suitable solvents are water, alkanols, glycols, polyethylene glycols, polypropylene glycols, glycerol, aromatic alcohols such as benzyl alcohol, phenylethanol, phenoxyethanol, esters such as ethyl acetate, butyl acetate, benzyl benzoate, ethers such as alkylene glycol alkyl ethers such as dipropylene glycol monomethyl ether, diethylene glycol mono-butyl ether, ketones such as acetone, methyl ethyl ketone, cyclic carbonates such as propylene carbonate, ethylene carbonate, aromatic and/or aliphatic hydrocarbons, vegetable or synthetic oils, DMF, dimethylacetamide, n-alkylpyrrolidones such as methylpyrrolidone, n-butylpyrrolidone or n-octylpyrrolidone, N methylpyrrolidone, 2-pyrrolidone, 2,2-dimethyl-4-oxy-methylene-1,3-dioxolane and glycerol formal.

Suitable colorants are all colorants permitted for use on animals and which can be dissolved or suspended.

Suitable absorption-promoting substances are, for example, DMSO, spreading oils such as isopropyl myristate, dipropylene glycol pelargonate, silicone oils and copolymers thereof with polyethers, fatty acid esters, triglycerides, fatty alcohols.

Suitable antioxidants are sulfites or metabisulfites such as potassium metabisulfite, ascorbic acid, butylhydroxytoluene, butylhydroxyanisole, tocopherol.

Suitable light stabilizers are, for example, novantisolic acid.

Suitable adhesives are, for example, cellulose derivatives, starch derivatives, polyacrylates, natural polymers such as alginates, gelatin.

Emulsions can be administered orally, dermally or as injections.

Emulsions are either of the water-in-oil type or of the oil-in-water type.

They are prepared by dissolving the active compound either in the hydrophobic or in the hydrophilic phase and homogenizing this with the solvent of the other phase with the aid of suitable emulsifiers and, if appropriate, other auxiliaries such as colorants, absorption-promoting substances, preservatives, antioxidants, light stabilizers, viscosity-enhancing substances.

Suitable hydrophobic phases (oils) are:

liquid paraffins, silicone oils, natural vegetable oils such as sesame oil, almond oil, castor oil, synthetic triglycerides such as caprylic/capric biglyceride, triglyceride mixture with vegetable fatty acids of the chain length C₈-C₁₂ or other specially selected natural fatty acids, partial glyceride mixtures of saturated or unsaturated fatty acids possibly also containing hydroxyl groups, mono- and diglycerides of the C₈-C₁₀ fatty acids, fatty acid esters such as ethyl stearate, di-n-butyryl adipate, hexyl laurate, dipropylene glycol perlargonate, esters of a branched fatty acid of medium chain length with saturated fatty alcohols of chain length C₁₆-C₁₈, isopropyl myristate, isopropyl palmitate, caprylic/capric acid esters of saturated fatty alcohols of chain length C₁₂-C₁₈, isopropyl stearate, oleyl oleate, decyl oleate, ethyl oleate, ethyl lactate, waxy fatty acid esters such as synthetic duck coccygeal gland fat, dibutyl phthalate, diisopropyl adipate, and ester mixtures related to the latter, fatty alcohols such as isotridecyl alcohol, 2-octyl-dodecanol, cetylstearyl alcohol, cetyleyl alcohol, and fatty acids such as oleic acid and mixtures thereof.

Suitable hydrophilic phases are: water, alcohols such as propylene glycol, glycerol, sorbitol and mixtures thereof.

Suitable emulsifiers are:

non-ionic surfactants, e.g. polyethoxylated castor oil, polyethoxylated sorbitan monooleate, sorbitan monostearate, glycerol monostearate, polyoxyethyl stearate, alkylphenol polyglycol ether; ampholytic surfactants such as di-sodium N-lauryl-p-iminodipropionate or lecithin;

anionic surfactants, such as sodium lauryl sulfate, fatty alcohol ether sulfates, mono/dialkyl polyglycol ether orthophosphoric acid ester monoethanolamine salt; cation-active surfactants, such as cetyltrimethylammonium chloride.

Suitable further auxiliaries are: substances which enhance the viscosity and stabilize the emulsion, such as carboxymethylcellulose, methylcellulose and other cellulose and starch derivatives, polyacrylates, alginates, gelatin, gum arabic, polyvinylpyrrolidone, polyvinyl alcohol, copolymers of methyl vinyl ether and maleic anhydride, polyethylene glycols, waxes, colloidal silicic acid or mixtures of the substances mentioned.

Suspensions can be administered orally or topically/dermally. They are prepared by suspending the active compound in a suspending agent, if appropriate with addition of other auxiliaries such as wetting agents, colorants, bioabsorption-promoting substances, preservatives, antioxidants, light stabilizers.

Liquid suspending agents are all homogeneous solvents and solvent mixtures.

Suitable wetting agents (dispersants) are the emulsifiers given above.

Other auxiliaries which may be mentioned are those given above.

Semi-solid preparations can be administered orally or topically/dermally. They differ from the suspensions and emulsions described above only by their higher viscosity.

For the production of solid preparations, the active compound is mixed with suitable excipients, if appropriate with addition of auxiliaries, and brought into the desired form.

Suitable excipients are all physiologically tolerable solid inert substances. Those used are inorganic and organic substances. Inorganic substances are, for example, sodium chloride, carbonates such as calcium carbonate, hydrogencarbonates, aluminium oxides, titanium oxide, silicic acids, argillaceous earths, precipitated or colloidal silica, or phosphates. Organic substances are, for example, sugar, cellulose, foodstuffs and feeds such as milk powder, animal meal, grain meals and shreds, starches.

Suitable auxiliaries are preservatives, antioxidants, and/or colorants which have been mentioned above.

Other suitable auxiliaries are lubricants and glidants such as magnesium stearate, stearic acid, talc, bentonites, disintegration-promoting substances such as starch or crosslinked polyvinylpyrrolidone, binders such as starch, gelatin or linear polyvinylpyrrolidone, and dry binders such as microcrystalline cellulose.

In general, “parasiticidally effective amount” means the amount of active ingredient needed to achieve an observable effect on growth, including the effects of necrosis, death, retardation, prevention, and removal, destruction, or otherwise diminishing the occurrence and activity of the target organism. The parasiticidally effective amount can vary for the various compounds/compositions used in the invention. A parasiticidally effective amount of the compositions will also vary according to the prevailing conditions such as desired parasiticidal effect and duration, target species, mode of application, and the like.

The compositions which can be used in the invention can comprise generally from about 0.001 to 95% of the compounds of formula I.

Generally, it is favorable to apply the compounds of formula I in total amounts of 0.5 mg/kg to 100 mg/kg per day, preferably 1 mg/kg to 50 mg/kg per day.

Ready-to-use preparations contain the compounds acting against parasites, preferably ectoparasites, in concentrations of 10 ppm to 80 percent by weight, preferably from 0.1 to 65 percent by weight, more preferably from 1 to 50 percent by weight, most preferably from 5 to 40 percent by weight.

Preparations which are diluted before use contain the compounds acting against ectoparasites in concentrations of 0.5 to 90 percent by weight, preferably of 1 to 50 percent by weight.

Furthermore, the preparations comprise the compounds of formula I against endoparasites in concentrations of 10 ppm to 2 percent by weight, preferably of 0.05 to 0.9 percent by weight, very particularly preferably of 0.005 to 0.25 percent by weight.

In a preferred embodiment of the present invention, the compositions comprising the compounds of formula I are applied dermally/topically.

In a further preferred embodiment, the topical application is conducted in the form of compound-containing shaped articles such as collars, medallions, ear tags, bands for fixing at body parts, and adhesive strips and foils.

Generally, it is favorable to apply solid formulations which release compounds of formula I in total amounts of 10 mg/kg to 300 mg/kg, preferably 20 mg/kg to 200 mg/kg, most preferably 25 mg/kg to 160 mg/kg body weight of the treated animal in the course of three weeks.

For the preparation of the shaped articles, thermoplastic and flexible plastics as well as elastomers and thermoplastic elastomers are used. Suitable plastics and elastomers are polyvinyl resins, polyurethane, polyacrylate, epoxy resins, cellulose, cellulose derivatives, polyamides and polyester which are sufficiently compatible with the compounds of formula I. A detailed list of plastics and elastomers as well as preparation procedures for the shaped articles is given e.g. in WO 03/086075.

Compositions to be used according to this invention may also contain other active ingredients, for example other pesticides, insecticides, herbicides, fungicides, other pesticides, or bactericides, fertilizers such as ammonium nitrate, urea, potash, and super-phosphate, phytotoxicants and plant growth regulators, safeners and nematicides. These additional ingredients may be used sequentially or in combination with the above-described compositions, if appropriate also added only immediately prior to use (tank mix). For example, the plant(s) may be sprayed with a composition of this invention either before or after being treated with other active ingredients.

These agents can be admixed with the agents used according to the invention in a weight ratio of 1:10 to 10:1. Mixing the compounds of formula I or the compositions comprising them in the use form as pesticides with other pesticides frequently results in a broader pesticidal spectrum of action.

The following list M of pesticides together with which the compounds according to the invention can be used and with which potential synergistic effects might be produced, is intended to illustrate the possible combinations, but not to impose any limitation:

M.1. Organo(thio)phosphate compounds: acephate, azamethiphos, azinphos-ethyl, azinphos-methyl, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, flupyrazophos, fosthiazate, heptenophos, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, trichlorfon, vamidothion;
M.2. Carbamate compounds: aldicarb, alanycarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, trimethacarb, XMC, xylylcarb, triazamate;
M.3. Pyrethroid compounds: acrinathrin, allethrin, d-cis-trans allethrin, d-trans allethrin, bifenthrin, bioallethrin, bioallethrin S-cylclopentenyl, bioresmethrin, cycloprothrin, cyfluthrin, beta-cyfluthrin, cyhalothrin, lambda-cyhalothrin, gamma-cyhalothrin, cypermethrin, alphacypermethrin, beta-cypermethrin, theta-cypermethrin, zetacypermethrin, cyphenothrin, deltamethrin, empenthrin, esfenvalerate, etofenprox, fenpropathrin, fenvalerate, flucythrinate, flumethrin, tau-fluvalinate, halfenprox, imiprothrin, metofluthrin, permethrin, phenothrin, prallethrin, profluthrin, pyrethrin (pyrethrum), resmethrin, silafluofen, tefluthrin, tetramethrin, tralomethrin, transfluthrin;
M.4. Juvenile hormone mimics: hydroprene, kinoprene, methoprene, fenoxycarb, pyriproxyfen;
M.5, Nicotinic receptor agonists/antagonists compounds: acetamiprid, bensultap, cartap hydrochloride, clothianidin, dinotefuran, imidacloprid, thiamethoxam, nitenpyram, nicotine, spinosad (allosteric agonist), spinetoram (allosteric agonist), thiacloprid, thiocyclam, thiosultap-sodium and AKD1022.

M.6. GABA gated chloride channel antagonist compounds: chlordane, endosulfan, gamma-HCH (lindane); ethiprole, fipronil, pyrafluprole, pyriprole

M.7. Chloride channel activators: abamectin, emamectin benzoate, milbemectin, lepimectin;

M.8. METI I compounds: fenazaquin, fenpyroximate, pyrimidifen, pyridaben, tebufenpyrad, tolfenpyrad, flufenerim, rotenone;

M.9. METI II and III compounds: acequinocyl, fluacyprim, hydramethylnon;

M.10. Uncouplers of oxidative phosphorylation: chlorfenapyr, DNOC;

M.11. Inhibitors of oxidative phosphorylation: azocyclotin, cyhexatin, diafenthiuron, fenbutatin oxide, propargite, tetradifon;

M.12. Moulting disruptors: cyromazine, chromafenozide, halofenozide, methoxyfenozide, tebufenozide;

M.13. Synergists: piperonyl butoxide, tribufos;

M.14. Sodium channel blocker compounds: indoxacarb, metaflumizone;

M.15. Fumigants: methyl bromide, chloropicrin sulfuryl fluoride;

M.16. Selective feeding blockers: crylotie, pymetrozine, flonicamid;

M.17. Mite growth inhibitors: clofentezine, hexythiazox, etoxazole;

M.18. Chitin synthesis inhibitors: buprofezin, bistrifluoron, chlorfluazuron, diflubenzuron, flucycloxuron, flufenoxuron, hexaflumuron, lufenuron, novaluron, noviflumuron, teflubenzuron, triflumuron;

M.19. Lipid biosynthesis inhibitors: spirodiclofen, spiromesifen, spirotetramat;

M.20. Octapaminergic agonsits: amitraz;

M.21. Ryanodine receptor modulators: flubendiamide and the phtalamid compound (R)-, (S)-3-Chlor-N1-{2-methyl-4-[1,2,2,2-tetrafluor-1-(trifluormethyl)ethyl]phenyl}-N2-(1-methyl-2-methylsulfonylethyl)phthalamid (M21.1)

M.22. Isoxazoline compounds: 415-(3,5-Dichloro-phenyl)-5-trifluoromethyl-4,5-di-hydroisoxazol-3-yl]-2-methyl-N-pyridin-2-ylmethyl-benzamide (M22.1), 4-[5-(3,5-Dichloro-phenyl)-5-trifluoromethyl-4,5-di-hydroisoxazol-3-yl]-2-methyl-N-(2,2,2-trifluoro-ethyl)-benzamide (M22.2), 4-[5-(3,5-Dichloro-phenyl)-5-trifluoromethyl-4,5-dihydro-isoxazol-3-yl]-2-methyl-N-[(2,2,2-trifluoro-ethylcarbamoyl)-methyl]-benzamide (M22.3), 4-[5-(3,5-Dichloro-phenyl)-5-trifluoromethyl-4,5-dihydro-isoxazol-3-yl]-naphthalene-1-carboxylic acid [(2,2,2-trifluoro-ethylcarbamoyl)-methyl]-amide (M22.4)-4-[5-(3,5-Dichlorophenyl)-5-trifluoromethyl-4,5-dihydro-isoxazol-3-yl]-N-[(methoxyimino)methyl]-2-methylbenzamide (M22.5), 4-[5-(3-Chloro-5-trifluoromethylphenyl)-5-trifluoromethyl-4,5-dihydro-isoxazol-3-yl]-2-methyl-N-[(2,2,2-trifluoroethylcarbamoyl)-methyl]-benzamide (M22.6), 4-[5-(3-Chloro-5-trifluoromethyl-phenyl)-5-trifluoromethyl-4,5-dihydro-isoxazol-3-yl]-naphthalene-1-carboxylic acid [(2,2,2-trifluoro-ethylcarbamoyl)-methyl]-amide (M22.7) and 545-(3,5-Dichloro-4-fluorophenyl)-5-trifluoromethyl-4,5-dihydro-isoxazol-3-yl]-2-[1,2,4]triazol-1-yl-benzonitrile (M22.8);

M.23. Anthranilamide compounds: chloranthraniliprole, cyantraniliprole, 5-Bromo-2-(3-chloro-pyridin-2-yl)-2H-pyrazole-3-carboxylic acid [4-cyano-2-(1-cyclopropyl-ethylcarbamoyl)-6-methyl-phenyl]-amide (M23.1), 5-Bromo-2-(3-chloro-pyridin-2-yl)-2H-pyrazole-3-carboxylic acid [2-chloro-4-cyano-6-(1-cyclopropyl-ethylcarbamoyl)-phenyl]-amide (M23.2), 5-Bromo-2-(3-chloro-pyridin-2-yl)-2H-pyrazole-3-carboxylic acid [2-bromo-4-cyano-6-(1-cyclopropyl-ethylcarbamoyl)-phenyl]-amide (M23.3), 5-Bromo-2-(3-chloro-pyridin-2-yl)-2H-pyrazole-3-carboxylic acid [2-bromo-4-chloro-6-(1-cyclopropyl-ethylcarbamoyl)-phenyl]-amide (M23.4), 5-Bromo-2-(3-chloro-pyridin-2-yl)-2H-pyrazole-3-carboxylic acid [2,4-dichloro-6-(1-cyclopropyl-ethylcarbamoyl)-phenyl]-amide (M23.5), 5-Bromo-2-(3-chloro-pyridin-2-yl)-2H-pyrazole-3-carboxylic acid [4-chloro-2-(1-cyclopropyl-ethylcarbamoyl)-6-methyl-phenyl]-amide (M23.6), N′-(2-{[5-Bromo-2-(3-chloro-pyridin-2-yl)-2H-pyrazole-3-carbonyl]-amino}-5-chloro-3-methyl-benzoyl)-hydrazinecarboxylic acid methyl ester (M23.7), N′-(2-{[5-Bromo-2-(3-chloro-pyridin-2-yl)-2H-pyrazole-3-carbonyl]-amino}-5-chloro-3-methyl-benzoyl)-N′-methyl-hydrazinecarboxylic acid methyl ester (M23.8),
N′-(2-{[5-Bromo-2-(3-chloro-pyridin-2-yl)-2H-pyrazole-3-carbonyl]-amino}-5-chloro-3-methyl-benzoyl)-N,N′-dimethyl-hydrazinecarboxylic acid methyl ester (M23.9), N′-(3,5-Dibromo-2-{[5-bromo-2-(3-chloro-pyridin-2-yl)-2H-pyrazole-3-carbonyl]-amino}-benzoyl)-hydrazinecarboxylic acid methyl ester (M23.10), N′-(3,5-Dibromo-2-{[5-bromo-2-(3-chloro-pyridin-2-yl)-2H-pyrazole-3-carbonyl]-amino}-benzoyl)-N′-methyl-hydrazinecarboxylic acid methyl ester (M23.11) and N′-(3,5-Dibromo-2-{[5-bromo-2-(3-chloro-pyridin-2-yl)-2H-pyrazole-3-carbonyl]-amino}-benzoyl)-N,N′-dimethyl-hydrazinecarboxylic acid methyl ester (M23.12);

M.24. Malononitrile compounds: 2-(2,2,3,3,4,4,5,5-octafluoropentyl)-2-(3,3,3-trifluoropropyl)malononitrile (CF₂H—CF₂—CF₂—CF₂—CH₂—C(CN)₂—CH₂—CH₂—CF₃) (M24.1) and 2-(2,2,3,3,4,4,5,5-octafluoropentyl)-2-(3,3,4,4,4-pentafluorobutyl)-malonodinitrile (CF₂H—CF₂—CF₂—CF₂—CH₂—C(CN)₂—CH₂—CH₂—CF₂—CF₃) (M24.2);

M.25. Microbial disruptors: Bacillus thuringiensis subsp. Israelensi, Bacillus sphaericus, Bacillus thuringiensis subsp. Aizawai, Bacillus thuringiensis subsp. Kurstaki, Bacillus thuringiensis subsp. Tenebrionis;
M.26. Aminofuranone compounds: 4-{[(6-Bromopyrid-3-yl)methyl](2-fluoroethyl)amino}furan-2(5H)-on (M26.1), 4-{[(6-Fluoropyrid-3-yl)methyl](2,2-difluoroethyl)amino}furan-2(5H)-on (M26.2), 4-{[(2-Chloro1,3-thiazolo-5-yl)methyl](2-fluoroethyl)amino}furan-2(5H)-on (M26.3), 4-{[(6-Chloropyrid-3-yl)methyl](2-fluoroethyl)amino}furan-2(5H)-on (M26.4), 4-{[(6-Chloropyrid-3-yl)methyl](2,2-difluoroethyl)amino}furan-2(5H)-on (M26.5), 4-{[(6-Chloro-5-fluoropyrid-3-yl)methyl](methyl)amino}furan-2(5H)-on (M26.6), 4-{[(5,6-Dichloropyrid-3-yl)methyl](2-fluoroethyl)amino}furan-2(5H)-on (M26.7), 4-{[(6-Chloro-5-fluoropyrid-3-yl)methyl](cyclopropyl)amino}furan-2(5H)-on (M26.8), 4-{[(6-Chloropyrid-3-yl)methyl](cyclopropyl)amino}furan-2(5H)-on (M26.9) and 4-{[(6-Chloropyrid-3-yl]methyl](methyl)amino}furan-2(5H)-on (M26.10);
M.27. Various compounds: aluminium phosphide, amidoflumet, benclothiaz, benzoximate, bifenazate, borax, bromopropylate, cyanide, cyenopyrafen, cyflumetofen, chinomethionate, dicofol, fluoroacetate, phosphine, pyridalyl, pyrifluquinazon, sulfur, organic sulfur compounds, tartar emetic, sulfoxaflor, N-R′-2,2-dihalo-1-R″cyclopropanecarboxamide-2-(2,6-dichloro-α,α,α-trifluoro-p-tolyl)hydrazone or N-R′-2,2-di(R″′)propionamide-2-(2,6-dichloro-α,α,α-trifluoro-p-tolyl)-hydrazone, wherein R′ is methyl or ethyl, halo is chloro or bromo, R″ is hydrogen or methyl and R″′ is methyl or ethyl, 4-But-2-ynyloxy-6-(3,5-dimethyl-piperidin-1-yl)-2-fluoro-pyrimidine (M27.1), Cyclopropaneacetic acid, 1,1′-[(3S,4R,4aR,6S,6aS,12R,12aS,12bS)-4-[[(2-cyclopropylacetyl)oxy]methyl]-1,3,4,4a,5,6,6a,12,12a,12b-decahydro-12-hydroxy-4,6a,12b-trimethyl-1′-oxo-9-(3-pyridinyl)-2H,11H-naphtho[2,1-b]pyrano[3,4-e]pyran-3,6-diyl]ester (M27.2) and 8-(2-Cyclopropylmethoxy-4-trifluoromethyl-phenoxy)-3-(6-trifluoromethyl-pyridazin-3-yl)-3-aza-bicyclo[3.2.1]octane (M27.3).

The commercially available compounds of the group M may be found in The Pesticide Manual, 13th Edition, British Crop Protection Council (2003) among other publications.

Paraoxon and their preparation have been described in Farm Chemicals Handbook, Volume 88, Meister Publishing Company, 2001. Flupyrazofos has been described in Pesticide Science 54, 1988, p. 237-243 and in U.S. Pat. No. 4,822,779. AKD 1022 and its preparation have been described in U.S. Pat. No. 6,300,348. The anthranilamides M23.1 to M23.6 have been described in WO 2008/72743 and WO 200872783, those M23.7 to M23.12 in WO2007/043677. The phthalamide M 21.1 is known from WO 2007/101540. The alkynylether compound M27.1 is described e.g. in JP 2006131529. Organic sulfur compounds have been described in WO 2007060839. The isoxazoline compounds M 22.1 to M 22.8 have been described in e.g. WO2005/085216, WO 2007/079162, WO 2007/026965, WO 2009/126668 and WO2009/051956. The aminofuranone compounds M 26.1 to M 26.10 have been described eg. in WO 2007/115644. The pyripyropene derivative M 27.2 has been described in WO 2008/66153 and WO 2008/108491. The pyridazin compound M 27.3 has been described in JP 2008/115155. Malononitrile compounds as those (M24.1) and (M24.2) have been described in WO 02/089579, WO 02/090320, WO 02/090321, WO 04/006677, WO 05/068423, WO 05/068432 and WO 05/063694.

Fungicidal mixing partners are those selected from the group consisting of acylalanines such as benalaxyl, metalaxyl, ofurace, oxadixyl,

amine derivatives such as aldimorph, dodine, dodemorph, fenpropimorph, fenpropidin, guazatine, iminoctadine, spiroxamin, tridemorph,

anilinopyrimidines such as pyrimethanil, mepanipyrim or cyrodinyl, antibiotics such as cycloheximid, griseofulvin, kasugamycin, natamycin, polyoxin or streptomycin,

azoles such as bitertanol, bromoconazole, cyproconazole, difenoconazole, diniconazole, epoxiconazole, fenbuconazole, fluquiconazole, flusilazole, hexaconazole, imazalil, metconazole, myclobutanil, penconazole, propiconazole, prochloraz, prothioconazole, tebuconazole, triadimefon, triadimenol, triflumizol, triticonazole, flutriafol,

dicarboximides such as iprodion, myclozolin, procymidon, vinclozolin, dithiocarbamates such as ferbam, nabam, maneb, mancozeb, metam, metiram, propineb, polycarbamate, thiram, ziram, zineb,

heterocyclic compounds such as anilazine, benomyl, boscalid, carbendazim, carboxin, oxycarboxin, cyazofamid, dazomet, dithianon, famoxadon, fenamidon, fenarimol, fuberidazole, flutolanil, furametpyr, isoprothiolane, mepronil, nuarimol, probenazole, proquinazid, pyrifenox, pyroquilon, quinoxyfen, silthiofam, thiabendazole, thifluzamid, thiophanate-methyl, tiadinil, tricyclazole, triforine,

copper fungicides such as Bordeaux mixture, copper acetate, copper oxychloride, basic copper sulfate,

nitrophenyl derivatives such as binapacryl, dinocap, dinobuton, nitrophthalisopropyl, phenylpyrroles such as fenpiclonil or fludioxonil,

sulfur,

other fungicides such as acibenzolar-S-methyl, benthiavalicarb, carpropamid, chlorothalonil, cyflufenamid, cymoxanil, diclomezin, diclocymet, diethofencarb, edifen-phos, ethaboxam, fenhexamid, fentin-acetate, fenoxanil, ferimzone, fluazinam, fosetyl, fosetyl-aluminum, iprovalicarb, hexachlorobenzene, metrafenon, pencycuron, propamocarb, phthalide, toloclofos-methyl, quintozene, zoxamid,

strobilurins such as azoxystrobin, dimoxystrobin, fluoxastrobin, kresoxim-methyl, metominostrobin, orysastrobin, picoxystrobin or trifloxystrobin,

sulfenic acid derivatives such as captafol, captan, dichlofluanid, folpet, tolyifluanid, cinnemamides and analogs such as dimethomorph, flumetover or flumorph.

The invertebrate pest, i.e. arthropodes and nematodes, the plant, soil or water in which the plant is growing can be contacted with the present compound(s) of formula I or composition(s) containing them by any application method known in the art. As such, “contacting” includes both direct contact (applying the compounds/compositions directly on the animal pest or plant—typically to the foliage, stem or roots of the plant) and indirect contact (applying the compounds/compositions to the locus of the animal pest or plant).

Moreover, invertebrate pests may be controlled by contacting the target pest, its food supply, habitat, breeding ground or its locus with a pesticidally effective amount of compounds of formula I. As such, the application may be carried out before or after the infection of the locus, growing crops, or harvested crops by the pest.

“Locus” means a habitat, breeding ground, cultivated plants, plant propagation material (such as seed), soil, area, material or environment in which a pest or parasite is growing or may grow.

In general “pesticidally effective amount” means the amount of active ingredient needed to achieve an observable effect on growth, including the effects of necrosis, death, retardation, prevention, and removal, destruction, or otherwise diminishing the occurrence and activity of the target organism. The pesticidally effective amount can vary for the various compounds/compositions used in the invention. A pesticidally effective amount of the compositions will also vary according to the prevailing conditions such as desired pesticidal effect and duration, weather, target species, locus, mode of application, and the like.

The compounds of formula I and their compositions can be used for protecting wooden materials such as trees, board fences, sleepers, etc. and buildings such as houses, outhouses, factories, but also construction materials, furniture, leathers, fibers, vinyl articles, electric wires and cables etc. from ants and/or termites, and for controlling ants and termites from doing harm to crops or human being (e.g. when the pests invade into houses and public facilities). The compounds of are applied not only to the surrounding soil surface or into the under-floor soil in order to protect wooden materials but it can also be applied to lumbered articles such as surfaces of the under-floor concrete, alcove posts, beams, plywood, furniture, etc., wooden articles such as particle boards, half boards, etc. and vinyl articles such as coated electric wires, vinyl sheets, heat insulating material such as styrene foams, etc. In case of application against ants doing harm to crops or human beings, the ant controller of the present invention is applied to the crops or the surrounding soil, or is directly applied to the nest of ants or the like.

The compounds of formula I can also be applied preventively to places at which occurrence of the pests is expected.

The compounds of formula I may be also used to protect growing plants from attack or infestation by pests by contacting the plant with a pesticidally effective amount of compounds of formula I. As such, “contacting” includes both direct contact (applying the compounds/compositions directly on the pest and/or plant—typically to the foliage, stem or roots of the plant) and indirect contact (applying the compounds/compositions to the locus of the pest and/or plant).

In the case of soil treatment or of application to the pests dwelling place or nest, the quantity of active ingredient ranges from 0.0001 to 500 g per 100 m², preferably from 0.001 to 20 g per 100 m².

Customary application rates in the protection of materials are, for example, from 0.01 g to 1000 g of active compound per m² treated material, desirably from 0.1 g to 50 g per m².

Insecticidal compositions for use in the impregnation of materials typically contain from 0.001 to 95% by weight, preferably from 0.1 to 45% by weight, and more preferably from 1 to 25% by weight of at least one repellent and/or insecticide.

For use in bait compositions, the typical content of active ingredient is from 0.001% by weight to 15% by weight, desirably from 0.001% by weight to 5% by weight of active compound.

For use in spray compositions, the content of active ingredient is from 0.001 to 80% by weight, preferably from 0.01 to 50% by weight and most preferably from 0.01 to 15% by weight.

For use in treating crop plants, the rate of application of the active ingredients of this invention may be in the range of 0.1 g to 4000 g per hectare, desirably from 25 g to 600 g per hectare, more desirably from 50 g to 500 g per hectare.

In the treatment of seed, the application rates of the active ingredients are generally from 0.1 g to 10 kg per 100 kg of seed, preferably from 1 g to 5 kg per 100 kg of seed, in particular from 1 g to 200 g per 100 kg of seed.

The present invention is now illustrated in further detail by the following examples.

### I. Preparation Examples

C. Compound examples

Compounds were characterized e.g. by coupled High Performance Liquid Chromatography/mass spectrometry (HPLC/MS), by ¹H-NMR and/or by their melting points.

Analytical HPLC column: RP-18 column Chromolith Speed ROD from Merck KgaA, Germany). Elution: acetonitrile+0.1% trifluoroacetic acid (TFA)/water+0.1% trifluoroacetic acid (TFA) in a ratio of from 5:95 to 95:5 in 5 minutes at 40° C.

¹H-NMR, respectively ¹³C-NMR: The signals are characterized by chemical shift (ppm) vs. tetramethylsilane, respectively CDCl₃ for ¹³C-NMR, by their multiplicity and by their integral (relative number of hydrogen atoms given). The following abbreviations are used to characterize the multiplicity of the signals: m=multiplett, q=quartett, t=triplett, d=doublet and s=singulett.

### C.1 Compound Examples 1

Compound examples 1-1 to 1-12 correspond to the compound formula C.1

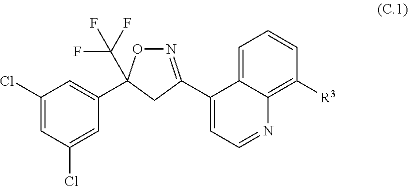

wherein R³ each compound example is defined table C.1 below.

TABLE C.1

| Compound Ex. | R³ | Analytics |
|---|---|---|
| 1-1 | Br | see Example S.1 |
| 1-2 | CN | see Example S.2 |
| 1-3 | CO₂CH₃ | see Example S.3 |
| 1-4 | CO₂H | ¹H-NMR (400 MHz, DMSO-d₆): δ = 4.57 (d, 1H), |
| | | 4.64 (d, 1H), 7.68 (s, 2H), 7.85 (s, 1H), 7.97 (m, |
| | | 1H), 8.06 (s, 1H), 8.60 (m, 1H), 9.01 (m, 1H), 9.18 |
| | | (s, 1H), 16.10 (br. s, 1H) ppm. |
| 1-5 | CONH—CH₃ | ¹H-NMR (400 MHz, DMSO-d₆): δ = 2.92 (s, 3H), |
| | | 4.57 (d, 1H), 4.64 (d, 1H), 7.69 (s, 2H), 7.85 (m, |
| | | 2H), 7.94 (m, 1H), 8.48 (m, 1H), 8.84 (m, 1H), 9.16 |
| | | (s, 1H), 10.12 (br. s, 1H) ppm. |
| 1-6 | CONH—CH₂-2- | ¹H-NMR (400 MHz, DMSO-d₆): δ = 4.58 (d, 1H), |
| | pyridyl | 4.64 (d, 1H), 4.76 (d, 2H), 7.28 (m, 1H), 7.51 (d, |
| | | 1H), 7.70 (s, 2H), 7.80 (m, 1H), 7.85 (m, 2H), 7.96 |
| | | (m, 1H), 8.55 (m, 1H), 8.87 (d, 1H), 9.19 (s, 1H), |
| | | 11.00 (br. s, 1H) ppm. |
| 1-7 | CONH—CH₂CF₃ | ¹H-NMR (400 MHz, DMSO-d₆): δ = 4.32 (m, 2H), |
| | | 4.58 (d, 1H), 4.63 (d, 1H), 7.69 (s, 2H), 7.86 (s, |
| | | 1H), 7.91 (m, 1H), 7.96 (m, 1H), 8.54 (d, 1H), 8.92 |
| | | (d, 1H), 9.19 (s, 1H), 10.92 (br. s, 1H) ppm. |
| 1-8 | CH₂NH₂ x HCl | ¹H-NMR (400 MHz, DMSO-d₆): δ = 4.55-4.68 (m, |
| | | 2H), 7.68 (s, 2H), 7.85-7.86 (m, 2H), 7.92 (d, 1H), |
| | | 7.99 (d, 1H), 8.40 (br. s, 3H), 8.78 (d, 1H), 9.15 (d, |
| | | 1H) ppm. |
| 1-9 | CH₂NH—C(═O)—CH₃ | ¹H-NMR (400 MHz, CDCl₃): δ = 1.98 (s, 3H), 3.91 |
| | | (d, 1H), 4.26 (d, 1H), 5.00 (m, 2H), 6.72 (br. s, 1H), |
| | | 7.40 (m, 2H), 7.48 (m, 2H), 7.63 (m, 1H), 7.81 (m, |
| | | 1H), 8.75 (m, 1H), 8.99 (m, 1H) ppm. |
| 1-10 | CH₂NH—C(═O)— | ¹H-NMR (400 MHz, CDCl₃): δ = 1.12 (m, 3H), 2.19 |
| | CH₂CH₃ | (m, 2H), 3.91 (d, 1H), 4.27 (d, 1H), 5.02 (m, 2H), |
| | | 6.73 (br. s, 1H), 7.41 (m, 1H), 7.48 (m, 1H), 7.56 |
| | | (m, 2H), 7.64 (m, 1H), 7.82 (m, 1H), 8.75 (m, 1H), |
| | | 8.99 (m, 1H) ppm. |
| 1-11 | CH₂NH—C(═O)— | ¹H-NMR (400 MHz, CDCl₃): δ = 0.88 (t, 3H), 1.63 |
| | CH₂CH₂CH₃ | (m, 2H), 2.17 (m, 2H), 3.91 (d, 1H), 4.28 (d, 1H), |
| | | 5.01 (m, 2H), 7.02 (br. s, 1H), 7.44-7.58 (m, 3H), |
| | | 7.67 (m, 1H), 7.88 (m, 1H), 8.78 (m, 1H), 9.06 (m, |
| | | 1H) ppm. |
| 1-12 | CH₂NH—C(═O)— | ¹H-NMR (400 MHz, CDCl₃): δ = 3.58 (s, 2H), 3.92 |
| | CH₂SCF₃ | (d, 1H), 4.28 (d, 1H), 5.05 (m, 2H), 7.43 (m, 1H), |
| | | 7.48 (m, 1H), 7.56 (m, 2H), 7.62-7.67 (m, 1H), 7.81 |
| | | (m, 1H), 7.84 (m, 1H), 8.79 (d, 1H), 9.00 (m, 1H) |
| | | ppm. |

### S. Synthesis Examples

### S.1 Preparation of 8-bromo-4-[5-(3,5-dichloro-phenyl)-5-trifluoromethyl-4,5-di-hydroisoxazol-3-yl]-quinoline (Compound 1-1 of table C.1)

### Step 1: Preparation of 8-bromo-4-methyl-quinoline

2-Bromoaniline (100 g, 689 mmol) was dissolved in acetic acid (500 mL) and concentrated sulfuric acid (101.7 g, 1033 mmol) was added dropwise to it. A solution of methyl vinylketone (223 g, 1.0 mol) in acetic acid (500 mL) was added dropwise to the reaction mixture over a period of 15 minutes, during which time the color of the solution became wine red. The reaction mixture was then heated to 90° C. for 16 h, cooled to 0° C., and basified to pH 7 by using 6N NaOH (2 L). The solution was extracted with ethyl acetate (3 L) and the organic layer was washed with brine (3 L). The organic layer was dried over anhydrous Na₂SO₄, filtered, and concentrated to give a black oil, which was purified by column chromatography. A gummy solid was obtained which was triturated with hexane (100 mL) to afford the title compound (58.0 g, 44%) as a white solid.

¹H-NMR (400 MHz, DMSO-d₆) δ=8.89 (d, J=4.0 Hz, 1H), 8.05 (dd, J=4.0 Hz, J=1.20 Hz, 1H), 7.97 (dd, J=4.0 Hz, J=1.20 Hz, 1H), 7.41 (dd, J=7.60 Hz, J=0.40 Hz, 1H), 7.29 (d, J=4.0 Hz, 1H), 2.72 (s, 3H); MS: ESI m/z 223 [M+H]⁺.

### Step 2: Preparation of 8-bromo-quinoline-4-carbaldehyde

8-Bromo-4-methyl-quinoline of Step 1 (40.0 g, 176 mmol) was dissolved in 1,4-dioxane (1.4 L) and selenium dioxide (387.2 g, 352 mmol) was added to it. The reaction mixture was heated to 90° C. for 30 min and then cooled to room temperature. The solution was filtered through celite. Saturated sodium bicarbonate solution (500 mL) was added and the mixture was extracted with ethyl acetate (2 L). The organic layer was washed with brine (3 L), dried over anhydrous sodium sulfate, filtered, and then concentrated to afford the title compound (35.0 g, 89%) as a red solid.

¹H-NMR (400 MHz, DMSO-d₆) δ=10.52 (s, 1H), 9.33 (d, J=4.0 Hz, 1H), 9.05 (dd, J=8.0 Hz, J=1.2 Hz, 1H), 9.05 (dd, J=1.2 Hz, J=8.0 Hz, 1H), 8.18 (dd, J=4.0 Hz, J=0.8 Hz, 1H), 7.88 (d, J=4.0 Hz, 1H), 7.60 (dd, J=8.0, J=0.4 Hz, Hz, 1H); MS: ESI m/z 236 [M+H]⁺.

### Step 3: Preparation of 8-bromo-quinoline-4-carbaldehyde oxime

8-Bromo-quinoline-4-carbaldehyde of Step 2 (13.0 g, 63.1 mmol) was dissolved in ethanol (1.20 L) and 50% hydroxylamine (13.0 mL, 194.6 mmol) in water was added dropwise to it. The reaction mixture was stirred at room temperature for 16 h and then poured in ice water (1 L). The solid that precipitated was filtered, washed with water (1 L), and dried to afford the title compound (35.0 g, 86%) as a yellow solid.

¹H-NMR (400 MHz, DMSO-d₆) δ=12.13 (s, 1H), 9.02 (d, J=8.0 Hz, 1H), 8.87 (s, 1H), 8.64 (d, J=8.0 Hz, 1H), 8.19 (d, J=8.0 Hz, 1H), 7.84 (d, J=8.0 Hz, 1H), 7.58-7.54 (m, 1H); MS: ESI m/z 252 [M+H]⁺.

### Step 4: Preparation of 1,3-dichloro-5-(1-trifluoromethyl-vinyl)-benzene

3,5-Dichlorophenylboronic acid (25.0 g, 13.1 mmol) was dissolved in THF/H₂O (200 mL:50 mL) in a sealed tube. Potassium carbonate (37.8 g, 273 mmol) was added and the solution was cooled to 0° C. 2-Bromo-3,3,3-trifluoroprop-1-ene (20.1 mL, 155 mmol) and dichlorobis(triphenylphosphine) palladium(II) (1.83 g, 2.62 mmol) were added and the solution was heated to 90° C. for 6 h. The mixture was cooled to room temperature, diluted with 4 ethyl acetate (250 mL), and then filtered through a bed of celite. The organic layer was washed with water (200 mL) and brine (200 mL). The combined organic layers were dried over anhydrous sodium sulfate, filtered, concentrated under reduced pressure, and purified by silica-gel column chromatography. A gummy white gel-like substance was obtained. Upon addition of MTBE (methyl-tert-butyl ether), a white solid precipitated which was removed by filtration; the filtrate was concentrated to afford the title compound (26.0 g, 83%) as a yellow oil.

¹H-NMR (400 MHz, CDCl₃) δ=7.40 (d, J=2.0 Hz, 1H), 7.34 (d, J=1.6 Hz, 2H), 6.05 (d, J=1.2 Hz, 1H), 5.83 (d, J=1.6 Hz, 1H).

### Step 5: Preparation of 8-bromo-4-[5-(3,5-dichloro-phenyl)-5-trifluoromethyl-4,5-di-hydroisoxazol-3-yl]-quinoline

8-Bromo-quinoline-4-carbaldehyde oxime of Step 3 (10.0 g, 44.0 mmol) was dissolved in dry THF (200 mL) and dry DMF (50 mL). N-Chlorosuccinimide (6.80 g, 44.0 mol) was added and the reaction mixture was stirred at room temperature for 1 h. After the disappearance of the starting material (monitored by TLC), potassium bicarbonate (5.28 g, 52.8 mmol) was added followed by the addition of 1,3-dichloro-5-(1-trifluoromethyl-vinyl)-benzene (i.e. the product of Step 4, 13.8 g, 57.2 mmol) in dry THF (20.0 mL). The reaction mixture was heated to 70° C. for 24 h. The reaction mixture was cooled to room temperature, and filtered through celite. Water (200 mL) was added to the filtrate and the solution was extracted with ethyl acetate (200 mL). The organic layer was dried over anhydrous sodium sulfate, filtered, and then concentrated to afford a colorless liquid which was purified by column chromatography to afford the title compound (10.0 g, 33%).

¹H-NMR (400 MHz, DMSO-d₆) δ=9.10 (d, J=4.0 Hz, 1H), 8.87 (d, J=8.0 Hz, 1H), 8.15 (d, J=7.2 Hz, 1H), 7.57-7.53 (m, 3H), 7.47 (s, 1H), 7.43 (d, J=12.0 Hz, 1H), 4.31 (d, J=17.6 Hz, 1H), 3.92 (d, J=17.2 Hz, 1H).

### Example 2

### Preparation of 4-[5-(3,5-dichloro-phenyl)-5-trifluoromethyl-4,5-di-hydroisoxazol-3-yl]-quinoline-8-carbonitrile (Compound 1-2 of table C.1)

8-Bromo-4-[5-(3,5-dichloro-phenyl)-5-trifluoromethyl-4,5-dihydro-isoxazol-3-yl]-quinoline (i.e. the product of Step 5, Example 1, 0.300 g, 0.612 mmol) was dissolved in N,N-dimethylformamide (15 mL) and the solution was degassed using N2. Zinc(II) cyanide (0.244 g, 2.080 mmol), copper(I) iodide (0.005 g, 0.030 mmol), bis(diphenylphosphino) ferrocene (0.037 g, 0.0673 mmol), and Pd₂(dba)₃ (0.056 g, 0.0612 mmol) were added successively over a period of 15 min and then the solution was heated to 95° C. for 16 h. The reaction mixture was cooled to room temperature, diluted with ethyl acetate (25 mL) and filtered through celite. The filtrate was washed with water (20 mL) and brine (20 mL). The combined organic layers were dried over anhydrous sodium sulfate, filtered, concentrated under reduced pressure, and purified by silica-gel column chromatography to afford the title compound (0.150 g, 69%).

¹H-NMR (400 MHz, CDCl₃) δ=9.24 (d, J=8.8 Hz, 1H), 9.16 (d, J=4.4 Hz, 1H), 8.23 (dd, J=6.0 Hz, J=1.2 Hz, 1H), 7.80-7.76 (m, 1H), 7.52 (s, 1H), 7.51-7.48 (m, 3H), 4.28 (d, J=17.2 Hz, 1H), 3.93 (d, J=17.2 Hz, 1H).

### Example 3

### Preparation of 4-[5-(3,5-dichloro-phenyl)-5-trifluoromethyl-4,5-di-hydroisoxazol-3-yl]-quinoline-8-carboxylic acid methyl ester (Compound 1-3 of table C.1)

8-Bromo-4-[5-(3,5-dichloro-phenyl)-5-trifluoromethyl-4,5-dihydro-isoxazol-3-yl]-quinoline (i.e. the product of Step 5, Example 1, 3.50 g, 7.20 mmol) was dissolved in dry methanol (500 mL) in a steel autoclave and sodium carbonate (188 mg, 17.2 mmol), palladium bis(diphenylphosphino) ferrocene dichloromethane complex (117 mg, 0.144 mmol) and bis(diphenylphosphino) ferrocene (159 mg, 0.208 mmol) were added. The autoclave was filled with nitrogen up to 100 psi, purged, and then filled with carbon monoxide gas up to 70 psi. The reaction mixture was heated to 70° C. for 24 h. The reaction mixture was cooled to room temperature and filtered through celite. The filtrate was concentrated and purified by column chromatography to afford The title compound (2.10 g, 61.9%).

¹H-NMR (400 MHz, DMSO-d₆) δ=9.09 (d, J=4.0 Hz, 1H), 9.03 (dd, J=7.6 Hz, J=1.2 Hz, 1H), 8.06 (dd, J=6.0 Hz, J=1.2 Hz, 1H), 7.74 (dd, J=7.2 Hz, J=1.2 Hz, 1H), 7.55 (d, J=1.2 Hz, 2H), 7.47 (d, J=4.0 Hz, 1H), 7.41 (d, J=4.0 Hz, 1H), 1H, 4.27 (d, J=17.2 Hz, 1H), 4.06 (s, 3H), 3.92 (d, J=17.2 Hz, 1H).

### II. Evaluation of Pesticidal Activity:

The activity of the compounds of formula I of the present invention can be demonstrated and evaluated by the following biological test.

The test solution was prepared the day it was used and in general at concentrations of ppm (wt/vol).

B.1 Green Peach Aphid (Myzus persicae)

For evaluating control of green peach aphid (Myzus persicae) through systemic means the test unit consisted of 96-well-microtiter plates containing liquid artificial diet under an artificial membrane.

The compounds were formulated using a solution containing 75% v/v water and 25% v/v DMSO. Different concentrations of formulated compounds were pipetted into the aphid diet, using a custom built pipetter, at two replications.

After application, 5-8 adult aphids were placed on the artificial membrane inside the microtiter plate wells. The aphids were then allowed to suck on the treated aphid diet and incubated at about 23±1° C. and about 50±5% relative humidity for 3 days. Aphid mortality and fecundity was then visually assessed.

In this test, compounds 1-1, 1-4, 1-6, 1-7, 1-9, 1-10 and 1-12 respectively, at 2500 ppm showed over 75% mortality in comparison with untreated controls.

B.2 Vetch Aphid (Megoura viciae)

For evaluating control of vetch aphid (Megoura viciae) through contact or systemic means the test unit consisted of 24-well-microtiter plates containing broad bean leaf disks.

The compounds were formulated using a solution containing 75% v/v water and 25% v/v DMSO. Different concentrations of formulated compounds were sprayed onto the leaf disks at 2.5 μl, using a custom built micro atomizer, at two replications. After application, the leaf disks were air-dried and 5-8 adult aphids placed on the leaf disks inside the microtiter plate wells. The aphids were then allowed to suck on the treated leaf disks and incubated at about 23±1° C. and about 50±5% relative humidity for 5 days. Aphid mortality and fecundity was then visually assessed.

In this test, compounds 1-6, 1-7 and 1-10 respectively, at 2500 ppm showed over 75% mortality in comparison with untreated controls.

B.3 Tobacco Budworm (Heliothis virescens) I

For evaluating control of tobacco budworm (Heliothis virescens) the test unit consisted of 96-well-microtiter plates containing an insect diet and 15-25 H. virescens eggs. The compounds were formulated using a solution containing 75% v/v water and 25% v/v DMSO. Different concentrations of formulated compounds were sprayed onto the insect diet at 10 μl, using a custom built micro atomizer, at two replications. After application, microtiter plates were incubated at about 28±1° C. and about 80±5% relative humidity for 5 days. Egg and larval mortality was then visually assessed. In this test, compounds 1-1, 1-2, 1-5, 1-7, 1-9, 1-10 and 1-12, respectively, at 2500 ppm showed over 75% mortality in comparison with untreated controls.

B.4 Boll Weevil (Anthonomus grandis)

For evaluating control of boll weevil (Anthonomus grandis) the test unit consisted of 24-well-microtiter plates containing an insect diet and 20-30 A. grandis eggs.

The compounds were formulated using a solution containing 75% v/v water and 25% v/v DMSO. Different concentrations of formulated compounds were sprayed onto the insect diet at 20 μl, using a custom built micro atomizer, at two replications.

After application, microtiter plates were incubated at about 23±1° C. and about 50±5% relative humidity for 5 days. Egg and larval mortality was then visually assessed.

In this test, compounds 1-1, 1-2, 1-3, 1-4, 1-5, 1-6, 1-7, 1-9, 1-10 and 1-12, respectively, at 2500 ppm showed over 75% mortality in comparison with untreated controls.

B.5 Mediterranean Fruitfly (Ceratitis capitata)

For evaluating control of Mediterranean fruitfly (Ceratitis capitata) the test unit consisted of microtiter plates containing an insect diet and 50-80 C. capitata eggs.

The compounds were formulated using a solution containing 75% v/v water and 25% v/v DMSO. Different concentrations of formulated compounds were sprayed onto the insect diet at 5 μl, using a custom built micro atomizer, at two replications. After application, microtiter plates were incubated at about 28±1° C. and about 80±5% relative humidity for 5 days. Egg and larval mortality was then visually assessed.

In this test, compounds 1-1, 1-2, 1-3, 1-4, 1-5, 1-6, 1-9, 1-10 and 1-12, respectively, at 2500 ppm showed over 75% mortality in comparison with untreated controls.

B.6 Southern Armyworm (Spodoptera eridania)

The active compounds were formulated in cyclohexanone as a 10,000 ppm solution supplied in tubes. The tubes were inserted into an automated electrostatic sprayer equipped with an atomizing nozzle and they served as stock solutions for which lower dilutions were made in 50% acetone:50% water (v/v). A nonionic surfactant (Kinetic®) was included in the solution at a volume of 0.01% (v/v).

Lima bean plants (variety Sieva) were grown 2 plants to a pot and selected for treatment at the 1St true leaf stage. Test solutions were sprayed onto the foliage by an automated electrostatic plant sprayer equipped with an atomizing spray nozzle. The plants were dried in the sprayer fume hood and then removed from the sprayer. Each pot was placed into perforated plastic bags with a zip closure. About 10 to 11 armyworm larvae were placed into the bag and the bags zipped closed. Test plants were maintained in a growth room at about 25° C. and about 20-40% relative humidity for 4 days, avoiding direct exposure to fluorescent light (24 hour photoperiod) to prevent trapping of heat inside the bags. Mortality and reduced feeding were assessed 4 days after treatment, compared to untreated control plants.

In this test, compounds 1-1 and 1-2, respectively, at 300 ppm showed over 75 mortality in comparison with untreated controls.

### BA. Animal Health

General test conditions of animal health glass vial contact assays

If not otherwise specified the tests are usually to be conducted as glass vial contact assays. Glass vials (20 ml scintillation vials) are used. Treatment solutions are mixed with technical grade chemicals diluted in acetone. Treatment solutions needed for the assays include generally 1 and 10 ppm (0.01 and 0.1 μg/cm², respectively), but optionally also 100 and/or 1000 ppm for first tier vials. As commercial standard, alphacypermethrin, is run at 1 ppm. As solvent control, acetone is used for the assay. Treatment solution is pipetted into the bottom of each vial. Each vial is turned on its side and placed onto a commercial grade hot dog roller without applying heat. The uncapped vials are allowed to roll to allow for the acetone treatment to vent off. After drying, the vials are placed into the compartmented vial shipping boxes. The workstation is prepared by chilling the table and plastic Petri dishes with the inside wall coated with Fluon. A weigh boat of 10% sugar water saturated cotton dental pellets is also prepared. The animal pests are collected into a tube with a rechargeable insect vacuum. The tube of animal pests is placed in a laboratory refrigerator until the animal pests are incapacitated. The animal pests are emptied into chilled Petri dish. A small cotton dental pellet is soaked in water or in 10 wt % sugar water, whereas the excess solution is gently squeezed out. The cotton dental pellet is placed into the bottom of each vial. For the test the animal pests are added to each vial and then the cap is loosely put on the vial to allow for ventilation. The test vials are hold at ambient room temperature in compartmented boxes. In general, the animal pests are observed for incapacitation at least at 4, 24, and 48 hours after infestation, or for a longer period, if required. Mortality is defined as an insect incapable of coordinated movement when agitated.

### BA.1 Acrobat Ant Workers (Crematogaster Sp.)

Treatment solutions are mixed with test compound diluted in acetone at concentrations of 10 and 100 ppm. Collected ants for placement in the vials are typically not chilled prior to infesting vials. Data are collected at 1, 2, and 4 days after infestation.

BA.2 Brown Dog Tick Adults (Rhipicephalus sanguineus)

Treatment solutions are mixed with test compound diluted in acetone at concentrations of 10 and 100 ppm. No food or water source is provided in the vials. Data are collected out to 5 days after infestation. Ticks are evaluated by rolling the vials on a preheated hotdog roller. Tick activity is stimulated within approx. 1-2 minutes.

BA.3 Cat Flea Adults (Ctenocephalides felis)

Treatment solutions are mixed with test compound diluted in acetone at concentrations of 10 and 100 ppm. The standard (alphacypermethrin) is used at 10 ppm. The cat flea adults fleas are incapacitated by placing the shipment vials of fleas in a laboratory freezer (−20° C.) for about 3 minutes. Upon removal from the freezer, the fleas are then emptied into a chilled Petri dish and covered. No food or water source is provided in the vials. Fleas are observed for incapacitation at 1, 2, and 3 days after infestation.

BA.4 German cockroach adults (Blattella germanica)

Treatment solutions are mixed with test compound diluted in acetone at concentrations of 10 and 100 ppm. The cockroaches are incapacitated by chilling them in a Fluon or petroleum jelly/mineral oil treated plastic tray that is placed onto a bed of ice set into a large plastic tub. Then the cockroaches are emptied into chilled Petri dish. The small cotton dental pellet soaked in water is added into the bottom of each vial. Test vials are hold at ambient room temperature in compartmented boxes, whereas the compartmented box are stored with the vials on its side. Data are collected at 4 hours, and 1, 2 days after infestation.

BA.5 House Fly Adults (Musca domestica)

Treatment solutions are mixed with test compound diluted in acetone at concentrations of 1 and 10 ppm. The small cotton dental pellet added is soaked in 10% sugar water. Flies are observed for incapacitation at 4, 24, and 48 hours after infestation.

BA.6 Yellowfever Mosquito Adults (Aedes aegypti)

### BA.6.a Glass Vial Contact Assay (Test Conditions as Described Above)

Treatment solutions are mixed with test compound diluted in acetone at concentrations of 1 and 10 ppm. The mosquitoes are incapacitated by placing the entire cage into a walk-in cooler or lab refrigerator for 5-10 minutes. Chilled plastic Petri dish with the inside wall coated with Fluon and bottom lined with a piece of paper towel are placed within the cage. Mosquitoes are collected with a mouth aspirator and emptied into the Petri dish, quickly replacing the lid on the dish. The dish is removed from the cage and the small cotton dental pellet soaked in 10% sugar water is added into the bottom of each vial then placed onto the chill table. Flies are observed for incapacitation at 4, 24, and 48 hours after infestation.

BA.6.b Larval Mosquito Water Treatment Assay

The assay is conducted in 6-well polystyrene plates using one plate per treatment rate. Stock solutions are prepared at 100 and 1000 ppm. Screen rates are at 1 and 10 ppm. Distilled water is added to each well, control wells are treated with acetone. Temephos (Abate technical) is used as the standard at 0.1 ppm. Ten late third-instar yellowfever mosquito larvae (Aedes aegypti) in water are added to each well. One drop of liver powder solution (6 g in 100 ml distilled water) is added to each well as a food source daily. Plates are maintained at 22-25° C. and 25-50% RH (relative humidity) and observed daily for dead larvae and pupae at 1, 2, 3, and 5 days after treatment. Dead larvae and all pupae are removed daily. Mortality is defined as an insect incapable of coordinated movement when agitated.

1-32. (canceled)

33. A compound of formula (I) 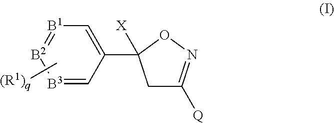
wherein
Q is an aromatic or heteroaromatic radical of the formula (II) 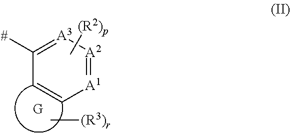
G is a condensed phenyl ring or is a condensed 5-, 6-, 7- or 8-membered saturated, partially unsaturated or completely unsaturated heterocyclic ring containing 1, 2 or 3 heteroatoms selected from the group consisting of O, S and N as ring members;
A¹, A² and A³ are N or CH, with the proviso that at most two of A¹, A² and A³ are N;
B¹, B² and B³ are N or CH, with the proviso that at most two of B¹, B² and B³ are N;
X is selected from the group consisting of C₁-C₄-alkyl, C₁-C₄-alkoxy-C₁-C₄-alkyl, C₁-C₄-haloalkoxy-C₁-C₄-alkyl, C₂-C₄-alkenyl, C₂-C₄-haloalkenyl, C₂-C₄-alkynyl, C₂-C₄-haloalkynyl, C₃-C₆-cycloalkyl and C₃-C₆-halocycloalkyl;
each R¹ is independently selected from the group consisting of halogen; cyano; azido; nitro; —SCN; SF₅; C₁-C₆-alkyl which may be partially or fully halogenated and/or may be substituted by one or more radicals R⁴; C₃-C₈-cycloalkyl which may be partially or fully halogenated and/or may be substituted by one or more radicals R⁵; C₂-C₆-alkenyl which may be partially or fully halogenated and/or may be substituted by one or more radicals R⁴; C₂-C₆-alkynyl which may be partially or fully halogenated and/or may be substituted by one or more radicals R⁴; —Si(R¹⁴)₂R¹³; —OR⁷; —OS(O)ₙR⁷; —S(O)ₙOR⁷; —SR⁷; —S(O)ₘR⁷; —S(O)ₙN(R⁸)R⁹; —N(R⁸)R⁹; —N(R⁸)C(═O)R⁶; —C(═O)R⁶; —C(═O)OR⁷; —C(═S)R⁶; —C(═S)OR⁷; —C(═NR⁸)R⁶; —C(═O)N(R⁸)R⁹; —C(═S)N(R⁸)R⁹; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
each R² is independently selected from the group consisting of halogen; cyano; azido; nitro; —SCN; SF₅; C₁-C₆-alkyl which may be partially or fully halogenated and/or may be substituted by one or more radicals R⁴; C₃-C₈-cycloalkyl which may be partially or fully halogenated and/or may be substituted by one or more radicals R⁵; C₂-C₆-alkenyl which may be partially or fully halogenated and/or may be substituted by one or more radicals R⁴; C₂-C₆-alkynyl which may be partially or fully halogenated and/or may be substituted by one or more radicals R⁴; —Si(R¹⁴)₂R¹³; —OS(O)ₙR⁷; —S(O)ₙR⁷; —S(O)ₘOR⁷, —SR⁷; —S(O)ₘR⁷; —S(O)ₙN(R⁸)R⁹; —N(R⁸)R⁹; —N(R⁸)C(═O)R⁶; —C(═O)R⁶; —C(═O)OR⁷; —C(═S)R⁶, —C(═S)OR⁷, —C(═NR⁸)R⁶; —C(═O)N(R⁸)R⁹; —C(═S)N(R⁸)R⁹; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
with the proviso that R² is not halogen, cyano, nitro, methyl which carries one halogen atom or carries one group OH or one methylcarbonyloxy group; OH, methoxy, —OS(O)ₙR⁷, —NH₂, —CHO, C₁-C₆-alkylcarbonyl; —C(═O)OR⁷, wherein R⁷ is hydrogen, C₁-C₆-alkyl or benzyl; —C(═O)R⁶, wherein R⁶ is —N(R⁸)R⁹; —C(═S)R⁶, wherein R⁶ is —N(R⁸)R⁹, —C(═NR⁸)R⁶, —C(═O)N(R⁸)R⁹ or —C(═S)N(R⁸)R⁹; when A¹, A² and A³ are CH and R² is bound to A¹;
or two radicals R² bound on adjacent carbon atoms may be together a group selected from the group consisting of —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR⁸—, —CH₂CH═N—, —CH═CH—NR⁸—, —OCH═N— and —SCH═N—, thus forming, together with the carbon atoms to which they are bound, a 5- or 6-membered ring, where the hydrogen atoms of the above groups may be replaced by one or more substituents selected from the group consisting of halogen, methyl, halomethyl, hydroxyl, methoxy and halomethoxy or one or more CH₂ groups of the above groups may be replaced by a C═O group;
each R³ is independently selected from the group consisting of halogen; cyano; azido; nitro; —SCN; —SF₅; C₁-C₆-alkyl which may be partially or fully halogenated and/or may be substituted by one or more radicals R⁴; C₃-C₈-cycloalkyl which may be partially or fully halogenated and/or may be substituted by one or more radicals R⁵; C₂-C₆-alkenyl which may be partially or fully halogenated and/or may be substituted by one or more radicals R⁴; C₂-C₆-alkynyl which may be partially or fully halogenated and/or may be substituted by one or more radicals R⁴; —Si(R¹⁴)₂R¹³; —OR⁷; —OS(O)ₘR⁷; —S(O)ₙOR⁷; —SR⁷; —S(O)ₘR⁷; —S(O)ₙN(R⁸)R⁹; —N(R⁸)R⁹; —N(R⁸)C(═O)R⁶; —C(═O)R⁶; —C(═O)OR⁷; —C(═S)R⁶; —C(═S)OR⁷; —C(═NR⁸)R⁶; —C(═O)N(R⁸)R⁹; —C(═S)N(R⁸)R⁹; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
or two radicals R³ bound on adjacent carbon atoms may be together a group selected from the group consisting of —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR⁸—, —CH₂CH═N—, —CH═CH—NR⁸—, —OCH═N— and —SCH═N—, thus forming, together with the carbon atoms to which they are bound, a 5- or 6-membered ring, where the hydrogen atoms of the above groups may be replaced by one or more substituents selected from the group consisting of halogen, methyl, halomethyl, hydroxyl, methoxy and halomethoxy or one or more CH₂ groups of the above groups may be replaced by a C═O group;
each R⁴ is independently selected from the group consisting of cyano; azido; nitro; —SCN; —SF₅; C₃-C₈-cycloalkyl; C₃-C₈-halocycloalkyl; C₃-C₈-cycloalkyl substituted by a phenyl group or substituted by a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the phenyl ring or the heterocyclyl ring may be substituted by one or more radicals R¹⁰; —Si(R¹⁴)₂R¹³, —OR⁷, —OSO₂R⁷, —SO₂OR⁷, —SR⁷, —S(O)ₘR⁷, —S(O)ₙN(R⁸)R⁹, —N(R⁸)R⁹, —C(═O)N(R⁸)R⁹, —C(═S)N(R⁸)R⁹, —C(═O)OR⁷, —C(═O)R⁶, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
or two geminally bound radicals R⁴ together form a group selected from the group consisting of ═CR¹¹R¹², ═S(O)ₘR⁷, ═S(O)ₘN(R⁸)R⁹, ═NR⁸, ═NOR⁷ and ═NNR⁸;
or two radicals R⁴, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from the group consisting of N, O, S, NO, SO and SO₂, as ring members;
each R⁵ is independently selected from the group consisting of cyano; azido; nitro; —SCN; —SF₅; C₁-C₆-alkyl; C₁-C₆-haloalkyl; C₁-C₆-alkoxy-C₁-C₆-alkyl; C₁-C₆-haloalkoxy-C₁-C₆-alkyl; C₃-C₈-cycloalkyl; C₃-C₈-halocycloalkyl; C₃-C₈-cycloalkyl-C₁-C₄-alkyl; C₃-C₈-halocycloalkyl-C₁-C₄-alkyl; C₂-C₆-alkenyl; C₂-C₆-haloalkenyl; C₂-C₆-alkynyl; C₂-C₆-haloalkynyl; —Si(R¹⁴)₂R¹³; —OR⁷; —OSO₂R⁷; —SO₂OR⁷; —SR⁷; —S(O)ₘR⁷; —S(O)ₙN(R⁸)R⁹; —N(R⁸)R⁹; —C(═O)N(R⁸)R⁹; —C(═S)N(R⁸)R⁹; —C(═O)OR⁷; —C(═O)R⁶; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
or two geminally bound radicals R⁵ together form a group selected from the group consisting of ═CR¹¹R¹², ═S(O)ₘR⁷, ═S(O)ₘN(R⁸)R⁹, ═NR⁸, ═NOR⁷ and ═NNR⁸;
or two radicals R⁵, together with the carbon atoms to which they are bound, form a 3-, 4-, 5-, 6-, 7- or 8-membered saturated or partially unsaturated carbocyclic or heterocyclic ring containing 1, 2 or 3 heteroatoms or heteroatom groups selected from the group consisting of N, O, S, NO, SO and SO₂, as ring members;
each R⁶ is independently selected from the group consisting of hydrogen; cyano; azido; C₁-C₆-alkyl; C₁-C₆-haloalkyl; C₁-C₆-alkoxy-C₁-C₆-alkyl; C₁-C₆-haloalkoxy-C₁-C₆-alkyl; C₃-C₈-cycloalkyl; C₃-C₈-halocycloalkyl; C₂-C₆-alkenyl; C₂-C₆-haloalkenyl; C₂-C₆-alkynyl; C₂-C₆-haloalkynyl; —CH₂—(C₃-C₆-cycloalkyl); —CH₂—(C₃-C₆-halocycloalkyl); —CH₂—S(O)ₙ—(C₁-C₄-alkyl); —CH₂—S(O)ₙ—(C₁-C₄-haloalkyl); —CH₂-phenyl; —CH₂—S(O)ₙ-phenyl; —OR⁷; —OSO₂R⁷; —SR⁷; —S(O)ₘR⁷; —S(O)ₙN(R⁸)R⁹; —N(R⁸)R⁹; —C(═O)N(R⁸)R⁹; —C(═S)N(R⁸)R⁹; —C(═O)OR⁷; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
each R⁷ is independently selected from the group consisting of hydrogen; cyano; C₁-C₆-alkyl which may be partially or fully halogenated and/or may be substituted by one or more radicals R¹⁸; C₁-C₆-alkoxy; C₁-C₆-haloalkoxy; C₁-C₆-alkylthio; C₁-C₆-haloalkylthio; C₃-C₈-cycloalkyl; C₃-C₈-halocycloalkyl; C₃-C₈-cycloalkyl-C₁-C₄-alkyl; C₃-C₈-halocycloalkyl-C₁-C₄-alkyl; C₂-C₆-alkenyl; C₂-C₆-haloalkenyl; C₂-C₆-alkynyl; C₂-C₆-haloalkynyl; —Si(R¹⁴)₂R¹³; —SR⁸; —S(O)ₘR¹⁷; —S(O)ₙN(R⁸)R⁹; —N(R⁸)R⁹; —N═CR¹⁵R¹⁶; —C(═O)R¹⁷; —C(═O)N(R⁸)R⁹; —C(═S)N(R⁸)R⁹; —C(═O)OR¹⁷; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
with the proviso that R⁷ is not C₁-C₆-alkoxy or C₁-C₆-haloalkoxy when it is bound to an oxygen atom;
R⁸ and R⁹, independently of each other and independently of each occurrence, are selected from the group consisting of hydrogen; C₁-C₆-alkyl which may be partially or fully halogenated and/or may be substituted by one or more radicals R¹⁸; C₁-C₆-alkoxy; C₁-C₆-haloalkoxy; C₁-C₆-alkylthio; C₁-C₆-haloalkylthio; C₃-C₈-cycloalkyl; C₃-C₈-halocycloalkyl; C₃-C₈-cycloalkyl-C₁-C₄-alkyl; C₃-C₈-halocycloalkyl-C₁-C₄-alkyl; C₂-C₆-alkenyl; C₂-C₆-haloalkenyl; C₂-C₆-alkynyl; C₂-C₆-haloalkynyl; —C(═O)R¹⁷; —C(═O)OR¹⁷; —C(═O)N(R²⁰)R²¹; C(O)—CH₂—S(O)ₙ-(C₁-C₄-alkyl); —C(O)—CH₂—S(O)ₙ-(C₁-C₄-haloalkyl); phenylcarbonyl; —C(O)—CH₂-phenyl; —C(O)—CH₂—S(O)ₙ-phenyl; phenyl, where the phenyl moiety in the 4 last-mentioned radicals may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
each R¹⁰ is independently selected from the group consisting of halogen; cyano; azido; nitro; —SCN; —SF₅; C₁-C₁₀-alkyl which may be partially or fully halogenated and/or may be substituted by one or more radicals R¹⁸; C₃-C₈-cycloalkyl which may be partially or fully halogenated and/or may be substituted by one or more radicals R¹⁹; C₂-C₁₀-alkenyl which may be partially or fully halogenated and/or may be substituted by one or more radicals R¹⁸; C₂-C₁₀-alkynyl which may be partially or fully halogenated and/or may be substituted by one or more radicals R¹⁸; —Si(R¹⁴)₂R¹³; —OR¹⁷; —OS(O)ₙR¹⁷; —SR¹⁷; —S(O)ₘR¹⁷; —S(O)ₙN(R²⁰)R²¹; —N(R²⁰)R²¹; —C(═O)R¹⁷; —C(═O)OR¹⁷; —C(═NR²⁰)R¹⁷; —C(═O)N(R²⁰)R²¹; —C(═S)N(R²⁰)R²¹; phenyl which may be substituted by 1, 2, 3, 4 or 5 radicals independently selected from the group consisting of halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated or unsaturated heterocyclic ring containing 1, 2 or 3 heteroatoms or heteroatom groups selected from the group consisting of N, O, S, NO, SO and SO₂, as ring members, which may be substituted by one or more radicals independently selected from the group consisting of halogen, cyano, nitro, C₁-C₆-alkyl, C₁-C₆-haloalkyl, C₁-C₆-alkoxy and C₁-C₆-haloalkoxy; or two radicals R¹⁰ bound on adjacent atoms together form a group selected from the group consisting of —CH₂CH₂CH₂CH₂—, —CH═CH—CH═CH—, —N═CH—CH═CH—, —CH═N—CH═CH—, —N═CH—N═CH—, —OCH₂CH₂CH₂—, —OCH═CHCH₂—, —CH₂OCH₂CH₂—, —OCH₂CH₂O—, —OCH₂OCH₂—, —CH₂CH₂CH₂—, —CH═CHCH₂—, —CH₂CH₂O—, —CH═CHO—, —CH₂OCH₂—, —CH₂C(═O)O—, —C(═O)OCH₂—, —O(CH₂)O—, —SCH₂CH₂CH₂—, —SCH═CHCH₂—, —CH₂SCH₂CH₂—, —SCH₂CH₂S—, —SCH₂SCH₂—, —CH₂CH₂S—, —CH═CHS—, —CH₂SCH₂—, —CH₂C(═S)S—, —C(═S)SCH₂—, —S(CH₂)S—, —CH₂CH₂NR²⁰—, —CH₂CH═N—, —CH═CH—NR²⁰—, —OCH═N— and —SCH═N—, thus forming, together with the atoms to which they are bound, a 5- or 6-membered ring, where the hydrogen atoms of the above groups may be replaced by one or more substituents selected from the group consisting of halogen, methyl, halomethyl, hydroxyl, methoxy and halomethoxy or one or more CH₂ groups of the above groups may be replaced by a C═O group;
R¹¹ and R¹², independently of each other and independently of each occurrence, are selected from the group consisting of hydrogen; halogen; C₁-C₆-alkyl; C₁-C₆-haloalkyl; C₂-C₆-alkenyl; C₂-C₆-haloalkenyl; C₂-C₆-alkynyl; C₂-C₆-haloalkynyl; C₃-C₈-cycloalkyl; C₃-C₈-halocycloalkyl; C₁-C₆-alkoxy-C₁-C₆-alkyl; C₁-C₆-haloalkoxy-C₁-C₆-alkyl; phenyl which may be substituted by 1, 2, 3, 4, or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, which may be substituted by one or more radicals R¹⁰;
R¹³ and R¹⁴, independently of each other and independently of each occurrence, are selected from the group consisting of C₁-C₄-alkyl, C₃-C₆-cycloalkyl, C₁-C₄-alkoxy-C₁-C₄-alkyl, phenyl and benzyl;
R¹⁵ and R¹⁶, independently of each other and independently of each occurrence, are selected from the group consisting of C₁-C₆-alkyl; C₁-C₆-haloalkyl; C₂-C₆-alkenyl; C₂-C₆-haloalkenyl; C₂-C₆-alkynyl; C₂-C₆-haloalkynyl; C₃-C₈-cycloalkyl; C₃-C₈-halocycloalkyl; C₁-C₆-alkoxy-C₁-C₆-alkyl; C₁-C₆-haloalkoxy-C₁-C₆-alkyl; phenyl which may be substituted by 1, 2, 3, 4, or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, which may be substituted by one or more radicals R¹⁰;
each R¹⁷ is independently selected from the group consisting of hydrogen; C₁-C₆-alkyl; C₁-C₆-haloalkyl; C₂-C₆-alkenyl; C₂-C₆-haloalkenyl; C₂-C₆-alkynyl; C₂-C₆-haloalkynyl; C₃-C₆-cycloalkyl; C₃-C₆-halocycloalkyl; C₃-C₈-cycloalkyl-C₁-C₄-alkyl; C₃-C₈-halocycloalkyl-C₁-C₄-alkyl; C₁-C₆-alkoxy-C₁-C₆-alkyl; C₁-C₆-haloalkoxy-C₁-C₆-alkyl; phenyl and benzyl, where the phenyl moieties in the two last-mentioned radicals may be substituted by 1, 2 or 3 radicals selected from the group consisting of halogen, cyano, nitro, C₁-C₄-alkyl, C₁-C₄-haloalkyl, C₁-C₄-alkoxy and C₁-C₄-haloalkoxy;
each R¹⁸ is independently is selected from the group consisting of cyano; azido; nitro; —SCN; SF₅; C₃-C₆-cycloalkyl; C₃-C₆-halocycloalkyl; —Si(R¹⁴)₂R¹³; —OR⁷; —OSO₂R¹⁷; —SR¹⁷; —S(O)ₘR¹⁷; —S(O)ₙN(R²⁰)R²¹; —N(R²⁰)R²¹; —C(═O)N(R²⁰)R²¹; —C(═S)N(R²⁰)R²¹; —C(═O)OR¹⁷; phenyl which may be substituted by 1, 2, 3, 4 or 5 radicals independently selected from the group consisting of halogen, cyano, nitro, C₁-C₆-alkyl, C₁-C₆-alkoxy and C₁-C₆-haloalkoxy; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals independently selected from the group consisting of halogen, cyano, nitro, C₁-C₆-alkyl, C₁-C₆-haloalkyl, C₁-C₆-alkoxy and C₁-C₆-haloalkoxy;
each R¹⁹ is independently is selected from the group consisting of cyano; azido; nitro; —SCN; SF₅; C₁-C₆-alkyl; C₁-C₆-haloalkyl; C₁-C₆-alkoxy-C₁-C₆-alkyl; C₁-C₆-haloalkoxy-C₁-C₆-alkyl; C₂-C₆-alkenyl; C₂-C₆-haloalkenyl; C₂-C₆-alkynyl; C₂-C₆-haloalkynyl; C₃-C₈-cycloalkyl; C₃-C₈-halocycloalkyl; —Si(R¹⁴)₂R¹³; —OR¹⁷; —OSO₂R¹⁷; —SR¹⁷; —S(O)ₘR¹⁷; —S(O)ₙN(R²⁰)R²¹; —N(R²⁰)R²¹; —C(═O)N(R²⁰)R²¹; —C(═S)N(R²⁰)R²¹; —C(═O)OR¹⁷; phenyl which may be substituted by 1, 2, 3, 4 or 5 radicals independently selected from the group consisting of halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals independently selected from the group consisting of halogen, cyano, nitro, C₁-C₆-alkyl, C₁-C₆-haloalkyl, C₁-C₆-alkoxy and C₁-C₆-haloalkoxy;
R²⁰ and R²¹, independently of each other and independently of each occurrence, are selected from the group consisting of hydrogen; C₁-C₆-alkyl; C₁-C₆-haloalkyl; C₁-C₆-alkoxy; C₁-C₆-haloalkoxy; C₁-C₆-alkylthio; C₁-C₆-haloalkylthio; C₃-C₈-cycloalkyl; C₃-C₈-halocycloalkyl; C₃-C₈-cycloalkyl-C₁-C₄-alkyl; C₃-C₈-halocycloalkyl-C₁-C₄-alkyl; C₂-C₆-alkenyl; C₂-C₆-haloalkenyl; C₂-C₆-alkynyl; C₂-C₆-haloalkynyl; —C(═O)N(R²²)R²³; —C(═O)R¹⁷; —C(═O)OR¹⁷; phenyl; benzyl; where the phenyl moieties in the two last-mentioned radicals may be substituted by 1, 2, 3, 4 or 5 radicals independently selected from the group consisting of halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals independently selected from the group consisting of halogen, cyano, nitro, C₁-C₆-alkyl, C₁-C₆-haloalkyl, C₁-C₆-alkoxy and C₁-C₆-haloalkoxy;
or R²⁰ and R²¹, together with the nitrogen atom to which they are bound, may form a 3-, 4-, 5-, 6- or 7-membered saturated, partially unsaturated or aromatic heterocyclic ring which may additionally containing 1 or 2 further heteroatoms or heteroatom groups selected from the group consisting of N, O, S, NO, SO and SO₂, as ring members, where the heterocyclic ring may be substituted by one or more radicals selected from the group consisting of halogen, C₁-C₆-alkyl, C₁-C₆-haloalkyl, C₁-C₆-alkoxy and C₁-C₆-haloalkoxy;
R²² and R²³, independently of each other and independently of each occurrence, are selected from the group consisting of hydrogen; C₁-C₆-alkyl; C₁-C₆-haloalkyl; C₁-C₆-alkoxy; C₁-C₆-haloalkoxy; C₁-C₆-alkylthio; C₁-C₆-haloalkylthio; C₃-C₈-cycloalkyl; C₃-C₈-halocycloalkyl; C₃-C₈-cycloalkyl-C₁-C₄-alkyl; C₃-C₈-halocycloalkyl-C₁-C₄-alkyl; C₂-C₆-alkenyl; C₂-C₆-haloalkenyl; C₂-C₆-alkynyl; C₂-C₆-haloalkynyl; aminocarbonyl; C₁-C₄-alkylaminocarbonyl; di-(C₁-C₄-alkyl)-aminocarbonyl; —C(═O)R¹⁷; —C(═O)OR¹⁷; phenyl; benzyl, where the phenyl moieties in the two last-mentioned radicals may be substituted by 1, 2, 3, 4 or 5 radicals independently selected from the group consisting of halogen, cyano, nitro, C₁-C₆-alkyl, C₁-C₆-haloalkyl, C₁-C₆-alkoxy and C₁-C₆-haloalkoxy; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals independently selected from the group consisting of halogen, cyano, nitro, C₁-C₆-alkyl, C₁-C₆-haloalkyl, C₁-C₆-alkoxy and C₁-C₆-haloalkoxy;
each m is independently 1 or 2;
each n is independently 0, 1 or 2;
p is 0, 1, 2 or 3;
q is 0, 1, 2, 3, 4 or 5;
r is 0, 1, 2, 3, 4 or 5; and
# is the attachment point to the remainder of the molecule;
and the stereoisomers and agriculturally or veterinarily acceptable salts thereof.

34. The compound according to claim 33, wherein A¹ is N or CH and A² and A³ are CH.

35. The compound according to claim 34, wherein A¹ is N and A² and A³ are CH.

36. The compound according to claim 35, wherein B¹, B² and B³ are CH.

37. The compound according to claim 36, wherein X is selected from the group consisting of C₁-C₄-alkyl, C₁-C₄haloalkyl, C₁-C₄-alkoxy-C₁-C₄-alkyl, C₃-C₆-cycloalkyl and C₃-C₆-halocycloalkyl.

38. The compound according to claim 36, wherein X is selected from the group consisting of C₁-C₄-alkyl, C₁-C₄-haloalkyl, C₃-C₆-cycloalkyl and C₃-C₆-halocycloalkyl.

39. The compound according to claim 33, wherein G is a condensed phenyl ring, a condensed 5-membered heteroaromatic ring containing 1 heteroatom selected from the group consisting of O, S and N and optionally 1 or 2 further nitrogen atoms as ring members or a condensed 6-membered heteroaromatic ring containing 1, 2 or 3 nitrogen atoms as ring members.

40. The compound according to claim 39, where Q is selected from the radicals of following formulae: 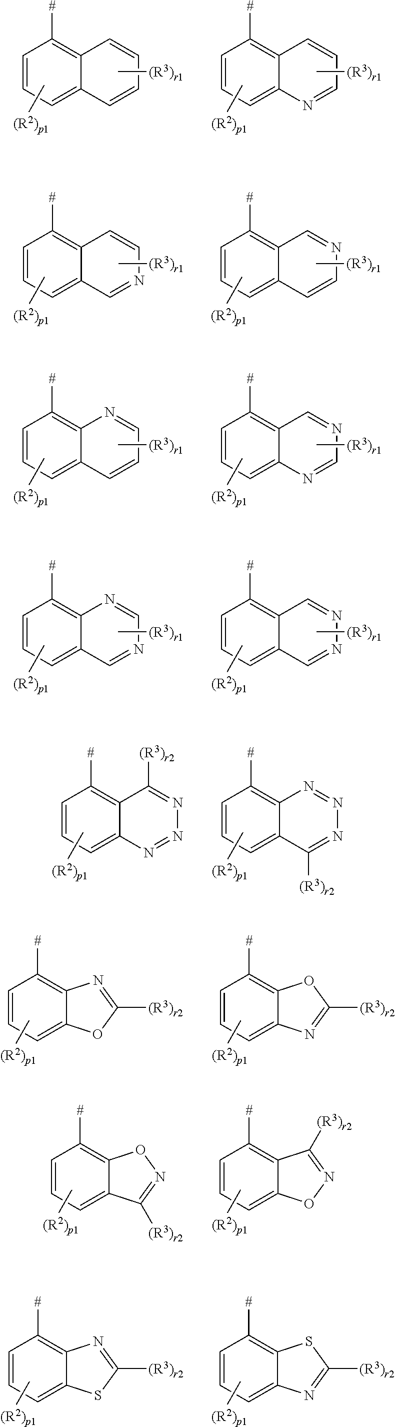 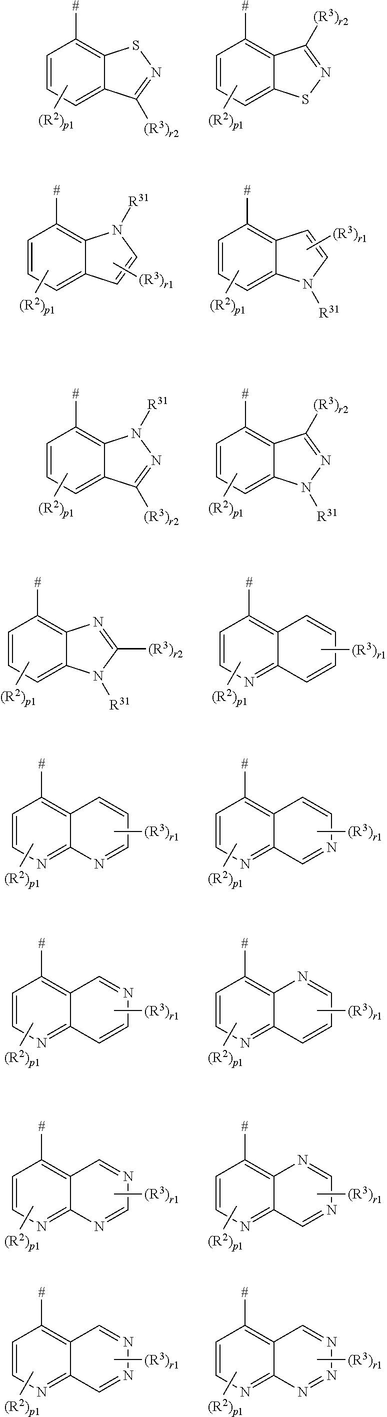 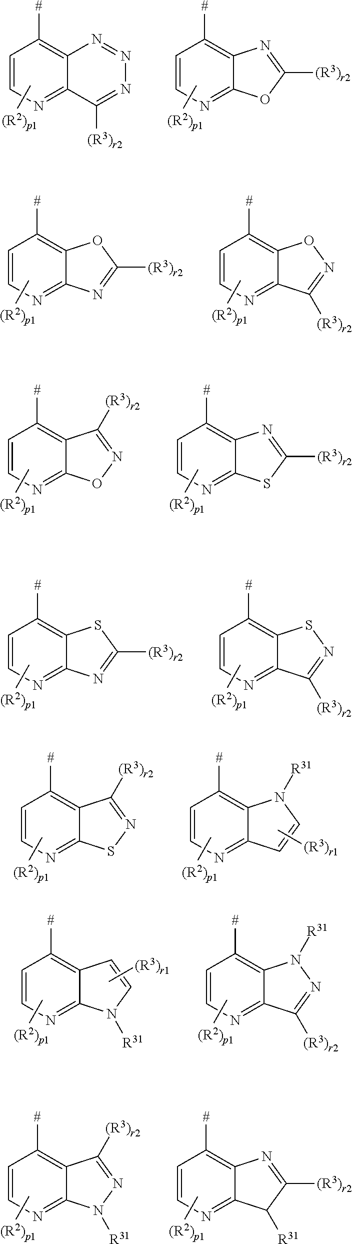 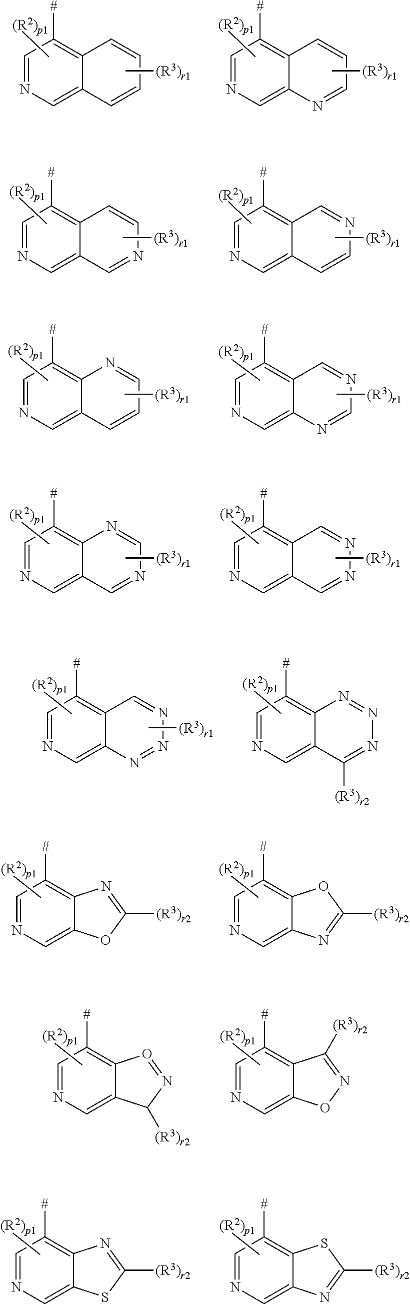 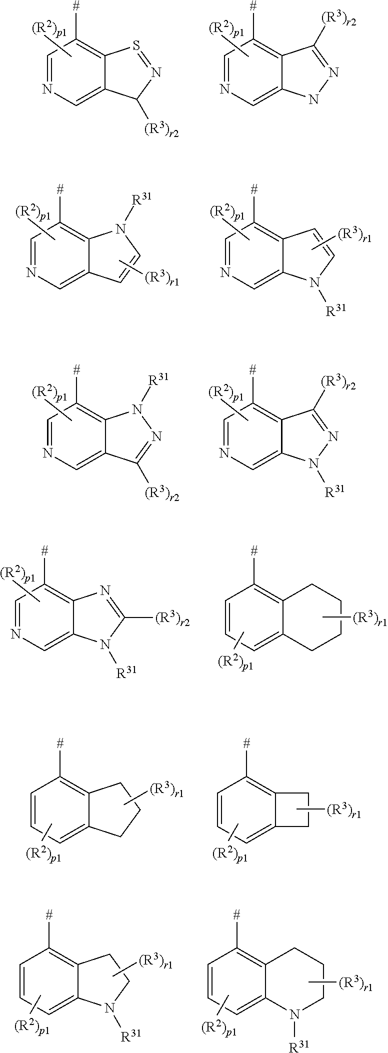
where
p′ is 0, 1 or 2;
r¹ is 0, 1 or 2;
r² is 0 or 1; and
# is the attachment point to the remainder of the molecule.

41. The compound according to claim 40, where Q has one of the following formulae: 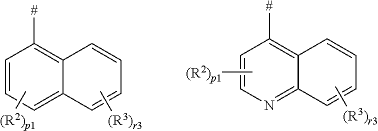
where
p¹ is 0, 1 or 2;
r³ is 0, 1 or 2; and
# is the attachment point to the remainder of the molecule.

42. The compound according to claim 33, wherein each R¹ is independently selected from the group consisting of halogen, cyano, nitro, —SCN, SF₅, C₁-C₆-alkyl, C₁-C₆-alkyl which may be partially or fully halogenated and/or may be substituted by one or more radicals R⁴, C₃-C₈-cycloalkyl which may be partially or fully halogenated and/or may be substituted by one or more radicals R⁵, C₂-C₆-alkenyl which may be partially or fully halogenated and/or may be substituted by one or more radicals R⁴, C₂-C₆-alkynyl which may be partially or fully halogenated and/or may be substituted by one or more radicals R⁴, —Si(R¹⁴)₂R¹³, —OR⁷, —OS(O)ₙR⁷, S(O)ₘR⁷, —NR⁸R⁹, —N(R⁸)C(═O)R⁶, —C(═O)R⁶, —C(═O)OR⁷, —C(═NR⁸)R⁶, —C(═S)R⁶, phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰.

43. The compound according to claim 42, wherein each R¹ is independently selected from the group consisting of halogen, cyano, nitro, hydroxyl, C₁-C₄-alkyl, C₁-C₄-haloalkyl, C₁-C₄-alkoxy and C₁-C₄-haloalkoxy.

44. The compound according to claim 43, wherein q is 0, 1, 2 or 3.

45. The compound according to claim 33, wherein each R² is independently selected from the group consisting of halogen; cyano; azido; nitro; —SCN; SF₅; C₁-C₆-alkyl which may be partially or fully halogenated and/or may be substituted by one or more radicals R⁴; C₃-C₆-cycloalkyl which may be partially or fully halogenated and/or may be substituted by one or more radicals R⁵; C₂-C₆-alkenyl which may be partially or fully halogenated and/or may be substituted by one or more radicals R⁴; C₂-C₆-alkynyl which may be partially or fully halogenated and/or may be substituted by one or more radicals R⁴; —Si(═O)₂(R¹⁴)₂R¹³; —OR⁷; —OS(O)ₙR⁷; —SR⁷; —S(O)ₘR⁷; —S(O)ₙN(R⁸)R⁹; —N(R⁸)R⁹; —N(R⁸)C(═O)R⁶; —C(═O)R⁶, wherein R⁶ is different from —N(R⁸)R⁹; —C(═O)OR⁷; —C(═S)R⁶, wherein R⁶ is different from —N(R⁸)R⁹; —C(═S)OR⁷, phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰;
with the proviso that R² is not halogen, cyano, nitro, methyl which carries one halogen atom or carries one group OH or one methylcarbonyloxy group; OH, methoxy, —OS(O)ₙR⁷, —NH₂, —CHO, C₁-C₆-alkylcarbonyl; or —C(═O)OR⁷, wherein R⁷ is hydrogen, C₁-C₆-alkyl or benzyl; when A¹, A² and A³ are CH and R² is bound to A¹.

46. The compound according to claim 45, wherein each R² is independently selected from the group consisting of azido; —SCN; SF₅; C₁-C₆-alkyl; C₁-C₆-alkyl which is substituted by one or more radicals R⁴ different from OR⁷; C₃-C₈-cycloalkyl which may be partially or fully halogenated and/or may be substituted by one or more radicals R⁵; C₂-C₆-alkenyl which may be partially or fully halogenated and/or may be substituted by one or more radicals R⁴; C₂-C₆-alkynyl which may be partially or fully halogenated and/or may be substituted by one or more radicals R⁴; —Si(R¹⁴)₂R¹³; —OR⁷, wherein R⁷ is not hydrogen or C₁-C₆-alkyl; —SR⁷; —S(O)ₘR⁷; —S(O)ₙN(R⁸)R⁹; —N(R⁸)C(═O)R⁶; —C(═S)OR⁷, phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰.

47. The compound according to claim 46, wherein each R² is independently selected from the group consisting of —SCN; —SF₅; C₁-C₆-alkyl; C₁-C₆-alkyl which is substituted by one or more radicals R⁴ different from OR⁷; C₃-C₈-cycloalkyl which may be partially or fully halogenated and/or may be substituted by one or more radicals R⁵; C₂-C₆-alkenyl which may be partially or fully halogenated and/or may be substituted by one or more radicals R⁴; C₂-C₆-alkynyl which may be partially or fully halogenated and/or may be substituted by one or more radicals R⁴; —Si(R¹⁴)₂R¹³; —OR⁷, wherein R⁷ is not hydrogen or C₁-C₆-alkyl; —SR⁷; —S(O)ₘR⁷; —S(O)ₙN(R⁸)R⁹; —N(R⁸)C(═O)R⁶; phenyl which may be substituted by 1, 2, 3, 4 or 5 radicals R¹⁰; and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰.

48. The compound according to claim 47, wherein each R² is independently selected from the group consisting of —SCN; C₁-C₆-alkyl; C₁-C₆-alkyl which is substituted by one or more radicals R⁴ different from OR⁷; C₃-C₈-cycloalkyl which may be partially or fully halogenated and/or may be substituted by one or more radicals R⁵; —OR⁷, wherein R⁷ is not hydrogen or C₁-C₆-alkyl; —SR⁷; —S(O)ₘR⁷; —S(O)ₙN(R⁸)R⁹; phenyl which may be substituted by 1, 2 or 3 radicals R¹⁰; and a 5- or 6-membered heteroaromatic ring containing 1, 2 or 3 heteroatoms selected from the group consisting of N, O and S as ring members, where the heteroaromatic ring may be substituted by 1 or 2 radicals R¹⁰.

49. The compound according to claim 48, wherein each R² is independently selected from the group consisting of C₁-C₄-alkyl, C₂-C₄-haloalkyl, C₂-C₄-alkoxy, haloalkoxy, phenyl which may be substituted by 1, 2 or 3 radicals R¹⁰; and a 5- or 6-membered heteroaromatic ring containing 1, 2 or 3 heteroatoms selected from the group consisting of N, O and S as ring members, where the heteroaromatic ring may be substituted by 1 or 2 radicals R¹⁰; where R¹⁰ is as defined in claim 1.

50. The compound according to claim 49, wherein p is 0 or 1.

51. The compound according to claim 50, wherein each R³ is independently selected from the group consisting of halogen, cyano, C₁-C₄-alkyl which may be substituted by one or more radicals R⁴, C₁-C₄-haloalkyl, —C(═O)OR⁷, —C(═O)N(R⁸)R⁹; —OS(O)₂R⁷; —S(O)₂OR⁷; —S(O)₂R⁷; —S(O)₂N(R⁸)R⁹; and —C(═NR⁸)R⁶.

52. The compounds according to claim 51, wherein each R³ is independently selected from the group consisting of halogen, cyano, C₁-C₄-alkyl which may be substituted by one or more radicals R⁴, C₁-C₄-haloalkyl, —C(═O)OR⁷ and —C(═O)N(R⁸)R⁹.

53. The compounds according to claim 52, wherein R⁴ is N(R⁸)R⁹, wherein
R⁸ is selected from the group consisting of hydrogen, C₁-C₆-alkyl, formyl, C₁-C₆-alkylcarbonyl, C₁-C₆-haloalkylcarbonyl, C₁-C₆-alkoxycarbonyl, C₁-C₆-haloalkoxycarbonyl, —C(O)—CH₂—S(O)ₙ—(C₁-C₄-alkyl) and —C(O)—CH₂—S(O)ₙ—(C₁-C₄-haloalkyl); and
R⁹ is selected from the group consisting of hydrogen, C₁-C₆-alkyl, C₁-C₆-haloalkyl, C₁-C₆-alkoxy, C₁-C₆-haloalkoxy, C₁-C₆-alkylthio, C₁-C₆-haloalkylthio, C₃-C₈-cycloalkyl, C₃-C₆-halocycloalkyl, C₂-C₆-alkenyl, C₂-C₆-haloalkenyl, C₂-C₆-alkynyl, C₂-C₆-haloalkynyl, phenyl which may be substituted by 1, 2, 3, 4 or 5 radicals R¹⁰, and a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members, where the heterocyclic ring may be substituted by one or more radicals R¹⁰.

54. The compounds according to claim 52, wherein R⁷ is hydrogen or C₁-C₆-alkyl.

55. The compounds according to claim 52, wherein R⁸ in —C(═O)N(R⁸)R⁹ is selected from the group consisting of hydrogen, C₁-C₆-alkyl, C₁-C₆-haloalkyl and C₁-C₄-alkyl substituted by a phenyl group or substituted by a 3-, 4-, 5-, 6- or 7-membered saturated, partially unsaturated or aromatic heterocyclic ring containing 1, 2 or 3 heteroatoms or heteroatom groups selected from the group consisting of N, O, S, NO, SO and SO₂, as ring members.

56. An agricultural composition comprising at least one compound of claim 33, a stereoisomer thereof and/or at least one agriculturally acceptable salt thereof, and at least one inert liquid and/or solid agriculturally acceptable carrier.

57. A veterinary composition comprising at least one compound of claim 33, a stereoisomer thereof and/or at least one veterinarily acceptable salt thereof, and at least one inert liquid and/or solid veterinarily acceptable carrier.

58. A method for controlling invertebrate pests which method comprises treating the pests, their food supply, their habitat or their breeding ground or a plant, plant propagation material, soil, area, material or environment in which the pests are growing or may grow, or the materials, plants, plant propagation material, soils, surfaces or spaces to be protected from invertebrate pest attack or infestation with a pesticidally effective amount of at least one imine compound of claim 33, a stereoisomer thereof and/or at least one agriculturally acceptable salt thereof.

59. The method as claimed in claim 58, for protecting plants from attack or infestation by invertebrate pests, which method comprises treating the plants with a pesticidally effective amount of at least one compound of the formula I as, a stereoisomer thereof and/or at least one agriculturally acceptable salt thereof.

60. The method as claimed in claim 58, for protecting plant propagation material and/or the plants which grow therefrom from attack or infestation by invertebrate pests, which method comprises treating the plant propagation material with a pesticidally effective amount of at least one compound of the formula I a stereoisomer thereof and/or at least one agriculturally acceptable salt thereof.

61. Plant propagation material, comprising at least one compound of claim 33, a stereoisomer thereof and/or at least one agriculturally acceptable salt thereof.

62. A method for treating, controlling, preventing or protecting animals against infestation or infection by parasites which comprises orally, topically or parenterally administering or applying to the animals a parasiticidally effective amount of at least one compound of claim 33, a stereoisomer thereof and/or at least one veterinarily acceptable salt thereof.
